(19) United States
(12) Patent Application Publication
(10) Pub. No.: US 2007/0254877 A1
(43) Pub. Date: Nov. 1, 2007

(54) Indole Derivative and Use for Treatment of Cancer

(75) Inventors: Yuji Nishikimi, Osaka-shi (JP); Hideto Fukushi, Osaka-shi (JP); Hiroshi Miki, Osaka-shi (JP)

Correspondence Address: HAMRE, SCHUMANN, MUELLER & LARSON, P.C., P.O. BOX 2902, MINNEAPOLIS, MN, 55402-0902, US

(73) Assignee: Takada Pharmaceutical Company Limited, Osaka-shi, Osaka (JP)

(21) Appl. No.: 11/628,386

(22) PCT Filed: Jun. 1, 2005
(86) PCT No.: PCT/JP05/10450

§ 371 Date: Dec. 1, 2006

(30) Foreign Application Priority Data
Jun. 2, 2004 (JP) 2004-165012
Dec. 8, 2004 (JP) 2004-355947

(51) Int. Cl. A61K 31/4162; A61K 31/454; A61K 31/5377; A61P 43/00; C07D 403/14; C07D 413/14; C07D 487/04

(52) U.S. Cl. 514/232.8; 514/322; 514/403; 544/140; 544/371; 548/358.5

(57) ABSTRACT

The present invention relates to a compound represented by the formula 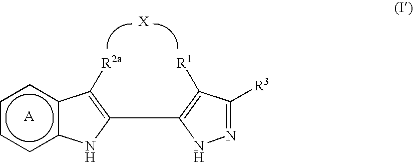
wherein A is a benzene ring optionally having substituents, R¹, R^{2a} and R³ are each a hydrogen atom, a hydrocarbon group optionally having substituents or a heterocyclic group optionally having substituents, R¹ and R^{2a} may form a ring via X, when R¹ and R^{2a} form a ring via X, R¹ and R^{2a} are each a bond or a divalent C₁₋₅ acyclic hydrocarbon group optionally having substituents, and X is a bond, an oxygen atom, an optionally oxidized sulfur atom or an imino group optionally having a substituent, provided that R¹, R^{2a} and X are not bonds at the same time, or a salt thereof, and an agent for inhibiting kinase (phosphorylation enzyme), which contains this compound or a prodrug thereof. The compound of the present invention has an inhibitory activity against kinase such as a vascular endothelial growth factor receptor (VEGFR) and the like, and is useful as an agent for the prophylaxis or treatment of cancer and the like.

### TECHNICAL FIELD

The present invention relates to an indole derivative having a superior kinase inhibitory action and use thereof.

### BACKGROUND ART

It has been known that a tumor requires oxygen and nutrients for its growth, and angiogenesis to supply them is indispensable for the tumor growth (e.g., non-patent document 1). The angiogenesis toward tumor is considered to start from binding of a vascular endothelial growth factor to a vascular endothelial growth factor receptor in the molecular level, which causes phosphorylation that transmits a growth signal (e.g., non-patent document 2).

As a pyrazoloindole wherein a pyrazole ring is condensed with an indole ring via a ring, patent document 1 describes 8-methoxy-4,5,6,11-tetrahydro-1H-pyrazolo[4′,3′:6,7]cyclohepta[1,2-b]indole represented by the formula 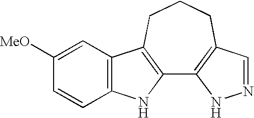
as an antifungal agent.

In addition, non-patent document 3 describes 7-methyl-3-phenyl-1,4,5,10-tetrahydropyrazolo[3,4-a]carbazole hydrochloride represented by the formula

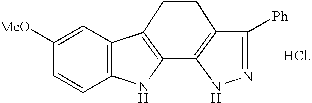

Non-patent document 4 describes 7-methoxy-1,10-dihydropyrazolo[3,4-a]carbazole, 9-methoxy-1,10-dihydropyrazolo[3,4-a]carbazole and 1,10-dihydropyrazolo[3,4-a]carbazole represented by the formulas 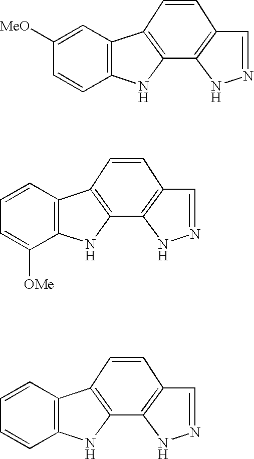
as antibacterial agents.

Non-patent document 5 describes 1,4,5,10-tetrahydropyrazolo[3,4-a]carbazole, 7-chloro-1,4,5,10-tetrahydropyrazolo[3,4-a]carbazole, 7-bromo-1,4,5,10-tetrahydropyrazolo[3,4-a]carbazole, 7-methyl-1,4,5,10-tetrahydropyrazolo[3,4-a]carbazole and 7-cyclohexyl-1,4,5,10-tetrahydropyrazolo[3,4-a]carbazole represented by the formula 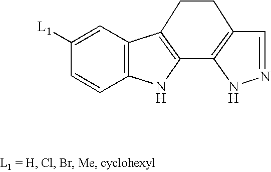
as antibacterial agents and antifungal agents.

Moreover, non-patent document 6 describes 1,10-dihydropyrazolo[3,4-a]carbazole, 9-methyl-1,10-dihydropyrazolo[3,4-a]carbazole, 8-methyl-1,10-dihydropyrazolo[3,4-a]carbazole, 7-methyl-1,10-dihydropyrazolo[3,4-a]carbazole, 7-chloro-1,10-dihydropyrazolo[3,4-a]carbazole and 7-bromo-1,10-dihydropyrazolo[3,4-a]carbazole represented by the formula

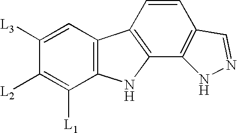

| L₁ | L₂ | L₃ |
|---|---|---|
| H | H | H |
| Me | H | H |
| H | Me | H |
| H | H | Me |
| H | H | Cl |
| H | H | Br |

as antimalarial agents and antibacterial agents.

As pyrazolylindole, patent document 2 describes a compound represented by the formula 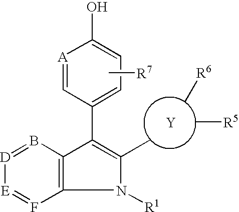
as a therapeutic drug for osteoporosis. However, in the formula, A is CH or N, Y is heteroaryl such as pyrazole and the like, and the like, the substituent at the 3-position of indole is limited to a 4-hydroxyphenyl group optionally having substituents or a 6-hydroxypyridin-3-yl group optionally having substituents, and R⁷ is a hydrogen atom, a halogen atom, a C₁₋₆ lower alkyl group or a C₁₋₆ lower alkoxy group.

Non-patent documents 7 and 8 describe 2,3-dihydro-5-[3-[[(4-methylphenyl)sulfonyl]oxy]-1H-indol-2-yl]-3-oxo-1H-pyrazole-4-carbonitrile represented by the formula

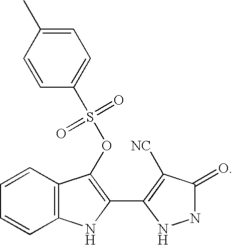

Patent document 3 describes that a tetracyclic pyrazole derivative represented by the formula 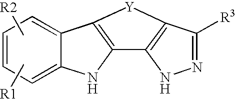
wherein R¹ and R² are the same or different and each is a hydrogen atom, a halogen atom, hydroxy, carboxy, a C₁₋₈ alkyl group, a heterocyclylcarbonyl group, a C₁₋₆ alkyloxycarbonyl group, a C₁₋₆ alkylaminocarbonyl group, a C₁₋₆ dialkylaminocarbonyl group and the like; Y=—(CH₂)ₙ— (n=1-3) or —CH═CH—; R3 is a hydrogen atom, a C₁₋₈ alkyl group and the like, has a protein kinase inhibitory activity and is useful for the treatment of cancer and the like.
patent document 1: U.S. Pat. No. 3,772,325
patent document 2: JP-A-2001-122855
patent document 3: WO2004/071507
non-patent document 1: New England Journal of Medicine, 1971, Vol. 285, No. 21, pp. 1182-1186
non-patent document 2: Science, 1984, Vol. 223, No. 4642, pp. 1296-1299
non-patent document 3: ChemBridge Product List, ChemBridge Corporation, 2002
non-patent document 4: Research Journal of Chemistry and Environment, 2001, Vol. 5, No. 1, pp. 31-33
non-patent document 5: Khim.-Farm. Zh., 1994, Vol. 28, No. 8, pp. 566-569
non-patent document 6: Indian J. Chem. Sect. B, 1998, Vol. 37B, No. 3, pp. 314-317
non-patent document 7: J. Pharm. Soc. Jpn. (YAKUGAKU ZASSHI), 1977, Vol. 97, No. 9, pp. 1033-1039
non-patent document 8: J. Pharm. Soc. Jpn. (YAKUGAKU ZASSHI), 1973, Vol. 93, No. 8, pp. 964-970

### DISCLOSURE OF THE INVENTION

It is considered that inhibition of a growth signal transduction pathway in a blood vessel enables suppression of the growth of blood vessels that supply oxygen and nutrients to tumor, which ultimately suppresses the growth of tumor. Moreover, if the elongation of blood vessels around a tumor can be suppressed, metastasis wherein a tumor cell is conveyed on a bloodstream to other tissues where it grows, can be also suppressed.

A kinase inhibitor superior in affinity for kinase and superior in expression of efficacy, pharmacokinetics, solubility, interaction with other pharmaceutical products, safety and stability is expected to show a superior therapeutic effect. As the situation stands, however, a drug superior in the affinity for kinase, which is satisfactory in expression of efficacy, pharmacokinetics, solubility, interaction with other pharmaceutical products, safety and stability has not been found. Therefore, the development of a compound having a superior kinase inhibitory activity, which is sufficient as a pharmaceutical product has been demanded.

The present inventors have conducted various studies, and first succeeded in the creation of a compound represented by the formula 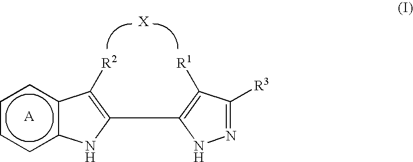
wherein A is a benzene ring optionally having substituents, R¹, R² and R³ are each a hydrogen atom, a hydrocarbon group optionally having substituents or a heterocyclic group optionally having substituents, provided that R² is not a 4-hydroxyphenyl group optionally having substituents selected from the group consisting of a halogen atom, a C₁₋₆ alkyl group and a C₁₋₆ alkoxy group; a 4-methoxyphenyl group optionally having substituents selected from the group consisting of a halogen atom, a C₁₋₆ alkyl group and a C₁₋₆ alkoxy group; and a 6-hydroxypyridin-3-yl group optionally having substituents selected from the group consisting of a halogen atom, a C₁₋₆ alkyl group and a C₁₋₆ alkoxy group, R¹ and R² optionally form a ring via X and when R¹ and R² form a ring via X, R¹ and R² are each a bond or a divalent C₁₋₅ acyclic hydrocarbon group optionally having substituents, and X is a bond, an oxygen atom, an optionally oxidized sulfur atom or an imino group optionally having a substituent, provided that R¹, R² and X are not bonds at the same time, or a salt thereof (excluding 7-methyl-3-phenyl-1,4,5,10-tetrahydropyrazolo[3,4-a]carbazole hydrochloride, 7-methoxy-1,10-dihydropyrazolo[3,4-a]carbazole, 9-methoxy-1,10-dihydropyrazolo[3,4-a]carbazole, 8-methoxy-4,5,6,11-tetrahydro-1H-pyrazolo[4′,3′:6,7]cyclohepta[1,2-b]indole, 1,4,5,10-tetrahydropyrazolo[3,4-a]carbazole, 7-chloro-1,4,5,10-tetrahydropyrazolo[3,4-a]carbazole, 7-bromo-1,4,5,10-tetrahydropyrazolo[3,4-a]carbazole, 7-methyl-1,4,5,10-tetrahydropyrazolo[3,4-a]carbazole, 7-cyclohexyl-1,4,5,10-tetrahydropyrazolo[3,4-a]carbazole, 1,10-dihydropyrazolo[3,4-a]carbazole, 9-methyl-1,10-dihydropyrazolo[3,4-a]carbazole, 8-methyl-1,10-dihydropyrazolo[3,4-a]carbazole, 7-methyl-1,10-dihydropyrazolo[3,4-a]carbazole, 7-chloro-1,10-dihydropyrazolo[3,4-a]carbazole and 7-bromo-1,10-dihydropyrazolo[3,4-a]carbazole (patent document 1 and non-patent documents 3-6)) [hereinafter to be abbreviated as compound (I)], more preferably, a compound represented by the formula 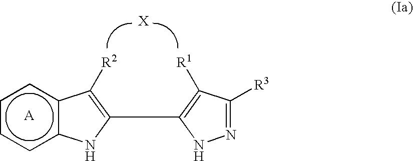
wherein A is a benzene ring optionally having substituents, R¹, R² and R³ are each a hydrogen atom, a hydrocarbon group optionally having substituents or a heterocyclic group optionally having substituents, provided that R² is not a 4-hydroxyphenyl group optionally having substituents selected from the group consisting of a halogen atom, a C₁₋₆ alkyl group and a C₁₋₆ alkoxy group; a 4-methoxyphenyl group optionally having substituents selected from the group consisting of a halogen atom, a C₁₋₆ alkyl group and a C₁₋₆ alkoxy group; and a 6-hydroxypyridin-3-yl group optionally having substituents selected from the group consisting of a halogen atom, a C₁₋₆ alkyl group and a C₁₋₆ alkoxy group, R¹ and R² optionally form a ring via X and when R¹ and R² form a ring via X, R¹ and R² are each a bond or a divalent C₁₋₅ acyclic hydrocarbon group optionally having substituents, and X is a bond, an oxygen atom, an optionally oxidized sulfur atom or an imino group optionally having a substituent, provided that when X is a bond, a ring formed by R¹ and R² via X is a 7 or more-membered ring, or a salt thereof (excluding 8-methoxy-4,5,6,11-tetrahydro-1H-pyrazolo[4′,3′:6,7]cyclohepta[1,2-b]indole (patent document 1), 4,5,6,11-tetrahydro-1H-pyrazolo[4′,3′:6,7]cyclohepta[1,2-b]indole-8-carboxamide, 3-methyl-N-(4,5,6,11-tetrahydro-1H-pyrazolo[4′,3′:6,7]cyclohepta[1,2-b]indol-8-yl)butanamide, 8-chloro-4,5,6,11-tetrahydro-1H-pyrazolo[4′,3′:6,7]cyclohepta[1,2-b]indole, N-benzyl-4,5,6,11-tetrahydro-1H-pyrazolo[4′,3′:6,7]cyclohepta[1,2-b]indole-8-carboxamide, N-isobutyl-4,5,6,11-tetrahydro-1H-pyrazolo[4′,3′:6,7]cyclohepta[1,2-b]indole-8-carboxamide, ethyl 4,5,6,11-tetrahydro-1H-pyrazolo[4′,3′:6,7]cyclohepta[1,2-b]indole-3-carboxylate, N-(4,5,6,11-tetrahydro-1H-pyrazolo[4′,3′:6,7]cyclohepta[1,2-b]indol-8-yl)acetamide and 2-phenyl-N-(4,5,6,11-tetrahydro-2H-pyrazolo[4′,3′:6,7]cyclohepta[1,2-b]indol-8-yl)acetamide (patent document 3)) [hereinafter to be abbreviated as compound (Ia)], and further found that the compound (I) and compound (Ia) unexpectedly possess superior properties as kinase inhibitors and are highly satisfactory as pharmaceutical agents, which resulted in the completion of the present invention.

Accordingly, the present invention provides
[1] a compound represented by the formula 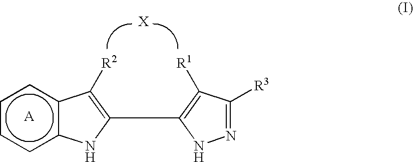
wherein A is a benzene ring optionally having substituents, R¹, R² and R³ are each a hydrogen atom, a hydrocarbon group optionally having substituents or a heterocyclic group optionally having substituents, provided that R² is not a 4-hydroxyphenyl group optionally having substituents selected from the group consisting of a halogen atom, a C₁₋₆ alkyl group and a C₁₋₆ alkoxy group; a 4-methoxyphenyl group optionally having substituents selected from the group consisting of a halogen atom, a C₁₋₆ alkyl group and a C₁₋₆ alkoxy group; and a 6-hydroxypyridin-3-yl group optionally having substituents selected from the group consisting of a halogen atom, a C₁₋₆ alkyl group and a C₁₋₆ alkoxy group, R¹ and R² optionally form a ring via X and when R¹ and R² form a ring via X, then R¹ and R² are each a bond or a divalent C₁₋₅ acyclic hydrocarbon group optionally having substituents, and X is a bond, an oxygen atom, an optionally oxidized sulfur atom or an imino group optionally having a substituent, provided that R¹, R² and X are not bonds at the same time, or a salt thereof (excluding 7-methyl-3-phenyl-1,4,5,10-tetrahydropyrazolo[3,4-a]carbazole hydrochloride, 7-methoxy-1,10-dihydropyrazolo[3,4-a]carbazole, 9-methoxy-1,10-dihydropyrazolo[3,4-a]carbazole, 8-methoxy-4,5,6,11-tetrahydro-1H-pyrazolo[4′,3′:6,7]cyclohepta[1,2-b]indole, 1,4,5,10-tetrahydropyrazolo[3,4-a]carbazole, 7-chloro-1,4,5,10-tetrahydropyrazolo[3,4-a]carbazole, 7-bromo-1,4,5,10-tetrahydropyrazolo[3,4-a]carbazole, 7-methyl-1,4,5,10-tetrahydropyrazolo[3,4-a]carbazole, 7-cyclohexyl-1,4,5,10-tetrahydropyrazolo[3,4-a]carbazole, 1,10-dihydropyrazolo[3,4-a]carbazole, 9-methyl-1,10-dihydropyrazolo[3,4-a]carbazole, 8-methyl-1,10-dihydropyrazolo[3,4-a]carbazole, 7-methyl-1,10-dihydropyrazolo[3,4-a]carbazole, 7-chloro-1,10-dihydropyrazolo[3,4-a]carbazole and 7-bromo-1,10-dihydropyrazolo[3,4-a]carbazole),
[2] the compound of the above-mentioned [1], which is represented by the formula 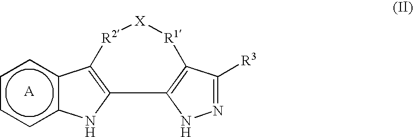
wherein R¹′ and R²′ are each a bond or a divalent C₁₋₅ acyclic hydrocarbon group optionally having substituents, and A, R³ and X are as defined in the above-mentioned [1], provided that R¹′, R²′ and X are not bonds at the same time,
[3] the compound of the above-mentioned [1], which is represented by the formula 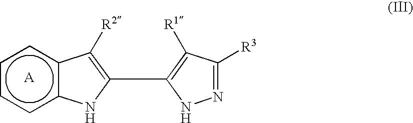
wherein R¹″ and R²″ are each a hydrogen atom, a hydrocarbon group optionally having substituents or a heterocyclic group optionally having substituents, and A and R³ are as defined in the above-mentioned [1], provided that R²″ is not a 4-hydroxyphenyl group optionally having substituents selected from the group consisting of a halogen atom, a C₁₋₆ alkyl group and a C₁₋₆ alkoxy group; a 4-methoxyphenyl group optionally having substituents selected from the group consisting of a halogen atom, a C₁₋₆ alkyl group and a C₁₋₆ alkoxy group; and a 6-hydroxypyridin-3-yl group optionally having substituents selected from the group consisting of a halogen atom, a C₁₋₆ alkyl group and a C₁₋₆ alkoxy group,
[4] the compound of the above-mentioned [3], which is represented by the formula 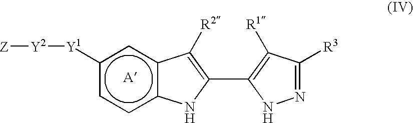
wherein A′ is a benzene ring optionally having substituents, Y¹ and Y² are each a bond, an oxygen atom, an optionally oxidized sulfur atom, an imino group optionally having a substituent or a carbonyl group, Z is a hydrogen atom, a hydrocarbon group optionally having substituents or a heterocyclic group optionally having substituents, and R¹″, R²″ and R³ are as defined in the above-mentioned [3],
[5] the compound of the above-mentioned [1], wherein the substituent of the benzene ring for A is a carbamoyl group optionally having substituents or an optionally substituted heterocycle-carbonyl group,
[6] the compound of the above-mentioned [2], wherein R¹′ is a —CH₂CH₂CH₂— group optionally having substituents, and R²′ and X are bonds,
[7] the compound of the above-mentioned [1], wherein, when X is a bond and R¹ and R² form a 7 or more-membered ring via X, then the substituent of the benzene ring for A is a heterocycle-carbonyl group having substituent(s),
[8] the compound of the above-mentioned [1], wherein, when X is a bond and R¹ and R² form a 7 or more-membered ring via X, then the substituent of the benzene ring for A is a 1-piperidinylcarbonyl group having substituent(s),
[9] a compound represented by the formula 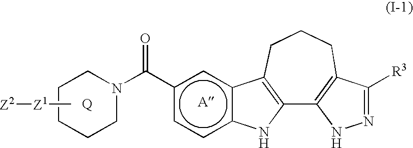
wherein Z¹ is a bond, methylene (CH₂), ethylene (CH₂CH₂), vinylene (CHCH) or ethynylene (CC), Z² is a phenyl group optionally having substituents or a heterocyclic group optionally having substituents, A″ is a benzene ring optionally further having substituents, R³ is a hydrogen atom, a hydrocarbon group optionally having substituents or a heterocyclic group optionally having substituents, and Q is a piperidine ring optionally further having substituents other than -Z¹-Z², or a salt thereof,
[10] the compound of the above-mentioned [9], which is represented by the formula 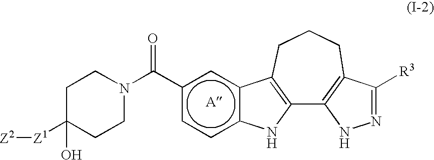
wherein each symbol is as defined in the above-mentioned [9],
[11] the compound of the above-mentioned [4], wherein Y¹ is a carbonyl group, Y² is a bond, and Z is 1-piperidinyl optionally having substituents,
[12] the compound of the above-mentioned [1], wherein R² is a hydrocarbon group optionally having substituents or a heterocyclic group optionally having substituents,
[13] the compound of the above-mentioned [1], which is represented by the formula 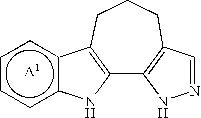
wherein A¹ is a benzene ring optionally having substituents selected from the following (i) to (vii):
(i) a C₁₋₆ alkyl group;
(ii) a carboxyl group;
(iii) a C₁₋₆ alkoxy-carbonyl group;
(iv) 5 to 7-membered heterocycle-carbonyl containing, besides carbon atom, 1 to 4 hetero atoms of 1 or 2 kinds selected from a nitrogen atom, a sulfur atom and an oxygen atom, which is optionally substituted by substituents selected from the following (a) to (g);

(a) hydroxy,

(b) C₁₋₆ alkoxy-carbonyl,

(c) a group represented by the formula -Z¹-Z²

wherein Z¹ is a bond, methylene (CH₂), ethylene (CH₂CH₂), vinylene (CHCH), ethynylene (CC) or methyleneoxy (CH₂O),

Z² is (i) a phenyl group optionally having substituents selected from the group consisting of (a) a halogen atom, (b) cyano, (c) an optionally halogenated C₁₋₆ alkyl group, (d) an optionally halogenated C₁₋₆ alkoxy group, (e) carbamoyl and (f) sulfamoyl, or

(ii) a 5 to 10-membered monocyclic or bicyclic heterocyclic group containing, besides carbon atom, 1 to 4 hetero atoms of 1 or 2 kinds selected from a nitrogen atom, a sulfur atom and an oxygen atom, which optionally has substituents selected from the group consisting of (a) a halogen atom, (b) cyano, (c) an optionally halogenated C₁₋₆ alkyl group, (d) an optionally halogenated C₁₋₆ alkoxy group, (e) carbamoyl and (f) sulfamoyl,

(d) mono- or di-C₁₋₆ alkylamino,

(e) mono- or di-C₇₋₁₆ aralkylamino,

(f) 5 to 7-membered heterocycle-C₁₋₆ alkyl(C₁₋₆ alkyl)amino wherein the heterocycle contains, besides carbon atom, 1 to 4 hetero atoms of 1 or 2 kinds selected from a nitrogen atom, a sulfur atom and an oxygen atom, and

(g) C₁₋₆ alkyl(C₇₋₁₆ aralkyl)amino wherein the C₁₋₆ alkyl is optionally substituted by hydroxy;

(v) a carbamoyl group optionally having 1 or 2 substituents selected from the following (a) to (c);

(a) C₁₋₆ alkyl,

(b) C₁₋₆ alkyl substituted by mono- or di-C₁₋₆ alkylamino, and

(c) C₁₋₆ alkyl substituted by a 5 to 7-membered heterocyclic group containing, besides carbon atom, 1 to 4 hetero atoms of 1 or 2 kinds selected from a nitrogen atom, a sulfur atom and an oxygen atom;

(vi) a halogen atom; and

(vii) 5 to 7-membered heterocycle-carbonyl-amino containing, besides carbon atom, 1 to 4 hetero atoms of 1 or 2 kinds selected from a nitrogen atom, a sulfur atom and an oxygen atom,

[14] the compound of the above-mentioned [1], which is represented by the formula 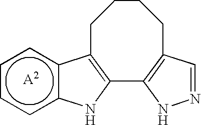
wherein A² is a benzene ring optionally having substituents selected from (i) a carboxyl group, (ii) a C₁₋₆ alkoxy-carbonyl group and (iii) 5 to 7-membered heterocycle-carbonyl containing, besides carbon atom, 1 to 4 hetero atoms of 1 or 2 kinds selected from a nitrogen atom, a sulfur atom and an oxygen atom, which is optionally substituted by a 5 to 7-membered heterocyclic group containing, besides carbon atom, 1 to 4 hetero atoms of 1 or 2 kinds selected from a nitrogen atom, a sulfur atom and an oxygen atom,
[15] the compound of the above-mentioned [1], which is represented by the formula 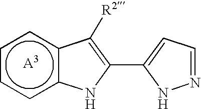
wherein R²″′ is a C₁₋₆ alkyl group, and A³ is a benzene ring optionally having 5 to 7-membered heterocycle-carbonyl containing, besides carbon atom, 1 to 4 hetero atoms of 1 or 2 kinds selected from a nitrogen atom, a sulfur atom and an oxygen atom, which is optionally substituted by a 5 to 10-membered heterocyclic group containing, besides carbon atom, 1 to 4 hetero atoms of 1 or 2 kinds selected from a nitrogen atom, a sulfur atom and an oxygen atom,
[16] the compound of the above-mentioned [1], which is represented by the formula 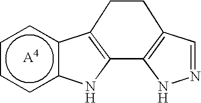
wherein A⁴ is a benzene ring optionally having substituents selected from a halogen atom and 5 to 7-membered heterocycle-carbonyl-amino containing, besides carbon atom, 1 to 4 hetero atoms of 1 or 2 kinds selected from a nitrogen atom, a sulfur atom and an oxygen atom,
[17] the compound of the above-mentioned [1], which is
(i) 4-(4-fluorophenyl)-1-(4,5,6,11-tetrahydro-1H-pyrazolo[4′,3′:6,7]cyclohepta[1,2-b]indol-8-ylcarbonyl)piperidin-4-ol,
(ii) 4-(3-fluorophenyl)-1-(4,5,6,11-tetrahydro-1H-pyrazolo[4′,3′:6,7]cyclohepta[1,2-b]indol-8-ylcarbonyl)piperidin-4-ol,
(iii) 4-(2-fluorophenyl)-1-(4,5,6,11-tetrahydro-1H-pyrazolo[4′,3′:6,7]cyclohepta[1,2-b]indol-8-ylcarbonyl)piperidin-4-ol,
(iv) 4-(4-chlorophenyl)-1-(4,5,6,11-tetrahydro-1H-pyrazolo[4′,3′:6,7]cyclohepta[1,2-b]indol-8-ylcarbonyl)piperidin-4-ol,
(v) 4-(3-chlorophenyl)-1-(4,5,6,11-tetrahydro-1H-pyrazolo[4′,3′:6,7]cyclohepta[1,2-b]indol-8-ylcarbonyl)piperidin-4-ol,
(vi) 4-(2-chlorophenyl)-1-(4,5,6,11-tetrahydro-1H-pyrazolo[4′,3′:6,7]cyclohepta[1,2-b]indol-8-ylcarbonyl)piperidin-4-ol,
(vii) 1-(4,5,6,11-tetrahydro-1H-pyrazolo[4′,3′:6,7]cyclohepta[1,2-b]indol-8-ylcarbonyl)-4-[4-(trifluoromethyl)phenyl]piperidin-4-ol,
(viii) 1-(4,5,6,11-tetrahydro-1H-pyrazolo[4′,3′:6,7]cyclohepta[1,2-b]indol-8-ylcarbonyl)-4-[3-(trifluoromethyl)phenyl]piperidin-4-ol,
(ix) 1-(4,5,6,11-tetrahydro-1H-pyrazolo[4′,3′:6,7]cyclohepta[1,2-b]indol-8-ylcarbonyl)-4-[2-(trifluoromethyl)phenyl]piperidin-4-ol, or a salt thereof,
   [18] a compound represented by the formula 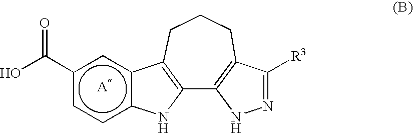
   wherein A″ is a benzene ring optionally further having substituents, and R³ is a hydrogen atom, a hydrocarbon group optionally having substituents or a heterocyclic group optionally having substituents, or a salt thereof or a reactive derivative thereof,
   [19] the compound of the above-mentioned [18], which is
(i) ethyl 4,5,6,11-tetrahydro-1H-pyrazolo[4′,3′:6,7]cyclohepta[1,2-b]indole-8-carboxylate,
(ii) 4,5,6,11-tetrahydro-1H-pyrazolo[4′,3′:6,7]cyclohepta[1,2-b]indole-8-carboxylic acid, or a salt thereof,
   [20] a prodrug of the compound of the above-mentioned [1] or [9],
   [21] a pharmaceutical agent comprising the compound of the above-mentioned [1] or [9] or a prodrug thereof,
   [22] a method of producing a compound represented by the formula 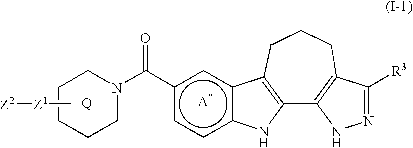
   wherein Z¹ is a bond, methylene (CH₂), ethylene (CH₂CH₂), vinylene (CHCH) or ethynylene (CC), Z² is a phenyl group optionally having substituents or a heterocyclic group optionally having substituents, Q is a piperidine ring optionally further having substituents other than -Z¹-Z², A″ is a benzene ring optionally further having substituents, and R³ is a hydrogen atom, a hydrocarbon group optionally having substituents or a heterocyclic group optionally having substituents, which comprises reacting a compound represented by the formula 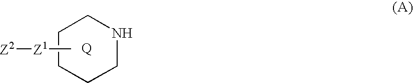
   wherein Z¹, Z² and Q are as defined above, or a salt thereof, with a compound represented by the formula 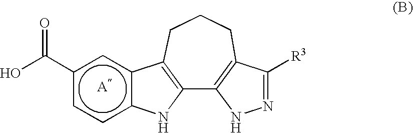
   wherein A″ and R³ are as defined above, or a salt thereof or a reactive derivative thereof,
   [23] an agent for inhibiting kinase (phosphorylation enzyme), which comprises a compound represented by the formula 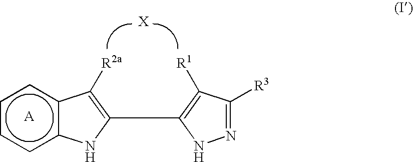
   wherein A is a benzene ring optionally having substituents, R¹, R^{2a} and R³ are each a hydrogen atom, a hydrocarbon group optionally having substituents or a heterocyclic group optionally having substituents, R¹ and R^{2a} optionally form a ring via X, and when R¹ and R^{2a} form a ring via X, R¹ and R^{2a} are each a bond or a divalent C₁₋₅ acyclic hydrocarbon group optionally having substituents, and X is a bond, an oxygen atom, an optionally oxidized sulfur atom or an imino group optionally having a substituent, provided that R¹, R^{2a} and X are not bonds at the same time, or a salt thereof, or a prodrug thereof,
   [24] the agent of the above-mentioned [23], wherein the kinase is a vascular endothelial growth factor receptor (VEGFR),
   [25] the agent of the above-mentioned [23], wherein the kinase is a vascular endothelial growth factor receptor (VEGFR) 2,
   [26] the agent of the above-mentioned [23], wherein the kinase is a fibroblast growth factor receptor (FGFR) 1,
   [27] an agent for inhibiting angiogenesis, which comprises a compound represented by the formula 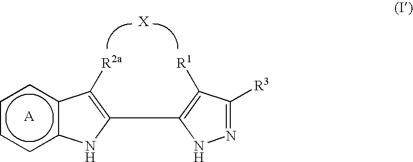
   wherein A is a benzene ring optionally having substituents, R¹, R^{2a} and R³ are each a hydrogen atom, a hydrocarbon group optionally having substituents or a heterocyclic group optionally having substituents, R¹ and R^{2a} optionally form a ring via X, and when R¹ and R^{2a} form a ring via X, R¹ and R^{2a} are each a bond or a divalent C₁₋₅ acyclic hydrocarbon group optionally having substituents, and X is a bond, an oxygen atom, an optionally oxidized sulfur atom or an imino group optionally having a substituent, provided that R¹, R^{2a} and X are not bonds at the same time, or a salt thereof, or a prodrug thereof,
   [28] an agent for the prophylaxis or treatment of cancer, which comprises a compound represented by the formula 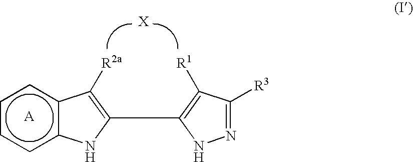
   wherein A is a benzene ring optionally having substituents, R¹, R^{2a} and R³ are each a hydrogen atom, a hydrocarbon group optionally having substituents or a heterocyclic group optionally having substituents, R¹ and R^{2a} optionally form a ring via X, and when R¹ and R^{2a} form a ring via X, R¹ and R^{2a} are each a bond or a divalent C₁₋₅ acyclic hydrocarbon group optionally having substituents, and X is a bond, an oxygen atom, an optionally oxidized sulfur atom or an imino group optionally having a substituent, provided that R¹, R^{2a} and X are not bonds at the same time, or a salt thereof, or a prodrug thereof,
   [29] an agent for inhibiting growth of cancer, which comprises a compound represented by the formula 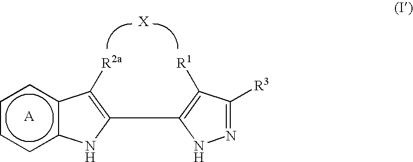
   wherein A is a benzene ring optionally having substituents, R¹, R^{2a} and R³ are each a hydrogen atom, a hydrocarbon group optionally having substituents or a heterocyclic group optionally having substituents, R¹ and R^{2a} optionally form a ring via X, and when R¹ and R^{2a} form a ring via X, R¹ and R^{2a} are each a bond or a divalent C₁₋₅ acyclic hydrocarbon group optionally having substituents, and X is a bond, an oxygen atom, an optionally oxidized sulfur atom or an imino group optionally having a substituent, provided that R¹, R^{2a} and X are not bonds at the same time, or a salt thereof, or a prodrug thereof,
   [30] an agent for suppressing metastasis of cancer, which comprises a compound represented by the formula 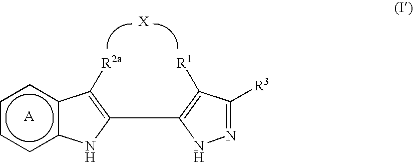
   wherein A is a benzene ring optionally having substituents, R¹, R^{2a} and R³ are each a hydrogen atom, a hydrocarbon group optionally having substituents or a heterocyclic group optionally having substituents, R¹ and R^{2a} optionally form a ring via X, and when R¹ and R^{2a} form a ring via X, R¹ and R^{2a} are each a bond or a divalent C₁₋₅ acyclic hydrocarbon group optionally having substituents, and X is a bond, an oxygen atom, an optionally oxidized sulfur atom or an imino group optionally having a substituent, provided that R¹, R^{2a} and X are not bonds at the same time, or a salt thereof, or a prodrug thereof,
   [31] a method for the prophylaxis or treatment of cancer in a mammal, which comprises administering, to said mammal, an effective amount of a compound represented by the formula 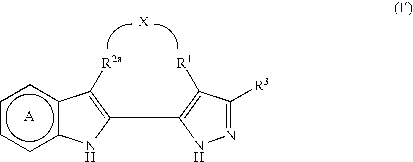
   wherein A is a benzene ring optionally having substituents, R¹, R^{2a} and R³ are each a hydrogen atom, a hydrocarbon group optionally having substituents or a heterocyclic group optionally having substituents, R¹ and R^{2a} optionally form a ring via X, and when R¹ and R^{2a} form a ring via X, R¹ and R^{2a} are each a bond or a divalent C₁₋₅ acyclic hydrocarbon group optionally having substituents, and X is a bond, an oxygen atom, an optionally oxidized sulfur atom or an imino group optionally having a substituent, provided that R¹, R^{2a} and X are not bonds at the same time, or a salt thereof, or a prodrug thereof, and
   [32] use of a compound represented by the formula 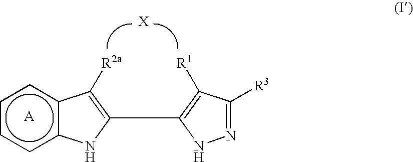
   wherein A is a benzene ring optionally having substituents, R¹, R^{2a} and R³ are each a hydrogen atom, a hydrocarbon group optionally having substituents or a heterocyclic group optionally having substituents, R¹ and R^{2a} optionally form a ring via X, and when R¹ and R^{2a} form a ring via X, R¹ and R^{2a} are each a bond or a divalent C₁₋₅ acyclic hydrocarbon group optionally having substituents, and X is a bond, an oxygen atom, an optionally oxidized sulfur atom or an imino group optionally having a substituent, provided that R¹, R^{2a} and X are not bonds at the same time, or a salt thereof, or a prodrug thereof, for the production of an agent for the prophylaxis or treatment of cancer.

Furthermore, the present invention provides
[33] a compound represented by the formula 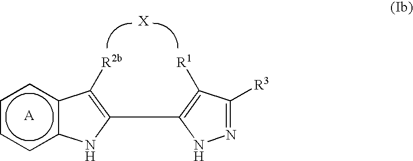
wherein A is a benzene ring optionally having substituents, R¹ and R³ are each a hydrogen atom, a hydrocarbon group optionally having substituents or a heterocyclic group optionally having substituents and R^{2b} is a hydrocarbon group optionally having substituents or a heterocyclic group optionally having substituents,

provided that R^{2b} is not a 4-hydroxyphenyl group optionally having substituents selected from the group consisting of a halogen atom, a C₁₋₆ alkyl group and a C₁₋₆ alkoxy group; a 4-methoxyphenyl group optionally having substituents selected from the group consisting of a halogen atom, a C₁₋₆ alkyl group and a C₁₋₆ alkoxy group; and a 6-hydroxypyridin-3-yl group optionally having substituents selected from the group consisting of a halogen atom, a C₁₋₆ alkyl group and a C₁₋₆ alkoxy group,

R¹ and R^{2b} optionally form a ring via X, and when R¹ and R^{2b} form a ring via X, R¹ and R^{2b} are each a bond or a divalent C₁₋₅ acyclic hydrocarbon group optionally having substituents, and X is a bond, an oxygen atom, an optionally oxidized sulfur atom or an imino group optionally having a substituent, provided that when X is a bond, the ring formed by R¹ and R^{2b} via X is a 7 or more-membered ring, or a salt thereof (excluding 8-methoxy-4,5,6,11-tetrahydro-1H-pyrazolo[4′,3′:6,7]cyclohepta[1,2-b]indole, 4,5,6,11-tetrahydro-1H-pyrazolo[4′,3′:6,7]cyclohepta[1,2-b]indole-8-carboxamide, 3-methyl-N-(4,5,6,11-tetrahydro-1H-pyrazolo[4′,3′:6,7]cyclohepta[1,2-b]indol-8-yl)butanamide, 8-chloro-4,5,6,11-tetrahydro-1H-pyrazolo[4′,3′:6,7]cyclohepta[1,2-b]indole, N-benzyl-4,5,6,11-tetrahydro-1H-pyrazolo[4′,3′:6,7]cyclohepta[1,2-b]indole-8-carboxamide, N-isobutyl-4,5,6,11-tetrahydro-1H-pyrazolo[4′,3′:6,7]cyclohepta[1,2-b]indole-8-carboxamide, ethyl 4,5,6,11-tetrahydro-1H-pyrazolo[4′,3′:6,7]cyclohepta[1,2-b]indole-3-carboxylate, N-(4,5,6,11-tetrahydro-1H-pyrazolo[4′,3′:6,7]cyclohepta[1,2-b]indol-8-yl)acetamide and 2-phenyl-N-(4,5,6,11-tetrahydro-2H-pyrazolo[4′,3′:6,7]cyclohepta[1,2-b]indol-8-yl)acetamide),
[34] the compound of the above-mentioned [33], which is represented by the formula 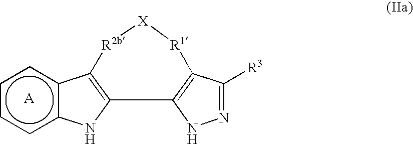
wherein R¹′ and R^{2b}′ are each a bond or a divalent C₁₋₅ acyclic hydrocarbon group optionally having substituents, and A, R³ and X are as defined in the above-mentioned [33], provided that when X is a bond, the ring formed by R¹′ and R^{2b}′ via X is a 7 or more-membered ring,
[35] the compound of the above-mentioned [33], which is represented by the formula 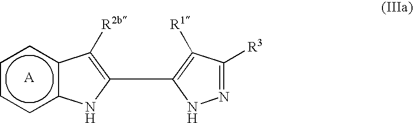
wherein R¹″ is a hydrogen atom, a hydrocarbon group optionally having substituents or a heterocyclic group optionally having substituents, R^{2b}″ is a hydrocarbon group optionally having substituents or a heterocyclic group optionally having substituents, and A and R³ are as defined in the above-mentioned [33], provided that R^{2b}″ is not a 4-hydroxyphenyl group optionally having substituents selected from the group consisting of a halogen atom, a C₁₋₆ alkyl group and a C₁₋₆ alkoxy group; a 4-methoxyphenyl group optionally having substituents selected from the group consisting of a halogen atom, a C₁₋₆ alkyl group and a C₁₋₆ alkoxy group; and a 6-hydroxypyridin-3-yl group optionally having substituents selected from the group consisting of a halogen atom, a C₁₋₆ alkyl group and a C₁₋₆ alkoxy group,
[36] the compound of the above-mentioned [35], which is represented by the formula 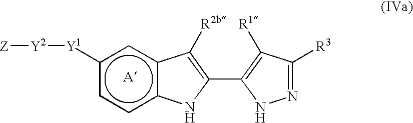
wherein A′ is a benzene ring optionally having substituents, Y¹ and Y² are each a bond, an oxygen atom, an optionally oxidized sulfur atom, an imino group optionally having a substituent or a carbonyl group, Z is a hydrogen atom, a hydrocarbon group optionally having substituents or a heterocyclic group optionally having substituents, and R¹″, R^{2b}″ and R³ are as defined in the above-mentioned [35],
[37] the compound of the above-mentioned [33], wherein the substituent of the benzene ring for A is a carbamoyl group optionally having substituents or an optionally substituted heterocycle-carbonyl group,
[38] the compound of the above-mentioned [34], wherein R¹′ is a —CH₂CH₂CH₂— group optionally having substituents, and R^{2b}′ and X are bonds,
[39] the compound of the above-mentioned [33], wherein, when X is a bond and R¹ and R^{2b} form a 7 or more-membered ring via X, the substituent of the benzene ring for A is a heterocycle-carbonyl group having substituent(s),
[40] the compound of the above-mentioned [33], wherein, when X is a bond and R¹ and R^{2b} form a 7 or more-membered ring via X, the substituent of the benzene ring for A is a 1-piperidinylcarbonyl group having substituent(s),
[41] a compound represented by the formula 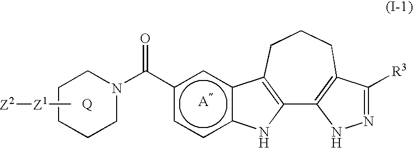
wherein Z¹ is a bond, methylene (CH₂), ethylene (CH₂CH₂), vinylene (CHCH) or ethynylene (CC), Z² is a phenyl group optionally having substituents or a heterocyclic group optionally having substituents, A″ is a benzene ring optionally further having substituents, R³ is a hydrogen atom, a hydrocarbon group optionally having substituents or a heterocyclic group optionally having substituents, and Q is a piperidine ring optionally further having substituents other than -Z¹-Z², or a salt thereof,
[42] the compound of the above-mentioned [41], which is represented by the formula 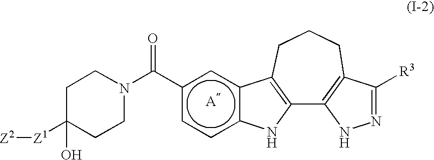
wherein each symbol is as defined in the above-mentioned [41],
[43] the compound of the above-mentioned [36], wherein Y¹ is a carbonyl group, Y² is a bond and Z is 1-piperidinyl optionally having substituents,
[44] a prodrug of the compound of the above-mentioned [33] or [41],
[45] a pharmaceutical agent comprising the compound of the above-mentioned [33] or [41], or a prodrug thereof,
[46] an agent for inhibiting kinase (phosphorylation enzyme) comprising a compound represented by the formula 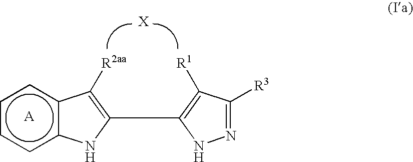
wherein A is a benzene ring optionally having substituents, R¹ and R³ are each a hydrogen atom, a hydrocarbon group optionally having substituents or a heterocyclic group optionally having substituents, R^{2aa} is a hydrocarbon group optionally having substituents or a heterocyclic group optionally having substituents, R¹ and R^{2aa} optionally form a ring via X, and when R¹ and R^{2aa} form a ring via X, R¹ and R^{2aa} are each a bond or a divalent C₁₋₅ acyclic hydrocarbon group optionally having substituents, and X is a bond, an oxygen atom, an optionally oxidized sulfur atom or an imino group optionally having a substituent, provided that when X is a bond, the ring formed by R¹ and R^{2aa} via X is a 7 or more-membered ring, or a salt thereof, or a prodrug thereof (excluding 4,5,6,11-tetrahydro-1H-pyrazolo[4′,3′:6,7]cyclohepta[1,2-b]indole-8-carboxamide, 3-methyl-N-(4,5,6,11-tetrahydro-1H-pyrazolo[4′,3′:6,7]cyclohepta[1,2-b]indol-8-yl)butanamide, 8-chloro-4,5,6,11-tetrahydro-1H-pyrazolo[4′,3′:6,7]cyclohepta[1,2-b]indole, N-benzyl-4,5,6,11-tetrahydro-1H-pyrazolo[4′,3′:6,7]cyclohepta[1,2-b]indole-8-carboxamide, N-isobutyl-4,5,6,11-tetrahydro-1H-pyrazolo[4′,3′:6,7]cyclohepta[1,2-b]indole-8-carboxamide, ethyl 4,5,6,11-tetrahydro-1H-pyrazolo[4′,3′:6,7]cyclohepta[1,2-b]indole-3-carboxylate, N-(4,5,6,11-tetrahydro-1H-pyrazolo[4′,3′:6,7]cyclohepta[1,2-b]indol-8-yl)acetamide and 2-phenyl-N-(4,5,6,11-tetrahydro-2H-pyrazolo[4′,3′:6,7]cyclohepta[1,2-b]indol-8-yl)acetamide),
[47] an agent for inhibiting vascular endothelial growth factor receptor (VEGFR) comprising a compound represented by the formula 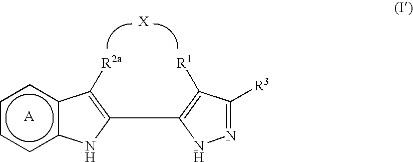
wherein A is a benzene ring optionally having substituents, R¹, R^{2a} and R³ are each a hydrogen atom, a hydrocarbon group optionally having substituents or a heterocyclic group optionally having substituents, R¹ and R^{2a} optionally form a ring via X, and when R¹ and R^{2a} form a ring via X, R¹ and R^{2a} are each a bond or a divalent C₁₋₅ acyclic hydrocarbon group optionally having substituents, and X is a bond, an oxygen atom, an optionally oxidized sulfur atom or an imino group optionally having a substituent, provided that R¹, R^{2a} and X are not bonds at the same time, or a salt thereof, or a prodrug thereof,
[48] an agent for inhibiting vascular endothelial growth factor receptor (VEGFR) 2, which comprises a compound represented by the formula 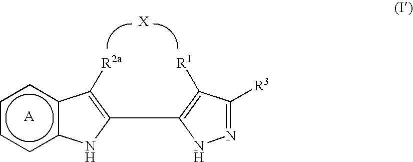
wherein A is a benzene ring optionally having substituents, R¹, R^{2a} and R³ are each a hydrogen atom, a hydrocarbon group optionally having substituents or a heterocyclic group optionally having substituents, R¹ and R^{2a} optionally form a ring via X, and when R¹ and R^{2a} form a ring via X, R¹ and R^{2a} are each a bond or a divalent C₁₋₅ acyclic hydrocarbon group optionally having substituents, and X is a bond, an oxygen atom, an optionally oxidized sulfur atom or an imino group optionally having a substituent, provided that R¹, R^{2a} and X are not bonds at the same time, or a salt thereof, or a prodrug hereof,
[49] an agent for inhibiting fibroblast growth factor receptor (FGFR) 1, which comprises a compound represented by the formula 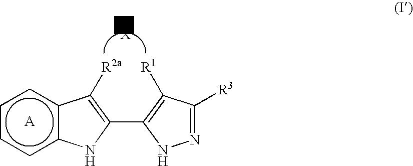
wherein A is a benzene ring optionally having substituents, R¹, R^{2a} and R³ are each a hydrogen atom, a hydrocarbon group optionally having substituents or a heterocyclic group optionally having substituents, R¹ and R^{2a} optionally form a ring via X, and when R¹ and R^{2a} form a ring via X, R¹ and R^{2a} are each a bond or a divalent C₁₋₅ acyclic hydrocarbon group optionally having substituents, and X is a bond, an oxygen atom, an optionally oxidized sulfur atom or an imino group optionally having a substituent, provided that R¹, R^{2a} and X are not bonds at the same time, or a salt thereof, or a prodrug thereof,
[50] an agent for inhibiting angiogenesis, which comprises a compound represented by the formula 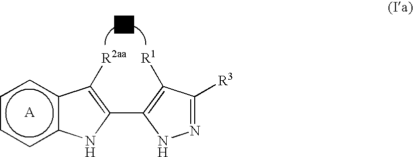
wherein A is a benzene ring optionally having substituents, R¹ and R³ are each a hydrogen atom, a hydrocarbon group optionally having substituents or a heterocyclic group optionally having substituents, R^{2aa} is a hydrocarbon group optionally having substituents or a heterocyclic group optionally having substituents, R¹ and R^{2aa} optionally form a ring via X, and when R¹ and R^{2aa} form a ring via X, R¹ and R^{2aa} are each a bond or a divalent C₁₋₅ acyclic hydrocarbon group optionally having substituents, and X is a bond, an oxygen atom, an optionally oxidized sulfur atom or an imino group optionally having a substituent, provided that when X is a bond, the ring formed by R¹ and R^{2aa} via X is a 7 or more-membered ring, or a salt thereof, or a prodrug thereof (excluding 4,5,6,11-tetrahydro-1H-pyrazolo[4′,3′:6,7]cyclohepta[1,2-b]indole-8-carboxamide, 3-methyl-N-(4,5,6,11-tetrahydro-1H-pyrazolo[4′,3′:6,7]cyclohepta[1,2-b]indol-8-yl)butanamide, 8-chloro-4,5,6,11-tetrahydro-1H-pyrazolo[4′,3′:6,7]cyclohepta[1,2-b]indole, N-benzyl-4,5,6,11-tetrahydro-1H-pyrazolo[4′,3′:6,7]cyclohepta[1,2-b]indole-8-carboxamide, N-isobutyl-4,5,6,11-tetrahydro-1H-pyrazolo[4′,3′:6,7]cyclohepta[1,2-b]indole-8-carboxamide, ethyl 4,5,6,11-tetrahydro-1H-pyrazolo[4′,3′:6,7]cyclohepta[1,2-b]indole-3-carboxylate, N-(4,5,6,11-tetrahydro-1H-pyrazolo[4′,3′:6,7]cyclohepta[1,2-b]indol-8-yl)acetamide and 2-phenyl-N-(4,5,6,11-tetrahydro-2H-pyrazolo[4′,3′:6,7]cyclohepta[1,2-b]indol-8-yl)acetamide),
[51] an agent for the prophylaxis or treatment of cancer (e.g., colorectal cancer, lung cancer, pancreatic cancer, gastric cancer, breast cancer, ovarian cancer, prostate cancer, liver cancer, thyroid cancer, kidney cancer, cerebral tumor, melanoma, urinary bladder cancer and the like), which comprises a compound represented by the formula 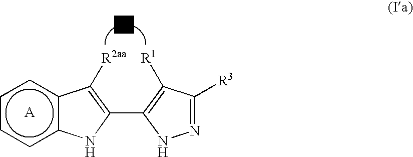
wherein A is a benzene ring optionally having substituents, R¹ and R³ are each a hydrogen atom, a hydrocarbon group optionally having substituents or a heterocyclic group optionally having substituents, R^{2aa} is a hydrocarbon group optionally having substituents or a heterocyclic group optionally having substituents, R¹ and R^{2aa} optionally form a ring via X, and when R¹ and R^{2aa} form a ring via X, R¹ and R^{2aa} are each a bond or a divalent C₁₋₅ acyclic hydrocarbon group optionally having substituents, and X is a bond, an oxygen atom, an optionally oxidized sulfur atom or an imino group optionally having a substituent, provided that when X is a bond, the ring formed by R¹ and R^{2aa} via X is a 7 or more-membered ring, or a salt thereof, or a prodrug thereof (excluding 4,5,6,11-tetrahydro-1H-pyrazolo[4′,3′:6,7]cyclohepta[1,2-b]indole-8-carboxamide, 3-methyl-N-(4,5,6,11-tetrahydro-1H-pyrazolo[4′,3′:6,7]cyclohepta[1,2-b]indol-8-yl)butanamide, 8-chloro-4,5,6,11-tetrahydro-1H-pyrazolo[4′,3′:6,7]cyclohepta[1,2-b]indole, N-benzyl-4,5,6,11-tetrahydro-1H-pyrazolo[4′,3′:6,7]cyclohepta[1,2-b]indole-8-carboxamide, N-isobutyl-4,5,6,1′-tetrahydro-1H-pyrazolo[4′,3′:6,7]cyclohepta[1,2-b]indole-8-carboxamide, ethyl 4,5,6,11-tetrahydro-1H-pyrazolo[4′,3′:6,7]cyclohepta[1,2-b]indole-3-carboxylate, N-(4,5,6,11-tetrahydro-1H-pyrazolo[4′,3′:6,7]cyclohepta[1,2-b]indol-8-yl)acetamide and 2-phenyl-N-(4,5,6,11-tetrahydro-2H-pyrazolo[4′,3′:6,7]cyclohepta[1,2-b]indol-8-yl)acetamide),
[52] an agent for inhibiting growth of cancer (e.g., colorectal cancer, lung cancer, pancreatic cancer, gastric cancer, breast cancer, ovarian cancer, prostate cancer, liver cancer, thyroid cancer, kidney cancer, cerebral tumor, melanoma, urinary bladder cancer and the like), which comprises a compound represented by the formula 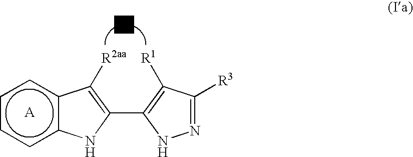
wherein A is a benzene ring optionally having substituents, R¹ and R³ are each a hydrogen atom, a hydrocarbon group optionally having substituents or a heterocyclic group optionally having substituents, R^{2aa} is a hydrocarbon group optionally having substituents or a heterocyclic group optionally having substituents, R¹ and R^{2aa} optionally form a ring via X, and when R¹ and R^{2aa} form a ring via X, R¹ and R^{2aa} are each a bond or a divalent C₁₋₅ acyclic hydrocarbon group optionally having substituents, and X is a bond, an oxygen atom, an optionally oxidized sulfur atom or an imino group optionally having a substituent, provided that when X is a bond, the ring formed by R¹ and R^{2aa} via X is a 7 or more-membered ring, or a salt thereof, or a prodrug thereof (excluding 4,5,6,11-tetrahydro-1H-pyrazolo[4′,3′:6,7]cyclohepta[1,2-b]indole-8-carboxamide, 3-methyl-N-(4,5,6,11-tetrahydro-1H-pyrazolo[4′,3′:6,7]cyclohepta[1,2-b]indol-8-yl)butanamide, 8-chloro-4,5,6,11-tetrahydro-1H-pyrazolo[4′,3′:6,7]cyclohepta[1,2-b]indole, N-benzyl-4,5,6,11-tetrahydro-1H-pyrazolo[4′,3′:6,7]cyclohepta[1,2-b]indole-8-carboxamide, N-isobutyl-4,5,6,11-tetrahydro-1H-pyrazolo[4′,3′:6,7]cyclohepta[1,2-b]indole-8-carboxamide, ethyl 4,5,6,11-tetrahydro-1H-pyrazolo[4′,3′:6,7]cyclohepta[1,2-b]indole-3-carboxylate, N-(4,5,6,11-tetrahydro-1H-pyrazolo[4′,3′:6,7]cyclohepta[1,2-b]indol-8-yl)acetamide and 2-phenyl-N-(4,5,6,11-tetrahydro-2H-pyrazolo[4′,3′:6,7]cyclohepta[1,2-b]indol-8-yl)acetamide),
[53] an agent for suppress metastasis of cancer (e.g., colorectal cancer, lung cancer, pancreatic cancer, gastric cancer, breast cancer, ovarian cancer, prostate cancer, liver cancer, thyroid cancer, kidney cancer, cerebral tumor, melanoma, urinary bladder cancer and the like), which comprises a compound represented by the formula 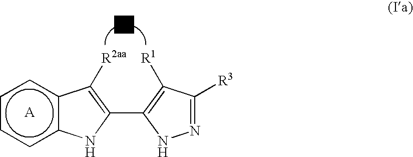
wherein A is a benzene ring optionally having substituents, R¹ and R³ are each a hydrogen atom, a hydrocarbon group optionally having substituents or a heterocyclic group optionally having substituents, R^{2aa} is a hydrocarbon group optionally having substituents or a heterocyclic group optionally having substituents, R¹ and R^{2aa} optionally form a ring via X, and when R¹ and R^{2aa} form a ring via X, R¹ and R^{2aa} are each a bond or a divalent C₁₋₅ acyclic hydrocarbon group optionally having substituents, and X is a bond, an oxygen atom, an optionally oxidized sulfur atom or an imino group optionally having a substituent, provided that when X is a bond, the ring formed by R¹ and R^{2aa} via X is a 7 or more-membered ring, or a salt thereof, or a prodrug thereof (excluding 4,5,6,11-tetrahydro-1H-pyrazolo[4′,3′:6,7]cyclohepta[1,2-b]indole-8-carboxamide, 3-methyl-N-(4,5,6,11-tetrahydro-1H-pyrazolo[4′,3′:6,7]cyclohepta[1,2-b]indol-8-yl)butanamide, 8-chloro-4,5,6,11-tetrahydro-1H-pyrazolo[4′,3′:6,7]cyclohepta[1,2-b]indole, N-benzyl-4,5,6,11-tetrahydro-1H-pyrazolo[4′,3′:6,7]cyclohepta[1,2-b]indole-8-carboxamide, N-isobutyl-4,5,6,11-tetrahydro-1H-pyrazolo[4′,3′:6,7]cyclohepta[1,2-b]indole-8-carboxamide, ethyl 4,5,6,11-tetrahydro-1H-pyrazolo[4′,3′:6,7]cyclohepta[1,2-b]indole-3-carboxylate, N-(4,5,6,11-tetrahydro-1H-pyrazolo[4′,3′:6,7]cyclohepta[1,2-b]indol-8-yl)acetamide and 2-phenyl-N-(4,5,6,11-tetrahydro-2H-pyrazolo[4′,3′:6,7]cyclohepta[1,2-b]indol-8-yl)acetamide),
[54] an agent for the prophylaxis or treatment of cancer in patients with a cancer (e.g., colorectal cancer, lung cancer, pancreatic cancer, gastric cancer, breast cancer, ovarian cancer, prostate cancer, liver cancer, thyroid cancer, kidney cancer, cerebral tumor, melanoma, urinary bladder cancer and the like) expressing a vascular endothelial growth factor receptor (VEGFR) and/or a fibroblast growth factor receptor (FGFR) 1, which comprises a compound represented by the formula 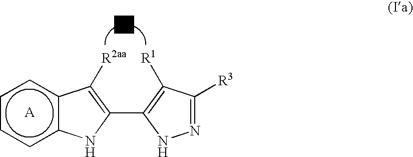
wherein A is a benzene ring optionally having substituents, R¹ and R³ are each a hydrogen atom, a hydrocarbon group optionally having substituents or a heterocyclic group optionally having substituents, R^{2aa} is a hydrocarbon group optionally having substituents or a heterocyclic group optionally having substituents, R¹ and R^{2aa} optionally form a ring via X, and when R¹ and R^{2aa} form a ring via X, R¹ and R^{2aa} are each a bond or a divalent C₁₋₅ acyclic hydrocarbon group optionally having substituents, and X is a bond, an oxygen atom, an optionally oxidized sulfur atom or an imino group optionally having a substituent, provided that when X is a bond, the ring formed by R¹ and R^{2aa} via X is a 7 or more-membered ring, or a salt thereof, or a prodrug thereof (excluding 4,5,6,11-tetrahydro-1H-pyrazolo[4′,3′:6,7]cyclohepta[1,2-b]indole-8-carboxamide, 3-methyl-N-(4,5,6,11-tetrahydro-1H-pyrazolo[4′,3′:6,7]cyclohepta[1,2-b]indol-8-yl)butanamide, 8-chloro-4,5,6,11-tetrahydro-1H-pyrazolo[4′,3′:6,7]cyclohepta[1,2-b]indole, N-benzyl-4,5,6,11-tetrahydro-1H-pyrazolo[4′,3′:6,7]cyclohepta[1,2-b]indole-8-carboxamide, N-isobutyl-4,5,6,11-tetrahydro-1H-pyrazolo[4′,3′:6,7]cyclohepta[1,2-b]indole-8-carboxamide, ethyl 4,5,6,11-tetrahydro-1H-pyrazolo[4′,3′:6,7]cyclohepta[1,2-b]indole-3-carboxylte, N-(4,5,6,11-tetrahydro-1H-pyrazolo[4′,3′:6,7]cyclohepta[1,2-b]indol-8-yl)acetamide and 2-phenyl-N-(4,5,6,11-tetrahydro-2H-pyrazolo[4′,3′:6,7]cyclohepta[1,2-b]indol-8-yl)acetamide),
[55] a method for the prophylaxis or treatment of a cancer (e.g., colorectal cancer, lung cancer, pancreatic cancer, gastric cancer, breast cancer, ovarian cancer, prostate cancer, liver cancer, thyroid cancer, kidney cancer, cerebral tumor, melanoma, urinary bladder cancer and the like) in a mammal, which comprises administering, to said mammal, an effective amount of a compound represented by the formula 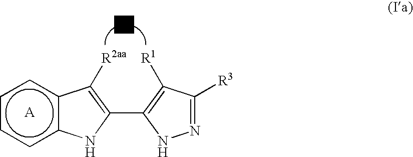
wherein A is a benzene ring optionally having substituents, R¹ and R³ are each a hydrogen atom, a hydrocarbon group optionally having substituents or a heterocyclic group optionally having substituents, R^{2aa} is a hydrocarbon group optionally having substituents or a heterocyclic group optionally having substituents, R¹ and R^{2aa} optionally form a ring via X, and when R¹ and R^{2aa} form a ring via X, R¹ and R^{2aa} are each a bond or a divalent C₁₋₅ acyclic hydrocarbon group optionally having substituents, and X is a bond, an oxygen atom, an optionally oxidized sulfur atom or an imino group optionally having a substituent, provided that when X is a bond, the ring formed by R¹ and R^{2aa} via X is a 7 or more-membered ring, or a salt thereof, or a prodrug thereof (excluding 4,5,6,11-tetrahydro-1H-pyrazolo[4′,3′:6,7]cyclohepta[1,2-b]indole-8-carboxamide, 3-methyl-N-(4,5,6,11-tetrahydro-1H-pyrazolo[4′,3′:6,7]cyclohepta[1,2-b]indol-8-yl)butanamide, 8-chloro-4,5,6,11-tetrahydro-1H-pyrazolo[4′,3′:6,7]cyclohepta[1,2-b]indole, N-benzyl-4,5,6,11-tetrahydro-1H-pyrazolo[4′,3′:6,7]cyclohepta[1,2-b]indole-8-carboxamide, N-isobutyl-4,5,6,11-tetrahydro-1H-pyrazolo[4′,3′:6,7]cyclohepta[1,2-b]indole-8-carboxamide, ethyl 4,5,6,11-tetrahydro-1H-pyrazolo[4′,3′:6,7]cyclohepta[1,2-b]indole-3-carboxylate, N-(4,5,6,11-tetrahydro-1H-pyrazolo[4′,3′:6,7]cyclohepta[1,2-b]indol-8-yl)acetamide and 2-phenyl-N-(4,5,6,11-tetrahydro-2H-pyrazolo[4′,3′:6,7]cyclohepta[1,2-b]indol-8-yl)acetamide),
[56] use of a compound represented by the formula 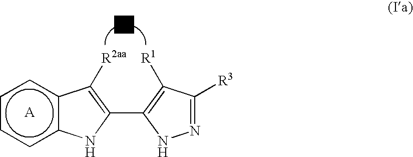
wherein A is a benzene ring optionally having substituents, R¹ and R³ are each a hydrogen atom, a hydrocarbon group optionally having substituents or a heterocyclic group optionally having substituents, R^{2aa} is a hydrocarbon group optionally having substituents or a heterocyclic group optionally having substituents, R¹ and R^{2aa} optionally form a ring via X, and when R¹ and R^{2aa} form a ring via X, R¹ and R^{2aa} are each a bond or a divalent C₁₋₅ acyclic hydrocarbon group optionally having substituents, and X is a bond, an oxygen atom, an optionally oxidized sulfur atom or an imino group optionally having a substituent, provided that when X is a bond, the ring formed by R¹ and R^{2aa} via X is a 7 or more-membered ring, or a salt thereof, or a prodrug thereof (excluding 4,5,6,11-tetrahydro-1H-pyrazolo[4′,3′:6,7]cyclohepta[1,2-b]indole-8-carboxamide, 3-methyl-N-(4,5,6,11-tetrahydro-1H-pyrazolo[4′,3′:6,7]cyclohepta[1,2-b]indol-8-yl)butanamide, 8-chloro-4,5,6,11-tetrahydro-1H-pyrazolo[4′,3′:6,7]cyclohepta[1,2-b]indole, N-benzyl-4,5,6,11-tetrahydro-1H-pyrazolo[4′,3′:6,7]cyclohepta[1,2-b]indole-8-carboxamide, N-isobutyl-4,5,6,11-tetrahydro-1H-pyrazolo[4′,3′:6,7]cyclohepta[1,2-b]indole-8-carboxamide, ethyl 4,5,6,11-tetrahydro-1H-pyrazolo[4′,3′:6,7]cyclohepta[1,2-b]indole-3-carboxylate, N-(4,5,6,11-tetrahydro-1H-pyrazolo[4′,3′:6,7]cyclohepta[1,2-b]indol-8-yl)acetamide and 2-phenyl-N-(4,5,6,11-tetrahydro-2H-pyrazolo[4′,3′:6,7]cyclohepta[1,2-b]indol-8-yl)acetamide) for the production of an agent for the prophylaxis or treatment of cancer (e.g., colorectal cancer, lung cancer, pancreatic cancer, gastric cancer, breast cancer, ovarian cancer, prostate cancer, liver cancer, thyroid cancer, kidney cancer, cerebral tumor, melanoma, urinary bladder cancer and the like),
[57] the compound of the above-mentioned [1], wherein A is a benzene ring optionally having substituents selected from substituent group A,
substituent group A consists of the following (i) to (xxxxvi),
(i) a halogen atom;
(ii) C₁₋₃ alkylenedioxy;
(iii) nitro;
(iv) cyano;
(v) optionally esterified carboxyl selected from
(a) carboxyl,
(b) C₁₋₆ alkoxy-carbonyl optionally substituted by 1 to 5 substituents selected from substituent group B,
(c) C₆₋₁₄ aryloxy-carbonyl optionally substituted by 1 to 5 substituents selected from substituent group B and
(d) C₇₋₁₆ aralkyloxy-carbonyl optionally substituted by 1 to 5 substituents selected from substituent group B;
   (vi) lower (C₁₋₆)alkyl optionally substituted by 1 to 5 substituents selected from substituent group B;
   (vii) lower (C₂₋₆)alkenyl optionally substituted by 1 to 5 substituents selected from substituent group B;
   (viii) lower (C₂₋₆)alkynyl optionally substituted by 1 to 5 substituents selected from substituent group B;
   (ix) C₃₋₈ cycloalkyl optionally substituted by 1 to 5 substituents selected from substituent group B;
   (x) C₆₋₁₄ aryl optionally substituted by 1 to 5 substituents selected from substituent group B;
   (xi) C₇₋₁₆ aralkyl optionally substituted by 1 to 5 substituents selected from substituent group B;
   (xii) C₆₋₁₄ aryl-C₂₋₆ alkenyl optionally substituted by 1 to 5 substituents selected from substituent group B;
   (xiii) a 3 to 14-membered (monocyclic, bicyclic or tricyclic) heterocyclic group containing, besides carbon atom, 1 to 4 hetero atoms of 1 or 2 kinds selected from a nitrogen atom, a sulfur atom and an oxygen atom, which optionally has 1 to 5 substituents selected from substituent group B;
   (xiv) hydroxy;
   (xv) lower (C₁₋₆)alkoxy optionally substituted by 1 to 5 substituents selected from substituent group B;
   (xvi) C₆₋₁₄ aryloxy optionally substituted by 1 to 5 substituents selected from substituent group B;
   (xvii) C₇₋₁₆ aralkyloxy optionally substituted by 1 to 5 substituents selected from substituent group B;
   (xviii) lower (C₁₋₆)alkyl-carbonyloxy optionally substituted by
   (xix) lower (C₁₋₆)alkoxy-carbonyloxy optionally substituted by 1 to 5 substituents selected from substituent group B;
   (xx) mono-lower(C₁₋₆)alkyl-carbamoyloxy optionally substituted by 1 to 5 substituents selected from substituent group B;
   (xxi) di-lower(C₁₋₆)alkyl-carbamoyloxy optionally substituted by 1 to 5 substituents selected from substituent group B;
   (xxii) C₆₋₁₄ aryl-carbonyloxy optionally substituted by 1 to 5 substituents selected from substituent group B;
   (xxiii) mono- or di-C₆₋₁₄ aryl-carbamoyloxy optionally substituted by 1 to 5 substituents selected from substituent group B;
   (xxiv) 3 to 14-membered (monocyclic, bicyclic or tricyclic) heterocycle-oxy containing, besides carbon atom, 1 to 4 hetero atoms of 1 or 2 kinds selected from a nitrogen atom, a sulfur atom and an oxygen atom, which is optionally substituted by 1 to 5 substituents selected from substituent group B (preferably, 5 to 10-membered aromatic heterocycle-oxy containing, besides carbon atom, 1 to 4 hetero atoms of 1 or 2 kinds selected from a nitrogen atom, a sulfur atom and an oxygen atom, which is optionally substituted by 1 to 5 substituents selected from substituent group B);
   (xxv) mercapto;
   (xxvi) lower (C₁₋₆)alkylthio optionally substituted by 1 to 5 substituents selected from substituent group B;
   (xxvii) C₆₋₁₄ arylthio optionally substituted by 1 to 5 substituents selected from substituent group B;
   (xxviii) C₇₋₁₆ aralkylthio optionally substituted by 1 to 5 substituents selected from substituent group B;
   (xxix) formyl;
   (xxx) lower (C₁₋₆)alkyl-carbonyl optionally substituted by 1 to 5 substituents selected from substituent group B;
   (xxxi) C₃₋₈ cycloalkyl-carbonyl optionally substituted by 1 to 5 substituents selected from substituent group B;
   (xxxii) C₆₋₁₄ aryl-carbonyl optionally substituted by 1 to 5 substituents selected from substituent group B;
   (xxxiii) C₇₋₁₆ aralkyl-carbonyl optionally substituted by 1 to 5 substituents selected from substituent group B;
   (xxxiv) 3 to 14-membered (monocyclic, bicyclic or tricyclic) heterocycle-carbonyl containing, besides carbon atom, 1 to 4 hetero atoms of 1 or 2 kinds selected from a nitrogen atom, a sulfur atom and an oxygen atom, which is optionally substituted by 1 to 5 substituents selected from the group consisting of the following (a) to (d);
(a) a substituent selected from substituent group B (except a phenyl group and a 5 to 10-membered heterocyclic group containing, besides carbon atom, 1 to 4 hetero atoms of 1 or 2 kinds selected from a nitrogen atom, a sulfur atom and an oxygen atom (wherein said heterocyclic group is optionally substituted by halogen atom, hydroxy, amino, mono- or di-C₁₋₆ alkylamino, mono- or di-C₆₋₁₄ arylamino, mono- or di-C₇₋₁₆ aralkylamino, C₃₋₈ cycloalkyl, C₁₋₆ alkoxy, formyl, C₁₋₆ alkyl-carbonyl, C₃₋₈ cycloalkyl-carbonyl, C₆₋₁₄ aryl-carbonyl, C₇₋₁₆ aralkyl-carbonyl, C₁₋₆ alkoxy-carbonyl, C₆₋₁₄ aryloxy-carbonyl, C₇₋₁₆ aralkyloxy-carbonyl, C₁₋₆ alkylthio, C₁₋₆ alkylsulfinyl, C₁₋₆ alkylsulfonyl, carbamoyl, thiocarbamoyl, mono- or di-C₁₋₆ alkyl-carbamoyl, mono- or di-C₆₋₁₄ aryl-carbamoyl or the like)),
(b) a phenyl group, a benzyl group, a phenethyl group, a styryl group, a phenylethynyl group or a phenoxymethyl group, each optionally having 1 to 5 substituents selected from substituent group B,
(c) a 3 to 14-membered (monocyclic, bicyclic or tricyclic) heterocyclic group containing, besides carbon atom, 1 to 4 hetero atoms of 1 or 2 kinds selected from a nitrogen atom, a sulfur atom and an oxygen atom, which optionally has 1 to 5 substituents selected from substituents B group, and
(d) a group represented by the formula -Z¹-Z²
wherein Z¹ is a bond, methylene (CH₂), ethylene (CH₂CH₂), vinylene (CHCH), ethynylene (CC) or methyleneoxy (CH₂O), Z² is (i) a phenyl group optionally having substituents selected from the group consisting of (a) a halogen atom, (b) cyano, (c) an optionally halogenated C₁₋₆ alkyl group, (d) an optionally halogenated C₁₋₆ alkoxy group, (e) carbamoyl and (f) sulfamoyl, or
(ii) a 5 to 10-membered monocyclic or bicyclic heterocyclic group containing, besides carbon atom, 1 to 4 hetero atoms of 1 or 2 kinds selected from a nitrogen atom, a sulfur atom and an oxygen atom, which optionally has substituents selected from the group consisting of (a) a halogen atom, (b) cyano, (c) an optionally halogenated C₁₋₆ alkyl group, (d) an optionally halogenated C₁₋₆ alkoxy group, (e) carbamoyl and (f) sulfamoyl;
   (xxxv) lower (C₁₋₆)alkylsulfonyl optionally substituted by 1 to 5 substituents selected from substituent group B;
   (xxxvi) C₆₋₁₄ arylsulfonyl optionally substituted by 1 to 5 substituents selected from substituent group B;
   (xxxvii) lower (C₁₋₆)alkylsulfinyl optionally substituted by 1 to 5 substituents selected from substituent group B;
   (xxxviii) C₆₋₁₄ arylsulfinyl optionally substituted by 1 to 5 substituents selected from substituent group B;
   (xxxix) sulfo;
   (xxxx) sulfamoyl;
   (xxxxi) sulfinamoyl;
   (xxxxii) sulfenamoyl;
   (xxxxiii) thiocarbamoyl;
   (xxxxiv) a carbamoyl group or a 5 to 7-membered cyclic carbamoyl group optionally having 1 or 2 substituents selected from the group consisting of the following (a) to (p);
(a) lower (C₁₋₆)alkyl optionally substituted by 1 to 5 substituents selected from substituent group B (particularly, lower (C₁₋₆)alkyl optionally substituted by an “optionally substituted amino group” or a “5 to 10-membered heterocyclic group containing, besides carbon atom, 1 to 4 hetero atoms of 1 or 2 kinds selected from a nitrogen atom, a sulfur atom and an oxygen atom” of substituent group B),
(b) lower (C₂₋₆)alkenyl optionally substituted by 1 to 5 substituents selected from substituent group B,
(c) lower (C₂₋₆)alkynyl optionally substituted by 1 to 5 substituents selected from substituent group B,
(d) C₃₋₈ cycloalkyl optionally substituted by 1 to 5 substituents selected from substituent group B,
(e) C₆₋₁₄ aryl optionally substituted by 1 to 5 substituents selected from substituent group B,
(f) C₇₋₁₆ aralkyl optionally substituted by 1 to 5 substituents selected from substituent group B,
(g) a 3 to 14-membered (monocyclic, bicyclic or tricyclic) heterocyclic group containing, besides carbon atom, 1 to 4 hetero atoms of 1 or 2 kinds selected from a nitrogen atom, a sulfur atom and an oxygen atom, which optionally has 1 to 5 substituents selected from substituent group B,
(h) lower (C₁₋₆)alkoxy optionally substituted by 1 to 5 substituents selected from substituent group B,
(i) formyl,
(j) lower (C₁₋₆)alkyl-carbonyl optionally substituted by 1 to 5 substituents selected from substituent group B,
(k) C₃₋₈ cycloalkyl-carbonyl optionally substituted by 1 to 5 substituents selected from substituent group B,
(l) C₆₋₁₄ aryl-carbonyl optionally substituted by 1 to 5 substituents selected from substituent group B,
(m) C₇₋₁₆ aralkyl-carbonyl optionally substituted by 1 to 5 substituents selected from substituent group B,
(n) 3 to 14-membered (monocyclic, bicyclic or tricyclic) heterocycle-carbonyl containing, besides carbon atom, 1 to 4 hetero atoms of 1 or 2 kinds selected from a nitrogen atom, a sulfur atom and an oxygen atom, which is optionally substituted by 1 to 5 substituents selected from the group consisting of the following (aa) to (dd);
   (aa) a substituent selected from substituent group B (except a phenyl group and a 5 to 10-membered heterocyclic group containing, besides carbon atom, 1 to 4 hetero atoms of 1 or 2 kinds selected from a nitrogen atom, a sulfur atom and an oxygen atom (wherein said heterocyclic group is optionally substituted by halogen atom, hydroxy, amino, mono- or di-C₁₋₆ alkylamino, mono- or di-C₆₋₁₄ arylamino, mono- or di-C₇₋₁₆ aralkylamino, C₃₋₈ cycloalkyl, C₁₋₆ alkoxy, formyl, C₁₋₆ alkyl-carbonyl, C₃₋₈ cycloalkyl-carbonyl, C₆₋₁₄ aryl-carbonyl, C₇₋₁₆ aralkyl-carbonyl, C₁₋₆ alkoxy-carbonyl, C₆₋₁₄ aryloxy-carbonyl, C₇₋₁₆ aralkyloxy-carbonyl, C₁₋₆ alkylthio, C₁₋₆ alkylsulfinyl, C₁₋₆ alkylsulfonyl, carbamoyl, thiocarbamoyl, mono- or di-C₁₋₆ alkyl-carbamoyl, mono- or di-C₆₋₁₄ aryl-carbamoyl or the like)),
   (bb) a phenyl group, a benzyl group, a phenethyl group, a styryl group, a phenylethynyl group or a phenoxymethyl group, each optionally having 1 to 5 substituents selected from substituent group B,
   (cc) a 3 to 14-membered (monocyclic, bicyclic or tricyclic) heterocyclic group containing, besides carbon atom, 1 to 4 hetero atoms of 1 or 2 kinds selected from a nitrogen atom, a sulfur atom and an oxygen atom, which optionally has 1 to 5 substituents selected from substituent group B, and
   (dd) a group represented by the formula -Z¹-Z²
   wherein Z¹ is a bond, methylene (CH₂), ethylene (CH₂CH₂), vinylene (CHCH), ethynylene (CC) or methyleneoxy (CH₂O), and
   Z² is (i) a phenyl group optionally having substituents selected from the group consisting of (a) a halogen atom, (b) cyano, (c) an optionally halogenated C₁₋₆ alkyl group, (d) an optionally halogenated C₁₋₆ alkoxy group, (e) carbamoyl and (f) sulfamoyl, or
   (ii) a 5 to 10-membered monocyclic or bicyclic heterocyclic group containing, besides carbon atom, 1 to 4 hetero atoms of 1 or 2 kinds selected from a nitrogen atom, a sulfur atom and an oxygen atom, which optionally has substituents selected from the group consisting of (a) a halogen atom, (b) cyano, (c) an optionally halogenated C₁₋₆ alkyl group, (d) an optionally halogenated C₁₋₆ alkoxy group, (e) carbamoyl and (f) sulfamoyl;
(o) lower (C₁₋₆)alkylsulfonyl optionally substituted by 1 to 5 substituents selected from substituent group B, and
(p) C₆₋₁₄ arylsulfonyl optionally substituted by 1 to 5 substituents selected from substituent group B;
   (xxxxv) an amino group optionally substituted by 1 or 2 substituents selected from the group consisting of the following (a) to (q);
(a) lower (C₁₋₆)alkyl optionally substituted by 1 to 5 substituents selected from substituent group B,
(b) lower (C₂₋₆)alkenyl optionally substituted by 1 to 5 substituents selected from substituent group B,
(c) lower (C₂₋₆)alkynyl optionally substituted by 1 to 5 substituents selected from substituent group B,
(d) C₃₋₈ cycloalkyl optionally substituted by 1 to 5 substituents selected from substituent group B,
(e) C₆₋₁₄ aryl optionally substituted by 1 to 5 substituents selected from substituent group B,
(f) C₇₋₁₆ aralkyl optionally substituted by 1 to 5 substituents selected from substituent group B,
(g) lower (C₁₋₆)alkoxy optionally substituted by 1 to 5 substituents selected from substituent group B,
(h) formyl,
(i) lower (C₁₋₆)alkyl-carbonyl optionally substituted by 1 to 5 substituents selected from substituent group B,
(j) C₃₋₈ cycloalkyl-carbonyl optionally substituted by 1 to 5 substituents selected from substituent group B,
(k) C₆₋₁₄ aryl-carbonyl optionally substituted by 1 to 5 substituents selected from substituent group B,
(l) C₇₋₁₆ aralkyl-carbonyl optionally substituted by 1 to 5 substituents selected from substituent group B,
(m) 3 to 14-membered (monocyclic, bicyclic or tricyclic) heterocycle-carbonyl containing, besides carbon atom, 1 to 4 hetero atoms of 1 or 2 kinds selected from a nitrogen atom, a sulfur atom and an oxygen atom, which is optionally substituted by 1 to 5 substituents selected from the group consisting of the following (aa) to (dd);
   (aa) a substituent selected from substituent group B (except a phenyl group and a 5 to 10-membered heterocyclic group containing, besides carbon atom, 1 to 4 hetero atoms of 1 or 2 kinds selected from a nitrogen atom, a sulfur atom and an oxygen atom (wherein said heterocyclic group is optionally substituted by halogen atom, hydroxy, amino, mono- or di-C₁₋₆ alkylamino, mono- or di-C₆₋₁₄ arylamino, mono- or di-C₇₋₁₆ aralkylamino, C₃₋₈ cycloalkyl, C₁₋₆ alkoxy, formyl, C₁₋₆ alkyl-carbonyl, C₃₋₈ cycloalkyl-carbonyl, C₆₋₁₄ aryl-carbonyl, C₇₋₁₆ aralkyl-carbonyl, C₁₋₆ alkoxy-carbonyl, C₆₋₁₄ aryloxy-carbonyl, C₇₋₁₆ aralkyloxy-carbonyl, C₁₋₆ alkylthio, C₁₋₆ alkylsulfinyl, C₁₋₆ alkylsulfonyl, carbamoyl, thiocarbamoyl, mono- or di-C₁₋₆ alkyl-carbamoyl, mono- or di-C₆₋₁₄ aryl-carbamoyl or the like)),
   (bb) a phenyl group, a benzyl group, a phenethyl group, a styryl group, a phenylethynyl group or a phenoxymethyl group, each optionally having 1 to 5 substituents selected from substituent group B,
   (cc) a 3 to 14-membered (monocyclic, bicyclic or tricyclic) heterocyclic group containing, besides carbon atom, 1 to 4 hetero atoms of 1 or 2 kinds selected from a nitrogen atom, a sulfur atom and an oxygen atom, which optionally has 1 to 5 substituents selected from substituent group B, and
   (dd) a group represented by the formula -Z¹-Z²
   wherein Z¹ is a bond, methylene (CH₂), ethylene (CH₂CH₂), vinylene (CHCH), ethynylene (CC) or methyleneoxy (CH₂O), and
   Z² is (i) a phenyl group optionally having substituents selected from the group consisting of (a) a halogen atom, (b) cyano, (c) an optionally halogenated C₁₋₆ alkyl group, (d) an optionally halogenated C₁₋₆ alkoxy group, (e) carbamoyl and (f) sulfamoyl, or
   (ii) a 5 to 10-membered monocyclic or bicyclic heterocyclic group containing, besides carbon atom, 1 to 4 hetero atoms of 1 or 2 kinds selected from a nitrogen atom, a sulfur atom and an oxygen atom, which optionally has substituents selected from the group consisting of (a) a halogen atom, (b) cyano, (c) an optionally halogenated C₁₋₆ alkyl group, (d) an optionally halogenated C₁₋₆ alkoxy group, (e) carbamoyl and (f) sulfamoyl;
(n) C₁₋₆ alkoxy-carbonyl optionally substituted by 1 to 5 substituents selected from substituent group B,
(o) lower (C₁₋₆)alkylsulfonyl optionally substituted by 1 to 5 substituents selected from substituent group B,
(p) C₆₋₁₄ arylsulfonyl optionally substituted by 1 to 5 substituents selected from substituent group B, and
(q) a carbamoyl group or a 5 to 7-membered cyclic carbamoyl group optionally having 1 or 2 substituents selected from the group consisting of the following (aa) to (pp);
   (aa) lower (C₁₋₆)alkyl optionally substituted by 1 to 5 substituents selected from substituent group B (particularly, lower (C₁₋₆)alkyl optionally substituted by an “optionally substituted amino group” of substituent group B),
   (bb) lower (C₂₋₆)alkenyl optionally substituted by 1 to 5 substituents selected from substituent group B,
   (cc) lower (C₂₋₆)alkynyl optionally substituted by 1 to 5 substituents selected from substituent group B,
   (dd) C₃₋₈ cycloalkyl optionally substituted by 1 to 5 substituents selected from substituent group B,
   (ee) C₆₋₁₄ aryl optionally substituted by 1 to 5 substituents selected from substituent group B,
   (ff) C₇₋₁₆ aralkyl optionally substituted by 1 to 5 substituents selected from substituent group B,
   (gg) a 3 to 14-membered (monocyclic, bicyclic or tricyclic) heterocyclic group containing, besides carbon atom, 1 to 4 hetero atoms of 1 or 2 kinds selected from a nitrogen atom, a sulfur atom and an oxygen atom, which optionally has 1 to 5 substituents selected from substituent group B,
   (hh) lower (C₁₋₆)alkoxy optionally substituted by 1 to 5 substituents selected from substituent group B,
   (ii) formyl,
   (jj) lower (C₁₋₆)alkyl-carbonyl optionally substituted by 1 to 5 substituents selected from substituent group B,
   (kk) C₃₋₈ cycloalkyl-carbonyl optionally substituted by 1 to 5 substituents selected from substituent group B,
   (ll) C₅₋₁₄ aryl-carbonyl optionally substituted by 1 to 5 substituents selected from substituent group B,
   (mm) C₇₋₁₆ aralkyl-carbonyl optionally substituted by 1 to 5 substituents selected from substituent group B,
   (nn) 3 to 14-membered (monocyclic, bicyclic or tricyclic) heterocycle-carbonyl containing, besides carbon atom, 1 to 4 hetero atoms of 1 or 2 kinds selected from a nitrogen atom, a sulfur atom and an oxygen atom, which is optionally substituted by 1 to 5 substituents selected from the group consisting of the following (aaa) to (ddd);
      (aaa) a substituent selected from substituent group B (except a phenyl group and a 5 to 10-membered heterocyclic group containing, besides carbon atom, 1 to 4 hetero atoms of 1 or 2 kinds selected from a nitrogen atom, a sulfur atom and an oxygen atom (wherein said heterocyclic group is optionally substituted by halogen atom, hydroxy, amino, mono- or di-C₁₋₆ alkylamino, mono- or di-C₆₋₁₄ arylamino, mono- or di-C₇₋₁₆ aralkylamino, C₃₋₈ cycloalkyl, C₁₋₆ alkoxy, formyl, C₁₋₆ alkyl-carbonyl, C₃₋₈ cycloalkyl-carbonyl, C₆₋₁₄ aryl-carbonyl, C₇₋₁₆ aralkyl-carbonyl, C₁₋₆ alkoxy-carbonyl, C₆₋₁₄ aryloxy-carbonyl, C₇₋₁₆ aralkyloxy-carbonyl, C₁₋₆ alkylthio, C₁₋₆ alkylsulfinyl, C₁₋₆ alkylsulfonyl, carbamoyl, thiocarbamoyl, mono- or di-C₁₋₆ alkyl-carbamoyl, mono- or di-C₆₋₁₄ aryl-carbamoyl or the like)),
      (bbb) a phenyl group, a benzyl group, a phenethyl group, a styryl group, a phenylethynyl group or a phenoxymethyl group, each optionally having 1 to 5 substituents selected from substituent group B,
      (ccc) a 3 to 14-membered (monocyclic, bicyclic or tricyclic) heterocyclic group containing, besides carbon atom, 1 to 4 hetero atoms of 1 or 2 kinds selected from a nitrogen atom, a sulfur atom and an oxygen atom, which optionally has 1 to 5 substituents selected from substituent group B, and
      (ddd) a group represented by the formula -Z¹-Z² wherein Z¹ is a bond, methylene (CH₂), ethylene (CH₂CH₂), vinylene (CHCH), ethynylene (CC) or methyleneoxy (CH₂O), and
      Z² is (i) a phenyl group optionally having substituents selected from the group consisting of (a) a halogen atom, (b) cyano, (c) an optionally halogenated C₁₋₆ alkyl group, (d) an optionally halogenated C₁₋₆ alkoxy group, (e) carbamoyl and (f) sulfamoyl, or
      (ii) a 5 to 10-membered monocyclic or bicyclic heterocyclic group containing, besides carbon atom, 1 to 4 hetero atoms of 1 or 2 kinds selected from a nitrogen atom, a sulfur atom and an oxygen atom, which optionally has substituents selected from the group consisting of (a) a halogen atom, (b) cyano, (c) an optionally halogenated C₁₋₆ alkyl group, (d) an optionally halogenated C₁₋₆ alkoxy group, (e) carbamoyl and (f) sulfamoyl;
   (oo) lower (C₁₋₆)alkylsulfonyl optionally substituted by 1 to 5 substituents selected from substituent group B, and
   (pp) C₆₋₁₄ arylsulfonyl optionally substituted by 1 to 5 substituents selected from substituent group B; and
      (xxxxvi) a group bonded to two or more (e.g., 2-3) substituents from these (i) to (xxxxv), substituent group B is a substituent group consisting of the following (i) to (xxxii):
      (i) a halogen atom;
      (ii) hydroxy;
      (iii) nitro;
      (iv) cyano;
      (v) C₁₋₆ alkyl (wherein said C₁₋₆ alkyl is optionally substituted by halogen atom, hydroxy, amino, mono- or di-C₁₋₆ alkylamino, mono- or di-C₆₋₁₄ arylamino, C₃₋₈ cycloalkyl, C₁₋₆ alkoxy, formyl, C₁₋₆ alkyl-carbonyl, C₃₋₈ cycloalkyl-carbonyl, C₆₋₁₄ aryl-carbonyl, C₇₋₁₆ aralkyl-carbonyl, C₁₋₆ alkoxy-carbonyl, C₆₋₁₄ aryloxy-carbonyl, C₇₋₁₆ aralkyloxy-carbonyl, C₁₋₆ alkylthio, C₁₋₆ alkylsulfinyl, C₁₋₆ alkylsulfonyl, carbamoyl, thiocarbamoyl, mono- or di-C₁₋₆ alkyl-carbamoyl, mono- or di-C₆₋₁₄ aryl-carbamoyl or the like);
      (vi) C₂₋₆ alkenyl (wherein said C₂₋₆ alkenyl is optionally substituted by halogen atom, hydroxy, amino, mono- or di-C₁₋₆ alkylamino, mono- or di-C₆₋₁₄ arylamino, C₃₋₈ cycloalkyl, C₁₋₆ alkoxy, formyl, C₁₋₆ alkyl-carbonyl, C₃₋₈ cycloalkyl-carbonyl, C₆₋₁₄ aryl-carbonyl, C₇₋₁₆ aralkyl-carbonyl, C₁₋₆ alkoxy-carbonyl, C₆₋₁₄ aryloxy-carbonyl, C₇₋₁₆ aralkyloxy-carbonyl, C₁₋₆ alkylthio, C₁₋₆ alkylsulfinyl, C₁₋₆ alkylsulfonyl, carbamoyl, thiocarbamoyl, mono- or di-C₁₋₆ alkyl-carbamoyl, mono- or di-C₆₋₁₄ aryl-carbamoyl or the like);
      (vii) C₂₋₆ alkynyl (wherein said C₂₋₆ alkynyl is optionally substituted by halogen atom, hydroxy, amino, mono- or di-C₁₋₆ alkylamino, mono- or di-C₆₋₁₄ arylamino, C₃₋₈ cycloalkyl, C₁₋₆ alkoxy, formyl, C₁₋₆ alkyl-carbonyl, C₃₋₈ cycloalkyl-carbonyl, C₆₋₁₄ aryl-carbonyl, C₇₋₁₆ aralkyl-carbonyl, C₁₋₆ alkoxy-carbonyl, C₆₋₁₄ aryloxy-carbonyl, C₇₋₁₆ aralkyloxy-carbonyl, C₁₋₆ alkylthio, C₁₋₆ alkylsulfinyl, C₁₋₆ alkylsulfonyl, carbamoyl, thiocarbamoyl, mono- or di-C₁₋₆ alkyl-carbamoyl, mono- or di-C₆₋₁₄ aryl-carbamoyl or the like);
      (viii) C₆₋₁₄ aryl (wherein said C₆₋₁₄ aryl is optionally substituted by halogen atom, hydroxy, amino, optionally halogenated C₁₋₆ alkyl, mono- or di-C₁₋₆ alkylamino, mono- or di-C₆₋₁₄ arylamino, C₃₋₈ cycloalkyl, C₁₋₆ alkoxy, formyl, C₁₋₆ alkyl-carbonyl, C₃₋₈ cycloalkyl-carbonyl, C₆₋₁₄ aryl-carbonyl, C₇₋₁₆ aralkyl-carbonyl, C₁₋₆ alkoxy-carbonyl, C₆₋₁₄ aryloxy-carbonyl, C₇₋₁₆ aralkyloxy-carbonyl, C₁₋₆ alkylthio, C₁₋₆ alkylsulfinyl, C₁₋₆ alkylsulfonyl, carbamoyl, thiocarbamoyl, mono- or di-C₁₋₆ alkyl-carbamoyl, mono- or di-C₆₋₁₄ aryl-carbamoyl or the like);
      (ix) C₆₋₁₄ aryloxy (wherein said C₆₋₁₄ aryloxy is optionally substituted by halogen atom, hydroxy, cyano, amino, optionally halogenated C₁₋₆ alkyl, mono- or di-C₁₋₆ alkylamino, mono- or di-C₆₋₁₄ arylamino, C₃₋₈ cycloalkyl, C₁₋₆ alkoxy, formyl, C₁₋₆ alkyl-carbonyl, C₃₋₈ cycloalkyl-carbonyl, C₆₋₁₄ aryl-carbonyl, C₇₋₁₆ aralkyl-carbonyl, C₁₋₆ alkoxy-carbonyl, C₆₋₁₄ aryloxy-carbonyl, C₇₋₁₆ aralkyloxy-carbonyl, C₁₋₆ alkylthio, C₁₋₆ alkylsulfinyl, C₁₋₆ alkylsulfonyl, carbamoyl, thiocarbamoyl, mono- or di-C₁₋₆ alkyl-carbamoyl, mono- or di-C₆₋₁₄ aryl-carbamoyl or the like);
      (x) C₇₋₁₆ aralkyloxy (wherein said C₇₋₁₆ aralkyloxy is optionally substituted by halogen atom, hydroxy, amino, optionally halogenated C₁₋₆ alkyl, mono- or di-C₁₋₆ alkylamino, mono- or di-C₆₋₁₄ arylamino, C₃₋₈ cycloalkyl, C₁₋₆ alkoxy, formyl, C₁₋₆ alkyl-carbonyl, C₃₋₈ cycloalkyl-carbonyl, C₆₋₁₄ aryl-carbonyl, C₇₋₁₆ aralkyl-carbonyl, C₁₋₆ alkoxy-carbonyl, C₆₋₁₄ aryloxy-carbonyl, C₇₋₁₆ aralkyloxy-carbonyl, C₁₋₆ alkylthio, C₁₋₆ alkylsulfinyl, C₁₋₆ alkylsulfonyl, carbamoyl, thiocarbamoyl, mono- or di-C₁₋₆ alkyl-carbamoyl, mono- or di-C₆₋₁₄ aryl-carbamoyl or the like);
      (xi) a 5 to 10-membered heterocyclic group containing, besides carbon atom, 1 to 4 hetero atoms of 1 or 2 kinds selected from a nitrogen atom, a sulfur atom and an oxygen atom (wherein said heterocyclic group is optionally substituted by halogen atom, hydroxy, amino, mono- or di-C₁₋₆ alkylamino, mono- or di-C₆₋₁₄ arylamino, mono- or di-C₇₋₁₆ aralkylamino, C₃₋₈ cycloalkyl, C₁₋₆ alkoxy, formyl, C₁₋₆ alkyl-carbonyl, C₃₋₈ cycloalkyl-carbonyl, C₆₋₁₄ aryl-carbonyl, C₇₋₁₆ aralkyl-carbonyl, C₁₋₆ alkoxy-carbonyl, C₆₋₁₄ aryloxy-carbonyl, C₇₋₁₆ aralkyloxy-carbonyl, C₁₋₆ alkylthio, C₁₋₆ alkylsulfinyl, C₁₋₆ alkylsulfonyl, carbamoyl, thiocarbamoyl, mono- or di-C₁₋₆ alkyl-carbamoyl, mono- or di-C₆₋₁₄ aryl-carbamoyl or the like);
      (xii) an amino group optionally substituted by 1 or 2 substituents selected from the group consisting of C₁₋₆ alkyl, C₂₋₆ alkenyl, C₆₋₁₄ aryl, C₇₋₁₆ aralkyl, a 3 to 14-membered (monocyclic, bicyclic or tricyclic) heterocyclic group containing, besides carbon atom, 1 to 4 hetero atoms of 1 or 2 kinds selected from a nitrogen atom, a sulfur atom and an oxygen atom and 3 to 14-membered (monocyclic, bicyclic or tricyclic) heterocycle-C₁₋₆ alkyl containing, besides carbon atom, 1 to 4 hetero atoms of 1 or 2 kinds selected from a nitrogen atom, a sulfur atom and an oxygen atom (wherein said C₁₋₆ alkyl, C₂₋₆ alkenyl, C₆₋₁₄ aryl, C₇₋₁₆ aralkyl, heterocyclic group and heterocycle-C₁₋₆ alkyl are each optionally substituted by halogen atom, hydroxy, cyano, amino, optionally halogenated C₁₋₆ alkyl, mono- or di-C₁₋₆ alkylamino, mono- or di-C₆₋₁₄ arylamino, C₃₋₈ cycloalkyl, C₁₋₆ alkoxy, formyl, C₁₋₆ alkyl-carbonyl, C₃₋₈ cycloalkyl-carbonyl, C₆₋₁₄ aryl-carbonyl, C₇₋₁₆ aralkyl-carbonyl, C₁₋₆ alkoxy-carbonyl, C₆₋₁₄ aryloxy-carbonyl, C₇₋₁₆ aralkyloxy-carbonyl, C₁₋₆ alkylthio, C₁₋₆ alkylsulfinyl, C₁₋₆ alkylsulfonyl, carbamoyl, thiocarbamoyl, mono- or di-C₁₋₆ alkyl-carbamoyl, mono- or di-C₆₋₁₄ aryl-carbamoyl or the like);
      (xiii) C₃₋₈ cycloalkyl;
      (xiv) C₁₋₆ alkoxy (wherein said C₁₋₆ alkoxy is optionally substituted by halogen atom, hydroxy, amino, mono- or di-C₁₋₆ alkylamino, mono- or di-C₆₋₁₄ arylamino, C₃₋₈ cycloalkyl, C₁₋₆ alkoxy, formyl, C₁₋₆ alkyl-carbonyl, C₃₋₈ cycloalkyl-carbonyl, C₆₋₁₄ aryl-carbonyl, C₇₋₁₆ aralkyl-carbonyl, C₁₋₆ alkoxy-carbonyl, C₆₋₁₄ aryloxy-carbonyl, C₇₋₁₆ aralkyloxy-carbonyl, C₁₋₆ alkylthio, C₁₋₆ alkylsulfinyl, C₁₋₆ alkylsulfonyl, carbamoyl, thiocarbamoyl, mono- or di-C₁₋₆ alkyl-carbamoyl, mono- or di-C₆₋₁₄ aryl-carbamoyl or the like);
      (xv) formyl;
      (xvi) C₁₋₆ alkyl-carbonyl;
      (xvii) C₃₋₈ cycloalkyl-carbonyl;
      (xviii) C₆₋₁₄ aryl-carbonyl;
      (xix) C₇₋₁₆ aralkyl-carbonyl;
      (xx) C₁₋₆ alkoxy-carbonyl;
      (xxi) C₆₋₁₄ aryloxy-carbonyl;
      (xxii) C₇₋₁₆ aralkyloxy-carbonyl;
      (xxiii) C₁₋₆ alkylthio;
      (xxiv) C₁₋₆ alkylsulfinyl;
      (xxv) C₁₋₆ alkylsulfonyl;
      (xxvi) carbamoyl;
      (xxvii) thiocarbamoyl;
      (xxviii) mono-C₁₋₆ alkyl-carbamoyl;
      (xxix) di-C₁₋₆ alkyl-carbamoyl;
      (xxx) mono- or di-C₆₋₁₄ aryl-carbamoyl;
      (xxxi) mono- or di-5 to 7-membered heterocycle-carbamoyl containing, besides carbon atom, 1 to 4 hetero atoms of 1 or 2 kinds selected from a nitrogen atom, a sulfur atom and an oxygen atom, and
      (xxxii) sulfamoyl.
      R¹, R² and R³ are each
      (i) a hydrogen atom,
      (ii) a linear or branched C₁₋₁₅ alkyl group, a C₃₋₈ cycloalkyl group, a C₂₋₁₈ alkenyl group, a C₃₋₁₀ cycloalkenyl group, a C₂₋₈ alkynyl group, a C₆₋₁₄ aryl group, a biphenyl group, a tolyl group or a C₇₋₁₆ aralkyl group, each optionally having 1 to 5 substituents selected from substituent group B, or
      (iii) a 3 to 14-membered (monocyclic, bicyclic or tricyclic) heterocyclic group containing, besides carbon atom, 1 to 4 hetero atoms of 1 or 2 kinds selected from a nitrogen atom, a sulfur atom and an oxygen atom, which optionally has 1 to 5 substituents selected from substituent group B, provided that R² is not a 4-hydroxyphenyl group optionally having substituents selected from the group consisting of a halogen atom, a C₁₋₆ alkyl group and a C₁₋₆ alkoxy group; a 4-methoxyphenyl group optionally having substituents selected from the group consisting of a halogen atom, a C₁₋₆ alkyl group and a C₁₋₆ alkoxy group; or a 6-hydroxypyridin-3-yl group optionally having substituents selected from the group consisting of a halogen atom, a C₁₋₆ alkyl group and a C₁₋₆ alkoxy group.
      R¹ and R² optionally form a 5- to 8-membered ring via X, and when R¹ and R² form a ring via X, R¹ and R² are each
      (i) a bond or
      (ii) a divalent C₁₋₅ acyclic hydrocarbon group optionally having substituents selected from substituent group B, and
      X is
      (i) a bond,
      (ii) an oxygen atom,
      (iii) an optionally oxidized sulfur atom, or
      (iv) an imino group optionally having a substituent selected
      from the group consisting of the following (a) to (o).
(a) lower (C₁₋₆)alkyl optionally substituted by 1 to 5 substituents selected from substituent group B;
(b) lower (C₂₋₆)alkenyl optionally substituted by 1 to 5 substituents selected from substituent group B;
(c) lower (C₂₋₆)alkynyl optionally substituted by 1 to 5 substituents selected from substituent group B;
(d) C₃₋₈ cycloalkyl optionally substituted by 1 to 5 substituents selected from substituent group B;
(e) C₆₋₁₄ aryl optionally substituted by 1 to 5 substituents selected from substituent group B;
(f) C₇₋₁₆ aralkyl optionally substituted by 1 to 5 substituents selected from substituent group B;
(g) lower (C₁₋₆)alkoxy optionally substituted by 1 to 5 substituents selected from substituent group B;
(h) lower (C₁₋₆)alkyl-carbonyl optionally substituted by 1 to 5 substituents selected from substituent group B;
(i) C₃₋₈ cycloalkyl-carbonyl optionally substituted by 1 to 5 substituents selected from substituent group B;
(j) C₆₋₁₄ aryl-carbonyl optionally substituted by 1 to 5 substituents selected from substituent group B;
(k) C₇₋₁₆ aralkyl-carbonyl optionally substituted by 1 to 5 substituents selected from substituent group B;
(l) a 3 to 14-membered (monocyclic, bicyclic or tricyclic) heterocycle-carbonyl containing, besides carbon atom, 1 to 4 hetero atoms of 1 or 2 kinds selected from a nitrogen atom, a sulfur atom and an oxygen atom, which is optionally substituted by 1 to 5 substituents selected from the group consisting of the following (aa) to (dd);
   (aa) a substituent selected from substituent group B (except a phenyl group and a 5 to 10-membered heterocyclic group containing, besides carbon atom, 1 to 4 hetero atoms of 1 or 2 kinds selected from a nitrogen atom, a sulfur atom and an oxygen atom (wherein said heterocyclic group is optionally substituted by halogen atom, hydroxy, amino, mono- or di-C₁₋₆ alkylamino, mono- or di-C₆₋₁₄ arylamino, mono- or di-C₇₋₁₆ aralkylamino, C₃₋₈ cycloalkyl, C₁₋₆ alkoxy, formyl, C₁₋₆ alkyl-carbonyl, C₃₋₈ cycloalkyl-carbonyl, C₆₋₁₄ aryl-carbonyl, C₇₋₁₆ aralkyl-carbonyl, C₁₋₆ alkoxy-carbonyl, C₆₋₁₄ aryloxy-carbonyl, C₇₋₁₆ aralkyloxy-carbonyl, C₁₋₆ alkylthio, C₁₋₆ alkylsulfinyl, C₁₋₆ alkylsulfonyl, carbamoyl, thiocarbamoyl, mono- or di-C₁₋₆ alkyl-carbamoyl, mono- or di-C₆₋₁₄ aryl-carbamoyl or the like)),
   (bb) a phenyl group, a benzyl group, a phenethyl group, a styryl group, a phenylethynyl group or a phenoxymethyl group, each optionally having 1 to 5 substituents selected from substituent group B,
   (cc) a 3 to 14-membered (monocyclic, bicyclic or tricyclic) heterocyclic group containing, besides carbon atom, 1 to 4 hetero atoms of 1 or 2 kinds selected from a nitrogen atom, a sulfur atom and an oxygen atom, which optionally has 1 to 5 substituents selected from substituent group B, and
   (dd) a group represented by the formula -Z¹-Z²
   wherein Z¹ is a bond, methylene (CH₂), ethylene (CH₂CH₂), vinylene (CHCH), ethynylene (CC) or methyleneoxy (CH₂O), Z² is (i) a phenyl group optionally having substituents selected from the group consisting of (a) a halogen atom, (b) cyano, (c) an optionally halogenated C₁₋₆ alkyl group, (d) an optionally halogenated C₁₋₆ alkoxy group, (e) carbamoyl and (f) sulfamoyl, or
   (ii) a 5 to 10-membered monocyclic or bicyclic heterocyclic group containing, besides carbon atom, 1 to 4 hetero atoms of 1 or 2 kinds selected from a nitrogen atom, a sulfur atom and an oxygen atom, which optionally has substituents selected from the group consisting of (a) a halogen atom, (b) cyano, (c) an optionally halogenated C₁₋₆ alkyl group, (d) an optionally halogenated C₁₋₆ alkoxy group, (e) carbamoyl and (f) sulfamoyl;
(m) lower (C₁₋₆)alkylsulfonyl optionally substituted by 1 to 5 substituents selected from substituent group B;
(n) C₆₋₁₄ arylsulfonyl optionally substituted by 1 to 5 substituents selected from substituent group B; and
(o) a 3 to 14-membered (monocyclic, bicyclic or tricyclic) heterocyclic group containing, besides carbon atom, 1 to 4 hetero atoms of 1 or 2 kinds selected from a nitrogen atom, a sulfur atom and an oxygen atom, which optionally has 1 to 5 substituents selected from substituent group B,
   provided that R¹, R² and X are not bonds at the same time,
   [58] the compound of the above-mentioned [2], wherein R¹, and R²′ are each
   (i) a bond or
   (ii) a divalent C₁₋₅ acyclic hydrocarbon group optionally having substituents selected from substituent group B, and A, R³ and X are as defined in the above-mentioned [57], provided that R¹′, R²′ and X are not bonds at the same time,
   [59] the compound of the above-mentioned [3], wherein R¹″ and R²″ are each
   (i) a hydrogen atom,
   (ii) a linear or branched C₁₋₁₅ alkyl group, a C₃₋₈ cycloalkyl group, a C₂₋₁₈ alkenyl group, a C₃₋₁₀ cycloalkenyl group, a C₂₋₈ alkynyl group, a C₆₋₁₄ aryl group, a biphenyl group, a tolyl group or a C₇₋₁₆ aralkyl group, each optionally having 1 to 5 substituents selected from substituent group B, or
   (iii) a 3 to 14-membered (monocyclic, bicyclic or tricyclic) heterocyclic group containing, besides carbon atom, 1 to 4 hetero atoms of 1 or 2 kinds selected from a nitrogen atom, a sulfur atom and an oxygen atom, which optionally has 1 to 5 substituents selected from substituent group B,
   A and R³ are as defined in the above-mentioned [57], provided that R²″ is not a 4-hydroxyphenyl group optionally having substituents selected from the group consisting of a halogen atom, a C₁₋₆ alkyl group and a C₁₋₆ alkoxy group; a 4-methoxyphenyl group optionally having substituents selected from the group consisting of a halogen atom, a C₁₋₆ alkyl group and a C₁₋₆ alkoxy group; and a 6-hydroxypyridin-3-yl group optionally having substituents selected from the group consisting of a halogen atom, a C₁₋₆ alkyl group and a C₁₋₆ alkoxy group,
   [60] the compound of the above-mentioned [4], wherein A′ is a benzene ring optionally having substituents described in the above-mentioned [57],
   Y¹ and Y² are each
   (i) a bond,
   (ii) an oxygen atom,
   (iii) an optionally oxidized sulfur atom,
   (iv) an imino group optionally having a substituent described in the above-mentioned [57], or
   (v) a carbonyl group,
   Z is
   (i) a hydrogen atom,
   (ii) a linear or branched C₁₋₁₅ alkyl group, a C₃₋₈ cycloalkyl group, a C₂₋₁₈ alkenyl group, a C₃₋₁₀ cycloalkenyl group, a C₂₋₈ alkynyl group, a C₆₋₁₄ aryl group, a biphenyl group, a tolyl group or a C₇₋₁₆ aralkyl group, each optionally having 1 to 5 substituents selected from substituent group B, or
   (iii) a 3 to 14-membered (monocyclic, bicyclic or tricyclic) heterocyclic group containing, besides carbon atom, 1 to 4 hetero atoms of 1 or 2 kinds selected from a nitrogen atom, a sulfur atom and an oxygen atom, which optionally has 1 to 5 substituents selected from substituent group B, and
   R¹″, R²″ and R³ are as defined in the above-mentioned [59],
   [61] the compound of the above-mentioned [9], wherein Z¹ is a bond, methylene (CH₂), ethylene (CH₂CH₂), vinylene (CHCH) or ethynylene (CC),
   Z² is
   (i) a phenyl group optionally having 1 to 5 substituents selected from substituent group B or
   (ii) a 3 to 14-membered (monocyclic, bicyclic or tricyclic) heterocyclic group containing, besides carbon atom, 1 to 4 hetero atoms of 1 or 2 kinds selected from a nitrogen atom, a sulfur atom and an oxygen atom, which optionally has 1 to 5 substituents selected from substituent group B,
   A″ is a benzene ring optionally further having substituents selected from substituent group A,
   R³ is
   (i) a hydrogen atom,
   (ii) a linear or branched C₁₋₁₅ alkyl group, a C₃₋₈ cycloalkyl group, a C₂₋₁₈ alkenyl group, a C₃₋₁₀ cycloalkenyl group, a C₂₋₈ alkynyl group, a C₆₋₁₄ aryl group, a biphenyl group, a tolyl group or a C₇₋₁₆ aralkyl group, each optionally having 1 to 5 substituents selected from substituent group B, or
   (iii) a 3 to 14-membered (monocyclic, bicyclic or tricyclic) heterocyclic group containing, besides carbon atom, 1 to 4 hetero atoms of 1 or 2 kinds selected from a nitrogen atom, a sulfur atom and an oxygen atom, which optionally has 1 to 5 substituents selected from substituent group B, and
   Q is a piperidine ring optionally further having, besides -Z¹-Z², substituents selected from substituent group A,
   [62] the compound of the above-mentioned [33], wherein A is a benzene ring optionally having substituents selected from substituent group A,
   substituent group A is a substituent group consisting of the following (i) to (xxxxvi)
   (i) a halogen atom;
   (ii) C₁₋₃ alkylenedioxy;
   (iii) nitro;
   (iv) cyano;
   (v) optionally esterified carboxyl selected from
(a) carboxyl,
(b) C₁₋₆ alkoxy-carbonyl optionally substituted by 1 to 5 substituents selected from substituent group B,
(c) C₆₋₁₄ aryloxy-carbonyl optionally substituted by 1 to 5 substituents selected from substituent group B and
(d) C₇₋₁₆ aralkyloxy-carbonyl optionally substituted by 1 to substituents selected from substituent group B;
   (vi) lower (C₁₋₆)alkyl optionally substituted by 1 to 5 substituents selected from substituent group B;
   (vii) lower (C₂₋₆)alkenyl optionally substituted by 1 to 5 substituents selected from substituent group B;
   (viii) lower (C₂₋₆)alkynyl optionally substituted by 1 to 5 substituents selected from substituent group B;
   (ix) C₃₋₈ cycloalkyl optionally substituted by 1 to 5 substituents selected from substituent group B;
   (x) C₆₋₁₄ aryl optionally substituted by 1 to 5 substituents selected from substituent group B;
   (xi) C₇₋₁₆ aralkyl optionally substituted by 1 to 5 substituents selected from substituent group B;
   (xii) C₆₋₁₄ aryl-C₂₋₆ alkenyl optionally substituted by 1 to 5 substituents selected from substituent group B;
   (xiii) a 3 to 14-membered (monocyclic, bicyclic or tricyclic) heterocyclic group containing, besides carbon atom, 1 to 4 hetero atoms of 1 or 2 kinds selected from a nitrogen atom, a sulfur atom and an oxygen atom, which optionally has 1 to 5 substituents selected from substituent group B;
   (xiv) hydroxy;
   (xv) lower (C₁₋₆)alkoxy optionally substituted by 1 to 5 substituents selected from substituent group B;
   (xvi) C₆₋₁₄ aryloxy optionally substituted by 1 to 5 substituents selected from substituent group B;
   (xvii) C₇₋₁₆ aralkyloxy optionally substituted by 1 to 5 substituents selected from substituent group B;
   (xviii) lower (C₁₋₆)alkyl-carbonyloxy optionally substituted by 1 to 5 substituents selected from substituent group B;
   (xix) lower (C₁₋₆)alkoxy-carbonyloxy optionally substituted by 1 to 5 substituents selected from substituent group B;
   (xx) mono-lower (C₁₋₆)alkyl-carbamoyloxy optionally substituted by 1 to 5 substituents selected from substituent group B;
   (xxi) di-lower (C₁₋₆)alkyl-carbamoyloxy optionally substituted by 1 to 5 substituents selected from substituent group B;
   (xxii) C₆₋₁₄ aryl-carbonyloxy optionally substituted by 1 to 5 substituents selected from substituent group B;
   (xxiii) mono- or di-C₆₋₁₄ aryl-carbamoyloxy optionally substituted by 1 to 5 substituents selected from substituent group B;
   (xxiv) 3 to 14-membered (monocyclic, bicyclic or tricyclic) heterocycle-oxy containing, besides carbon atom, 1 to 4 hetero atoms of 1 or 2 kinds selected from a nitrogen atom, a sulfur atom and an oxygen atom, which is optionally substituted by 1 to 5 substituents selected from substituent group B (preferably 5 to 10-membered aromatic heterocycle-oxy containing, besides carbon atom, 1 to 4 hetero atoms of 1 or 2 kinds selected from a nitrogen atom, a sulfur atom and an oxygen atom, which is optionally substituted by 1 to 5 substituents selected from substituent group B);
   (xxv) mercapto;
   (xxvi) lower (C₁₋₆)alkylthio optionally substituted by 1 to 5 substituents selected from substituent group B;
   (xxvii) C₆₋₁₄ arylthio optionally substituted by 1 to 5 substituents selected from substituent group B;
   (xxviii) C₇₋₁₆ aralkylthio optionally substituted by 1 to 5 substituents selected from substituent group B;
   (xxix) formyl;
   (xxx) lower (C₁₋₆)alkyl-carbonyl optionally substituted by 1 to 5 substituents selected from substituent group B;
   (xxxi) C₃₋₈ cycloalkyl-carbonyl optionally substituted by 1 to 5 substituents selected from substituent group B;
   (xxxii) C₆₋₁₄ aryl-carbonyl optionally substituted by 1 to 5 substituents selected from substituent group B;
   (xxxiii) C₇₋₁₆ aralkyl-carbonyl optionally substituted by 1 to 5 substituents selected from substituent group B;
   (xxxiv) 3 to 14-membered (monocyclic, bicyclic or tricyclic) heterocycle-carbonyl containing, besides carbon atom, 1 to 4 hetero atoms of 1 or 2 kinds selected from a nitrogen atom, a sulfur atom and an oxygen atom, which is optionally substituted by 1 to 5 substituents selected from the group consisting of the following (a) to (d);
(a) a substituent selected from substituent group B (except a phenyl group and a 5 to 10-membered heterocyclic group containing, besides carbon atom, 1 to 4 hetero atoms of 1 or 2 kinds selected from a nitrogen atom, a sulfur atom and an oxygen atom (wherein said heterocyclic group is optionally substituted by halogen atom, hydroxy, amino, mono- or di-C₁₋₆ alkylamino, mono- or di-C₆₋₁₄ arylamino, mono- or di-C₇₋₁₆ aralkylamino, C₃₋₈ cycloalkyl, C₁₋₆ alkoxy, formyl, C₁₋₅ alkyl-carbonyl, C₃₋₈ cycloalkyl-carbonyl, C₆₋₁₄ aryl-carbonyl, C₇₋₁₆ aralkyl-carbonyl, C₁₋₆ alkoxy-carbonyl, C₆₋₁₄ aryloxy-carbonyl, C₇₋₁₆ aralkyloxy-carbonyl, C₁₋₆ alkylthio, C₁₋₆ alkylsulfinyl, C₁₋₆ alkylsulfonyl, carbamoyl, thiocarbamoyl, mono- or di-C₁₋₆ alkyl-carbamoyl, mono- or di-C₆₋₁₄ aryl-carbamoyl or the like)),
(b) a phenyl group, a benzyl group, a phenethyl group, a styryl group, a phenylethynyl group or a phenoxymethyl group, each optionally having 1 to 5 substituents selected from substituent group B,
(c) a 3 to 14-membered (monocyclic, bicyclic or tricyclic) heterocyclic group containing, besides carbon atom, 1 to 4 hetero atoms of 1 or 2 kinds selected from a nitrogen atom, a sulfur atom and an oxygen atom, which optionally has 1 to 5 substituents selected from substituent group B; and
(d) a group represented by the formula -Z¹-Z²
wherein Z¹ is a bond, methylene (CH₂), ethylene (CH₂CH₂), vinylene (CHCH), ethynylene (CC) or methyleneoxy (CH₂O), and
Z² is (i) a phenyl group optionally having substituents selected from the group consisting of (a) a halogen atom, (b) cyano, (c) an optionally halogenated C₁₋₆ alkyl group, (d) an optionally halogenated C₁₋₆ alkoxy group, (e) carbamoyl and (f) sulfamoyl, or
   (ii) a 5 to 10-membered monocyclic or bicyclic heterocyclic group containing, besides carbon atom, 1 to 4 hetero atoms of 1 or 2 kinds selected from a nitrogen atom, a sulfur atom and an oxygen atom, which optionally has substituents selected from the group consisting of (a) a halogen atom, (b) cyano, (c) an optionally halogenated C₁₋₆ alkyl group, (d) an optionally halogenated C₁₋₆ alkoxy group, (e) carbamoyl and (f) sulfamoyl;
      (xxxv) lower (C₁₋₆)alkylsulfonyl optionally substituted by 1 to 5 substituents selected from substituent group B;
      (xxxvi) C₆₋₁₄ arylsulfonyl optionally substituted by 1 to 5 substituents selected from substituent group B;
      (xxxvii) lower (C₁₋₆)alkylsulfinyl optionally substituted by 1 to 5 substituents selected from substituent group B;
      (xxxviii) C₆₋₁₄ arylsulfinyl optionally substituted by 1 to 5 substituents selected from substituent group B;
      (xxxix) sulfo;
      (xxxx) sulfamoyl;
      (xxxxi) sulfinamoyl;
      (xxxxii) sulfenamoyl;
      (xxxxiii) thiocarbamoyl;
      (xxxxiv) a carbamoyl group or a 5 to 7-membered cyclic carbamoyl group optionally having 1 or 2 substituents selected from the group consisting of the following (a) to (p);
(a) lower (C₁₋₆)alkyl optionally substituted by 1 to 5 substituents selected from substituent group B (particularly, lower (C₁₋₆)alkyl optionally substituted by the “optionally substituted amino group” or the “5 to 10-membered heterocyclic group containing, besides carbon atom, 1 to 4 hetero atoms of 1 or 2 kinds selected from a nitrogen atom, a sulfur atom and an oxygen atom” of substituent group B),
(b) lower (C₂₋₆)alkenyl optionally substituted by 1 to 5 substituents selected from substituent group B,
(c) lower (C₂₋₆)alkynyl optionally substituted by 1 to 5 substituents selected from substituent group B,
(d) C₃₋₈ cycloalkyl optionally substituted by 1 to 5 substituents selected from substituent group B,
(e) C₆₋₁₄ aryl optionally substituted by 1 to 5 substituents selected from substituent group B,
(f) C₇₋₁₆ aralkyl optionally substituted by 1 to 5 substituents selected from substituent group B,
(g) a 3 to 14-membered (monocyclic, bicyclic or tricyclic) heterocyclic group containing, besides carbon atom, 1 to 4 hetero atoms of 1 or 2 kinds selected from a nitrogen atom, a sulfur atom and an oxygen atom, which optionally has 1 to 5 substituents selected from substituent group B,
(h) lower (C₁₋₆)alkoxy optionally substituted by 1 to 5 substituents selected from substituent group B,
(i) formyl,
(j) lower (C₁₋₆)alkyl-carbonyl optionally substituted by 1 to 5 substituents selected from substituent group B,
(k) C₃₋₈ cycloalkyl-carbonyl optionally substituted by 1 to 5 substituents selected from substituent group B,
(l) C₆₋₁₄ aryl-carbonyl optionally substituted by 1 to 5 substituents selected from substituent group B,
(m) C₇₋₁₆ aralkyl-carbonyl optionally substituted by 1 to 5 substituents selected from substituent group B,
(n) 3 to 14-membered (monocyclic, bicyclic or tricyclic) heterocycle-carbonyl containing, besides carbon atom, 1 to 4 hetero atoms of 1 or 2 kinds selected from a nitrogen atom, a sulfur atom and an oxygen atom, which is optionally substituted by 1 to 5 substituents selected from the group consisting of the following (aa) to (dd);
   (aa) a substituent selected from substituent group B (except a phenyl group and a 5 to 10-membered heterocyclic group containing, besides carbon atom, 1 to 4 hetero atoms of 1 or 2 kinds selected from a nitrogen atom, a sulfur atom and an oxygen atom (wherein said heterocyclic group is optionally substituted by halogen atom, hydroxy, amino, mono- or di-C₁₋₆ alkylamino, mono- or di-C₆₋₁₄ arylamino, mono- or di-C₇₋₁₆ aralkylamino, C₃₋₈ cycloalkyl, C₁₋₆ alkoxy, formyl, C₁₋₆ alkyl-carbonyl, C₃₋₈ cycloalkyl-carbonyl, C₆₋₁₄ aryl-carbonyl, C₇₋₁₆ aralkyl-carbonyl, C₁₋₆ alkoxy-carbonyl, C₆₋₁₄ aryloxy-carbonyl, C₇₋₁₆ aralkyloxy-carbonyl, C₁₋₆ alkylthio, C₁₋₆ alkylsulfinyl, C₁₋₆ alkylsulfonyl, carbamoyl, thiocarbamoyl, mono- or di-C₁₋₆ alkyl-carbamoyl, mono- or di-C₆₋₁₄ aryl-carbamoyl or the like)),
   (bb) a phenyl group, a benzyl group, a phenethyl group, a styryl group, a phenylethynyl group or a phenoxymethyl group, each optionally having 1 to 5 substituents selected from substituent group B,
   (cc) a 3 to 14-membered (monocyclic, bicyclic or tricyclic) heterocyclic group containing, besides carbon atom, 1 to 4 hetero atoms of 1 or 2 kinds selected from a nitrogen atom, a sulfur atom and an oxygen atom, which optionally has 1 to 5 substituents selected from substituent group B; and
   (dd) a group represented by the formula -Z¹-Z²
   wherein Z¹ is a bond, methylene (CH₂), ethylene (CH₂CH₂), vinylene (CHCH), ethynylene (CC) or methyleneoxy (CH₂O), and
   Z² is (i) a phenyl group optionally having substituents selected from the group consisting of (a) a halogen atom, (b) cyano, (c) an optionally halogenated C₁₋₆ alkyl group, (d) an optionally halogenated C₁₋₆ alkoxy group, (e) carbamoyl and (f) sulfamoyl, or
   (ii) a 5 to 10-membered monocyclic or bicyclic heterocyclic group containing, besides carbon atom, 1 to 4 hetero atoms of 1 or 2 kinds selected from a nitrogen atom, a sulfur atom and an oxygen atom, which optionally has substituents selected from the group consisting of (a) a halogen atom, (b) cyano, (c) an optionally halogenated C₁₋₆ alkyl group, (d) an optionally halogenated C₁₋₆ alkoxy group, (e) carbamoyl and (f) sulfamoyl;
(o) lower (C₁₋₆)alkylsulfonyl optionally substituted by 1 to 5 substituents selected from substituent group B, and
(p) C₆₋₁₄ arylsulfonyl optionally substituted by 1 to 5 substituents selected from substituent group B;
   (xxxxv) an amino group optionally substituted by 1 or 2 substituents selected from the group consisting of the following (a) to (q);
(a) lower (C₁₋₆)alkyl optionally substituted by 1 to 5 substituents selected from substituent group B,
(b) lower (C₂₋₆)alkenyl optionally substituted by 1 to 5 substituents selected from substituent group B,
(c) lower (C₂₋₆)alkynyl optionally substituted by 1 to 5 substituents selected from substituent group B,
(d) C₃₋₈ cycloalkyl optionally substituted by 1 to 5 substituents selected from substituent group B,
(e) C₆₋₁₄ aryl optionally substituted by 1 to 5 substituents selected from substituent group B,
(f) C₇₋₁₆ aralkyl optionally substituted by 1 to 5 substituents selected from substituent group B,
(g) lower (C₁₋₆)alkoxy optionally substituted by 1 to 5 substituents selected from substituent group B,
(h) formyl,
(i) lower (C₁₋₆)alkyl-carbonyl optionally substituted by 1 to 5 substituents selected from substituent group B,
(j) C₃₋₈ cycloalkyl-carbonyl optionally substituted by 1 to 5 substituents selected from substituent group B,
(k) C₆₋₁₄ aryl-carbonyl optionally substituted by 1 to 5 substituents selected from substituent group B,
(l) C₇₋₁₆ aralkyl-carbonyl optionally substituted by 1 to 5 substituents selected from substituent group B,
(m) 3 to 14-membered (monocyclic, bicyclic or tricyclic) heterocycle-carbonyl containing, besides carbon atom, 1 to 4 hetero atoms of 1 or 2 kinds selected from a nitrogen atom, a sulfur atom and an oxygen atom, which is optionally substituted by 1 to 5 substituents selected from the group consisting of the following (aa) to (dd);
   (aa) a substituent selected from substituent group B (except a phenyl group and a 5 to 10-membered heterocyclic group containing, besides carbon atom, 1 to 4 hetero atoms of 1 or 2 kinds selected from a nitrogen atom, a sulfur atom and an oxygen atom (wherein said heterocyclic group is optionally substituted by halogen atom, hydroxy, amino, mono- or di-C₁₋₆ alkylamino, mono- or di-C₆₋₁₄ arylamino, mono- or di-C₇₋₁₆ aralkylamino, C₃₋₈ cycloalkyl, C₁₋₆ alkoxy, formyl, C₁₋₆ alkyl-carbonyl, C₃₋₈ cycloalkyl-carbonyl, C₆₋₁₄ aryl-carbonyl, C₇₋₁₆ aralkyl-carbonyl, C₁₋₆ alkoxy-carbonyl, C₆₋₁₄ aryloxy-carbonyl, C₇₋₁₆ aralkyloxy-carbonyl, C₁₋₆ alkylthio, C₁₋₆ alkylsulfinyl, C₁₋₆ alkylsulfonyl, carbamoyl, thiocarbamoyl, mono- or di-C₁₋₆ alkyl-carbamoyl, mono- or di-C₆₋₁₄ aryl-carbamoyl or the like)),
   (bb) a phenyl group, a benzyl group, a phenethyl group, a styryl group, a phenylethynyl group or a phenoxymethyl group, each optionally having 1 to 5 substituents selected from substituent group B,
   (cc) a 3 to 14-membered (monocyclic, bicyclic or tricyclic) heterocyclic group containing, besides carbon atom, 1 to 4 hetero atoms of 1 or 2 kinds selected from a nitrogen atom, a sulfur atom and an oxygen atom, which optionally has 1 to 5 substituents selected from substituent group B; and
   (dd) a group represented by the formula -Z¹-Z²
   wherein Z¹ is a bond, methylene (CH₂), ethylene (CH₂CH₂), vinylene (CHCH), ethynylene (CC) or methyleneoxy (CH₂O), and
   Z² is (i) a phenyl group optionally having substituents selected from the group consisting of (a) a halogen atom, (b) cyano, (c) an optionally halogenated C₁₋₆ alkyl group, (d) an optionally halogenated C₁₋₆ alkoxy group, (e) carbamoyl and (f) sulfamoyl, or
   (ii) a 5 to 10-membered monocyclic or bicyclic heterocyclic group containing, besides carbon atom, 1 to 4 hetero atoms of 1 or 2 kinds selected from a nitrogen atom, a sulfur atom and an oxygen atom, which optionally has substituents selected from the group consisting of (a) a halogen atom, (b) cyano, (c) an optionally halogenated C₁₋₆ alkyl group, (d) an optionally halogenated C₁₋₆ alkoxy group, (e) carbamoyl and (f) sulfamoyl;
(n) C₁₋₆ alkoxy-carbonyl optionally substituted by 1 to 5 substituents selected from substituent group B,
(o) lower (C₁₋₆)alkylsulfonyl optionally substituted by 1 to 5 substituents selected from substituent group B,
(p) C₆₋₁₄ arylsulfonyl optionally substituted by 1 to 5 substituents selected from substituent group B, and
(q) a carbamoyl group or a 5 to 7-membered cyclic carbamoyl group optionally having 1 or 2 substituents selected from the group consisting of the following (aa) to (pp);
   (aa) lower (C₁₋₆)alkyl optionally substituted by 1 to 5 substituents selected from substituent group B (particularly, lower (C₁₋₆)alkyl optionally substituted by the “optionally substituted amino group” of substituent group B),
   (bb) lower (C₂₋₆)alkenyl optionally substituted by 1 to 5 substituents selected from substituent group B,
   (cc) lower (C₂₋₆)alkynyl optionally substituted by 1 to 5 substituents selected from substituent group B,
   (dd) C₃₋₈ cycloalkyl optionally substituted by 1 to 5 substituents selected from substituent group B,
   (ee) C₆₋₁₄ aryl optionally substituted by 1 to 5 substituents selected from substituent group B,
   (ff) C₇₋₁₆ aralkyl optionally substituted by 1 to 5 substituents selected from substituent group B,
   (gg) a 3 to 14-membered (monocyclic, bicyclic or tricyclic) heterocyclic group containing, besides carbon atom, 1 to 4 hetero atoms of 1 or 2 kinds selected from a nitrogen atom, a sulfur atom and an oxygen atom, which optionally has 1 to 5 substituents selected from substituent group B,
   (hh) lower (C₁₋₆)alkoxy optionally substituted by 1 to 5 substituents selected from substituent group B,
   (ii) formyl,
   (jj) lower (C₁₋₆)alkyl-carbonyl optionally substituted by 1 to 5 substituents selected from substituent group B,
   (kk) C₃₋₈ cycloalkyl-carbonyl optionally substituted by 1 to 5 substituents selected from substituent group B,
   (ll) C₆₋₁₄ aryl-carbonyl optionally substituted by 1 to 5 substituents selected from substituent group B,
   (mm) C₇₋₁₆ aralkyl-carbonyl optionally substituted by 1 to 5 substituents selected from substituent group B,
   (nn) 3 to 14-membered (monocyclic, bicyclic or tricyclic) heterocycle-carbonyl containing, besides carbon atom, 1 to 4 hetero atoms of 1 or 2 kinds selected from a nitrogen atom, a sulfur atom and an oxygen atom, which is optionally substituted by 1 to 5 substituents selected from the group consisting of the following (aaa) to (ddd);
      (aaa) a substituent selected from substituent group B (except a phenyl group and a 5 to 10-membered heterocyclic group containing, besides carbon atom, 1 to 4 hetero atoms of 1 or 2 kinds selected from a nitrogen atom, a sulfur atom and an oxygen atom (wherein said heterocyclic group is optionally substituted by halogen atom, hydroxy, amino, mono- or di-C₁₋₆ alkylamino, mono- or di-C₆₋₁₄ arylamino, mono- or di-C₇₋₁₆ aralkylamino, C₃₋₈ cycloalkyl, C₁₋₆ alkoxy, formyl, C₁₋₆ alkyl-carbonyl, C₃₋₈ cycloalkyl-carbonyl, C₆₋₁₄ aryl-carbonyl, C₇₋₁₆ aralkyl-carbonyl, C₁₋₆ alkoxy-carbonyl, C₆₋₁₄ aryloxy-carbonyl, C₇₋₁₆ aralkyloxy-carbonyl, C₁₋₆ alkylthio, C₁₋₆ alkylsulfinyl, C₁₋₆ alkylsulfonyl, carbamoyl, thiocarbamoyl, mono- or di-C₁₋₆ alkyl-carbamoyl, mono- or di-C₆₋₁₄ aryl-carbamoyl or the like)),
      (bbb) a phenyl group, a benzyl group, a phenethyl group, a styryl group, a phenylethynyl group or a phenoxymethyl group, each optionally having 1 to 5 substituents selected from substituent group B,
      (ccc) a 3 to 14-membered (monocyclic, bicyclic or tricyclic) heterocyclic group containing, besides carbon atom, 1 to 4 hetero atoms of 1 or 2 kinds selected from a nitrogen atom, a sulfur atom and an oxygen atom, which optionally has 1 to 5 substituents selected from substituent group B; and
      (ddd) a group represented by the formula -Z¹-Z² wherein Z¹ is a bond, methylene (CH₂), ethylene (CH₂CH₂), vinylene (CHCH), ethynylene (CC) or methyleneoxy (CH₂O), and
      Z² is (i) a phenyl group optionally having substituents selected from the group consisting of (a) a halogen atom, (b) cyano, (c) an optionally halogenated C₁₋₆ alkyl group, (d) an optionally halogenated C₁₋₆ alkoxy group, (e) carbamoyl and (f) sulfamoyl, or
      (ii) a 5 to 10-membered monocyclic or bicyclic heterocyclic group containing, besides carbon atom, 1 to 4 hetero atoms of 1 or 2 kinds selected from a nitrogen atom, a sulfur atom and an oxygen atom, which optionally has substituents selected from the group consisting of (a) a halogen atom, (b) cyano, (c) an optionally halogenated C₁₋₆ alkyl group, (d) an optionally halogenated C₁₋₆ alkoxy group, (e) carbamoyl and (f) sulfamoyl;
   (oo) lower (C₁₋₆)alkylsulfonyl optionally substituted by 1 to 5 substituents selected from substituent group B, and
   (pp) C₆₋₁₄ arylsulfonyl optionally substituted by 1 to 5 substituents selected from substituent group B; and
      (xxxxvi) a group bonded to two or more (e.g., 2-3) substituents from these (i) to (xxxxv), substituent group B is a substituent group consisting of the following (i) to (xxxii)
      (i) a halogen atom;
      (ii) hydroxy;
      (iii) nitro;
      (iv) cyano;
      (v) C₁₋₆ alkyl (wherein said C₁₋₆ alkyl is optionally substituted by halogen atom, hydroxy, amino, mono- or di-C₁₋₆ alkylamino, mono- or di-C₆₋₁₄ arylamino, C₃₋₈ cycloalkyl, C₁₋₆ alkoxy, formyl, C₁₋₆ alkyl-carbonyl, C₃₋₈ cycloalkyl-carbonyl, C₆₋₁₄ aryl-carbonyl, C₇₋₁₆ aralkyl-carbonyl, C₁₋₆ alkoxy-carbonyl, C₆₋₁₄ aryloxy-carbonyl, C₇₋₁₆ aralkyloxy-carbonyl, C₁₋₆ alkylthio, C₁₋₆ alkylsulfinyl, C₁₋₆ alkylsulfonyl, carbamoyl, thiocarbamoyl, mono- or di-C₁₋₆ alkyl-carbamoyl, mono- or di-C₆₋₁₄ aryl-carbamoyl or the like);
      (vi) C₂₋₆ alkenyl (wherein said C₂₋₆ alkenyl is optionally substituted by halogen atom, hydroxy, amino, mono- or di-C₁₋₆ alkylamino, mono- or di-C₆₋₁₄ arylamino, C₃₋₈ cycloalkyl, C₁₋₆ alkoxy, formyl, C₁₋₆ alkyl-carbonyl, C₃₋₈ cycloalkyl-carbonyl, C₆₋₁₄ aryl-carbonyl, C₇₋₁₆ aralkyl-carbonyl, C₁₋₆ alkoxy-carbonyl, C₆₋₁₄ aryloxy-carbonyl, C₇₋₁₆ aralkyloxy-carbonyl, C₁₋₆ alkylthio, C₁₋₆ alkylsulfinyl, C₁₋₆ alkylsulfonyl, carbamoyl, thiocarbamoyl, mono- or di-C₁₋₆ alkyl-carbamoyl, mono- or di-C₆₋₁₄ aryl-carbamoyl or the like);
      (vii) C₂₋₆ alkynyl (wherein said C₂₋₆ alkynyl is optionally substituted by halogen atom, hydroxy, amino, mono- or di-C₁₋₆ alkylamino, mono- or di-C₆₋₁₄ arylamino, C₃₋₈ cycloalkyl, C₁₋₆ alkoxy, formyl, C₁₋₆ alkyl-carbonyl, C₃₋₈ cycloalkyl-carbonyl, C₆₋₁₄ aryl-carbonyl, C₇₋₁₆ aralkyl-carbonyl, C₁₋₆ alkoxy-carbonyl, C₆₋₁₄ aryloxy-carbonyl, C₇₋₁₆ aralkyloxy-carbonyl, C₁₋₆ alkylthio, C₁₋₆ alkylsulfinyl, C₁₋₆ alkylsulfonyl, carbamoyl, thiocarbamoyl, mono- or di-C₁₋₆ alkyl-carbamoyl, mono- or di-C₆₋₁₄ aryl-carbamoyl or the like);
      (viii) C₆₋₁₄ aryl (wherein said C₆₋₁₄ aryl is optionally substituted by halogen atom, hydroxy, amino, optionally halogenated C₁₋₆ alkyl, mono- or di-C₁₋₆ alkylamino, mono- or di-C₆₋₁₄ arylamino, C₃₋₈ cycloalkyl, C₁₋₆ alkoxy, formyl, C₁₋₆ alkyl-carbonyl, C₃₋₈ cycloalkyl-carbonyl, C₆₋₁₄ aryl-carbonyl, C₇₋₁₆ aralkyl-carbonyl, C₁₋₆ alkoxy-carbonyl, C₆₋₁₄ aryloxy-carbonyl, C₇₋₁₆ aralkyloxy-carbonyl, C₁₋₆ alkylthio, C₁₋₆ alkylsulfinyl, C₁₋₆ alkylsulfonyl, carbamoyl, thiocarbamoyl, mono- or di-C₁₋₆ alkyl-carbamoyl, mono- or di-C₆₋₁₄ aryl-carbamoyl or the like);
      (ix) C₆₋₁₄ aryloxy (wherein said C₆₋₁₄ aryloxy is optionally substituted by halogen atom, hydroxy, cyano, amino, optionally halogenated C₁₋₆ alkyl, mono- or di-C₁₋₆ alkylamino, mono- or di-C₆₋₁₄ arylamino, C₃₋₈ cycloalkyl, C₁₋₆ alkoxy, formyl, C₁₋₆ alkyl-carbonyl, C₃₋₈ cycloalkyl-carbonyl, C₆₋₁₄ aryl-carbonyl, C₇₋₁₆ aralkyl-carbonyl, C₁₋₆ alkoxy-carbonyl, C₆₋₁₄ aryloxy-carbonyl, C₇₋₁₆ aralkyloxy-carbonyl, C₁₋₆ alkylthio, C₁₋₆ alkylsulfinyl, C₁₋₆ alkylsulfonyl, carbamoyl, thiocarbamoyl, mono- or di-C₁₋₆ alkyl-carbamoyl, mono- or di-C₆₋₁₄ aryl-carbamoyl or the like);
      (x) C₇₋₁₆ aralkyloxy (wherein said C₇₋₁₆ aralkyloxy is optionally substituted by halogen atom, hydroxy, amino, optionally halogenated C₁₋₆ alkyl, mono- or di-C₁₋₅ alkylamino, mono- or di-C₆₋₁₄ arylamino, C₃₋₈ cycloalkyl, C₁₋₆ alkoxy, formyl, C₁₋₆ alkyl-carbonyl, C₃₋₈ cycloalkyl-carbonyl, C₆₋₁₄ aryl-carbonyl, C₇₋₁₆ aralkyl-carbonyl, C₁₋₆ alkoxy-carbonyl, C₆₋₁₄ aryloxy-carbonyl, C₇₋₁₆ aralkyloxy-carbonyl, C₁₋₆ alkylthio, C₁₋₆ alkylsulfinyl, C₁₋₆ alkylsulfonyl, carbamoyl, thiocarbamoyl, mono- or di-C₁₋₆ alkyl-carbamoyl, mono- or di-C₆₋₁₄ aryl-carbamoyl or the like);
      (xi) a 5 to 10-membered heterocyclic group containing, besides carbon atom, 1 to 4 hetero atoms of 1 or 2 kinds selected from a nitrogen atom, a sulfur atom and an oxygen atom (wherein said heterocyclic group is optionally substituted by halogen atom, hydroxy, amino, mono- or di-C₁₋₆ alkylamino, mono- or di-C₆₋₁₄ arylamino, mono- or di-C₇₋₁₆ aralkylamino, C₃₋₈ cycloalkyl, C₁₋₆ alkoxy, formyl, C₁₋₆ alkyl-carbonyl, C₃₋₈ cycloalkyl-carbonyl, C₆₋₁₄ aryl-carbonyl, C₇₋₁₆ aralkyl-carbonyl, C₁₋₆ alkoxy-carbonyl, C₆₋₁₄ aryloxy-carbonyl, C₇₋₁₆ aralkyloxy-carbonyl, C₁₋₆ alkylthio, C₁₋₆ alkylsulfinyl, C₁₋₆ alkylsulfonyl, carbamoyl, thiocarbamoyl, mono- or di-C₁₋₆ alkyl-carbamoyl, mono- or di-C₆₋₁₄ aryl-carbamoyl or the like);
      (xii) an amino group optionally substituted by 1 or 2 substituents selected from C₁₋₆ alkyl, C₂₋₆ alkenyl, C₆₋₁₄ aryl, C₇₋₁₆ aralkyl, a 3 to 14-membered (monocyclic, bicyclic or tricyclic) heterocyclic group containing, besides carbon atom, 1 to 4 hetero atoms of 1 or 2 kinds selected from a nitrogen atom, a sulfur atom and an oxygen atom and 3 to 14-membered (monocyclic, bicyclic or tricyclic) heterocycle-C₁₋₆ alkyl containing, besides carbon atom, 1 to 4 hetero atoms of 1 or 2 kinds selected from a nitrogen atom, a sulfur atom and an oxygen atom (wherein said C₁₋₆ alkyl, C₂₋₆ alkenyl, C₆₋₁₄ aryl, C₇₋₁₆ aralkyl, heterocyclic group and heterocycle-C₁₋₆ alkyl are each optionally substituted by halogen atom, hydroxy, cyano, amino, optionally halogenated C₁₋₆ alkyl, mono- or di-C₁₋₆ alkylamino, mono- or di-C₆₋₁₄ arylamino, C₃₋₈ cycloalkyl, C₁₋₆ alkoxy, formyl, C₁₋₆ alkyl-carbonyl, C₃₋₈ cycloalkyl-carbonyl, C₆₋₁₄ aryl-carbonyl, C₇₋₁₆ aralkyl-carbonyl, C₁₋₆ alkoxy-carbonyl, C₆₋₁₄ aryloxy-carbonyl, C₇₋₁₆ aralkyloxy-carbonyl, C₁₋₆ alkylthio, C₁₋₆ alkylsulfinyl, C₁₋₆ alkylsulfonyl, carbamoyl, thiocarbamoyl, mono- or di-C₁₋₆ alkyl-carbamoyl, mono- or di-C₆₋₁₄ aryl-carbamoyl or the like);
      (xiii) C₃₋₈ cycloalkyl;
      (xiv) C₁₋₆ alkoxy (wherein said C₁₋₆ alkoxy is optionally substituted by halogen atom, hydroxy, amino, mono- or di-C₁₋₆ alkylamino, mono- or di-C₆₋₁₄ arylamino, C₃₋₈ cycloalkyl, C₁₋₆ alkoxy, formyl, C₁₋₆ alkyl-carbonyl, C₃₋₈ cycloalkyl-carbonyl, C₆₋₁₄ aryl-carbonyl, C₇₋₁₆ aralkyl-carbonyl, C₁₋₆ alkoxy-carbonyl, C₆₋₁₄ aryloxy-carbonyl, C₇₋₁₆ aralkyloxy-carbonyl, C₁₋₆ alkylthio, C₁₋₆ alkylsulfinyl, C₁₋₆ alkylsulfonyl, carbamoyl, thiocarbamoyl, mono- or di-C₁₋₆ alkyl-carbamoyl, mono- or di-C₆₋₁₄ aryl-carbamoyl or the like);
      (xv) formyl;
      (xvi) C₁₋₆ alkyl-carbonyl;
      (xvii) C₃₋₈ cycloalkyl-carbonyl;
      (xviii) C₆₋₁₄ aryl-carbonyl;
      (xix) C₇₋₁₆ aralkyl-carbonyl;
      (xx) C₁₋₆ alkoxy-carbonyl;
      (xxi) C₆₋₁₄ aryloxy-carbonyl;
      (xxii) C₇₋₁₆ aralkyloxy-carbonyl;
      (xxiii) C₁₋₆ alkylthio;
      (xxiv) C₁₋₆ alkylsulfinyl;
      (xxv) C₁₋₆ alkylsulfonyl;
      (xxvi) carbamoyl;
      (xxvii) thiocarbamoyl;
      (xxviii) mono-C₁₋₆ alkyl-carbamoyl;
      (xxix) di-C₁₋₆ alkyl-carbamoyl;
      (xxx) mono- or di-C₆₋₁₄ aryl-carbamoyl;
      (xxxi) mono- or di-5 to 7-membered heterocycle-carbamoyl containing, besides carbon atom, 1 to 4 hetero atoms of 1 or 2 kinds selected from a nitrogen atom, a sulfur atom and an oxygen atom, and
      (xxxii) sulfamoyl.
      R¹ and R³ are each
      (i) a hydrogen atom,
      (ii) a linear or branched C₁₋₅ alkyl group, a C₃₋₈ cycloalkyl group, a C₂₋₁₈ alkenyl group, a C₃₋₁₀ cycloalkenyl group, a C₂₋₈ alkynyl group, a C₆₋₁₄ aryl group, a biphenyl group, a tolyl group or a C₇₋₁₆ aralkyl group, each optionally having 1 to 5 substituents selected from substituent group B, or
      (iii) a 3 to 14-membered (monocyclic, bicyclic or tricyclic) heterocyclic group containing, besides carbon atom, 1 to 4 hetero atoms of 1 or 2 kinds selected from a nitrogen atom, a sulfur atom and an oxygen atom, which optionally has 1 to 5 substituents selected from substituent group B. R^{2b} is
      (i) a linear or branched C₁₋₁₅ alkyl group, a C₃₋₈ cycloalkyl group, a C₂₋₁₈ alkenyl group, a C₃₋₁₀ cycloalkenyl group, a C₂₋₈ alkynyl group, a C₆₋₁₄ aryl group, a biphenyl group, a tolyl group or a C₇₋₁₆ aralkyl group, each optionally having 1 to 5 substituents selected from substituent group B, or
      (ii) a 3 to 14-membered (monocyclic, bicyclic or tricyclic) heterocyclic group containing, besides carbon atom, 1 to 4 hetero atoms of 1 or 2 kinds selected from a nitrogen atom, a sulfur atom and an oxygen atom, which optionally has 1 to 5 substituents selected from substituent group B,
      provided that R^{2b} is not a 4-hydroxyphenyl group optionally having substituents selected from the group consisting of a halogen atom, a C₁₋₆ alkyl group and a C₁₋₆ alkoxy group; a 4-methoxyphenyl group optionally having substituents selected from the group consisting of a halogen atom, a C₁₋₆ alkyl group and a C₁₋₆ alkoxy group; and a 6-hydroxypyridin-3-yl group optionally having substituents selected from the group consisting of a halogen atom, a C₁₋₆ alkyl group and a C₁₋₆ alkoxy group,
      R¹ and R^{2b} optionally form a 7 or 8-membered ring via X, and when R¹ and R^{2b} form a ring via X, then R¹ and R^{2b} are each
      (i) a bond or
      (ii) a divalent C₁₋₅ acyclic hydrocarbon group optionally having substituents selected from substituent group B, and
      X is
      (i) a bond,
      (ii) an oxygen atom,
      (iii) an optionally oxidized sulfur atom, or
      (iv) an imino group optionally having a substituent selected from the group consisting of the following (a) to (o).
(a) lower (C₁₋₆)alkyl optionally substituted by 1 to 5 substituents selected from substituent group B;
(b) lower (C₂₋₆)alkenyl optionally substituted by 1 to 5 substituents selected from substituent group B;
(c) lower (C₂₋₆)alkynyl optionally substituted by 1 to 5 substituents selected from substituent group B;
(d) C₃₋₈ cycloalkyl optionally substituted by 1 to 5 substituents selected from substituent group B;
(e) C₆₋₁₄ aryl optionally substituted by 1 to 5 substituents selected from substituent group B;
(f) C₇₋₁₆ aralkyl optionally substituted by 1 to 5 substituents selected from substituent group B;
(g) lower (C₁₋₆)alkoxy optionally substituted by 1 to 5 substituents selected from substituent group B;
(h) lower (C₁₋₆)alkyl-carbonyl optionally substituted by 1 to 5 substituents selected from substituent group B;
(i) C₃₋₈ cycloalkyl-carbonyl optionally substituted by 1 to 5 substituents selected from substituent group B;
(j) C₆₋₁₄ aryl-carbonyl optionally substituted by 1 to 5 substituents selected from substituent group B;
(k) C₇₋₁₆ aralkyl-carbonyl optionally substituted by 1 to 5 substituents selected from substituent group B;
(l) a 3 to 14-membered (monocyclic, bicyclic or tricyclic) heterocycle-carbonyl containing, besides carbon atom, 1 to 4 hetero atoms of 1 or 2 kinds selected from a nitrogen atom, a sulfur atom and an oxygen atom, which is optionally substituted by 1 to 5 substituents selected from the group consisting of the following (aa) to (dd);
   (aa) a substituent selected from substituent group B (except a phenyl group and a 5 to 10-membered heterocyclic group containing, besides carbon atom, 1 to 4 hetero atoms of 1 or 2 kinds selected from a nitrogen atom, a sulfur atom and an oxygen atom (wherein said heterocyclic group is optionally substituted by halogen atom, hydroxy, amino, mono- or di-C₁₋₆ alkylamino, mono- or di-C₆₋₁₄ arylamino, mono- or di-C₇₋₁₆ aralkylamino, C₃₋₈ cycloalkyl, C₁₋₆ alkoxy, formyl, C₁₋₆ alkyl-carbonyl, C₃₋₈ cycloalkyl-carbonyl, C₆₋₁₄ aryl-carbonyl, C₇₋₁₆ aralkyl-carbonyl, C₁₋₆ alkoxy-carbonyl, C₆₋₁₄ aryloxy-carbonyl, C₇₋₁₆ aralkyloxy-carbonyl, C₁₋₆ alkylthio, C₁₋₆ alkylsulfinyl, C₁₋₆ alkylsulfonyl, carbamoyl, thiocarbamoyl, mono- or di-C₁₋₆ alkyl-carbamoyl, mono- or di-C₆₋₁₄ aryl-carbamoyl or the like)),
   (bb) a phenyl group, a benzyl group, a phenethyl group, a styryl group, a phenylethynyl group or a phenoxymethyl group, each optionally having 1 to 5 substituents selected from substituent group B,
   (cc) a 3 to 14-membered (monocyclic, bicyclic or tricyclic) heterocyclic group containing, besides carbon atom, 1 to 4 hetero atoms of 1 or 2 kinds selected from a nitrogen atom, a sulfur atom and an oxygen atom optionally having 1 to 5 substituents selected from substituent group B; and
   (dd) a group represented by the formula -Z¹-Z²
   wherein Z¹ is a bond, methylene (CH₂), ethylene (CH₂CH₂), vinylene (CHCH), ethynylene (CC) or methyleneoxy (CH₂O), Z² is (i) a phenyl group optionally having substituents selected from the group consisting of (a) a halogen atom, (b) cyano, (c) an optionally halogenated C₁₋₆ alkyl group, (d) an optionally halogenated C₁₋₆ alkoxy group, (e) carbamoyl and (f) sulfamoyl, or
   (ii) a 5 to 10-membered monocyclic or bicyclic heterocyclic group containing, besides carbon atom, 1 to 4 hetero atoms of 1 or 2 kinds selected from a nitrogen atom, a sulfur atom and an oxygen atom, which optionally has substituents selected from the group consisting of (a) a halogen atom, (b) cyano, (c) an optionally halogenated C₁₋₆ alkyl group, (d) an optionally halogenated C₁₋₆ alkoxy group, (e) carbamoyl and (f) sulfamoyl;
(m) lower (C₁₋₆)alkylsulfonyl optionally substituted by 1 to 5 substituents selected from substituent group B;
(n) C₆₋₁₄ arylsulfonyl optionally substituted by 1 to 5 substituents selected from substituent group B; and
(o) a 3 to 14-membered (monocyclic, bicyclic or tricyclic) heterocyclic group containing, besides carbon atom, 1 to 4 hetero atoms of 1 or 2 kinds selected from a nitrogen atom, a sulfur atom and an oxygen atom, which optionally has substituents 1 to 5 substituents selected from substituent group B,
   provided that when X is a bond, then the ring formed by R¹ and R^{2b} via X is a 7 or more-membered ring,
   [63] the compound of the above-mentioned [34], wherein R¹′ and R^{2b}′ are each
   (i) a bond or
   (ii) a divalent C₁₋₅ acyclic hydrocarbon group optionally having substituents selected from substituent group B, and A, R³ and X are as defined in the above-mentioned [62],
   provided that when X is a bond, the ring formed by R¹′ and R^{2b}′ via X is a 7 or more-membered ring,
   [64] the compound of the above-mentioned [35], wherein R¹″ is
   (i) a hydrogen atom,
   (ii) a linear or branched C₁₋₁₅ alkyl group, C₃₋₈ cycloalkyl group, C₂₋₁₈ alkenyl group, C₃₋₁₀ cycloalkenyl group, C₂₋₈ alkynyl group, C₆₋₁₄ aryl group, biphenyl group, tolyl group or C₇₋₁₆ aralkyl group, each optionally having 1 to 5 substituents selected from substituent group B, or
   (iii) a 3 to 14-membered (monocyclic, bicyclic or tricyclic) heterocyclic group containing, besides carbon atom, 1 to 4 sulfur atom and an oxygen atom, which optionally has 1 to 5 substituents selected from substituent group B, R^{2b}″is
   (i) a linear or branched C₁₋₁₅ alkyl group, a C₃₋₈ cycloalkyl group, a C₂₋₁₈ alkenyl group, a C₃₋₁₀ cycloalkenyl group, a C₂₋₈ alkynyl group, a C₆₋₁₄ aryl group, a biphenyl group, a tolyl group or a C₇₋₁₆ aralkyl group, each optionally having 1 to 5 substituents selected from substituent group B, or
   (ii) a 3 to 14-membered (monocyclic, bicyclic or tricyclic) heterocyclic group containing, besides carbon atom, 1 to 4 hetero atoms of 1 or 2 kinds selected from a nitrogen atom, a sulfur atom and an oxygen atom, which optionally has 1 to 5 substituents selected from substituent B, and
   A and R³ are as defined in the above-mentioned [62], provided that R^{2b}″ is not a 4-hydroxyphenyl group optionally having substituents selected from the group consisting of a halogen atom, a C₁₋₆ alkyl group and a C₁₋₆ alkoxy group; a 4-methoxyphenyl group optionally having substituents selected from the group consisting of a halogen atom, a C₁₋₆ alkyl group and a C₁₋₆ alkoxy group; and a 6-hydroxypyridin-3-yl group optionally having substituents selected from the group consisting of a halogen atom, a C₁₋₆ alkyl group and a C₁₋₆ alkoxy group,
   [65] the compound of the above-mentioned [36], wherein A′ is a benzene ring optionally having substituents selected from substituent group A,
   Y¹ and Y² are each
   (i) a bond,
   (ii) an oxygen atom,
   (iii) an optionally oxidized sulfur atom,
   (iv) an imino group optionally having a substituent described in the above-mentioned [62], or
   (v) a carbonyl group,
   Z is
   (i) a hydrogen atom,
   (ii) a linear or branched C₁₋₁₅ alkyl group, a C₃₋₈ cycloalkyl group, a C₂₋₁₈ alkenyl group, a C₃₋₁₀ cycloalkenyl group, a C₂₋₈ alkynyl group, a C₆₋₁₄ aryl group, a biphenyl group, a tolyl group or a C₇₋₁₆ aralkyl group, each optionally having 1 to 5 substituents selected from substituent group B, or
   (iii) a 3 to 14-membered (monocyclic, bicyclic or tricyclic) heterocyclic group containing, besides carbon atom, 1 to 4 hetero atoms of 1 or 2 kinds selected from a nitrogen atom, a sulfur atom and an oxygen atom, which optionally has 1 to 5 substituents selected from substituent group B, and R¹″, R^{2b}″ and R³ are as defined in the above-mentioned [64], and
   [66] the compound of the above-mentioned [41], wherein Z¹ is a bond, methylene (CH₂), ethylene (CH₂CH₂), vinylene (CHCH) or ethynylene (CC), and
   Z² is
   (i) a phenyl group optionally having 1 to 5 substituents selected from substituent group B or
   (ii) a 3 to 14-membered (monocyclic, bicyclic or tricyclic) heterocyclic group containing, besides carbon atom, 1 to 4 hetero atoms of 1 or 2 kinds selected from a nitrogen atom, a sulfur atom and an oxygen atom, which optionally has 1 to 5 substituents selected from substituent group B,
   A″ is a benzene ring optionally further having substituents selected from substituent group A,
   R³ is
   (i) a hydrogen atom,
   (ii) a linear or branched C₁₋₁₅ alkyl group, a C₃₋₈ cycloalkyl group, a C₂₋₁₈ alkenyl group, a C₃₋₁₀ cycloalkenyl group, a C₂₋₈ alkynyl group, a C₆₋₁₄ aryl group, a biphenyl group, a tolyl group or a C₇₋₁₆ aralkyl group, each optionally having 1 to 5 substituents selected from substituent group B, or
   (iii) a 3 to 14-membered (monocyclic, bicyclic or tricyclic) heterocyclic group containing, besides carbon atom, 1 to 4 hetero atoms of 1 or 2 kinds selected from a nitrogen atom, a sulfur atom and an oxygen atom, which optionally has 1 to 5 substituents selected from substituent group B, and
   Q is a piperidine ring optionally further having, besides -Z¹-Z², substituents selected from substituent group A.
   [Compound (I)]

In the above-mentioned formula, A is a benzene ring optionally having substituents.

As the substituent of the benzene ring for ring A, for example, substituents selected from

(i) a halogen atom (e.g., fluorine, chlorine, bromine, iodine etc.);

(ii) C₁₋₃ alkylenedioxy (e.g., methylenedioxy, ethylenedioxy etc.);

(iii) nitro;

(iv) cyano;

(v) optionally esterified carboxyl [e.g., carboxyl, optionally substituted C₁₋₆ alkoxy-carbonyl, optionally substituted C₆₋₁₄ aryloxy-carbonyl, optionally substituted C₇₋₁₆ aralkyloxy-carbonyl and the like];

(vi) optionally substituted lower (C₁₋₆)alkyl;

(vii) optionally substituted lower (C₂₋₆)alkenyl;

(viii) optionally substituted lower (C₂₋₆)alkynyl;

(ix) optionally substituted C₃₋₈ cycloalkyl;

(x) optionally substituted C₆₋₁₄ aryl;

(xi) optionally substituted C₇₋₁₆ aralkyl;

(xii) optionally substituted C₆₋₁₄ aryl-C₂₋₆ alkenyl;

(xiii) a heterocyclic group optionally having substituents;

(xiv) hydroxy;

(xv) optionally substituted lower (C₁₋₆)alkoxy;

(xvi) optionally substituted C₆₋₁₄ aryloxy;

(xvii) optionally substituted C₇₋₁₆ aralkyloxy;

(xviii) optionally substituted lower (C₁₋₆)alkyl-carbonyloxy;

(xix) optionally substituted lower (C₁₋₆)alkoxy-carbonyloxy;

(xx) optionally substituted mono-lower (C₁₋₆)alkyl-carbamoyloxy;

(xxi) optionally substituted di-lower (C₁₋₆)alkyl-carbamoyloxy;

(xxii) optionally substituted C₆₋₁₄ aryl-carbonyloxy;

(xxiii) optionally substituted mono- or di-C₆₋₁₄ aryl-carbamoyloxy;

(xxiv) optionally substituted heterocycle-oxy (preferably, optionally substituted aromatic heterocycle-oxy);

(xxv) mercapto;

(xxvi) optionally substituted lower (C₁₋₆)alkylthio;

(xxvii) optionally substituted C₆₋₁₄ arylthio;

(xxviii) optionally substituted C₇₋₁₆ aralkylthio;

(xxix) formyl;

(xxx) optionally substituted lower (C₁₋₆)alkyl-carbonyl;

(xxxi) optionally substituted C₃₋₈ cycloalkyl-carbonyl;

(xxxii) optionally substituted C₆₋₁₄ aryl-carbonyl;

(xxxiii) optionally substituted C₇₋₁₆ aralkyl-carbonyl;

(xxxiv) optionally substituted heterocycle-carbonyl;

(xxxv) optionally substituted lower (C₁₋₆)alkylsulfonyl;

(xxxvi) optionally substituted C₆₋₁₄ arylsulfonyl;

(xxxvii) optionally substituted lower (C₁₋₆)alkylsulfinyl;

(xxxviii) optionally substituted C₆₋₁₄ arylsulfinyl;

(xxxix) sulfo;

(xxxx) sulfamoyl;

(xxxxi) sulfinamoyl;

(xxxxii) sulfenamoyl;

(xxxxiii) thiocarbamoyl;

(xxxxiv) a carbamoyl group optionally having substituents [particularly, optionally substituted lower (C₁₋₆)alkyl-carbamoyl and the like];

(xxxxv) optionally substituted amino group [e.g., amino, optionally substituted mono- or di-lower (C₁₋₆)alkylamino, optionally substituted mono- or di-C₃₋₈ cycloalkylamino, optionally substituted mono- or di-C₆₋₁₄ arylamino, optionally substituted mono- or di-C₇₋₁₆ aralkylamino, optionally substituted C₆₋₁₄ aryl-carbonylamino, formylamino, optionally substituted lower (C₁₋₆)alkyl-carbonylamino, optionally substituted C₃₋₈ cycloalkyl-carbonylamino, optionally substituted heterocycle-carbonylamino, optionally substituted lower (C₁₋₆)alkoxy-carbonylamino, a carbamoylamino group optionally having substituents, optionally substituted lower (C₁₋₆) alkylsulfonylamino, optionally substituted C₆₋₁₄ arylsulfonylamino (e.g., phenylsulfonylamino, 2-naphthylsulfonylamino, 1-naphthylsulfonylamino etc.)]; and the like; a group bonded to two or more (e.g., 2-3) of these substituents; and the like (hereinafter to be abbreviated as substituent group A) can be used.

The ring A may have 1 to 4, preferably 1 to 3, of the above-mentioned substituents at substitutable positions, and when the number of substituents is two or more, the respective substituents may be the same or different.

While the position of the substituents on ring A may be any of the 4-position, 5-position, 6-position and 7-position of the following structural formula, the 4-position, 5-position and 7-position are preferable, and the 5-position is particularly preferable. When ring A has two substituents, the combinations of the 4-position and the 5-position, and the 4-position and the 7-position are preferable. When R¹ and R² form a ring via X, the numbering may change each time. Therefore, the numbering of the indole ring has been employed.

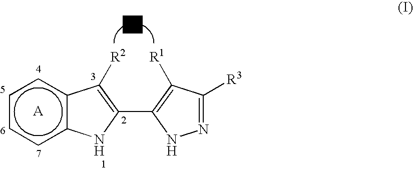

As the “C₁₋₆ alkoxy-carbonyl” of the “optionally substituted C₁₋₆ alkoxy-carbonyl” as the “optionally esterified carboxyl group” of the substituent group A, for example, methoxycarbonyl, ethoxycarbonyl, propoxycarbonyl, tert-butoxy carbonyl and the like can be used.

As the “C₆₋₁₄ aryloxy-carbonyl” of the “optionally substituted C₆₋₁₄ aryloxy-carbonyl” as the “optionally esterified carboxyl group” of the substituent group A, for example, phenoxycarbonyl and the like can be used.

As the “C₇₋₁₆ aralkyloxy-carbonyl” of the “optionally substituted C₇₋₁₆ aralkyloxy-carbonyl” as the “optionally esterified carboxyl group” of the substituent group A, for example, benzyloxycarbonyl, phenethyloxycarbonyl and the like can be used.

As the “lower (C₁₋₆)alkyl” of the “optionally substituted lower (C₁₋₆)alkyl” of the substituent group A, for example, methyl, ethyl, propyl, isopropyl, butyl, isobutyl, sec-butyl, tert-butyl, pentyl, isopentyl, neopentyl, hexyl and the like can be used.

As the “lower (C₂₋₆)alkenyl” of the “optionally substituted lower (C₂₋₆)alkenyl” of the substituent group A, for example, vinyl, propenyl, isopropenyl, 2-buten-1-yl, 4-penten-1-yl, 5-hexen-1-yl and the like can be used.

As the “lower (C₂₋₆)alkynyl” of the “optionally substituted lower (C₂₋₆)alkynyl” of the substituent group A, for example, 2-butyn-1-yl, 4-pentyn-1-yl, 5-hexyn-1-yl and the like can be used.

As the “C₃₋₈ cycloalkyl” of the “optionally substituted C₃₋₈ cycloalkyl” of the substituent group A, for example, cyclopropyl, cyclobutyl, cyclopentyl, cyclohexyl and the like can be used.

As the “C₆₋₁₄ aryl” of the “optionally substituted C₆₋₁₄ aryl” of the substituent group A, for example, phenyl, 1-naphthyl, 2-naphthyl, 2-biphenylyl, 3-biphenylyl, 4-biphenylyl, 2-anthryl and the like can be used. Said C₆₋₁₄ aryl may be partially saturated, and as the partially saturated C₆₋₁₄ aryl, for example, tetrahydronaphthyl and the like can be mentioned.

As the “C₇₋₁₆ aralkyl” of the “optionally substituted C₇₋₁₆ aralkyl” of the substituent group A, for example, benzyl, phenethyl, diphenylmethyl, 1-naphthylmethyl, 2-naphthylmethyl, 2,2-diphenylethyl, 3-phenylpropyl, 4-phenylbutyl, 5-phenylpentyl, 2-biphenylylmethyl, 3-biphenylylmethyl, 4-biphenylylmethyl) and the like can be used.

As the “C₆₋₁₄ aryl-C₂₋₆ alkenyl” of the “optionally substituted C₆₋₁₄ aryl-C₂₋₆ alkenyl” of the substituent group A, for example, styryl and the like can be used.

As the “heterocyclic group” of the “heterocyclic group optionally having substituents” of the substituent group A, for example, a 3 to 14-membered (monocyclic, bicyclic or tricyclic) heterocyclic group containing, besides carbon atom, 1 to 4 hetero atoms of 1 or 2 kinds selected from a nitrogen atom, a sulfur atom and an oxygen atom, preferably (i) a 5 to 14-membered (preferably 5 to 10-membered, more preferably 5 to 7-membered) (monocyclic, bicyclic or tricyclic) aromatic heterocyclic group, (ii) a 5 to 10-membered (preferably 5 to 7-membered) non-aromatic heterocyclic group, (iii) a monovalent group obtained by removing any one hydrogen atom from a 7 to 10-membered bridged heterocycle and the like are used. Of these, a 5-membered aromatic heterocyclic group is preferably used. Specifically, for example, thienyl (e.g., 2-thienyl, 3-thienyl), furyl (e.g., 2-furyl, 3-furyl), pyridyl (e.g., 2-pyridyl, 3-pyridyl, 4-pyridyl), thiazolyl (e.g., 2-thiazolyl, 4-thiazolyl, 5-thiazolyl), oxazolyl (e.g., 2-oxazolyl, 4-oxazolyl, 5-oxazolyl), quinolyl (e.g., 2-quinolyl, 3-quinolyl, 4-quinolyl, 5-quinolyl, 6-quinolyl, 7-quinolyl, 8-quinolyl), isoquinolyl (e.g., 1-isoquinolyl, 3-isoquinolyl, 4-isoquinolyl, 5-isoquinolyl, 6-isoquinolyl, 7-isoquinolyl, 8-isoquinolyl), pyrazinyl, pyrimidinyl (e.g., 2-pyrimidinyl, 4-pyrimidinyl), pyrrolyl (e.g., 1-pyrrolyl, 2-pyrrolyl, 3-pyrrolyl), imidazolyl (e.g., 1-imidazolyl, 2-imidazolyl, 4-imidazolyl), pyrazolyl (e.g., 1-pyrazolyl, 3-pyrazolyl, 4-pyrazolyl), pyridazinyl (e.g., 3-pyridazinyl, 4-pyridazinyl), pyrimidinyl (e.g., 2-pyrimidinyl, 4-pyrimidinyl), pyrazinyl, isoxazolyl (e.g., 3-isoxazolyl, 4-isoxazolyl, 5-isoxazolyl), isothiazolyl (e.g., 3-isothiazolyl, 4-isothiazolyl, 5-isothiazolyl), indolyl (e.g., 1-indolyl, 2-indolyl, 3-indolyl, 4-indolyl, 5-indolyl, 6-indolyl, 7-indolyl), isoindolyl (e.g., 1-isoindolyl, 2-isoindolyl, 3-isoindolyl, 4-isoindolyl, 5-isoindolyl, 6-isoindolyl, 7-isoindolyl), 2-benzothiazolyl, benzo[b]thienyl (e.g., 2-benzo[b]thienyl, 3-benzo[b]thienyl, 4-benzo[b]thienyl, 5-benzo[b]thienyl, 6-benzo[b]thienyl, 7-benzo[b]thienyl), benzo[c]thienyl (e.g., 1-benzo[c]thienyl, 4-benzo[c]thienyl, 5-benzo[c]thienyl), benzo[b]furanyl (e.g., 2-benzo[b]furanyl, 3-benzo[b]furanyl, 4-benzo[b]furanyl, 5-benzo[b]furanyl, 6-benzo[b]furanyl, 7-benzo[b]furanyl), benzo[c]furanyl (e.g., 1-benzo[c]furanyl, 4-benzo[c]furanyl, 5-benzo[c]furanyl), benzimidazolyl (e.g., 1-benzimidazolyl, 2-benzimidazolyl, 4-benzimidazolyl, 5-benzimidazolyl), benzoxazolyl (e.g., 2-benzoxazolyl, 4-benzoxazolyl, 5-benzoxazolyl, 6-benzoxazolyl, 7-benzoxazolyl), benzothiazolyl (e.g., 2-benzothiazolyl, 4-benzothiazolyl, 5-benzothiazolyl, 6-benzothiazolyl, 7-benzothiazolyl), indazolyl (e.g., 1-indazolyl, 2-indazolyl, 3-indazolyl, 4-indazolyl, 5-indazolyl, 6-indazolyl, 7-indazolyl), 1,2-benzisoxazolyl (e.g., 1,2-benzisoxazol-3-yl, 1,2-benzisoxazol-4-yl, 1,2-benzisoxazol-5-yl, 1,2-benzisoxazol-6-yl, 1,2-benzisoxazol-7-yl), 1,2-benzisothiazolyl (e.g., 1,2-benzisothiazol-3-yl, 1,2-benzisothiazol-4-yl, 1,2-benzisothiazol-5-yl, 1,2-benzisothiazol-6-yl, 1,2-benzisothiazol-7-yl), cinnolinyl (e.g., 3-cinnolinyl, 4-cinnolinyl, 5-cinnolinyl, 6-cinnolinyl, 7-cinnolinyl, 8-cinnolinyl), phthalazinyl (e.g., 1-phthalazinyl, 4-phthalazinyl, 5-phthalazinyl, 6-phthalazinyl, 7-phthalazinyl, 8-phthalazinyl), quinazolinyl (e.g., 2-quinazolinyl, 4-quinazolinyl, 5-quinazolinyl, 6-quinazolinyl, 7-quinazolinyl, 8-quinazolinyl), quinoxalinyl (e.g., 2-quinoxalinyl, 3-quinoxalinyl, 5-quinoxalinyl, 6-quinoxalinyl, 7-quinoxalinyl, 8-quinoxalinyl) and the like can be mentioned, particularly, aromatic heterocyclic groups such as quinolyl (e.g., 2-quinolyl, 3-quinolyl, 4-quinolyl, 5-quinolyl, 6-quinolyl, 7-quinolyl, 8-quinolyl), isoquinolyl (e.g., 1-isoquinolyl, 3-isoquinolyl, 4-isoquinolyl, 5-isoquinolyl, 6-isoquinolyl, 7-isoquinolyl, 8-isoquinolyl) and the like; non-aromatic heterocyclic groups such as oxazolidinyl (e.g., 2-oxazolidinyl), imidazolinyl (e.g., 1-imidazolinyl, 2-imidazolinyl, 4-imidazolinyl), aziridinyl (e.g., 1-aziridinyl, 2-aziridinyl), azetidinyl (e.g., 1-azetidinyl, 2-azetidinyl), pyrrolidinyl (e.g., 1-pyrrolidinyl, 2-pyrrolidinyl, 3-pyrrolidinyl), piperidinyl (e.g., 1-piperidinyl, 2-piperidinyl, 3-piperidinyl), azepanyl (e.g., 1-azepanyl, 2-azepanyl, 3-azepanyl, 4-azepanyl), azocanyl (e.g., 1-azocanyl, 2-azocanyl, 3-azocanyl, 4-azocanyl), azonanyl (e.g., 1-azonanyl, 2-azonanyl, 3-azonanyl, 4-azonanyl, 5-azonanyl), piperazinyl (e.g., 1,4-piperazin-1-yl, 1,4-piperazin-2-yl), diazepanyl (e.g., 1,4-diazepan-1-yl, 1,4-diazepan-2-yl, 1,4-diazepan-5-yl, 1,4-diazepan-6-yl), diazocanyl (e.g., 1,4-diazocan-1-yl, 1,4-diazocan-2-yl, 1,4-diazocan-5-yl, 1,4-diazocan-6-yl, 1,5-diazocan-1-yl, 1,5-diazocan-2-yl, 1,5-diazocan-3-yl), 4-morpholinyl, 4-thiomorpholinyl, tetrahydroquinolinyl (e.g., 3,4-dihydro-2H-quinolin-1-yl, 1,2,3,4-tetrahydroquinolin-2-yl), tetrahydroisoquinolinyl (e.g., 1,2,3,4-tetrahydroisoquinolin-1-yl, 3,4-dihydro-1H-isoquinolin-2-yl) and the like; and the like are used.

As the “lower (C₁₋₆)alkoxy” of the “optionally substituted lower (C₁₋₆)alkoxy” of the substituent group A, for example, methoxy, ethoxy, propoxy, isopropoxy, butoxy, isobutoxy, sec-butoxy, pentyloxy, hexyloxy and the like can be used.

As the “C₆₋₁₄ aryloxy” of the “optionally substituted C₆₋₁₄ aryloxy” of the substituent group A, for example, phenyloxy, 1-naphthyloxy, 2-naphthyloxy and the like can be used.

As the “C₇₋₁₆ aralkyloxy” of the “optionally substituted C₇₋₁₆ aralkyloxy” of the substituent group A, for example, benzyloxy, phenethyloxy and the like can be used.

As the “lower (C₁₋₆)alkyl-carbonyloxy” of the “optionally substituted lower (C₁₋₆)alkyl-carbonyloxy” of the substituent group A, for example, acetoxy, propionyloxy and the like can be used.

As the “lower (C₁₋₆)alkoxy-carbonyloxy” of the “optionally substituted lower (C₁₋₆)alkoxy-carbonyloxy” of the substituent group A, for example, methoxycarbonyloxy, ethoxycarbonyloxy, propoxycarbonyloxy, butoxy carbonyloxy and the like can be used.

As the “mono-lower (C₁₋₆)alkyl-carbamoyloxy” of the “optionally substituted mono-lower (C₁₋₆)alkyl-carbamoyloxy” of the substituent group A, for example, methylcarbamoyloxy, ethylcarbamoyloxy and the like can be used.

As the “di-lower (C₁₋₆)alkyl-carbamoyloxy” of the “optionally substituted di-lower (C₁₋₆)alkyl-carbamoyloxy” of the substituent group A, for example, dimethylcarbamoyloxy, diethylcarbamoyloxy and the like can be used.

As the “C₆₋₁₄ aryl-carbonyloxy” of the “optionally substituted C₆₋₁₄ aryl-carbonyloxy” of the substituent group A, for example, benzoyloxy, naphthylcarbonyloxy and the like can be used.

As the “mono- or di-C₆₋₁₄ aryl-carbamoyloxy” of the “optionally substituted mono- or di-C₆₋₁₄ aryl-carbamoyloxy” of the substituent group A, for example, phenylcarbamoyloxy, naphthylcarbamoyloxy and the like can be used.

As the heterocycle moiety of the “heterocycle-oxy” of the “optionally substituted heterocycle-oxy” of the substituent group A, those similar to the “heterocyclic group” of the aforementioned “heterocyclic group optionally having substituents” can be used. Specifically, for example, 5 to 10-membered heterocycle-oxy containing, besides carbon atom, 1 to 4 hetero atoms of 1 or 2 kinds selected from a nitrogen atom, a sulfur atom and an oxygen atom and the like can be used.

As the “aromatic heterocycle-oxy” of the “optionally substituted aromatic heterocycle-oxy” of the substituent group A, for example, 5 to 10-membered aromatic heterocycle-oxy containing, besides carbon atom, 1 to 4 hetero atoms of 1 or 2 kinds selected from a nitrogen atom, a sulfur atom and an oxygen atom and the like can be used.

As the “lower (C₁₋₆)alkylthio” of the “optionally substituted lower (C₁₋₆)alkylthio” of the substituent group A, for example, methylthio, ethylthio, propylthio, isopropylthio, butylthio, sec-butylthio, tert-butylthio and the like can be used.

As the “C₆₋₁₄ arylthio” of the “optionally substituted C₆₋₁₄ arylthio” of the substituent group A, for example, phenylthio, 1-naphthylthio, 2-naphthylthio and the like can be used.

As the “C₇₋₁₆ aralkylthio” of the “optionally substituted C₇₋₁₆ aralkylthio” of the substituent group A, for example, benzylthio, phenethylthio and the like can be used.

As the “lower (C₁₋₆)alkyl-carbonyl” of the “optionally substituted lower (C₁₋₆)alkyl-carbonyl” of the substituent group A, for example, acetyl, propionyl, pivaloyl and the like can be used.

As the “C₃₋₈ cycloalkyl-carbonyl” of the “optionally substituted C₃₋₈ cycloalkyl-carbonyl” of the substituent group A, for example, cyclopropylcarbonyl, cyclopentylcarbonyl, cyclohexylcarbonyl and the like can be used.

As the “C₆₋₁₄ aryl-carbonyl” of the “optionally substituted C₆₋₁₄ aryl-carbonyl” of the substituent group A, for example, benzoyl, 1-naphthoyl, 2-naphthoyl and the like can be used.

As the “C₇₋₁₆ aralkyl-carbonyl” of the “optionally substituted C₇₋₁₆ aralkyl-carbonyl” substituent group A, for example, phenylacetyl, 3-phenylpropionyl and the like can be used.

As the heterocycle moiety of the “optionally substituted heterocycle-carbonyl” of the substituent group A, those similar to the “heterocyclic group” of the aforementioned “heterocyclic group optionally having substituents” can be used. Specifically, “optionally substituted 3 to 8-membered heterocycle-carbonyl containing, besides carbon atom, 1 to 4 hetero atoms of 1 or 2 kinds selected from a nitrogen atom, a sulfur atom and an oxygen atom” can be used and, for example, nicotinoyl, isonicotinoyl, thenoyl, furoyl, 4-morpholinylcarbonyl, 4-thiomorpholinylcarbonyl, aziridin-1-ylcarbonyl, aziridin-2-ylcarbonyl, azetidin-1-ylcarbonyl, azetidin-2-ylcarbonyl, pyrrolidin-1-ylcarbonyl, pyrrolidin-2-ylcarbonyl, pyrrolidin-3-ylcarbonyl, piperidin-1-ylcarbonyl, piperidin-2-ylcarbonyl, piperidin-3-ylcarbonyl, azepan-1-ylcarbonyl, azepan-2-ylcarbonyl, azepan-3-ylcarbonyl, azepan-4-ylcarbonyl, azocan-1-ylcarbonyl, azocan-2-ylcarbonyl, azocan-3-ylcarbonyl, azocan-4-ylcarbonyl, 1,4-piperazin-1-ylcarbonyl, 1,4-piperazin-2-ylcarbonyl, 1,4-diazepan-1-ylcarbonyl, 1,4-diazepan-2-ylcarbonyl, 1,4-diazepan-5-ylcarbonyl, 1,4-diazepan-6-ylcarbonyl, 1,4-diazocan-1-ylcarbonyl, 1,4-diazocan-2-ylcarbonyl, 1,4-diazocan-5-ylcarbonyl, 1,4-diazocan-6-ylcarbonyl, 1,5-diazocan-1-ylcarbonyl, 1,5-diazocan-2-ylcarbonyl, 1,5-diazocan-3-ylcarbonyl and the like can be used. Of these, “optionally substituted 5 to 7-membered heterocycle-carbonyl containing, besides carbon atom, 1 to 4 hetero atoms of 1 or 2 kinds selected from a nitrogen atom, a sulfur atom and an oxygen atom” is preferably used, and 6-membered non-aromatic nitrogen-containing heterocycle-carbonyl such as piperidin-1-ylcarbonyl, 1,4-piperazin-1-ylcarbonyl and the like is particularly preferably used.

As the “optionally substituted lower (C₁₋₆)alkylsulfonyl” of the substituent group A, for example, methylsulfonyl, ethylsulfonyl and the like can be used.

As the “C₆₋₁₄ arylsulfonyl” of the “optionally substituted C₆₋₁₄ arylsulfonyl” of the substituent group A, for example, phenylsulfonyl, 1-naphthylsulfonyl, 2-naphthylsulfonyl and the like can be used.

As the “optionally substituted lower (C₁₋₆)alkylsulfinyl” of the substituent group A, for example, methylsulfinyl, ethylsulfinyl and the like can be used.

As the “C₆₋₁₄ arylsulfinyl” of the “optionally substituted C₆₋₁₄ arylsulfinyl” of the substituent group A, for example, phenylsulfinyl, 1-naphthylsulfinyl, 2-naphthylsulfinyl and the like can be used.

As the “lower (C₁₋₆)alkyl-carbamoyl” of the “optionally substituted lower (C₁₋₆)alkyl-carbamoyl” of the substituent group A, for example, methylcarbamoyl, ethylcarbamoyl, propylcarbamoyl and the like can be used.

As the “mono- or di-lower (C₁₋₆)alkylamino” of the “optionally substituted mono- or di-lower (C₁₋₆)alkylamino” of the substituent group A, for example, methylamino, ethylamino, propylamino, dimethylamino, diethylamino and the like can be used.

As the “lower (C₁₋₆)alkyl-carbonylamino” of the “optionally substituted lower (C₁₋₆)alkyl-carbonylamino” of the substituent group A, for example, acetylamino, propionylamino, pivaloylamino and the like can be used.

As the “heterocycle-carbonyl” of the “heterocycle-carbonylamino” of the “optionally substituted heterocycle-carbonylamino” of the substituent group A, those similar to the “heterocycle-carbonyl” of the above-mentioned “optionally substituted heterocycle-carbonyl” can be used and, for example, pyridyl-carbonylamino and the like can be used.

As the “lower (C₁₋₆)alkoxy-carbonylamino” of the “optionally substituted lower (C₁₋₆)alkoxy-carbonylamino” of the substituent group A, for example, methoxycarbonylamino, ethoxycarbonylamino, propoxycarbonylamino, butoxycarbonylamino and the like can be used.

As the “lower (C₁₋₆)alkylsulfonylamino” of the “optionally substituted lower (C₁₋₆)alkylsulfonylamino” of the substituent group A, for example, methylsulfonylamino, ethylsulfonylamino and the like can be used.

As the “mono- or di-C₃₋₈ cycloalkylamino” of the “optionally substituted mono- or di-C₃₋₈ cycloalkylamino” of the substituent group A, for example, cyclopropylamino, cyclopentylamino, cyclohexylamino and the like can be used.

As the “C₃₋₈ cycloalkyl-carbonylamino” of the “optionally substituted C₃₋₈ cycloalkyl-carbonylamino” of the substituent group A, for example, cyclopropylcarbonylamino, cyclopentylcarbonylamino, cyclohexylcarbonylamino and the like can be used.

As the “mono- or di-C₆₋₁₄ arylamino” of the “optionally substituted mono- or di-C₅₋₁₄ arylamino” of the substituent group A, for example, phenylamino, diphenylamino and the like can be used.

As the “mono- or di-C₇₋₁₆ aralkylamino” of the “optionally substituted mono- or di-C₇₋₁₆ aralkylamino” of the substituent group A, for example, benzylamino and the like can be used.

As the “C₆₋₁₄ aryl-carbonylamino” of the “optionally substituted C₆₋₁₄ aryl-carbonylamino” of the substituent group A, for example, benzoylamino, naphthoylamino and the like can be used.

As the “C₆₋₁₄ arylsulfonylamino” of the “optionally substituted C₆₋₁₄ arylsulfonylamino” of the substituent group A, for example, phenylsulfonylamino, 2-naphthylsulfonylamino, 1-naphthylsulfonylamino and the like can be used.

These “C₁₋₆ alkoxy-carbonyl”, “C₆₋₁₄ aryloxy-carbonyl”, “C₇₋₁₆ aralkyloxy-carbonyl”, “lower (C₁₋₆)alkyl”, “lower (C₂₋₆)alkenyl”, “lower (C₂₋₆)alkynyl”, “C₃₋₈ cycloalkyl”, “C₆₋₁₄ aryl”, “C₇₋₁₆ aralkyl”, “C₆₋₁₄ aryl-C₂₋₆ alkenyl”, “heterocyclic group”, “lower (C₁₋₆)alkoxy”, “C₆₋₁₄ aryloxy”, “C₇₋₁₆ aralkyloxy”, “lower (C₁₋₆)alkyl-carbonyloxy”, “lower (C₁₋₆)alkoxy-carbonyloxy”, “mono-lower (C₁₋₆)alkyl-carbamoyloxy”, “di-lower (C₁₋₆)alkyl-carbamoyloxy”, “C₆₋₁₄ aryl-carbonyloxy”, “mono- or di-C₆₋₁₄ aryl-carbamoyloxy”, “heterocycle-oxy”, “aromatic heterocycle-oxy”, “lower (C₁₋₆)alkylthio”, “C₆₋₁₄ arylthio”, “C₇₋₁₆ aralkylthio”, “lower (C₁₋₆)alkyl-carbonyl”, “C₃₋₈ cycloalkyl-carbonyl”, “C₆₋₁₄ aryl-carbonyl”, “C₇₋₁₆ aralkyl-carbonyl”, “lower (C₁₋₆)alkylsulfonyl”, “C₆₋₁₄ arylsulfonyl”, “lower (C₁₋₆)alkylsulfinyl”, “C₆₋₁₄ arylsulfinyl”, “lower (C₁₋₆)alkyl-carbamoyl”, “mono- or di-lower (C₁₋₆)alkylamino”, “lower (C₁₋₆)alkyl-carbonylamino”, “lower (C₁₋₆)alkoxy-carbonylamino”, “lower (C₁₋₆)alkylsulfonylamino”, “mono- or di-C₃₋₈ cycloalkylamino”, “C₃₋₈ cycloalkyl-carbonylamino”, “heterocycle-carbonylamino”, “mono- or di-C₆₋₁₄ arylamino”, “mono- or di-C₇₋₁₆ aralkylamino”, “C₆₋₁₄ aryl-carbonylamino” and “C₆₋₁₄ arylsulfonylamino” may have 1 to 5 substituents selected from, for example,

halogen atoms (e.g., fluorine atom, chlorine atom, bromine atom, iodine atom);

hydroxy;

nitro;

cyano;

C₁₋₆ alkyl (wherein said C₁₋₆ alkyl is optionally substituted by halogen atom, hydroxy, amino, mono- or di-C₁₋₆ alkylamino, mono- or di-C₆₋₁₄ arylamino, C₃₋₈ cycloalkyl, C₁₋₆ alkoxy, formyl, C₁₋₆ alkyl-carbonyl, C₃₋₈ cycloalkyl-carbonyl, C₆₋₁₄ aryl-carbonyl, C₇₋₁₆ aralkyl-carbonyl, C₁₋₆ alkoxy-carbonyl, C₆₋₁₄ aryloxy-carbonyl, C₇₋₁₆ aralkyloxy-carbonyl, C₁₋₆ alkylthio, C₁₋₆ alkylsulfinyl, C₁₋₆ alkylsulfonyl, carbamoyl, thiocarbamoyl, mono- or di-C₁₋₆ alkyl-carbamoyl, mono- or di-C₆₋₁₄ aryl-carbamoyl or the like);

C₂₋₆ alkenyl (wherein said C₂₋₆ alkenyl is optionally substituted by halogen atom, hydroxy, amino, mono- or di-C₁₋₆ alkylamino, mono- or di-C₆₋₁₄ arylamino, C₃₋₈ cycloalkyl, C₁₋₆ alkoxy, formyl, C₁₋₆ alkyl-carbonyl, C₃₋₈ cycloalkyl-carbonyl, C₆₋₁₄ aryl-carbonyl, C₇₋₁₆ aralkyl-carbonyl, C₁₋₆ alkoxy-carbonyl, C₆₋₁₄ aryloxy-carbonyl, C₇₋₁₆ aralkyloxy-carbonyl, C₁₋₆ alkylthio, C₁₋₆ alkylsulfinyl, C₁₋₆ alkylsulfonyl, carbamoyl, thiocarbamoyl, mono- or di-C₁₋₆ alkyl-carbamoyl, mono- or di-C₆₋₁₄ aryl-carbamoyl or the like);

C₂₋₆ alkynyl (wherein said C₂₋₆ alkynyl is optionally substituted by halogen atom, hydroxy, amino, mono- or di-C₁₋₆ alkylamino, mono- or di-C₆₋₁₄ arylamino, C₃₋₈ cycloalkyl, C₁₋₆ alkoxy, formyl, C₁₋₆ alkyl-carbonyl, C₃₋₈ cycloalkyl-carbonyl, C₆₋₁₄ aryl-carbonyl, C₇₋₁₆ aralkyl-carbonyl, C₁₋₆ alkoxy-carbonyl, C₆₋₁₄ aryloxy-carbonyl, C₇₋₁₆ aralkyloxy-carbonyl, C₁₋₆ alkylthio, C₁₋₆ alkylsulfinyl, C₁₋₆ alkylsulfonyl, carbamoyl, thiocarbamoyl, mono- or di-C₁₋₆ alkyl-carbamoyl, mono- or di-C₆₋₁₄ aryl-carbamoyl or the like);

C₆₋₁₄ aryl (wherein said C₆₋₁₄ aryl is optionally substituted by halogen atom, hydroxy, amino, optionally halogenated C₁₋₆ alkyl, mono- or di-C₁₋₆ alkylamino, mono- or di-C₆₋₁₄ arylamino, C₃₋₈ cycloalkyl, C₁₋₆ alkoxy, formyl, C₁₋₆ alkyl-carbonyl, C₃₋₈ cycloalkyl-carbonyl, C₆₋₁₄ aryl-carbonyl, C₇₋₁₆ aralkyl-carbonyl, C₁₋₆ alkoxy-carbonyl, C₆₋₁₄ aryloxy-carbonyl, C₇₋₁₆ aralkyloxy-carbonyl, C₁₋₆ alkylthio, C₁₋₆ alkylsulfinyl, C₁₋₆ alkylsulfonyl, carbamoyl, thiocarbamoyl, mono- or di-C₁₋₆ alkyl-carbamoyl, mono- or di-C₆₋₁₄ aryl-carbamoyl or the like);

C₆₋₁₄ aryloxy (wherein said C₆₋₁₄ aryloxy is optionally substituted by halogen atom, hydroxy, cyano, amino, optionally halogenated C₁₋₆ alkyl, mono- or di-C₁₋₆ alkylamino, mono- or di-C₆₋₁₄ arylamino, C₃₋₈ cycloalkyl, C₁₋₆ alkoxy, formyl, C₁₋₆ alkyl-carbonyl, C₃₋₈ cycloalkyl-carbonyl, C₆₋₁₄ aryl-carbonyl, C₇₋₁₆ aralkyl-carbonyl, C₁₋₆ alkoxy-carbonyl, C₆₋₁₄ aryloxy-carbonyl, C₇₋₁₆ aralkyloxy-carbonyl, C₁₋₆ alkylthio, C₁₋₆ alkylsulfinyl, C₁₋₆ alkylsulfonyl, carbamoyl, thiocarbamoyl, mono- or di-C₁₋₆ alkyl-carbamoyl, mono- or di-C₆₋₁₄ aryl-carbamoyl or the like);

C₇₋₁₆ aralkyloxy (wherein said C₇₋₁₆ aralkyloxy is optionally substituted by halogen atom, hydroxy, amino, optionally halogenated C₁₋₆ alkyl, mono- or di-C₁₋₆ alkylamino, mono- or di-C₆₋₁₄ arylamino, C₃₋₈ cycloalkyl, C₁₋₆ alkoxy, formyl, C₁₋₆ alkyl-carbonyl, C₃₋₈ cycloalkyl-carbonyl, C₆₋₁₄ aryl-carbonyl, C₇₋₁₆ aralkyl-carbonyl, C₁₋₆ alkoxy-carbonyl, C₆₋₁₄ aryloxy-carbonyl, C₇₋₁₆ aralkyloxy-carbonyl, C₁₋₆ alkylthio, C₁₋₆ alkylsulfinyl, C₁₋₆ alkylsulfonyl, carbamoyl, thiocarbamoyl, mono- or di-C₁₋₆ alkyl-carbamoyl, mono- or di-C₆₋₁₄ aryl-carbamoyl or the like);

a 5 to 10-membered heterocyclic group containing, besides carbon atom, 1 to 4 hetero atoms of 1 or 2 kinds selected from a nitrogen atom, a sulfur atom and an oxygen atom (e.g., furyl, pyridyl, thienyl, pyrrolidino (1-pyrrolidinyl), 1-piperidinyl, 4-piperidinyl, piperazinyl, 4-morpholinyl, 4-thiomorpholinyl, azepan-1-yl, azocan-1-yl, azonan-1-yl, 3,4-dihydroisoquinolin-2-yl etc.) (wherein said heterocyclic group is optionally substituted by halogen atom, hydroxy, amino, mono- or di-C₁₋₆ alkylamino, mono- or di-C₆₋₁₄ arylamino, mono- or di-C₇₋₁₆ aralkylamino, C₃₋₈ cycloalkyl, C₁₋₆ alkoxy, formyl, C₁₋₆ alkyl-carbonyl, C₃₋₈ cycloalkyl-carbonyl, C₆₋₁₄ aryl-carbonyl, C₇₋₁₆ aralkyl-carbonyl, C₁₋₆ alkoxy-carbonyl, C₆₋₁₄ aryloxy-carbonyl, C₇₋₁₆ aralkyloxy-carbonyl, C₁₋₆ alkylthio, C₁₋₆ alkylsulfinyl, C₁₋₆ alkylsulfonyl, carbamoyl, thiocarbamoyl, mono- or di-C₁₋₆ alkyl-carbamoyl, mono- or di-C₆₋₁₄ aryl-carbamoyl or the like); optionally substituted amino group [e.g., amino group optionally substituted by 1 or 2 substituents selected from the group consisting of C₁₋₆ alkyl, C₂₋₆ alkenyl, C₆₋₁₄ aryl, C₇₋₁₆ aralkyl, heterocyclic group and heterocycle-C₁₋₆ alkyl (wherein said C₁₋₆ alkyl, C₂₋₆ alkenyl, C₆₋₁₄ aryl, C₇₋₁₆ aralkyl, heterocyclic group and heterocycle-C₁₋₆ alkyl are each optionally substituted by halogen atom, hydroxy, cyano, amino, optionally halogenated C₁₋₆ alkyl, mono- or di-C₁₋₆ alkylamino, mono- or di-C₆₋₁₄ arylamino, C₃₋₈ cycloalkyl, C₁₋₆ alkoxy, formyl, C₁₋₆ alkyl-carbonyl, C₃₋₈ cycloalkyl-carbonyl, C₆₋₁₄ aryl-carbonyl, C₇₋₁₆ aralkyl-carbonyl, C₁₋₆ alkoxy-carbonyl, C₆₋₁₄ aryloxy-carbonyl, C₇₋₁₆ aralkyloxy-carbonyl, C₁₋₆ alkylthio, C₁₋₆ alkylsulfinyl, C₁₋₆ alkylsulfonyl, carbamoyl, thiocarbamoyl, mono- or di-C₁₋₆ alkyl-carbamoyl, mono- or di-C₆₋₁₄ aryl-carbamoyl or the like, wherein, as the “heterocycle” of the “heterocycle” and “heterocycle-C₁₋₆ alkyl”, those similar to the “heterocyclic group” of the aforementioned “heterocyclic group optionally having substituents” are used);

C₃₋₈ cycloalkyl;

C₁₋₆ alkoxy (wherein said C₁₋₆ alkoxy is optionally substituted by halogen atom, hydroxy, amino, mono- or di-C₁₋₆ alkylamino, mono- or di-C₆₋₁₄ arylamino, C₃₋₈ cycloalkyl, C₁₋₆ alkoxy, formyl, C₁₋₆ alkyl-carbonyl, C₃₋₈ cycloalkyl-carbonyl, C₆₋₁₄ aryl-carbonyl, C₇₋₁₆ aralkyl-carbonyl, C₁₋₆ alkoxy-carbonyl, C₆₋₁₄ aryloxy-carbonyl, C₇₋₁₆ aralkyloxy-carbonyl, C₁₋₆ alkylthio, C₁₋₆ alkylsulfinyl, C₁₋₆ alkylsulfonyl, carbamoyl, thiocarbamoyl, mono- or di-C₁₋₆ alkyl-carbamoyl, mono- or di-C₆₋₁₄ aryl-carbamoyl or the like);

formyl;

C₁₋₆ alkyl-carbonyl (e.g., acetyl);

C₃₋₈ cycloalkyl-carbonyl;

C₆₋₁₄ aryl-carbonyl;

C₇₋₁₆ aralkyl-carbonyl;

C₁₋₆ alkoxy-carbonyl;

C₆₋₁₄ aryloxy-carbonyl;

C₇₋₁₆ aralkyloxy-carbonyl;

C₁₋₆ alkylthio;

C₁₋₆ alkylsulfinyl;

C₁₋₆ alkylsulfonyl;

carbamoyl;

thiocarbamoyl;

mono-C₁₋₆ alkyl-carbamoyl (e.g., methylcarbamoyl, ethylcarbamoyl etc.);

di-C₁₋₆ alkyl-carbamoyl (e.g., dimethylcarbamoyl, diethylcarbamoyl, ethylmethylcarbamoyl etc.);

mono- or di-C₆₋₁₄ aryl-carbamoyl (e.g., phenylcarbamoyl, 1-naphthylcarbamoyl, 2-naphthylcarbamoyl etc.);

mono- or di-5 to 7-membered heterocycle-carbamoyl containing, besides carbon atom, 1 to 4 hetero atoms of 1 or 2 kinds selected from a nitrogen atom, a sulfur atom and an oxygen atom (e.g., 2-pyridylcarbamoyl, 3-pyridylcarbamoyl, 4-pyridylcarbamoyl, 2-thienylcarbamoyl, 3-thienylcarbamoyl etc.);

sulfamoyl;

and the like (hereinafter to be abbreviated as substituent group B) at respective substitutable positions.

In addition, the “heterocycle-carbonyl” of the “optionally substituted heterocycle-carbonyl” of the substituent group A may have 1 to 5 substituents selected from (i) substituents selected from the above-mentioned substituent group B (except a phenyl group and a 5 to 10-membered heterocyclic group containing, besides carbon atom, 1 to 4 hetero atoms of 1 or 2 kinds selected from a nitrogen atom, a sulfur atom and an oxygen atom (wherein said heterocyclic group is optionally substituted by halogen atom, hydroxy, amino, mono- or di-C₁₋₆ alkylamino, mono- or di-C₆₋₁₄ arylamino, mono- or di-C₇₋₁₆ aralkylamino, C₃₋₈ cycloalkyl, C₁₋₆ alkoxy, formyl, C₁₋₆ alkyl-carbonyl, C₃₋₈ cycloalkyl-carbonyl, C₆₋₁₄ aryl-carbonyl, C₇₋₁₆ aralkyl-carbonyl, C₁₋₆ alkoxy-carbonyl, C₆₋₁₄ aryloxy-carbonyl, C₇₋₁₆ aralkyloxy-carbonyl, C₁₋₆ alkylthio, C₁₋₆ alkylsulfinyl, C₁₋₆ alkylsulfonyl, carbamoyl, thiocarbamoyl, mono- or di-C₁₋₆ alkyl-carbamoyl, mono- or di-C₆₋₁₄ aryl-carbamoyl or the like)), (ii) a phenyl group, a benzyl group, a phenethyl group, a styryl group, a phenylethynyl group or a phenoxymethyl group each optionally having 1 to 5 substituents selected from the above-mentioned substituent group B and the like, (iii) the aforementioned “heterocyclic group optionally having substituents”, (iv) a group represented by the formula -Z¹-Z² wherein Z¹ is a bond, methylene (CH₂), ethylene (CH₂CH₂), vinylene (CHCH), ethynylene (CC) or methyleneoxy (CH₂O), and Z² is a phenyl group optionally having substituents or a heterocyclic group optionally having substituents, and the like at substitutable positions. As Z², (i) a phenyl group optionally having substituents selected from the group consisting of (a) a halogen atom, (b) cyano, (c) an optionally halogenated C₁₋₆ alkyl group, (d) an optionally halogenated C₁₋₆ alkoxy group, (e) carbamoyl and (f) sulfamoyl, (ii) a 5 to 10-membered monocyclic or bicyclic heterocyclic group containing, besides carbon atom, 1 to 4 hetero atoms of 1 or 2 kinds selected from a nitrogen atom, a sulfur atom and an oxygen atom, which optionally has substituents selected from the group consisting of (a) a halogen atom, (b) cyano, (c) an optionally halogenated C₁₋₆ alkyl group, (d) an optionally halogenated C₁₋₆ alkoxy group, (e) carbamoyl and (f) sulfamoyl and the like are preferable.

As the “carbamoyl group optionally having substituents” of the substituent group A, carbamoyl group optionally having 1 or 2 substituents selected from the above-mentioned optionally substituted lower (C₁₋₆)alkyl (particularly, lower (C₁₋₆)alkyl optionally substituted by the “optionally substituted amino group” of the substituent group B), the above-mentioned optionally substituted lower (C₂₋₆)alkenyl, the above-mentioned optionally substituted lower (C₂₋₆)alkynyl, the above-mentioned optionally substituted C₃₋₈ cycloalkyl, the above-mentioned optionally substituted C₆₋₁₄ aryl, the above-mentioned optionally substituted C₇₋₁₆ aralkyl, the above-mentioned heterocyclic group optionally having substituents, the above-mentioned optionally substituted lower (C₁₋₆)alkoxy, formyl, the above-mentioned optionally substituted lower (C₁₋₆)alkyl-carbonyl, the above-mentioned optionally substituted C₃₋₈ cycloalkyl-carbonyl, the above-mentioned optionally substituted C₆₋₁₄ aryl-carbonyl, the above-mentioned optionally substituted C₇₋₁₆ aralkyl-carbonyl, the above-mentioned heterocycle-carbonyl, the above-mentioned optionally substituted lower (C₁₋₆)alkylsulfonyl, the above-mentioned optionally substituted C₆₋₁₄ arylsulfonyl and the like can be used. Specifically, the above-mentioned optionally substituted lower (C₁₋₆)alkyl-carbamoyl is preferable, such as carbamoyl, mono-C₁₋₆ alkyl-carbamoyl (e.g., methylcarbamoyl, ethylcarbamoyl, propylcarbamoyl etc.), di-C₁₋₆ alkyl-carbamoyl(e.g., dimethylcarbamoyl, diethylcarbamoyl, ethylmethylcarbamoyl etc.), 5 to 7-membered heterocycle-C₁₋₆ alkyl(C₁₋₆ alkyl)-carbamoyl wherein the heterocycle contains, besides carbon atom, 1 to 4 hetero atoms of 1 or 2 kinds selected from a nitrogen atom, a sulfur atom and an oxygen atom (e.g., ethyl(thienylmethyl)carbamoyl etc.), mono- or di-C₁₋₆ alkylamino-C₁₋₆ alkyl-carbamoyl (e.g., dimethylamino-ethylcarbamoyl, dimethylamino-propylcarbamoyl, dimethylamino-butylcarbamoyl etc.), mono- or di-5 to 7-membered heterocycle-C₁₋₆ alkyl-carbamoyl containing, besides carbon atom, 1 to 4 hetero atoms of 1 or 2 kinds selected from a nitrogen atom, a sulfur atom and an oxygen atom (e.g., 1-piperidinylethyl-carbamoyl, 4-morpholinylethyl-carbamoyl, 4-morpholinylpropyl-carbamoyl, pyridylmethyl-carbamoyl etc.), mono- or di-5 to 7-membered heterocyclecarbamoyl containing, besides carbon atom, 1 to 4 hetero atoms of 1 or 2 kinds selected from a nitrogen atom, a sulfur atom and an oxygen atom (e.g., 2-pyridylcarbamoyl, 3-pyridylcarbamoyl, 4-pyridylcarbamoyl, 2-thienylcarbamoyl, 3-thienylcarbamoyl etc.) and the like. As the “carbamoyl group optionally having substituents”, 5 to 7-membered cyclic carbamoyl optionally having similar substituents (e.g., 1-pyrrolidinylcarbonyl, 1-piperidinylcarbonyl, 1-piperazinylcarbonyl, hexamethyleneiminocarbonyl) and the like can be also used.

As the “optionally substituted amino” of the substituent group A, amino optionally substituted by 1 or 2 substituents selected from the above-mentioned optionally substituted lower (C₁₋₆) alkyl, the above-mentioned optionally substituted lower (C₂₋₆)alkenyl, the above-mentioned optionally substituted lower (C₂₋₆)alkynyl, the above-mentioned optionally substituted C₃₋₈ cycloalkyl, the above-mentioned optionally substituted C₆₋₁₄ aryl, the above-mentioned optionally substituted C₇₋₁₆ aralkyl, the above-mentioned optionally substituted lower (C₁₋₆)alkoxy, formyl, the above-mentioned optionally substituted lower (C₁₋₆)alkyl-carbonyl, the above-mentioned optionally substituted C₃₋₈ cycloalkyl-carbonyl, the above-mentioned optionally substituted C₆₋₁₄ aryl-carbonyl, the above-mentioned optionally substituted C₇₋₁₆ aralkyl-carbonyl, the above-mentioned optionally substituted heterocycle-carbonyl, the above-mentioned optionally substituted lower (C₁₋₆)alkoxy-carbonyl, the above-mentioned optionally substituted lower (C₁₋₆)alkylsulfonyl, the above-mentioned optionally substituted C₆₋₁₄ arylsulfonyl, the above-mentioned carbamoyl optionally having substituents and the like can be used, and particularly, amino, the above-mentioned optionally substituted mono- or di-lower (C₁₋₆)alkylamino, the above-mentioned optionally substituted mono- or di-C₆₋₁₄ arylamino, the above-mentioned optionally substituted mono- or di-C₇₋₁₆ aralkylamino, the above-mentioned optionally substituted C₆₋₁₄ aryl-carbonylamino, formylamino, the above-mentioned optionally substituted lower (C₁₋₆)alkyl-carbonylamino, the above-mentioned optionally substituted C₃₋₈ cycloalkyl-carbonylamino, the above-mentioned optionally substituted heterocycle-carbonylamino, the above-mentioned optionally substituted lower (C₁₋₆)alkoxy-carbonylamino, the above-mentioned optionally substituted lower (C₁₋₆)alkylsulfonylamino, the above-mentioned optionally substituted C₆₋₁₄ arylsulfonylamino and the like are preferably used.

In addition, as the substituent of the benzene ring for A, a group represented by the formula Z-Y²—Y¹— wherein Y¹ and Y² are each a bond, an oxygen atom, an optionally oxidized sulfur atom, an imino group optionally having a substituent or a carbonyl group and Z is a hydrogen atom, a hydrocarbon group optionally having substituents or a heterocyclic group optionally having substituents and the like can be also used.

As the “optionally oxidized sulfur atom” for Y¹ or Y², S, SO or SO₂ can be used.

As the substituent of the “imino group optionally having a substituent” for Y¹ or Y², the “optionally substituted lower (C₁₋₆)alkyl”, “optionally substituted lower (C₂₋₆)alkenyl”, “optionally substituted lower (C₂₋₆)alkynyl”, “optionally substituted C₃₋₈ cycloalkyl”, “optionally substituted C₆₋₁₄ aryl”, “optionally substituted C₇₋₁₆ aralkyl”, “optionally substituted lower (C₁₋₆)alkoxy”, “optionally substituted lower (C₁₋₆)alkyl-carbonyl”, “optionally substituted C₃₋₈ cycloalkyl-carbonyl”, “optionally substituted C₆₋₁₄ aryl-carbonyl”, “optionally substituted C₇₋₁₆ aralkyl-carbonyl”, “optionally substituted heterocycle-carbonyl”, “optionally substituted lower (C₁₋₆) alkylsulfonyl”, “optionally substituted C₆₋₁₄ arylsulfonyl”, “heterocyclic group optionally having substituents” and the like of the substituent group A can be used. Of these, the “optionally substituted lower (C₁₋₆)alkyl” is preferable, and a C₁₋₆ alkyl group such as methyl, ethyl and the like is particularly preferable.

As the “hydrocarbon group optionally having substituents” for Z, those similar to the below-mentioned “hydrocarbon group optionally having substituents” for R¹ can be used.

As the “heterocyclic group optionally having substituents” for Z, those similar to the “heterocyclic group optionally having substituents” of the aforementioned substituent group A can be used.

As Y¹, an oxygen atom, —NH—, a carbonyl group and the like are preferable, and a carbonyl group is particularly preferable.

As Y², a bond, an imino group optionally having a substituent, a carbonyl group and the like are preferable, and a bond, an imino group optionally having a substituent and the like are particularly preferable. As the imino group optionally having a substituent, —NR⁴— (R⁴ is a hydrogen atom or C₁₋₆ alkyl group) and the like are preferable. As Y², a bond, —NH— or a carbonyl group is particularly preferable, and a bond or —NH— is preferable.

As Z, a hydrogen atom, the above-mentioned C₁₋₆ alkyl group optionally having substituents, the above-mentioned C₆₋₁₄ aryl group optionally having substituents (particularly, phenyl group), the above-mentioned C₇₋₁₆ aralkyl group optionally having substituents (particularly, benzyl group), and the above-mentioned heterocyclic group optionally having substituents are preferable.

As said C₁₋₆ alkyl group optionally having substituents, a C₁₋₆ alkyl group (e.g., methyl, ethyl, propyl, butyl) optionally having substituents selected from, for example, mono- or di-C₁₋₆ alkylamino (e.g., dimethylamino), a 5 to 7-membered heterocyclic group containing, besides carbon atom, 1 to 4 hetero atoms of 1 or 2 kinds selected from a nitrogen atom, a sulfur atom and an oxygen atom (e.g., thienyl, pyridyl, 1-piperidinyl, 4-morpholinyl) and the like can be used, and dimethylamino-ethyl, dimethylamino-propyl, dimethylamino-butyl and the like can be specifically used.

As said C₆₋₁₄ aryl group optionally having substituents, for example, a phenyl group optionally having substituents selected from a halogen atom, C₁₋₆ alkyl, amino, mono- or di-C₁₋₆ alkylamino and the like, and the like can be used.

As said C₇₋₁₆ aralkyl group optionally having substituents, for example, a benzyl group optionally having substituents selected from a halogen atom, C₁₋₆ alkyl, amino, mono- or di-C₁₋₆ alkylamino and the like, and the like can be used.

As the “heterocyclic group” of said heterocyclic group optionally having substituents, for example, a 5 to 7-membered heterocyclic group containing, besides carbon atom, 1 to 4 hetero atoms of 1 or 2 kinds selected from a nitrogen atom, a sulfur atom and an oxygen atom (particularly, a group free of a hydrogen atom on a nitrogen atom of heterocycle) can be used. Of these, a 5 to 7-membered, preferably 6-membered, non-aromatic nitrogen-containing heterocyclic group such as 1-piperidinyl, 1-piperazinyl and the like is preferable, and 1-piperidinyl is particularly preferable.

As the “substituent” of said “heterocyclic group optionally having substituents”, for example,

(a) hydroxy,

(b) C₁₋₆ alkoxy-carbonyl (e.g., tert-butoxy-carbonyl),

(c) a 5 to 10-membered heterocyclic group containing, besides carbon atom, 1 to 4 hetero atoms of 1 or 2 kinds selected from a nitrogen atom, a sulfur atom and an oxygen atom (e.g., pyrrolidino (1-pyrrolidinyl), 1-piperidinyl, 4-morpholinyl, 4-thiomorpholinyl, azepan-1-yl, azocan-1-yl, azonan-1-yl, 3,4-dihydroisoquinolin-2-yl),

(d) mono- or di-C₁₋₆ alkylamino (e.g., diethylamino),

(e) mono- or di-C₇₋₁₆ aralkylamino (e.g., benzylamino)

(f) 5 to 7-membered heterocycle-C₁₋₆ alkyl(C₁₋₆ alkyl)amino wherein the heterocycle contains, besides carbon atom, 1 to 4 hetero atoms of 1 or 2 kinds selected from a nitrogen atom, a sulfur atom and an oxygen atom (e.g., ethyl(thienylmethyl)amino),

(g) C₁₋₆ alkyl(C₇₋₁₆ aralkyl)amino wherein the C₁₋₆ alkyl is optionally substituted by hydroxy (e.g., methyl(benzyl)amino, ethyl(benzyl)amino, 2-hydroxyethyl(benzyl)amino) and the like can be preferably used.

As a preferable combination of Y¹, Y² and Z,

(1) Y¹ is a carbonyl group, Y² is an imino group optionally having a substituent, and Z is a hydrocarbon group optionally having substituents (particularly, a C₁₋₆ alkyl group optionally having substituents such as the “optionally substituted amino group” of substituent group B and the like);

(2) Y¹ is a carbonyl group, Y² is a bond, and Z is a heterocyclic group optionally having substituents;

(3) Y¹ is —NH—, Y² is a carbonyl group, and Z is a hydrocarbon group optionally having substituents (particularly, a C₁₋₆ alkyl group optionally having substituents, a C₆₋₁₄ aryl group optionally having substituents);

(4) Y¹ is an oxygen atom, Y² is a bond, and Z is a C₁₋₆ alkyl group optionally having substituents, a C₇₋₁₆ aralkyl group optionally having substituents and the like can be mentioned.

As the substituent of the benzene ring for A, from among the above-mentioned substituent group A, for example, an optionally substituted C₁₋₆ alkyl group, an optionally esterified carboxyl group, an optionally substituted heterocycle-carbonyl (particularly 5 to 7-membered heterocycle-carbonyl containing, besides carbon atom, 1 to 4 hetero atoms of 1 or 2 kinds selected from a nitrogen atom, a sulfur atom and an oxygen atom) and a carbamoyl group optionally having substituents are preferable, and of these, optionally substituted heterocycle-carbonyl (particularly, 5 to 7-membered heterocycle-carbonyl containing, besides carbon atom, 1 to 4 hetero atoms of 1 or 2 kinds selected from a nitrogen atom, a sulfur atom and an oxygen atom) and a carbamoyl group optionally having substituents are preferable, and a 1-piperidinylcarbonyl group optionally having substituents is particularly preferable.

As the substituent of the benzene ring for A, the above-mentioned group represented by the formula Z-Y²—Y¹— is also preferable.

Particularly, as the substituent of the benzene ring for A,

(i) a C₁₋₆ alkyl group (e.g., methyl),

(ii) a carboxyl group,

(iii) a C₁₋₆ alkoxy-carbonyl group (e.g., methoxycarbonyl, ethoxycarbonyl),

(iv) 5 to 7-membered heterocycle-carbonyl containing, besides carbon atom, 1 to 4 hetero atoms of 1 or 2 kinds selected from a nitrogen atom, a sulfur atom and an oxygen atom (e.g., 1-piperidinylcarbonyl), which is optionally substituted by substituents selected from the following (a) to (g);
(a) hydroxy,
(b) C₁₋₆ alkoxy-carbonyl (e.g., tert-butoxy-carbonyl),
(c) a group represented by the formula -Z¹-Z²
   (wherein Z¹ and Z² are as defined below, and preferably, Z² is (i) a phenyl group optionally having substituents selected from the group consisting of (a) a halogen atom (e.g., fluorine, chlorine), (b) cyano, (c) an optionally halogenated C₁₋₆ alkyl group (e.g., methyl, trifluoromethyl), (d) an optionally halogenated C₁₋₆ alkoxy group (e.g., methoxy, trifluoromethoxy), (e) carbamoyl, (f) sulfamoyl and the like, or (ii) a 5 to 10-membered monocyclic or bicyclic heterocyclic group containing, besides carbon atom, 1 to 4 hetero atoms of 1 or 2 kinds selected from a nitrogen atom, a sulfur atom and an oxygen atom (particularly, pyrrolyl (e.g., 1-pyrrolyl, 2-pyrrolyl, 3-pyrrolyl), furyl (e.g., 2-furyl, 3-furyl), thienyl (e.g., 2-thienyl, 3-thienyl), imidazolyl (e.g., 1-imidazolyl, 2-imidazolyl, 4-imidazolyl), oxazolyl (e.g., 2-oxazolyl, 4-oxazolyl, 5-oxazolyl), thiazolyl (e.g., 2-thiazolyl, 4-thiazolyl, 5-thiazolyl), pyrazolyl (e.g., 1-pyrazolyl, 3-pyrazolyl, 4-pyrazolyl), isoxazolyl (e.g., 3-isoxazolyl, 4-isoxazolyl, 5-isoxazolyl), isothiazolyl (e.g., 3-isothiazolyl, 4-isothiazolyl, 5-isothiazolyl), pyridyl (e.g., 2-pyridyl, 3-pyridyl, 4-pyridyl), pyridazinyl (e.g., 3-pyridazinyl, 4-pyridazinyl), pyrimidinyl (e.g., 2-pyrimidinyl, 4-pyrimidinyl), pyrazinyl and the like can be mentioned, particularly a 5 to 10-membered monocyclic aromatic heterocyclic group containing, besides carbon atom, 1 to 4 hetero atoms of 1 or 2 kinds selected from a nitrogen atom, a sulfur atom and an oxygen atom, such as furyl (e.g., 2-furyl, 3-furyl), thienyl (e.g., 2-thienyl, 3-thienyl) and the like; a 5 to 10-membered bicyclic aromatic heterocyclic group containing, besides 5 carbon atom, 1 to 4 hetero atoms of 1 or 2 kinds selected from a nitrogen atom, a sulfur atom and an oxygen atom, such as indolyl (e.g., 1-indolyl, 2-indolyl, 3-indolyl, 4-indolyl, 5-indolyl, 6-indolyl, 7-indolyl), isoindolyl (e.g., 1-isoindolyl, 2-isoindolyl, 3-isoindolyl, 4-isoindolyl, 5-isoindolyl, 6-isoindolyl, 7-isoindolyl), benzo[b]furanyl (e.g., 2-benzo[b]furanyl, 3-benzo[b]furanyl, 4-benzo[b]furanyl, 5-benzo[b]furanyl, 6-benzo[b]furanyl, 7-benzo[b]furanyl), benzo[c]furanyl (e.g., 1-benzo[c]furanyl, 4-benzo[c]uranyl, 5-benzo[c]furanyl), benzo[b]thienyl (e.g., 2-benzo[b]thienyl, 3-benzo[b]thienyl, 4-benzo[b]thienyl, 5-benzo[b]thienyl, 6-benzo[b]thienyl, 7-benzo[b]thienyl), benzo[c]thienyl (e.g., 1-benzo[c]thienyl, 4-benzo[c]thienyl, 5-benzo[c]thienyl), benzimidazolyl (e.g., 1-benzimidazolyl, 2-benzimidazolyl, 4-benzimidazolyl, 5-benzimidazolyl), benzoxazolyl (e.g., 2-benzoxazolyl, 4-benzoxazolyl, 5-benzoxazolyl, 6-benzoxazolyl, 7-benzoxazolyl), benzothiazolyl (e.g., 2-benzothiazolyl, 4-benzothiazolyl, 5-benzothiazolyl, 6-benzothiazolyl, 7-benzothiazolyl), indazolyl (e.g., 1-indazolyl, 2-indazolyl, 3-indazolyl, 4-indazolyl, 5-indazolyl, 6-indazolyl, 7-indazolyl), 1,2-benzisoxazolyl (e.g., 1,2-benzisoxazol-3-yl, 1,2-benzisoxazol-4-yl, 1,2-benzisoxazol-5-yl, 1,2-benzisoxazol-6-yl, 1,2-benzisoxazol-7-yl), 1,2-benzisothiazolyl (e.g., 1,2-benzisothiazol-3-yl, 1,2-benzisothiazol-4-yl, 1,2-benzisothiazol-5-yl, 1,2-benzisothiazol-6-yl, 1,2-benzisothiazol-7-yl), quinolyl (e.g., 2-quinolyl, 3-quinolyl, 4-quinolyl, 5-quinolyl, 6-quinolyl, 7-quinolyl, 8-quinolyl), isoquinolyl (e.g., 1-isoquinolyl, 3-isoquinolyl, 4-isoquinolyl, 5-isoquinolyl, 6-isoquinolyl, 7-isoquinolyl, 8-isoquinolyl), cinnolinyl (e.g., 3-cinnolinyl, 4-cinnolinyl, 5-cinnolinyl, 6-cinnolinyl, 7-cinnolinyl, 8-cinnolinyl), phthalazinyl (e.g., 1-phthalazinyl, 4-phthalazinyl, 5-phthalazinyl, 6-phthalazinyl, 7-phthalazinyl, 8-phthalazinyl), quinazolinyl (e.g., 2-quinazolinyl, 4-quinazolinyl, 5-quinazolinyl, 6-quinazolinyl, 7-quinazolinyl, 8-quinazolinyl), quinoxalinyl (e.g., 2-quinoxalinyl, 3-quinoxalinyl, 5-quinoxalinyl, 6-quinoxalinyl, 7-quinoxalinyl, 8-quinoxalinyl) and the like can be mentioned, particularly, quinolyl (e.g., 2-quinolyl, 3-quinolyl, 4-quinolyl, 5-quinolyl, 6-quinolyl, 7-quinolyl, 8-quinolyl), isoquinolyl (e.g., 1-isoquinolyl, 3-isoquinolyl, 4-isoquinolyl, 5-isoquinolyl, 6-isoquinolyl, 7-isoquinolyl, 8-isoquinolyl); a 5 to 10-membered non-aromatic heterocyclic group containing, besides carbon atom, 1 to 4 hetero atoms of 1 or 2 kinds selected from a nitrogen atom, a sulfur atom and an oxygen atom such as pyrrolidino (1-pyrrolidinyl), 1-piperidinyl, 4-morpholinyl, 4-thiomorpholinyl, azepan-1-yl, azocan-1-yl, azonan-1-yl, 3,4-dihydroisoquinolin-2-yl; and the like), which optionally has substituents selected from the group consisting of (a) a halogen atom (e.g., fluorine, chlorine), (b) cyano, (c) an optionally halogenated C₁₋₆ alkyl group (e.g., methyl, trifluoromethyl), (d) an optionally halogenated C₁₋₆ alkoxy group (e.g., methoxy, trifluoromethoxy), (e) carbamoyl, (f) sulfamoyl and the like),
(d) mono- or di-C₁₋₆ alkylamino (e.g., diethylamino),
(e) mono- or di-C₇₋₁₆ aralkylamino (e.g., benzylamino)
(f) 5 to 7-membered heterocycle-C₁₋₆ alkyl(C₁₋₆ alkyl)amino wherein the heterocycle contains, besides carbon atom, 1 to 4 hetero atoms of 1 or 2 kinds selected from a nitrogen atom, a sulfur atom and an oxygen atom (e.g., ethyl(thienylmethyl)amino),
(g) C₁₋₆ alkyl(C₇₋₁₆ aralkyl)amino wherein the C₁₋₆ alkyl is optionally substituted by hydroxy (e.g., methyl(benzyl)amino, ethyl(benzyl)amino, 2-hydroxyethyl(benzyl)amino);
   (v) a carbamoyl group optionally having 1 or 2 substituents selected from the following (a) to (c);
(a) C₁₋₆ alkyl (e.g., methyl, ethyl),
(b) C₁₋₆ alkyl (e.g., methyl, ethyl, propyl, butyl) substituted by mono- or di-C₁₋₆ alkylamino (e.g., dimethylamino), specifically, dimethylamino-ethyl, dimethylamino-propyl, dimethylamino-butyl,
(c) C₁₋₆ alkyl (e.g., methyl, ethyl, propyl) substituted by a 5 to 7-membered heterocyclic group containing, besides carbon atom, 1 to 4 hetero atoms of 1 or 2 kinds selected from a nitrogen atom, a sulfur atom and an oxygen atom (e.g., thienyl, pyridyl, 1-piperidinyl, 4-morpholinyl), specifically, thienylmethyl, pyridylmethyl, 1-piperidinylethyl, 4-morpholinylethyl, 4-morpholinylpropyl;
   (vi) a halogen atom (e.g., chlorine);
   (vii) 5 to 7-membered heterocycle-carbonyl-amino containing, besides carbon atom, 1 to 4 hetero atoms of 1 or 2 kinds selected from a nitrogen atom, a sulfur atom and an oxygen atom (e.g., pyridylcarbonylamino); and the like can be preferably used.

R¹, R² and R³ are each a hydrogen atom, a hydrocarbon group optionally having substituents or a heterocyclic group optionally having substituents.

As R², a hydrocarbon group optionally having substituents or a heterocyclic group optionally having substituents is preferable.

As the “hydrocarbon group” of the “hydrocarbon group optionally having substituents” for R¹, R² or R³, for example, an alkyl group, a cycloalkyl group, an alkenyl group, a cycloalkenyl group, an alkynyl group, an aralkyl group, an aryl group and the like can be mentioned.

As said “alkyl group”, for example, a “linear or branched C₁₋₁₅ alkyl group” such as methyl, ethyl, propyl, isopropyl, butyl, isobutyl, sec-butyl, tert-butyl, pentyl, neopentyl, hexyl, heptyl, octyl, nonyl, decyl, undecyl, tridecyl, tetradecyl, pentadecyl and the like, and the like can be used, a C₁₋₈ alkyl group can be preferably used, a C₁₋₆ alkyl group can be more preferably used, and a C₁₋₄ alkyl group can be still more preferably used.

As said “cycloalkyl group”, for example, a “C₃₋₁₀ cycloalkyl group” such as cyclopropyl, cyclobutyl, cyclopentyl, cyclohexyl, cycloheptyl, cyclooctyl, adamantyl and the like, and the like can be used, a C₃₋₈ cycloalkyl group can be more preferably used, and a C₅₋₇ cycloalkyl group can be still more preferably used.

As said “alkenyl group”, for example, a “C₂₋₁₈ alkenyl group” such as vinyl, allyl, isopropenyl, 3-butenyl, 3-octenyl, 9-octadecenyl and the like, and the like can be used, a C₂₋₆ alkenyl group can be more preferably used, and a C₂₋₄ alkenyl group can be still more preferably used.

As said “cycloalkenyl group”, for example, a “C₃₋₁₀ cycloalkenyl group” such as cyclopropenyl, cyclobutenyl, cyclopentenyl, cyclohexenyl, cycloheptenyl, cyclooctenyl and the like, and the like can be used, a C₃₋₈ cycloalkenyl group can be more preferably used, and a C₅₋₇ cycloalkenyl group can be still more preferably used.

As said “alkynyl group”, for example, a “C₂₋₈ alkynyl group” such as ethynyl, 1-propynyl, propargyl, 1-butynyl, 2-butynyl, 1-pentynyl, 2-pentynyl, 3-pentynyl and the like, and the like can be used, a C₂₋₆ alkynyl group can be more preferably used, and a C₂₋₄ alkynyl group can be still more preferably used.

As said “aryl group”, for example, an aromatic monocyclic, bicyclic or tricyclic C₆₋₁₄ aryl group such as phenyl, 1-naphthyl, 2-naphthyl, phenanthryl, anthryl and the like, a biphenyl group, a tolyl group and the like can be used, a C₆₋₁₀ aryl group such as phenyl, naphthyl and the like can be preferably used, and phenyl can be more preferably used.

As said “aralkyl group”, a C₇₋₁₆ aralkyl group and the like can be used. Specifically, for example, a phenyl-C₁₋₆ alkyl group such as benzyl, phenethyl, 3-phenylpropyl, 4-phenylbutyl and the like and, for example, a naphthyl-C₁₋₆ alkyl group such as (1-naphthyl)methyl, 2-(1-naphthyl)ethyl, 2-(2-naphthyl)ethyl and the like, and the like can be used.

As the substituent of the hydrocarbon group of these “alkyl group”, “alkenyl group”, “alkynyl group”, “cycloalkyl group”, “cycloalkenyl group”, “aralkyl group” and “aryl group”, substituents similar to the aforementioned substituent group B can be used.

As the “heterocyclic group optionally having substituents” for R¹, R² or R³, those similar to the “heterocyclic group optionally having substituents” of the aforementioned substituent group A can be used.

Note that R² is not a 4-hydroxyphenyl group optionally having substituents selected from the group consisting of a halogen atom, a C₁₋₆ alkyl group and a C₁₋₆ alkoxy group; a 4-methoxyphenyl group optionally having substituents selected from the group consisting of a halogen atom, a C₁₋₆ alkyl group and a C₁₋₆ alkoxy group; and a 6-hydroxypyridin-3-yl group optionally having substituents selected from the group consisting of a halogen atom, a C₁₋₆ alkyl group and a C₁₋₆ alkoxy group.

As R¹, a hydrogen atom is preferable.

As R², a hydrogen atom, a C₁₋₆ alkyl group (e.g., methyl, ethyl, propyl) and the like are preferable, and a C₁₋₆ alkyl group (e.g., methyl, ethyl, propyl) is particularly preferable.

As R³, a hydrogen atom is preferable.

[Compound (II)]

R¹ and R² optionally form a ring via X, when R¹ and R² form a ring via X, R¹ and R² are each a bond or a divalent C₁₋₅ acyclic hydrocarbon group optionally having substituents, and X is a bond, an oxygen atom, an optionally oxidized sulfur atom or an imino group optionally having a substituent. In this case, the compound of the present invention is represented by the above-mentioned formula (II).

In the formula (II), R¹′ and R²′ are each a bond or a divalent C₁₋₅ acyclic hydrocarbon group optionally having substituents.

As the “divalent C₁₋₅ acyclic hydrocarbon group” of the “divalent C₁₋₅ acyclic hydrocarbon group optionally having substituents” for R¹, R², R¹∝ or R²′, for example, a C₁₋₅ alkylene group (e.g., methylene, ethylene, propylene, butylene, —CH₂CH₂CH₂—, —CH₂CH₂CH₂CH₂— and the like), a C₂₋₅ alkenylene group (e.g., vinylene, propenylene, isopropenylene, 2-butene-1-ylene, 4-pentene-1-ylene, 5-hexen-1-ylene) and the like can be used, and a C₁₋₅ alkylene group (e.g., methylene, ethylene, propylene, butylene, —CH₂CH₂CH₂—, —CH₂CH₂CH₂CH₂—) is particularly preferable.

As the substituent of the “divalent C₁₋₅ acyclic hydrocarbon group” for R¹, R², R¹′ or R²′, the substituents similar to the aforementioned substituent group B can be used, and a C₁₋₆ alkyl group and the like are particularly preferable.

As the “optionally oxidized sulfur atom” and “imino group optionally having a substituent” for X, those similar to the aforementioned “optionally oxidized sulfur atom” and “imino group optionally having a substituent” for Z can be used.

As X, a bond or an oxygen atom is preferable, and a bond is particularly preferable.

When R¹, R² and X are all bonds, however, R¹′, R²′ and X are not bonds at the same time. In other words, the compound of the present invention (I) does not have a structure represented by the formula

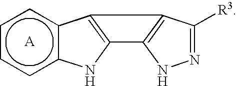

As the ring formed by R¹ and R² via X, for example, a 5- to 8-membered ring is used. Specifically, a compound having a structural formula represented by 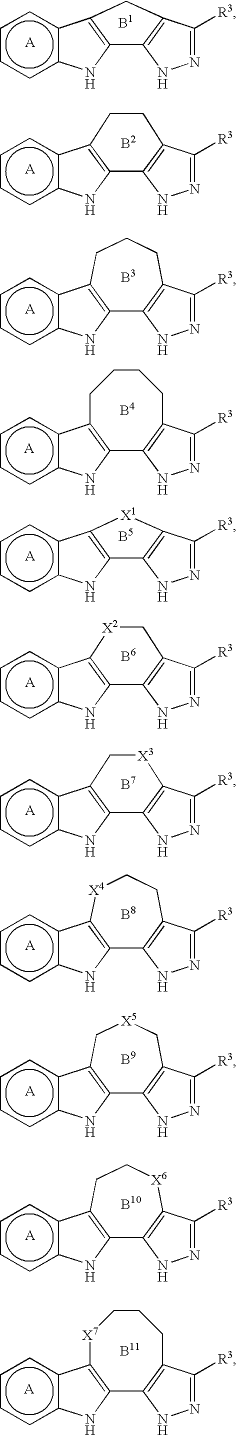 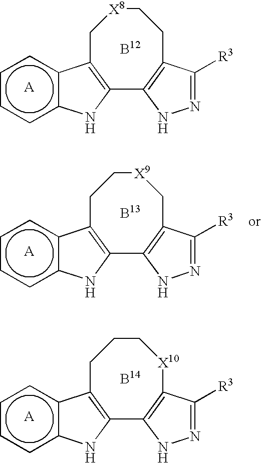
and the like can be used. Of these, a compound having a structural formula represented by 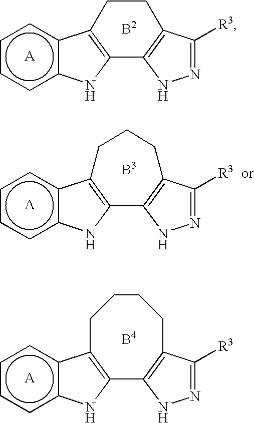
and the like are preferable, and a compound having a structural formula represented by 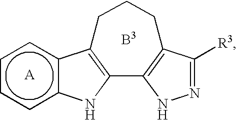
which is a compound wherein R¹′ is a —CH₂CH₂CH₂— group optionally having substituents of substituent group B and the like, and R²′ and X are bonds in the compound (II) is particularly preferable.

Moreover, when X is a bond, a ring formed by R¹ and R² via X and a ring formed by R¹′ and R²′ via X are preferably 7 or more-membered rings. In this case, the compound of the present invention (I) does not have a structural formula represented by

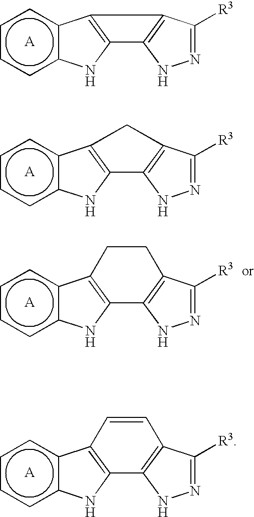

In this case, as the ring formed by R¹ and R² via X, for example, a 7 or 8-membered carbon ring or a 5 to 8-membered heterocycle can be used. Specifically, a compound having a structural formula represented by 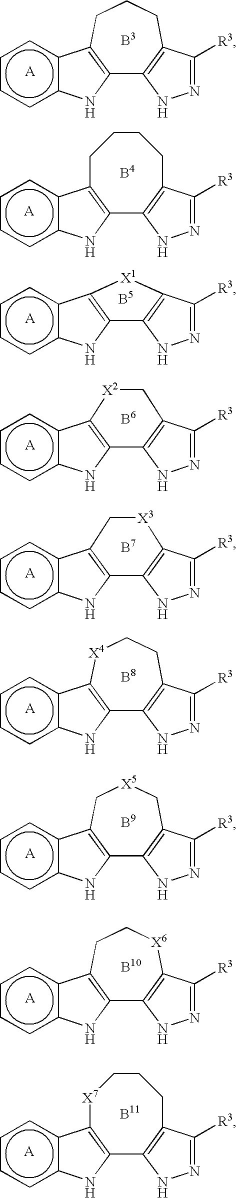 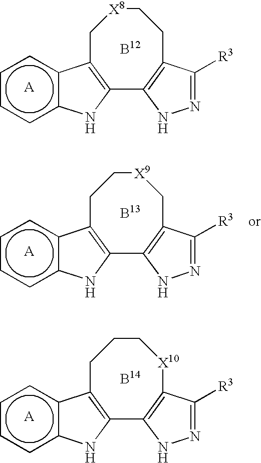
and the like can be used. Particularly, a compound having a structural formula represented by 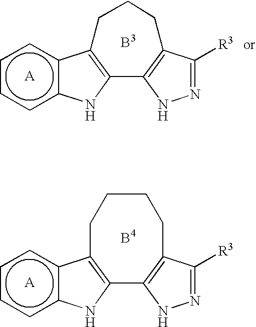
and the like are preferable, and a compound having a structural formula represented by 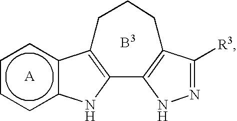
which is a compound wherein R¹′ is a —CH₂CH₂CH₂— group optionally having substituents of substituent group B and the like, and R²′ and X are bonds in the compound (II) is particularly preferable.

X¹—X¹⁰ are each an oxygen atom, an optionally oxidized sulfur atom or an imino group optionally having a substituent.

The ring B¹-ring B¹⁴ each optionally has substituents at a substitutable position.

As the “optionally oxidized sulfur atom” and “imino group optionally having a substituent” for X¹-X¹⁰, those similar to the aforementioned “optionally oxidized sulfur atom” and “imino group optionally having a substituent” for Z can be used.

As X¹-X¹⁰, an oxygen atom, an imino group optionally having C₁₋₆ alkyl and the like are preferable, and an oxygen atom is particularly preferable.

As the substituent for ring B¹-ring B¹⁴, those similar to the substituent of the “divalent C₁₋₅ acyclic hydrocarbon group” for R¹, R², R¹′ or R²′, namely, the substituents similar to the aforementioned substituent group B can be used, and a C₁₋₆ alkyl group and the like are particularly preferable.

A and R³ are as defined above.

The compound of the present invention (I) is preferably a compound wherein, when X is a bond and R¹ and R² form a 7 or more-membered ring via X, the substituent of the benzene ring for A is a heterocycle-carbonyl group having substituent(s) or a 1-piperidinylcarbonyl group having substituent(s).

In the formula (II), as the substituent of the benzene ring for A,

(i) a C₁₋₆ alkyl group (e.g., methyl),

(ii) a carboxyl group,

(iii) a C₁₋₆ alkoxy-carbonyl group (e.g., methoxycarbonyl, ethoxycarbonyl),

(iv) 5 to 7-membered heterocycle-carbonyl containing, besides carbon atom, 1 to 4 hetero atoms of 1 or 2 kinds selected from a nitrogen atom, a sulfur atom and an oxygen atom (e.g., 1-piperidinylcarbonyl), which is optionally substituted by substituents selected from the following (a) to (g);

(a) hydroxy,

(b) C₁₋₆ alkoxy-carbonyl (e.g., tert-butoxy-carbonyl),

(c) a group represented by the formula -Z¹-Z²

(wherein Z¹ and Z² are as defined below, and preferably, Z² is (i) a phenyl group optionally having substituents selected from the group consisting of (a) a halogen atom (e.g., fluorine, chlorine), (b) cyano, (c) an optionally halogenated C₁₋₆ alkyl group (e.g., methyl, trifluoromethyl), (d) an optionally halogenated C₁₋₆ alkoxy group (e.g., methoxy, trifluoromethoxy), (e) carbamoyl, (f) sulfamoyl and the like, or (ii) a 5 to 10-membered monocyclic or bicyclic heterocyclic group containing, besides carbon atom, 1 to 4 hetero atoms of 1 or 2 kinds selected from a nitrogen atom, a sulfur atom and an oxygen atom (particularly, pyrrolyl (e.g., 1-pyrrolyl, 2-pyrrolyl, 3-pyrrolyl), furyl (e.g., 2-furyl, 3-furyl), thienyl (e.g., 2-thienyl, 3-thienyl), imidazolyl (e.g., 1-imidazolyl, 2-imidazolyl, 4-imidazolyl), oxazolyl (e.g., 2-oxazolyl, 4-oxazolyl, 5-oxazolyl), thiazolyl (e.g., 2-thiazolyl, 4-thiazolyl, 5-thiazolyl), pyrazolyl (e.g., 1-pyrazolyl, 3-pyrazolyl, 4-pyrazolyl), isoxazolyl (e.g., 3-isoxazolyl, 4-isoxazolyl, 5-isoxazolyl), isothiazolyl (e.g., 3-isothiazolyl, 4-isothiazolyl, 5-isothiazolyl), pyridyl (e.g., 2-pyridyl, 3-pyridyl, 4-pyridyl), pyridazinyl (e.g., 3-pyridazinyl, 4-pyridazinyl), pyrimidinyl (e.g., 2-pyrimidinyl, 4-pyrimidinyl), pyrazinyl and the like can be mentioned, particularly a 5 to 10-membered monocyclic aromatic heterocyclic group containing, besides carbon atom, 1 to 4 hetero atoms of 1 or 2 kinds selected from a nitrogen atom, a sulfur atom and an oxygen atom, such as furyl (e.g., 2-furyl, 3-furyl), thienyl (e.g., 2-thienyl, 3-thienyl) and the like; a 5 to 10-membered bicyclic aromatic heterocyclic group containing, besides carbon atom, 1 to 4 hetero atoms of 1 or 2 kinds selected from a nitrogen atom, a sulfur atom and an oxygen atom, such as indolyl (e.g., 1-indolyl, 2-indolyl, 3-indolyl, 4-indolyl, 5-indolyl, 6-indolyl, 7-indolyl), isoindolyl (e.g., 1-isoindolyl, 2-isoindolyl, 3-isoindolyl, 4-isoindolyl, 5-isoindolyl, 6-isoindolyl, 7-isoindolyl), benzo[b]furanyl (e.g., 2-benzo[b]furanyl, 3-benzo[b]furanyl, 4-benzo[b]furanyl, 5-benzo[b]furanyl, 6-benzo[b]furanyl, 7-benzo[b]furanyl), benzo[c]furanyl (e.g., 1-benzo[c]furanyl, 4-benzo[c]furanyl, 5-benzo[c]furanyl), benzo[b]thienyl (e.g., 2-benzo[b]thienyl, 3-benzo[b]thienyl, 4-benzo[b]thienyl, 5-benzo[b]thienyl, 6-benzo[b]thienyl, 7-benzo[b]thienyl), benzo[c]thienyl (e.g., 1-benzo[c]thienyl, 4-benzo[c]thienyl, 5-benzo[c]thienyl), benzimidazolyl (e.g., 1-benzimidazolyl, 2-benzimidazolyl, 4-benzimidazolyl, 5-benzimidazolyl), benzoxazolyl (e.g., 2-benzoxazolyl, 4-benzoxazolyl, 5-benzoxazolyl, 6-benzoxazolyl, 7-benzoxazolyl), benzothiazolyl (e.g., 2-benzothiazolyl, 4-benzothiazolyl, 5-benzothiazolyl, 6-benzothiazolyl, 7-benzothiazolyl), indazolyl (e.g., 1-indazolyl, 2-indazolyl, 3-indazolyl, 4-indazolyl, 5-indazolyl, 6-indazolyl, 7-indazolyl), 1,2-benzisoxazolyl (e.g., 1,2-benzisoxazol-3-yl, 1,2-benzisoxazol-4-yl, 1,2-benzisoxazol-5-yl, 1,2-benzisoxazol-6-yl, 1,2-benzisoxazol-7-yl), 1,2-benzisothiazolyl (e.g., 1,2-benzisothiazol-3-yl, 1,2-benzisothiazol-4-yl, 1,2-benzisothiazol-5-yl, 1,2-benzisothiazol-6-yl, 1,2-benzisothiazol-7-yl), quinolyl (e.g., 2-quinolyl, 3-quinolyl, 4-quinolyl, 5-quinolyl, 6-quinolyl, 7-quinolyl, 8-quinolyl), isoquinolyl (e.g., 1-isoquinolyl, 3-isoquinolyl, 4-isoquinolyl, 5-isoquinolyl, 6-isoquinolyl, 7-isoquinolyl, 8-isoquinolyl), cinnolinyl (e.g., 3-cinnolinyl, 4-cinnolinyl, 5-cinnolinyl, 6-cinnolinyl, 7-cinnolinyl, 8-cinnolinyl), phthalazinyl (e.g., 1-phthalazinyl, 4-phthalazinyl, 5-phthalazinyl, 6-phthalazinyl, 7-phthalazinyl, 8-phthalazinyl), quinazolinyl (e.g., 2-quinazolinyl, 4-quinazolinyl, 5-quinazolinyl, 6-quinazolinyl, 7-quinazolinyl, 8-quinazolinyl), quinoxalinyl (e.g., 2-quinoxalinyl, 3-quinoxalinyl, 5-quinoxalinyl, 6-quinoxalinyl, 7-quinoxalinyl, 8-quinoxalinyl) and the like can be mentioned, particularly, quinolyl (e.g., 2-quinolyl, 3-quinolyl, 4-quinolyl, 5-quinolyl, 6-quinolyl, 7-quinolyl, 8-quinolyl), isoquinolyl (e.g., 1-isoquinolyl, 3-isoquinolyl, 4-isoquinolyl, 5-isoquinolyl, 6-isoquinolyl, 7-isoquinolyl, 8-isoquinolyl); a 5 to 10-membered non-aromatic heterocyclic group containing, besides carbon atom, 1 to 4 hetero atoms of 1 or 2 kinds selected from a nitrogen atom, a sulfur atom and an oxygen atom such as pyrrolidino (1-pyrrolidinyl), 1-piperidinyl, 4-morpholinyl, 4-thiomorpholinyl, azepan-1-yl, azocan-1-yl, azonan-1-yl, 3,4-dihydroisoquinolin-2-yl; and the like), which optionally has substituents selected from the group consisting of (a) a halogen atom (e.g., fluorine, chlorine), (b) cyano, (c) an optionally halogenated C₁₋₆ alkyl group (e.g., methyl, trifluoromethyl), (d) an optionally halogenated C₁₋₆ alkoxy group (e.g., methoxy, trifluoromethoxy), (e) carbamoyl, (f) sulfamoyl and the like),

(d) mono- or di-C₁₋₆ alkylamino (e.g., diethylamino),

(e) mono- or di-C₇₋₁₆ aralkylamino (e.g., benzylamino),

(f) 5 to 7-membered heterocycle-C₁₋₆ alkyl(C₁₋₆ alkyl)amino wherein the heterocycle contains, besides carbon atom, 1 to 4 hetero atoms of 1 or 2 kinds selected from a nitrogen atom, a sulfur atom and an oxygen atom (e.g., ethyl(thienylmethyl)amino),

(g) C₁₋₆ alkyl(C₇₋₁₆ aralkyl)amino wherein the C₁₋₆ alkyl is optionally substituted by hydroxy (e.g., methyl(benzyl)amino, ethyl(benzyl)amino, 2-hydroxyethyl(benzyl)amino);

(v) a carbamoyl group optionally having substituents selected from the following (a) to (c);

(a) C₁₋₆ alkyl (e.g., methyl, ethyl),

(b) C₁₋₆ alkyl (e.g., methyl, ethyl, propyl, butyl) substituted by mono- or di-C₁₋₆ alkylamino (e.g., dimethylamino), specifically dimethylamino-ethyl, dimethylamino-propyl, dimethylamino-butyl,

(c) C₁₋₆ alkyl (e.g., methyl, ethyl, propyl) substituted by a 5 to 7-membered heterocyclic group containing, besides carbon atom, 1 to 4 hetero atoms of 1 or 2 kinds selected from a nitrogen atom, a sulfur atom and an oxygen atom (e.g., thienyl, pyridyl, 1-piperidinyl, 4-morpholinyl), specifically thienylmethyl, pyridylmethyl, 1-piperidinylethyl, 4-morpholinylethyl, 4-morpholinylpropyl;

(vi) a halogen atom (e.g., chlorine);

(vii) 5 to 7-membered heterocycle-carbonyl-amino containing, besides carbon atom, 1 to 4 hetero atoms of 1 or 2 kinds selected from a nitrogen atom, a sulfur atom and an oxygen atom (e.g., pyridylcarbonylamino);

and the like can be preferably used.

In the formula (II), as the substituent of the benzene ring for A, heterocycle-carbonyl having substituent(s) and the like are also preferable, and particularly, 5 to 7-membered heterocycle-carbonyl containing, besides carbon atom, 1 to 4 hetero atoms of 1 or 2 kinds selected from a nitrogen atom, a sulfur atom and an oxygen atom (particularly 1-piperidinylcarbonyl), which has substituent(s) selected from the following (a) to (g) is preferable:

(a) hydroxy,

(b) C₁₋₆ alkoxy-carbonyl (e.g., tert-butoxy-carbonyl),

(c) a group represented by the formula -Z¹-Z²

(wherein Z¹ and Z² are as defined below, and preferably, Z² is (i) a phenyl group optionally having substituents selected from the group consisting of (a) a halogen atom (e.g., fluorine, chlorine), (b) cyano, (c) an optionally halogenated C₁₋₆ alkyl group (e.g., methyl, trifluoromethyl), (d) an optionally halogenated C₁₋₆ alkoxy group (e.g., methoxy, trifluoromethoxy), (e) carbamoyl, (f) sulfamoyl and the like, or (ii) a 5 to 10-membered monocyclic or bicyclic heterocyclic group containing, besides carbon atom, 1 to 4 hetero atoms of 1 or 2 kinds selected from a nitrogen atom, a sulfur atom and an oxygen atom (particularly, pyrrolyl (e.g., 1-pyrrolyl, 2-pyrrolyl, 3-pyrrolyl), furyl (e.g., 2-furyl, 3-furyl), thienyl (e.g., 2-thienyl, 3-thienyl), imidazolyl (e.g., 1-imidazolyl, 2-imidazolyl, 4-imidazolyl), oxazolyl (e.g., 2-oxazolyl, 4-oxazolyl, 5-oxazolyl), thiazolyl (e.g., 2-thiazolyl, 4-thiazolyl, 5-thiazolyl), pyrazolyl (e.g., 1-pyrazolyl, 3-pyrazolyl, 4-pyrazolyl), isoxazolyl (e.g., 3-isoxazolyl, 4-isoxazolyl, 5-isoxazolyl), isothiazolyl (e.g., 3-isothiazolyl, 4-isothiazolyl, 5-isothiazolyl), pyridyl (e.g., 2-pyridyl, 3-pyridyl, 4-pyridyl), pyridazinyl (e.g., 3-pyridazinyl, 4-pyridazinyl), pyrimidinyl (e.g., 2-pyrimidinyl, 4-pyrimidinyl), pyrazinyl and the like can be mentioned, particularly a 5 to 10-membered monocyclic aromatic heterocyclic group containing, besides carbon atom, 1 to 4 hetero atoms of 1 or 2 kinds selected from a nitrogen atom, a sulfur atom and an oxygen atom, such as furyl (e.g., 2-furyl, 3-furyl), thienyl (e.g., 2-thienyl, 3-thienyl) and the like; a 5 to 10-membered bicyclic aromatic heterocyclic group containing, besides carbon atom, 1 to 4 hetero atoms of 1 or 2 kinds selected from a nitrogen atom, a sulfur atom and an oxygen atom, such as indolyl (e.g., 1-indolyl, 2-indolyl, 3-indolyl, 4-indolyl, 5-indolyl, 6-indolyl, 7-indolyl), isoindolyl (e.g., 1-isoindolyl, 2-isoindolyl, 3-isoindolyl, 4-isoindolyl, 5-isoindolyl, 6-isoindolyl, 7-isoindolyl), benzo[b]furanyl (e.g., 2-benzo[b]furanyl, 3-benzo[b]furanyl, 4-benzo[b]furanyl, 5-benzo[b]furanyl, 6-benzo[b]furanyl, 7-benzo[b]furanyl), benzo[c]furanyl (e.g., 1-benzo[c]furanyl, 4-benzo[c]furanyl, 5-benzo[c]furanyl), benzo[b]thienyl (e.g., 2-benzo[b]thienyl, 3-benzo[b]thienyl, 4-benzo[b]thienyl, 5-benzo[b]thienyl, 6-benzo[b]thienyl, 7-benzo[b]thienyl), benzo[c]thienyl (e.g., 1-benzo[c]thienyl, 4-benzo[c]thienyl, 5-benzo[c]thienyl), benzimidazolyl (e.g., 1-benzimidazolyl, 2-benzimidazolyl, 4-benzimidazolyl, 5-benzimidazolyl), benzoxazolyl (e.g., 2-benzoxazolyl, 4-benzoxazolyl, 5-benzoxazolyl, 6-benzoxazolyl, 7-benzoxazolyl), benzothiazolyl (e.g., 2-benzothiazolyl, 4-benzothiazolyl, 5-benzothiazolyl, 6-benzothiazolyl, 7-benzothiazolyl), indazolyl (e.g., 1-indazolyl, 2-indazolyl, 3-indazolyl, 4-indazolyl, 5-indazolyl, 6-indazolyl, 7-indazolyl), 1,2-benzisoxazolyl (e.g., 1,2-benzisoxazol-3-yl, 1,2-benzisoxazol-4-yl, 1,2-benzisoxazol-5-yl, 1,2-benzisoxazol-6-yl, 1,2-benzisoxazol-7-yl), 1,2-benzisothiazolyl (e.g., 1,2-benzisothiazol-3-yl, 1,2-benzisothiazol-4-yl, 1,2-benzisothiazol-5-yl, 1,2-benzisothiazol-6-yl, 1,2-benzisothiazol-7-yl), quinolyl (e.g., 2-quinolyl, 3-quinolyl, 4-quinolyl, 5-quinolyl, 6-quinolyl, 7-quinolyl, 8-quinolyl), isoquinolyl (e.g., 1-isoquinolyl, 3-isoquinolyl, 4-isoquinolyl, 5-isoquinolyl, 6-isoquinolyl, 7-isoquinolyl, 8-isoquinolyl), cinnolinyl (e.g., 3-cinnolinyl, 4-cinnolinyl, 5-cinnolinyl, 6-cinnolinyl, 7-cinnolinyl, 8-cinnolinyl), phthalazinyl (e.g., 1-phthalazinyl, 4-phthalazinyl, 5-phthalazinyl, 6-phthalazinyl, 7-phthalazinyl, 8-phthalazinyl), quinazolinyl (e.g., 2-quinazolinyl, 4-quinazolinyl, 5-quinazolinyl, 6-quinazolinyl, 7-quinazolinyl, 8-quinazolinyl), quinoxalinyl (e.g., 2-quinoxalinyl, 3-quinoxalinyl, 5-quinoxalinyl, 6-quinoxalinyl, 7-quinoxalinyl, 8-quinoxalinyl) and the like can be mentioned, particularly, quinolyl (e.g., 2-quinolyl, 3-quinolyl, 4-quinolyl, 5-quinolyl, 6-quinolyl, 7-quinolyl, 8-quinolyl), isoquinolyl (e.g., 1-isoquinolyl, 3-isoquinolyl, 4-isoquinolyl, 5-isoquinolyl, 6-isoquinolyl, 7-isoquinolyl, 8-isoquinolyl); a 5 to 10-membered non-aromatic heterocyclic group containing, besides carbon atom, 1 to 4 hetero atoms of 1 or 2 kinds selected from a nitrogen atom, a sulfur atom and an oxygen atom such as pyrrolidino (1-pyrrolidinyl), 1-piperidinyl, 4-morpholinyl, 4-thiomorpholinyl, azepan-1-yl, azocan-1-yl, azonan-1-yl, 3,4-dihydroisoquinolin-2-yl; and the like), which optionally has substituents selected from the group consisting of (a) a halogen atom (e.g., fluorine, chlorine), (b) cyano, (c) an optionally halogenated C₁₋₆ alkyl group (e.g., methyl, trifluoromethyl), (d) an optionally halogenated C₁₋₆ alkoxy group (e.g., methoxy, trifluoromethoxy), (e) carbamoyl, (f) sulfamoyl and the like),

(d) mono- or di-C₁₋₆ alkylamino (e.g., diethylamino),

(e) mono- or di-C₇₋₁₆ aralkylamino (e.g., benzylamino),

(f) 5 to 7-membered heterocycle-C₁₋₆ alkyl(C₁₋₆ alkyl)amino herein the heterocycle contains, besides carbon atom, 1 to 4 hetero atoms of 1 or 2 kinds selected from a nitrogen atom, a sulfur atom and an oxygen atom (e.g., ethyl(thienylmethyl)amino),

(g) C₁₋₆ alkyl(C₇₋₁₆ aralkyl)amino wherein the C₁₋₆ alkyl is optionally substituted by hydroxy (e.g., methyl(benzyl)amino, ethyl(benzyl)amino, 2-hydroxyethyl(benzyl)amino).

particularly, when X is a bond and R¹ and R² form a 7 or more-membered ring (particularly, 7-membered ring) via X, or when X is a bond and R¹′ and R²′ form a 7 or more-membered ring (particularly, 7-membered ring) via X, the substituent of the benzene ring for A is preferably heterocycle-carbonyl having substituent(s) and, for example, 5 to 7-membered heterocycle-carbonyl containing, besides carbon atom, 1 to 4 hetero atoms of 1 or 2 kinds selected from a nitrogen atom, a sulfur atom and an oxygen atom, which has substituent(s) selected from the above-mentioned (a) to (g) (particularly 1-piperidinylcarbonyl) is preferable.

In the formula (II), R³ is preferably a hydrogen atom.

[Compound (I-1) and Compound (I-2)]

As compound (I-1), compound (I-1) and compound (I-2) are preferable, and compound (I-2) is particularly preferable.

Z¹ is a bond, methylene (CH₂), ethylene (CH₂CH₂), vinylene (CHCH), ethynylene (CC) or methyleneoxy (CH₂O).

Z² is a phenyl group optionally having substituents or a heterocyclic group optionally having substituents.

As the phenyl group optionally having substituents for Z², for example, a phenyl group optionally having substituents selected from substituent group B can be used. Particularly, a phenyl group optionally having substituents selected from the group consisting of (a) a halogen atom (e.g., fluorine, chlorine), (b) cyano, (c) an optionally halogenated C₁₋₆ alkyl group (e.g., methyl, trifluoromethyl), (d) an optionally halogenated C₁₋₆ alkoxy group (e.g., methoxy, trifluoromethoxy), (e) carbamoyl, (f) sulfamoyl and the like, and the like is preferably used, and a phenyl group optionally having substituents selected from the group consisting of (a) a halogen atom (e.g., fluorine, chlorine), (b) cyano, (c) an optionally halogenated C₁₋₆ alkyl group (e.g., methyl, trifluoromethyl), (d) an optionally halogenated C₁₋₆ alkoxy group (e.g., methoxy, trifluoromethoxy) and the like, and the like can be particularly preferably used.

As the heterocyclic group optionally having substituents for Z², for example, those similar to the “heterocyclic group optionally having substituents” of the substituent group A can be used. Of these, a heterocyclic group optionally having substituents selected from the group consisting of (a) a halogen atom (e.g., fluorine), (b) cyano, (c) an optionally halogenated C₁₋₆ alkyl group (e.g., methyl, trifluoromethyl), (d) an optionally halogenated C₁₋₆ alkoxy group (e.g., methoxy, trifluoromethoxy), (e) carbamoyl, (f) sulfamoyl and the like is preferably used, and a 5 to 10-membered monocyclic or bicyclic heterocyclic group containing, besides carbon atom, 1 to 4 hetero atoms of 1 or 2 kinds selected from a nitrogen atom, a sulfur atom and an oxygen atom (e.g., aromatic heterocyclic group or non-aromatic heterocyclic group, particularly, aromatic heterocyclic group), which optionally has substituents selected from the group consisting of (a) a halogen atom (e.g., fluorine), (b) cyano, (c) an optionally halogenated C₁₋₆ alkyl group (e.g., methyl, trifluoromethyl), (d) an optionally halogenated C₁₋₆ alkoxy group (e.g., methoxy, trifluoromethoxy), (e) carbamoyl, (f) sulfamoyl and the like, and the like can be particularly preferably used.

As the 5 to 10-membered monocyclic aromatic heterocyclic group containing, besides carbon atom, 1 to 4 hetero atoms of 1 or 2 kinds selected from a nitrogen atom, a sulfur atom and an oxygen atom, for example, pyrrolyl (e.g., 1-pyrrolyl, 2-pyrrolyl, 3-pyrrolyl), furyl (e.g., 2-furyl, 3-furyl), thienyl (e.g., 2-thienyl, 3-thienyl), imidazolyl (e.g., 1-imidazolyl, 2-imidazolyl, 4-imidazolyl), oxazolyl (e.g., 2-oxazolyl, 4-oxazolyl, 5-oxazolyl), thiazolyl (e.g., 2-thiazolyl, 4-thiazolyl, 5-thiazolyl), pyrazolyl (e.g., 1-pyrazolyl, 3-pyrazolyl, 4-pyrazolyl), isoxazolyl (e.g., 3-isoxazolyl, 4-isoxazolyl, 5-isoxazolyl), isothiazolyl (e.g., 3-isothiazolyl, 4-isothiazolyl, 5-isothiazolyl), pyridyl (e.g., 2-pyridyl, 3-pyridyl, 4-pyridyl), pyridazinyl (e.g., 3-pyridazinyl, 4-pyridazinyl), pyrimidinyl (e.g., 2-pyrimidinyl, 4-pyrimidinyl), pyrazinyl and the like can be mentioned, particularly furyl (e.g., 2-furyl, 3-furyl), thienyl (e.g., 2-thienyl, 3-thienyl) and the like are preferable.

As the 5 to 10-membered bicyclic aromatic heterocyclic group containing, besides carbon atom, 1 to 4 hetero atoms of 1 or 2 kinds selected from a nitrogen atom, a sulfur atom and an oxygen atom, for example, indolyl (e.g., 1-indolyl, 2-indolyl, 3-indolyl, 4-indolyl, 5-indolyl, 6-indolyl, 7-indolyl), isoindolyl (e.g., 1-isoindolyl, 2-isoindolyl, 3-isoindolyl, 4-isoindolyl, 5-isoindolyl, 6-isoindolyl, 7-isoindolyl), benzo[b]furanyl (e.g., 2-benzo[b]furanyl, 3-benzo[b]furanyl, 4-benzo[b]furanyl, 5-benzo[b]furanyl, 6-benzo[b]furanyl, 7-benzo[b]furanyl), benzo[c]furanyl (e.g., 1-benzo[c]furanyl, 4-benzo[c]furanyl, 5-benzo[c]furanyl), benzo[b]thienyl (e.g., 2-benzo[b]thienyl, 3-benzo[b]thienyl, 4-benzo[b]thienyl, 5-benzo[b]thienyl, 6-benzo[b]thienyl, 7-benzo[b]thienyl), benzo[c]thienyl (e.g., 1-benzo[c]thienyl, 4-benzo[c]thienyl, 5-benzo[c]thienyl), benzimidazolyl (e.g., 1-benzimidazolyl, 2-benzimidazolyl, 4-benzimidazolyl, 5-benzimidazolyl), benzoxazolyl (e.g., 2-benzoxazolyl, 4-benzoxazolyl, 5-benzoxazolyl, 6-benzoxazolyl, 7-benzoxazolyl), benzothiazolyl (e.g., 2-benzothiazolyl, 4-benzothiazolyl, 5-benzothiazolyl, 6-benzothiazolyl, 7-benzothiazolyl), indazolyl (e.g., 1-indazolyl, 2-indazolyl, 3-indazolyl, 4-indazolyl, 5-indazolyl, 6-indazolyl, 7-indazolyl), 1,2-benzisoxazolyl (e.g., 1,2-benzisoxazol-3-yl, 1,2-benzisoxazol-4-yl, 1,2-benzisoxazol-5-yl, 1,2-benzisoxazol-6-yl, 1,2-benzisoxazol-7-yl), 1,2-benzisothiazolyl (e.g., 1,2-benzisothiazol-3-yl, 1,2-benzisothiazol-4-yl, 1,2-benzisothiazol-5-yl, 1,2-benzisothiazol-6-yl, 1,2-benzisothiazol-7-yl), quinolyl (e.g., 2-quinolyl, 3-quinolyl, 4-quinolyl, 5-quinolyl, 6-quinolyl, 7-quinolyl, 8-quinolyl), isoquinolyl (e.g., 1-isoquinolyl, 3-isoquinolyl, 4-isoquinolyl, 5-isoquinolyl, 6-isoquinolyl, 7-isoquinolyl, 8-isoquinolyl), cinnolinyl (e.g., 3-cinnolinyl, 4-cinnolinyl, 5-cinnolinyl, 6-cinnolinyl, 7-cinnolinyl, 8-cinnolinyl), phthalazinyl (e.g., 1-phthalazinyl, 4-phthalazinyl, 5-phthalazinyl, 6-phthalazinyl, 7-phthalazinyl, 8-phthalazinyl), quinazolinyl (e.g., 2-quinazolinyl, 4-quinazolinyl, 5-quinazolinyl, 6-quinazolinyl, 7-quinazolinyl, 8-quinazolinyl), quinoxalinyl (e.g., 2-quinoxalinyl, 3-quinoxalinyl, 5-quinoxalinyl, 6-quinoxalinyl, 7-quinoxalinyl, 8-quinoxalinyl) and the like can be mentioned, particularly, quinolyl (e.g., 2-quinolyl, 3-quinolyl, 4-quinolyl, 5-quinolyl, 6-quinolyl, 7-quinolyl, 8-quinolyl), isoquinolyl (e.g., 1-isoquinolyl, 3-isoquinolyl, 4-isoquinolyl, 5-isoquinolyl, 6-isoquinolyl, 7-isoquinolyl, 8-isoquinolyl) and the like are preferable.

As the 5 to 10-membered non-aromatic heterocyclic group containing, besides carbon atom, 1 to 4 hetero atoms of 1 or 2 kinds selected from a nitrogen atom, a sulfur atom and an oxygen atom, for example, pyrrolidino (1-pyrrolidinyl), 1-piperidinyl, 4-morpholinyl, 4-thiomorpholinyl, azepan-1-yl, azocan-1-yl, azonan-1-yl, 3,4-dihydroisoquinolin-2-yl and the like are preferable.

The ring for Q optionally further has substituents. As the substituents, for example, those similar to the substituents that the benzene ring for A optionally has can be used, with preference given to hydroxy.

A″ is a benzene ring optionally further having substituents. As the substituent that the benzene ring for A″ optionally has, those similar to the substituents that the benzene ring for A optionally has can be used, which is preferably unsubstituted.

R³ is as defined above and, for example, a hydrogen atom and the like are preferable.

[Compound (III)]

When R¹ and R² do not form a ring via X, the compound of the present invention is represented by the above-mentioned formula (III).

In the formula (III), R¹″ and R²″ are each a hydrogen atom, a hydrocarbon group optionally having substituents or a heterocyclic group optionally having substituents.

As the “hydrocarbon group optionally having substituents” and “heterocyclic group optionally having substituents” for R¹″ or R²″, those similar to the aforementioned “hydrocarbon group optionally having substituents” and “heterocyclic group optionally having substituents” for R¹ or R² can be preferably used.

Note that R²″ is not a 4-hydroxyphenyl group optionally having substituents selected from the group consisting of a halogen atom, a C₁₋₆ alkyl group and a C₁₋₆ alkoxy group; a 4-methoxyphenyl group optionally having substituents selected from the group consisting of a halogen atom, a C₁₋₆ alkyl group and a C₁₋₆ alkoxy group; and a 6-hydroxypyridin-3-yl group optionally having substituents selected from the group consisting of a halogen atom, a C₁₋₆ alkyl group and a C₁₋₆ alkoxy group.

As R¹″, a hydrogen atom is preferable.

As R²″, a hydrogen atom, a C₁₋₆ alkyl group (e.g., methyl, ethyl, propyl) and the like are preferable.

A and R³ are as defined above.

As compound (III), compound (IIIa) wherein R²″ is R^{2b}″ which is a hydrocarbon group optionally having substituents or a heterocyclic group optionally having substituents is preferable.

As R^{2b}″, a C₁₋₆ alkyl group (e.g., methyl, ethyl, propyl) and the like are preferable.

In the formula (III), as the substituent of the benzene ring for A, 5 to 7-membered heterocycle-carbonyl containing, besides carbon atom, 1 to 4 hetero atoms of 1 or 2 kinds selected from a nitrogen atom, a sulfur atom and an oxygen atom (e.g., 1-piperidinylcarbonyl), which is optionally substituted by a 5 to 10-membered heterocyclic group containing, besides carbon atom, 1 to 4 hetero atoms of 1 or 2 kinds selected from a nitrogen atom, a sulfur atom and an oxygen atom (e.g., 3,4-dihydroisoquinolin-2-yl) and the like, and the like are preferable.

In the formula (III), R³ is preferably a hydrogen atom.

[Compound (IV)]

In the above-mentioned formula (IV), A′ is a benzene ring optionally further having substituents besides a group represented by the formula Z-Y²—Y¹—.

As the substituent that the benzene ring for A′ optionally further has besides a group represented by the formula Z-Y²—Y¹—, those similar to the substituent that the aforementioned benzene ring for A optionally has can be used.

R¹″, R²″, R³, Y¹, Y² and Z are as defined above.

In the formula (IV), as R¹″, a hydrogen atom is preferable.

In the formula (IV), as R²″, a C₁₋₆ alkyl group (e.g., methyl, ethyl, propyl) and the like are preferable.

In the formula (IV), as R³, a hydrogen atom is preferable.

As Y¹, a carbonyl group and the like are preferable.

As Y², a bond, an imino group optionally having a substituent (e.g., —NH—) and the like are preferable. Of these, an imino group optionally having a substituent is preferable. Particularly, as Y², a bond, —NR⁴— (R⁴ is a hydrogen atom or a C₁₋₆ alkyl group) and the like are preferable, and a bond or —NH— is particularly preferable.

As Z, a hydrogen atom, the above-mentioned C₁₋₆ alkyl group optionally having substituents and the above-mentioned heterocyclic group optionally having substituents are preferable.

As said C₁₋₆ alkyl group optionally having substituents, a C₁₋₆ alkyl group (e.g., methyl, ethyl, propyl, butyl) optionally having substituents selected from, for example, mono- or di-C₁₋₆ alkylamino (e.g., dimethylamino), a 5 to 7-membered heterocyclic group containing, besides carbon atom, 1 to 4 hetero atoms of 1 or 2 kinds selected from a nitrogen atom, a sulfur atom and an oxygen atom (e.g., thienyl, pyridyl, 1-piperidinyl, 4-morpholinyl) and the like can be used, and dimethylamino-ethyl, dimethylamino-propyl, dimethylamino-butyl and the like can be specifically used.

As the “heterocyclic group” of said heterocyclic group optionally having substituents, for example, a 5 to 7-membered heterocyclic group containing, besides carbon atom, 1 to 4 hetero atoms of 1 or 2 kinds selected from a nitrogen atom, a sulfur atom and an oxygen atom can be used. Of these, a 5 to 7-membered non-aromatic nitrogen-containing heterocyclic group such as 1-piperidinyl, 1-piperazinyl and the like is preferable, and 1-piperidinyl is particularly preferable.

As the “substituent” of said “heterocyclic group optionally having substituents”, for example,

(a) hydroxy,

(b) C₁₋₆ alkoxy-carbonyl (e.g., tert-butoxy-carbonyl),

(c) a group represented by the formula -Z¹-Z²

(wherein Z¹ and Z² are as defined below, and preferably, Z² is (i) a phenyl group optionally having substituents selected from the group consisting of (a) a halogen atom (e.g., fluorine, chlorine), (b) cyano, (c) an optionally halogenated C₁₋₆ alkyl group (e.g., methyl, trifluoromethyl), (d) an optionally halogenated C₁₋₆ alkoxy group (e.g., methoxy, trifluoromethoxy), (e) carbamoyl, (f) sulfamoyl and the like, or (ii) a 5 to 10-membered monocyclic or bicyclic heterocyclic group containing, besides carbon atom, 1 to 4 hetero atoms of 1 or 2 kinds selected from a nitrogen atom, a sulfur atom and an oxygen atom (particularly, pyrrolyl (e.g., 1-pyrrolyl, 2-pyrrolyl, 3-pyrrolyl), furyl (e.g., 2-furyl, 3-furyl), thienyl (e.g., 2-thienyl, 3-thienyl), imidazolyl (e.g., 1-imidazolyl, 2-imidazolyl, 4-imidazolyl), oxazolyl (e.g., 2-oxazolyl, 4-oxazolyl, 5-oxazolyl), thiazolyl (e.g., 2-thiazolyl, 4-thiazolyl, 5-thiazolyl), pyrazolyl (e.g., 1-pyrazolyl, 3-pyrazolyl, 4-pyrazolyl), isoxazolyl (e.g., 3-isoxazolyl, 4-isoxazolyl, 5-isoxazolyl), isothiazolyl (e.g., 3-isothiazolyl, 4-isothiazolyl, 5-isothiazolyl), pyridyl (e.g., 2-pyridyl, 3-pyridyl, 4-pyridyl), pyridazinyl (e.g., 3-pyridazinyl, 4-pyridazinyl), pyrimidinyl (e.g., 2-pyrimidinyl, 4-pyrimidinyl), pyrazinyl and the like can be mentioned, particularly a 5 to 10-membered monocyclic aromatic heterocyclic group containing, besides carbon atom, 1 to 4 hetero atoms of 1 or 2 kinds selected from a nitrogen atom, a sulfur atom and an oxygen atom, such as furyl (e.g., 2-furyl, 3-furyl), thienyl (e.g., 2-thienyl, 3-thienyl) and the like; a 5 to 10-membered bicyclic aromatic heterocyclic group containing, besides carbon atom, 1 to 4 hetero atoms of 1 or 2 kinds selected from a nitrogen atom, a sulfur atom and an oxygen atom, such as indolyl (e.g., 1-indolyl, 2-indolyl, 3-indolyl, 4-indolyl, 5-indolyl, 6-indolyl, 7-indolyl), isoindolyl (e.g., 1-isoindolyl, 2-isoindolyl, 3-isoindolyl, 4-isoindolyl, 5-isoindolyl, 6-isoindolyl, 7-isoindolyl), benzo[b]furanyl (e.g., 2-benzo[b]furanyl, 3-benzo[b]furanyl, 4-benzo[b]furanyl, 5-benzo[b]furanyl, 6-benzo[b]furanyl, 7-benzo[b]furanyl), benzo[c]furanyl (e.g., 1-benzo[c]furanyl, 4-benzo[c]furanyl, 5-benzo[c]furanyl), benzo[b]thienyl (e.g., 2-benzo[b]thienyl, 3-benzo[b]thienyl, 4-benzo[b]thienyl, 5-benzo[b]thienyl, 6-benzo[b]thienyl, 7-benzo[b]thienyl), benzo[c]thienyl (e.g., 1-benzo[c]thienyl, 4-benzo[c]thienyl, 5-benzo[c]thienyl), benzimidazolyl (e.g., 1-benzimidazolyl, 2-benzimidazolyl, 4-benzimidazolyl, 5-benzimidazolyl), benzoxazolyl (e.g., 2-benzoxazolyl, 4-benzoxazolyl, 5-benzoxazolyl, 6-benzoxazolyl, 7-benzoxazolyl), benzothiazolyl (e.g., 2-benzothiazolyl, 4-benzothiazolyl, 5-benzothiazolyl, 6-benzothiazolyl, 7-benzothiazolyl), indazolyl (e.g., 1-indazolyl, 2-indazolyl, 3-indazolyl, 4-indazolyl, 5-indazolyl, 6-indazolyl, 7-indazolyl), 1,2-benzisoxazolyl (e.g., 1,2-benzisoxazol-3-yl, 1,2-benzisoxazol-4-yl, 1,2-benzisoxazol-5-yl, 1,2-benzisoxazol-6-yl, 1,2-benzisoxazol-7-yl), 1,2-benzisothiazolyl (e.g., 1,2-benzisothiazol-3-yl, 1,2-benzisothiazol-4-yl, 1,2-benzisothiazol-5-yl, 1,2-benzisothiazol-6-yl, 1,2-benzisothiazol-7-yl), quinolyl (e.g., 2-quinolyl, 3-quinolyl, 4-quinolyl, 5-quinolyl, 6-quinolyl, 7-quinolyl, 8-quinolyl), isoquinolyl (e.g., 1-isoquinolyl, 3-isoquinolyl, 4-isoquinolyl, 5-isoquinolyl, 6-isoquinolyl, 7-isoquinolyl, 8-isoquinolyl), cinnolinyl (e.g., 3-cinnolinyl, 4-cinnolinyl, 5-cinnolinyl, 6-cinnolinyl, 7-cinnolinyl, 8-cinnolinyl), phthalazinyl (e.g., 1-phthalazinyl, 4-phthalazinyl, 5-phthalazinyl, 6-phthalazinyl, 7-phthalazinyl, 8-phthalazinyl), quinazolinyl (e.g., 2-quinazolinyl, 4-quinazolinyl, 5-quinazolinyl, 6-quinazolinyl, 7-quinazolinyl, 8-quinazolinyl), quinoxalinyl (e.g., 2-quinoxalinyl, 3-quinoxalinyl, 5-quinoxalinyl, 6-quinoxalinyl, 7-quinoxalinyl, 8-quinoxalinyl) and the like can be mentioned, particularly, quinolyl (e.g., 2-quinolyl, 3-quinolyl, 4-quinolyl, 5-quinolyl, 6-quinolyl, 7-quinolyl, 8-quinolyl), isoquinolyl (e.g., 1-isoquinolyl, 3-isoquinolyl, 4-isoquinolyl, 5-isoquinolyl, 6-isoquinolyl, 7-isoquinolyl, 8-isoquinolyl); a 5 to 10-membered non-aromatic heterocyclic group containing, besides carbon atom, 1 to 4 hetero atoms of 1 or 2 kinds selected from a nitrogen atom, a sulfur atom and an oxygen atom such as pyrrolidino (1-pyrrolidinyl), 1-piperidinyl, 4-morpholinyl, 4-thiomorpholinyl, azepan-1-yl, azocan-1-yl, azonan-1-yl, 3,4-dihydroisoquinolin-2-yl; and the like), which optionally has substituents selected from the group consisting of (a) a halogen atom (e.g., fluorine, chlorine), (b) cyano, (c) an optionally halogenated C₁₋₆ alkyl group (e.g., methyl, trifluoromethyl), (d) an optionally halogenated C₁₋₆ alkoxy group (e.g., methoxy, trifluoromethoxy), (e) carbamoyl, (f) sulfamoyl and the like),

(d) mono- or di-C₁₋₆ alkylamino (e.g., diethylamino),

(e) mono- or di-C₇₋₁₆ aralkylamino (e.g., benzylamino),

(f) 5 to 7-membered heterocycle-C₁₋₆ alkyl(C₁₋₆ alkyl)amino wherein the heterocycle contains, besides carbon atom, 1 to 4 hetero atoms of 1 or 2 kinds selected from a nitrogen atom, a sulfur atom and an oxygen atom (e.g., ethyl(thienylmethyl)amino),

(g) C₁₋₆ alkyl(C₇₋₁₆ aralkyl)amino wherein the C₁₋₆ alkyl is optionally substituted by hydroxy (e.g., methyl(benzyl)amino, ethyl(benzyl)amino, 2-hydroxyethyl(benzyl)amino); and the like can be preferably used.

As Z, 1-piperidinyl optionally having substituents and the like are also preferable. As the substituent of said 1-piperidinyl, a 5 to 10-membered heterocyclic group containing, besides carbon atom, 1 to 4 hetero atoms of 1 or 2 kinds selected from a nitrogen atom, a sulfur atom and an oxygen atom (e.g., 3,4-dihydroisoquinolin-2-yl) and the like are preferable.

As a preferable combination of Y¹, Y² and Z,

(1) Y¹ is a carbonyl group, Y² is an imino group optionally having a substituent, and Z is a hydrocarbon group optionally having substituents (particularly, a C₁₋₆ alkyl group optionally having substituents such as the “optionally substituted amino group” of substituent group B, and the like);

(2) Y¹ is a carbonyl group, Y² is a bond, and Z is a heterocyclic group optionally having substituents and the like can be mentioned, particularly, a combination of Y¹ is a carbonyl group, Y² is a bond, and Z is a heterocyclic group optionally having substituents is preferable.

As compound (IV), compound (IVa) wherein R² is R^{2b}″ which is a hydrocarbon group optionally having substituents or a heterocyclic group optionally having substituents is preferable.

As R^{2b}″, a C₁₋₆ alkyl group (e.g., methyl, ethyl, propyl) and the like are preferable.

[Compound (I′)]

In the above-mentioned formula (I′), R^{2a} is a hydrogen atom, a hydrocarbon group optionally having substituents or a heterocyclic group optionally having substituents.

As the “hydrocarbon group optionally having substituents” and “heterocyclic group optionally having substituents” for R^{2a}, those similar to the “hydrocarbon group optionally having substituents” and “heterocyclic group optionally having substituents” for R² can be used.

As A, R¹, R³ and X, the groups similar to those exemplified in the above-mentioned formula (I) can be used.

As the ring formed by R¹ and R^{2a} via X, those similar to a ring formed by R¹ and R² via X can be used.

However, R¹, R^{2a} and X are not bonds at the same time.

[Compound (Ia)]

In the above-mentioned formula (Ia), R^{2b} is a hydrocarbon group optionally having substituents or a heterocyclic group optionally having substituents.

As the “hydrocarbon group optionally having substituents” and “heterocyclic group optionally having substituents” for R^{2b}, those similar to the “hydrocarbon group optionally having substituents” and “heterocyclic group optionally having substituents” for R² can be used.

As A, R¹, R³ and X, the groups similar to those exemplified in the above-mentioned formula (I) can be used.

As the ring formed by R¹ and R^{2b} via X, those similar to the ring formed by R¹ and R² via X can be used.

However, when X is a bond, the ring formed by R¹ and R^{2b} via X is a 7 or more-membered ring.

[Compound (I′a)]

In the above-mentioned formula (I′a), R^{2aa} is a hydrocarbon group optionally having substituents or a heterocyclic group optionally having substituents.

As the “hydrocarbon group optionally having substituents” and “heterocyclic group optionally having substituents” for R^{2aa}, those similar to the “hydrocarbon group optionally having substituents” and “heterocyclic group optionally having substituents” for R² can be used.

As A, R¹, R³ and X, the groups similar to those exemplified in the above-mentioned formula (I) can be used.

As the ring formed by R¹ and R^{2aa} via X, those similar to the ring formed by R¹ and R² via X can be used.

However, when X is a bond, the ring formed by R¹ and R^{2aa} via X is a 7 or more-membered ring.

More preferably, when compound (I′) (including compound (I′a)) is used as an agent for inhibiting vascular endothelial growth factor receptor (VEGFR), an agent for inhibiting vascular endothelial growth factor receptor 1 (VEGFR1, Flt-1), an agent for inhibiting vascular endothelial growth factor receptor 2 (VEGFR2), an agent for inhibiting vascular endothelial growth factor receptor 3 (VEGFR3, Flt-4), an agent for inhibiting fibroblast growth factor receptor 1 (FGFR1), an agent for inhibiting vascular endothelial cell growth, or an agent for the prophylaxis or treatment of diabetic retinopathy, rheumatoid arthritis, psoriasis, atherosclerosis, Kaposi sarcoma, COPD, pain, inflammation or hypertension mentioned below, R¹, R^{2a} and X are not bonds at the same time, and when compound (I′) is used as an agent for inhibiting kinase (phosphorylation enzyme), an agent for inhibiting fibroblast growth factor receptor (FGFR), an agent for inhibiting fibroblast growth factor receptor 2 (FGFR2), an agent for inhibiting fibroblast growth factor receptor 3 (FGFR3), an agent for inhibiting fibroblast growth factor receptor 4 (FGFR4), an agent for inhibiting angiogenesis, an agent for the prophylaxis or treatment of cancer (e.g., colorectal cancer, lung cancer, pancreatic cancer, gastric cancer, breast cancer, ovarian cancer, prostate cancer, liver cancer, thyroid cancer, kidney cancer, cerebral tumor, melanoma, urinary bladder cancer and the like), an agent for inhibiting growth of cancer, or an agent for suppressing metastasis of cancer mentioned below and when X is a bond, then the ring formed by R¹ and R^{2a} via X is preferably a 7 or more-membered ring (namely, compound (I′a)).

Of the compounds mentioned above, the compound (I) of the resent invention is preferably
(1) a compound represented by the formula 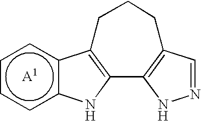
wherein A¹ is a benzene ring optionally having substituents selected from the following (i) to (vii).
(i) a C₁₋₆ alkyl group (e.g., methyl),
(ii) a carboxyl group,
(iii) a C₁₋₆ alkoxy-carbonyl group (e.g., methoxycarbonyl, ethoxycarbonyl),
(iv) 5 to 7-membered heterocycle-carbonyl containing, besides carbon atom, 1 to 4 hetero atoms of 1 or 2 kinds selected from a nitrogen atom, a sulfur atom and an oxygen atom (e.g., 1-piperidinylcarbonyl), which is optionally substituted by substituents selected from the following (a) to (g);

(a) hydroxy,

(b) C₁₋₆ alkoxy-carbonyl (e.g., tert-butoxy-carbonyl),

(c) a group represented by the formula -Z¹-Z²

(wherein Z¹ and Z² are as defined below, and preferably, Z² is (i) a phenyl group optionally having substituents selected from the group consisting of (a) a halogen atom (e.g., fluorine, chlorine), (b) cyano, (c) an optionally halogenated C₁₋₆ alkyl group (e.g., methyl, trifluoromethyl), (d) an optionally halogenated C₁₋₆ alkoxy group (e.g., methoxy, trifluoromethoxy), (e) carbamoyl, (f) sulfamoyl and the like, or (ii) a 5 to 10-membered monocyclic or bicyclic heterocyclic group containing, besides carbon atom, 1 to 4 hetero atoms of 1 or 2 kinds selected from a nitrogen atom, a sulfur atom and an oxygen atom (particularly, pyrrolyl (e.g., 1-pyrrolyl, 2-pyrrolyl, 3-pyrrolyl), furyl (e.g., 2-furyl, 3-furyl), thienyl (e.g., 2-thienyl, 3-thienyl), imidazolyl (e.g., 1-imidazolyl, 2-imidazolyl, 4-imidazolyl), oxazolyl (e.g., 2-oxazolyl, 4-oxazolyl, 5-oxazolyl), thiazolyl (e.g., 2-thiazolyl, 4-thiazolyl, 5-thiazolyl), pyrazolyl (e.g., 1-pyrazolyl, 3-pyrazolyl, 4-pyrazolyl), isoxazolyl (e.g., 3-isoxazolyl, 4-isoxazolyl, 5-isoxazolyl), isothiazolyl (e.g., 3-isothiazolyl, 4-isothiazolyl, 5-isothiazolyl), pyridyl (e.g., 2-pyridyl, 3-pyridyl, 4-pyridyl), pyridazinyl (e.g., 3-pyridazinyl, 4-pyridazinyl), pyrimidinyl (e.g., 2-pyrimidinyl, 4-pyrimidinyl), pyrazinyl and the like can be mentioned, particularly a 5 to 10-membered monocyclic aromatic heterocyclic group containing, besides carbon atom, 1 to 4 hetero atoms of 1 or 2 kinds selected from a nitrogen atom, a sulfur atom and an oxygen atom, such as furyl (e.g., 2-furyl, 3-furyl), thienyl (e.g., 2-thienyl, 3-thienyl) and the like; a 5 to 10-membered bicyclic aromatic heterocyclic group containing, besides carbon atom, 1 to 4 hetero atoms of 1 or 2 kinds selected from a nitrogen atom, a sulfur atom and an oxygen atom, such as indolyl (e.g., 1-indolyl, 2-indolyl, 3-indolyl, 4-indolyl, 5-indolyl, 6-indolyl, 7-indolyl), isoindolyl (e.g., 1-isoindolyl, 2-isoindolyl, 3-isoindolyl, 4-isoindolyl, 5-isoindolyl, 6-isoindolyl, 7-isoindolyl), benzo[b]furanyl (e.g., 2-benzo[b]furanyl, 3-benzo[b]furanyl, 4-benzo[b]furanyl, 5-benzo[b]furanyl, 6-benzo[b]furanyl, 7-benzo[b]furanyl), benzo[c]furanyl (e.g., 1-benzo[c]furanyl, 4-benzo[c]furanyl, 5-benzo[c]furanyl), benzo[b]thienyl (e.g., 2-benzo[b]thienyl, 3-benzo[b]thienyl, 4-benzo[b]thienyl, 5-benzo[b]thienyl, 6-benzo[b]thienyl, 7-benzo[b]thienyl), benzo[c]thienyl (e.g., 1-benzo[c]thienyl, 4-benzo[c]thienyl, 5-benzo[c]thienyl), benzimidazolyl (e.g., 1-benzimidazolyl, 2-benzimidazolyl, 4-benzimidazolyl, 5-benzimidazolyl), benzoxazolyl (e.g., 2-benzoxazolyl, 4-benzoxazolyl, 5-benzoxazolyl, 6-benzoxazolyl, 7-benzoxazolyl), benzothiazolyl (e.g., 2-benzothiazolyl, 4-benzothiazolyl, 5-benzothiazolyl, 6-benzothiazolyl, 7-benzothiazolyl), indazolyl (e.g., 1-indazolyl, 2-indazolyl, 3-indazolyl, 4-indazolyl, 5-indazolyl, 6-indazolyl, 7-indazolyl), 1,2-benzisoxazolyl (e.g., 1,2-benzisoxazol-3-yl, 1,2-benzisoxazol-4-yl, 1,2-benzisoxazol-5-yl, 1,2-benzisoxazol-6-yl, 1,2-benzisoxazol-7-yl), 1,2-benzisothiazolyl (e.g., 1,2-benzisothiazol-3-yl, 1,2-benzisothiazol-4-yl, 1,2-benzisothiazol-5-yl, 1,2-benzisothiazol-6-yl, 1,2-benzisothiazol-7-yl), quinolyl (e.g., 2-quinolyl, 3-quinolyl, 4-quinolyl, 5-quinolyl, 6-quinolyl, 7-quinolyl, 8-quinolyl), isoquinolyl (e.g., 1-isoquinolyl, 3-isoquinolyl, 4-isoquinolyl, 5-isoquinolyl, 6-isoquinolyl, 7-isoquinolyl, 8-isoquinolyl), cinnolinyl (e.g., 3-cinnolinyl, 4-cinnolinyl, 5-cinnolinyl, 6-cinnolinyl, 7-cinnolinyl, 8-cinnolinyl), phthalazinyl (e.g., 1-phthalazinyl, 4-phthalazinyl, 5-phthalazinyl, 6-phthalazinyl, 7-phthalazinyl, 8-phthalazinyl), quinazolinyl (e.g., 2-quinazolinyl, 4-quinazolinyl, 5-quinazolinyl, 6-quinazolinyl, 7-quinazolinyl, 8-quinazolinyl), quinoxalinyl (e.g., 2-quinoxalinyl, 3-quinoxalinyl, 5-quinoxalinyl, 6-quinoxalinyl, 7-quinoxalinyl, 8-quinoxalinyl) and the like can be mentioned, particularly, quinolyl (e.g., 2-quinolyl, 3-quinolyl, 4-quinolyl, 5-quinolyl, 6-quinolyl, 7-quinolyl, 8-quinolyl), isoquinolyl (e.g., 1-isoquinolyl, 3-isoquinolyl, 4-isoquinolyl, 5-isoquinolyl, 6-isoquinolyl, 7-isoquinolyl, 8-isoquinolyl); a 5 to 10-membered non-aromatic heterocyclic group containing, besides carbon atom, 1 to 4 hetero atoms of 1 or 2 kinds selected from a nitrogen atom, a sulfur atom and an oxygen atom such as pyrrolidino (1-pyrrolidinyl), 1-piperidinyl, 4-morpholinyl, 4-thiomorpholinyl, azepan-1-yl, azocan-1-yl, azonan-1-yl, 3,4-dihydroisoquinolin-2-yl; and the like), which optionally has substituents selected from the group consisting of (a) a halogen atom (e.g., fluorine, chlorine), (b) cyano, (c) an optionally halogenated C₁₋₆ alkyl group (e.g., methyl, trifluoromethyl), (d) an optionally halogenated C₁₋₆ alkoxy group (e.g., methoxy, trifluoromethoxy), (e) carbamoyl, (f) sulfamoyl and the like),

(d) mono- or di-C₁₋₆ alkylamino (e.g., diethylamino),

(e) mono- or di-C₇₋₁₆ aralkylamino (e.g., benzylamino),

(f) 5 to 7-membered heterocycle-C₁₋₆ alkyl(C₁₋₆ alkyl)amino wherein the heterocycle contains, besides carbon atom, 1 to 4 sulfur atom and an oxygen atom (e.g., ethyl(thienylmethyl)amino),

(g) C₁₋₆ alkyl(C₇₋₁₆ aralkyl)amino wherein the C₁₋₆ alkyl is optionally substituted by hydroxy (e.g., methyl(benzyl)amino, ethyl(benzyl)amino, 2-hydroxyethyl(benzyl)amino), ethyl(thienylmethyl)amino;

(v) a carbamoyl group optionally having substituents selected from the following (a) to (c);

(a) C₁₋₆ alkyl (e.g., methyl, ethyl),

(b) C₁₋₆ alkyl (e.g., methyl, ethyl, propyl, butyl) substituted by mono- or di-C₁₋₆ alkylamino (e.g., dimethylamino), specifically dimethylamino-ethyl, dimethylamino-propyl, dimethylamino-butyl,

(c) C₁₋₆ alkyl (e.g., methyl, ethyl, propyl) substituted by a 5 to 7-membered heterocyclic group containing, besides carbon atom, 1 to 4 hetero atoms of 1 or 2 kinds selected from a nitrogen atom, a sulfur atom and an oxygen atom (e.g., thienyl, pyridyl, 1-piperidinyl, 4-morpholinyl), specifically thienylmethyl, pyridylmethyl, 1-piperidinylethyl, 4-morpholinylethyl, 4-morpholinylpropyl;

(vi) a halogen atom (e.g., chlorine);

(vii) 5 to 7-membered heterocycle-carbonyl-amino containing, besides carbon atom, 1 to 4 hetero atoms of 1 or 2 kinds selected from a nitrogen atom, a sulfur atom and an oxygen atom (e.g., pyridylcarbonylamino),

more preferably, A¹ is a benzene ring optionally having substituents selected from the following (i) to (vii).

(i) C₁₋₆ alkyl group (e.g., methyl),

(ii) a carboxyl group,

(iii) a C₁₋₆ alkoxy-carbonyl group (e.g., methoxycarbonyl, ethoxycarbonyl),

(iv) 5 to 7-membered heterocycle-carbonyl containing, besides carbon atom, 1 to 4 hetero atoms of 1 or 2 kinds selected from a nitrogen atom, a sulfur atom and an oxygen atom (e.g., 1-piperidinylcarbonyl), which is optionally substituted by substituents selected from the following (a) to (g);

(a) hydroxy,

(b) C₁₋₆ alkoxy-carbonyl (e.g., tert-butoxy-carbonyl),

(c) a 5 to 10-membered heterocyclic group containing, besides carbon atom, 1 to 4 hetero atoms of 1 or 2 kinds selected from a nitrogen atom, a sulfur atom and an oxygen atom (e.g., pyrrolidino (1-pyrrolidinyl), 1-piperidinyl, 4-morpholinyl, 4-thiomorpholinyl, azepan-1-yl, azocan-1-yl, azonan-1-yl),

(d) mono- or di-C₁₋₆ alkylamino (e.g., diethylamino),

(e) mono- or di-C₇₋₁₆ aralkylamino (e.g., benzylamino),

(f) 5 to 7-membered heterocycle-C₁₋₆ alkyl(C₁₋₆ alkyl)amino wherein the heterocycle contains, besides carbon atom, 1 to 4 hetero atoms of 1 or 2 kinds selected from a nitrogen atom, a sulfur atom and an oxygen atom (e.g., ethyl(thienylmethyl)amino),

(g) C₁₋₆ alkyl(C₇₋₁₆ aralkyl)amino wherein the C₁₋₆ alkyl is optionally substituted by hydroxy (e.g., methyl(benzyl)amino, ethyl(benzyl)amino, 2-hydroxyethyl(benzyl)amino);

(v) a carbamoyl group optionally having 1 or 2 substituents selected from the following (a) to (c);

(a) C₁₋₆ alkyl (e.g., methyl, ethyl),

(b) C₁₋₆ alkyl (e.g., methyl, ethyl, propyl, butyl) substituted by mono- or di-C₁₋₆ alkylamino (e.g., dimethylamino), specifically, dimethylamino-ethyl, dimethylamino-propyl, dimethylamino-butyl,

(c) C₁₋₆ alkyl (e.g., methyl, ethyl, propyl) substituted by a 5 to 7-membered heterocyclic group containing, besides carbon atom, 1 to 4 hetero atoms of 1 or 2 kinds selected from a nitrogen atom, a sulfur atom and an oxygen atom (e.g., thienyl, pyridyl, 1-piperidinyl, 4-morpholinyl), specifically, thienylmethyl, pyridylmethyl, 1-piperidinylethyl, 4-morpholinylethyl, 4-morpholinylpropyl;

(vi) a halogen atom (e.g., chlorine);

(vii) 5 to 7-membered heterocycle-carbonyl-amino containing, besides carbon atom, 1 to 4 hetero atoms of 1 or 2 kinds selected from a nitrogen atom, a sulfur atom and an oxygen atom (e.g., pyridylcarbonylamino),

(2) a compound represented by the formula 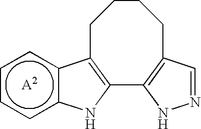
wherein A² is a benzene ring optionally having substituents selected from (i) a carboxyl group, (ii) a C₁₋₆ alkoxy-carbonyl group (e.g., ethoxycarbonyl), (iii) 5 to 7-membered heterocycle-carbonyl containing, besides carbon atom, 1 to 4 hetero atoms of 1 or 2 kinds selected from a nitrogen atom, a sulfur atom and an oxygen atom (e.g., 1-piperidinylcarbonyl), which is optionally substituted by a 5 to 7-membered heterocyclic group containing, besides carbon atom, 1 to 4 hetero atoms of 1 or 2 kinds selected from a nitrogen atom, a sulfur atom and an oxygen atom (e.g., 1-pyrrolidinyl), and the like,
(3) a compound represented by the formula 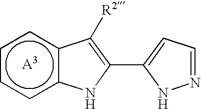
wherein R²′″ is a C₁₋₆ alkyl group (e.g., methyl, propyl), and A³ is a benzene ring optionally having substituents such as 5 to 7-membered heterocycle-carbonyl containing, besides carbon atom, 1 to 4 hetero atoms of 1 or 2 kinds selected from a nitrogen atom, a sulfur atom and an oxygen atom (e.g., 1-piperidinylcarbonyl), which is optionally substituted by a 5 to 10-membered heterocyclic group containing, besides carbon atom, 1 to 4 hetero atoms of 1 or 2 kinds selected from a nitrogen atom, a sulfur atom and an oxygen atom (e.g., 3,4-dihydroisoquinolin-2-yl), and the like,
(4) a compound represented by the formula 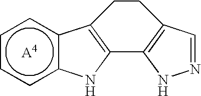
wherein A⁴ is a benzene ring optionally having substituents selected from a halogen atom (e.g., chlorine), 5 to 7-membered heterocycle-carbonyl-amino containing, besides carbon atom, 1 to 4 hetero atoms of 1 or 2 kinds selected from a nitrogen atom, a sulfur atom and an oxygen atom (e.g., pyridylcarbonylamino) and the like,
(5) compounds of Examples 1-69, preferably Examples 1-68, more preferably compounds of Examples 1-39,
(6) compounds of Examples 1-38 and 40-69, particularly compounds of Examples 1-38,
(7) compounds of Examples 41-49 or the like.

However, 7-methyl-3-phenyl-1,4,5,10-tetrahydropyrazolo[3,4-a]carbazole hydrochloride, 7-methoxy-1,10-dihydropyrazolo[3,4-a]carbazole, 9-methoxy-1,10-dihydropyrazolo[3,4-a]carbazole, 8-methoxy-4,5,6,11-tetrahydro-1H-pyrazolo[4′,3′:6,7]cyclohepta[1,2-b]indole, 1,4,5,10-tetrahydropyrazolo[3,4-a]carbazole, 7-chloro-1,4,5,10-tetrahydropyrazolo[3,4-a]carbazole, 7-bromo-1,4,5,10-tetrahydropyrazolo[3,4-a]carbazole, 7-methyl-1,4,5,10-tetrahydropyrazolo[3,4-a]carbazole, 7-cyclohexyl-1,4,5,10-tetrahydropyrazolo[3,4-a]carbazole, 1,10-dihydropyrazolo[3,4-a]carbazole, 9-methyl-1,10-dihydropyrazolo[3,4-a]carbazole, 8-methyl-1,10-dihydropyrazolo[3,4-a]carbazole, 7-methyl-1,10-dihydropyrazolo[3,4-a]carbazole, 7-chloro-1,10-dihydropyrazolo[3,4-a]carbazole and 7-bromo-1,10-dihydropyrazolo[3,4-a]carbazole (from patent document 1 and non-patent documents 3-6) are excluded from the scope of the compounds of the present invention (I).

In addition, 8-methoxy-4,5,6,11-tetrahydro-1H-pyrazolo[4′,3′:6,7]cyclohepta[1,2-b]indole (patent document 1), 4,5,6,11-tetrahydro-1H-pyrazolo[4′,3′:6,7]cyclohepta[1,2-b]indole-8-carboxamide, 3-methyl-N-(4,5,6,11-tetrahydro-1H-pyrazolo[4′,3′:6,7]cyclohepta[1,2-b]indol-8-yl)butanamide, 8-chloro-4,5,6,11-tetrahydro-1H-pyrazolo[4′,3′:6,7]cyclohepta[1,2-b]indole, N-benzyl-4,5,6,11-tetrahydro-1H-pyrazolo[4′,3′:6,7]cyclohepta[1,2-b]indole-8-carboxamide, N-isobutyl-4,5,6,11-tetrahydro-1H-pyrazolo[4′,3′:6,7]cyclohepta[1,2-b]indole-8-carboxamide, ethyl 4,5,6,11-tetrahydro-1H-pyrazolo[4′,3′:6,7]cyclohepta[1,2-b]indole-3-carboxylate, N-(4,5,6,11-tetrahydro-1H-pyrazolo[4′,3′:6,7]cyclohepta[1,2-b]indol-8-yl)acetamide and 2-phenyl-N-(4,5,6,11-tetrahydro-2H-pyrazolo[4′,3′:6,7]cyclohepta[1,2-b]indol-8-yl)acetamide (patent document 3) are excluded from the scope of the novel compounds (Ia) of the present invention.

As the salts of compound (I) or compound (I′), for example, a metal salt, an ammonium salt, salts with organic bases, salts with inorganic acids, salts with organic acids, salts with basic or acidic amino acids and the like can be mentioned. As preferable examples of the metal salt, alkali metal salts such as sodium salt, potassium salt and the like; alkaline earth metal salts such as calcium salt, magnesium salt, barium salt and the like; aluminum salt and the like can be mentioned. As preferable examples of the salts with organic bases, salts with trimethylamine, triethylamine, pyridine, picoline, 2,6-lutidine, ethanolamine, diethanolamine, triethanolamine, cyclohexylamine, dicyclohexylamine, N,N′-dibenzylethylenediamine and the like can be mentioned. As preferable examples of the salts with inorganic acids, salts with hydrochloric acid, hydrobromic acid, nitric acid, sulfuric acid, phosphoric acid and the like can be mentioned. As preferable examples of the salts with organic acids, salts with formic acid, acetic acid, trifluoroacetic acid, phthalic acid, fumaric acid, oxalic acid, tartaric acid, maleic acid, citric acid, succinic acid, malic acid, methanesulfonic acid, benzenesulfonic acid, p-toluenesulfonic acid and the like can be mentioned. As preferable examples of the salts with basic amino acids, salts with arginine, lysine, ornithine and the like can be mentioned, and as preferable examples of the salts with acidic amino acids, salts with aspartic acid, glutamic acid and the like can be mentioned.

Of these, pharmaceutically acceptable salts are preferable. For example, when the compound has an acidic functional group, inorganic salts such as alkali metal salts (e.g., sodium salt, potassium salt etc.), alkaline earth metal salts (e.g., calcium salt, magnesium salt, barium salt etc.) and the like, ammonium salt and the like can be mentioned, and when the compound has a basic functional group, for example, salts with inorganic acids such as hydrochloric acid, hydrobromic acid, nitric acid, sulfuric acid, phosphoric acid and the like, and salts with organic acids such as acetic acid, phthalic acid, fumaric acid, oxalic acid, tartaric acid, maleic acid, citric acid, succinic acid, methanesulfonic acid, p-toluenesulfonic acid and the like can be mentioned.

The production methods of the compounds (I) of the present invention are described in the following. The compounds (I′) of the present invention can be also produced in the same manner.

The compound of the present invention (I) can be obtained by, for example, the method shown in the scheme below, a method analogous thereto and the like.

The compounds in the scheme include salts and as such salts, for example, those similar to the salts of the compound (1) and the like can be mentioned.

The compound obtained in each step can be used for the next reaction in the form of a reaction mixture or as a crude product. In addition, it can be isolated from a reaction mixture by a conventional method, and can be easily purified by a separation means such as recrystallization, distillation, chromatography and the like.

When the compound in the scheme is commercially available, a commercially available product can be also used as it is.

The compound (I) can be produced by construction of a pyrazole ring after construction of an indole ring. A schematic representation of the reaction scheme is shown below, herein each symbol of the compound is as defined above 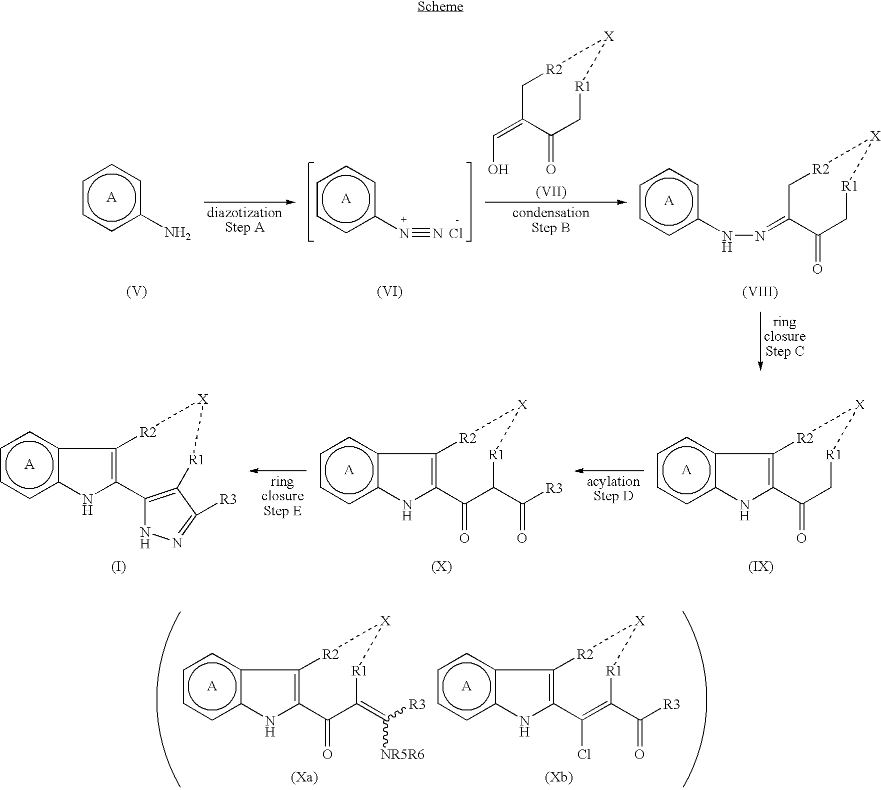
wherein R¹, R², R³ and X are as defined for the formula (I), and R⁵ and R⁶ are each a lower (C₁₋₆)alkyl group, preferably a methyl group.

An indole ring can be constructed by Fischer's indole synthesis described in Ber. 1883, Vol. 17, p. 559 and the like or a method analogous thereto. It can be also synthesized by indole synthesis other than the Fischer's indole synthesis, which is described in Ber. 1912, Vol. 45, p. 1128, Ber. 1908, Vol. 41, p. 3925 and the like or a method analogous thereto. Of the Fischer's indole syntheses, the method of Jappe-Klingemann et al. as described in J. Chem. Soc., 1927, p. 1 and the like is most preferable. To be specific, aniline (V) is diazotized to produce a diazonium salt (VI) in the reaction system, which is condensed with 1,3-dicarbonyl compound (VII) to give hydrazone (VIII), which is then treated with an acid, whereby an indole ring can be produced [Steps A, B and C].

Aniline (V) can be produced by a method known per se, such as a method described in, for example, Shin Jikken Kagaku Koza, The Chemical Society of Japan Ed., Maruzen Co., Ltd., vol. 14, Synthesis and Reaction of Organic Compounds III, pp. 1333-1399 and the like, or a method analogous thereto.

The 1,3-dicarbonyl compound (VII) can be produced by a method known per se, such as a method described in, for example, Helv. Chim. Acta., 1947, Vol. 30, p. 1883, Org. Lett., 2001, Vol. 3, No. 6, p. 861, J. Am. Chem. Soc., 1953, Vol. 75, p. 2050 and the like, or a method analogous thereto.

[Step A]

As a method of producing diazonium salt (VI) from aniline (V), a method of producing diazonium salt (VI) using aniline (V) as a reaction agent and nitrite such as sodium nitrite and the like, in the presence of an acid such as hydrochloric acid and the like in a polar solvent such as water and the like or a mixed solvent thereof can be mentioned. Sodium nitrite is used in a proportion of about 1-10 mol, preferably about 1-2 mol, per 1 mol of aniline (V). The reaction temperature is generally about −20° C. to 25° C., preferably about 0° C. While the reaction time varies depending on the reagent, solvent and reaction temperature to be employed, it is generally about 5 min-2 hrs, preferably about 10 min-1 hr.

[Step B]

The produced diazonium salt (VI) can be reacted with 1,3-dicarbonyl compound (VII) in the presence of a base. As the base, inorganic salts represented by hydroxide such as sodium hydroxide, potassium hydroxide and the like, carbonate such as sodium carbonate, potassium carbonate and the like can be used. As the solvent, polar solvents such as water, alcohols (e.g., methanol, ethanol etc.) and the like or mixed solvents thereof can be used. The base is used in a proportion of about 1-10 mol, preferably about 1-1.2 mol, per 1 mol of 1,3-dicarbonyl compound (VII). The reaction temperature is generally about −20° C. to 50° C., preferably about 0° C. to 25° C. While the reaction time varies depending on the reagent, solvent and reaction temperature to be employed, it is generally about 5 min-24 hrs, preferably about 30 min-2 hrs.

[Step C]

An indole ring can be constructed by treating hydrazone (VIII) with an acid. As the acid, mineral acids such as hydrochloric acid, sulfuric acid and polyphosphoric acid or organic acids such as formic acid, acetic acid, p-toluenesulfonic acid and methanesulfonic acid can be used. While a solvent may or may not be used, a solvent such as water, toluene and the like or a mixed solvent thereof can be used. The acid is used in a proportion of about 1-1000 mol, preferably about 10-100 mol, per 1 mol of hydrazone (VIII).

The reaction temperature is generally about −20° C. to 200° C., preferably about 25° C. to 100° C. While the reaction time varies depending on the reagent, solvent and reaction temperature to be employed, it is generally about 5 min-24 hrs, preferably about 30 min-2 hrs.

[Step D]

The acylation reaction of ketone (IX) can be performed by the reaction with ester, anhydride and the like in the presence of a base. As the base, sodium hydride, potassium hydride, sodium methoxide, sodium ethoxide and the like can be used. As the solvent, solvents such as tetrahydrofuran, diethyl ether, N,N-dimethylformamide and the like or mixed solvents thereof can be used. The base is used in a proportion of about 1-10 mol, preferably about 1-1.2 mol, per 1 mol of ketone (IX). The reaction temperature is generally about −75° C. to 50° C., preferably about 0° C. to 25° C. While the reaction time varies depending on the reagent, solvent and reaction temperature to be employed, it is generally about 5 min-24 hrs, preferably about 10 min-2 hrs.

[Step E]

The object compound (I) can be obtained by reacting acyl compound (X), or compound (Xa) or compound (Xb) and the like obtained from acyl compound (X), which has equivalent reactivity to that of acyl compound (X), with hydrazine. As the solvent, solvents such as water, ethanol, methanol and the like or mixed solvents thereof can be used. Hydrazine or a hydrate thereof is used in a proportion of about 1-10 mol, preferably about 1-1.2 mol, per 1 mol of acyl compound (X), or compound (Xa) or compound (Xb) having equivalent reactivity thereto, and the like. The reaction temperature is generally about 0° C. to 200° C., preferably about 25° C. to 100° C. While the reaction time varies depending on the reagent, solvent and reaction temperature to employed, it is generally about 5 min-24 hrs, preferably about 10 min-2 hrs.

The compound (I) can be also produced by constructing an indole ring after constructing a pyrazole ring, and can be produced by a method comprising directly binding the indole ring to the pyrazole ring.

The compound of the present invention (I-1) can be also produced by reacting a compound represented by the formula 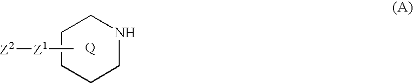
wherein each symbol is as defined above, or a salt thereof with a compound represented by the formula 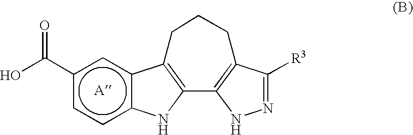
wherein each symbol is as defined above, or a salt thereof or a reactive derivative thereof.

As the reactive derivative of carboxylic acid (B) or a salt thereof, any can be used as long as it has equivalent reactivity to that of the carboxylic acid (B) and, for example, an ester compound of carboxylic acid (B) and the like can be used. As the ester compound of carboxylic acid (B), carboxylic acid (B) esterified by an alkyl group such as a C₁₋₆ alkyl group (e.g., methyl, ethyl and the like) and the like can be used. Particularly, compounds of Examples 2, 4 and 32 and the like can be preferably used.

The amine (A) can be produced by a method known per se, such as a method described in J. Med. Chem., 1992, Vol. 35, p. 4020 and the like or a method analogous thereto.

The carboxylic acid (B), a salt thereof and a reactive derivative thereof can be produced according to the aforementioned production method of the compound of the present invention (I).

The carboxylic acid (B) can be produced by hydrolysis of the corresponding ester. The ester can be hydrolyzed in the presence of a base using a mixed solvent of water and an organic solvent. As the base, sodium hydroxide, potassium hydroxide and the like can be used. As the solvent, solvents such as water, tetrahydrofuran, methanol, ethanol and the like or mixed solvents thereof can be used. Generally, the base is used in a proportion of about 1-10 mol, preferably about 1-3 mol, per 1 mol of the ester. The reaction temperature is generally about −25° C. to 100° C., preferably about 0° C. to 25° C. While the reaction time varies depending on the reagent, solvent and reaction temperature to be employed, it is generally about 5 min-24 hrs, preferably about 10 min-2 hrs.

The reaction between amine (A) and carboxylic acid (B) or a salt thereof or a reactive derivative thereof can be carried out using a condensation agent generally used for amidation or peptide synthesis.

As the condensation agent, for example, carbodiimide condensation reagents such as dicyclohexylcarbodiimide, diisopropylcarbodiimide, 1-ethyl-3-dimethylaminopropylcarbodiimide (EDC) and hydrochloride thereof and the like; phosphoric acid condensation reagents such as diethyl cyanophosphate, diphenylphosphoryl azide and the like; carbonyldiimidazole, 2-chloro-1,3-dimethylimidazolium tetrafluoroborate; a combination of 1H-1,2,3-benzotriazol-1-ol and N-[3-(dimethylamino)propyl]-N′-ethylcarbodiimide hydrochloride; azolides such as N,N′-carbonyldiimidazole and the like; dehydrating agents such as N-ethoxycarbonyl-2-ethoxy-1,2-dihydroquinoline, phosphorus oxychloride, acetic anhydride and the like; 2-halogenopyridinium salts such as 2-chloromethylpyridinium iodide, 2-fluoro-1-chloromethylpyridinium iodide and the like; and the like can be used. Of these, diethyl cyanophosphate, or a combination of 1H-1,2,3-benzotriazol-1-ol and N-[3-(dimethylamino)propyl]-N′-ethylcarbodiimide hydrochloride can be preferably used.

As the solvent usable for the reaction using a condensation agent, for example, amides such as N,N-dimethylformamide, N,N-dimethylacetamide, 1-methyl-2-pyrrolidone and the like; halogenated hydrocarbons such as chloroform, dichloromethane and the like; aromatic hydrocarbons such as benzene, toluene and the like; ethers such as tetrahydrofuran, dioxane, diethyl ether and the like; esters such as ethyl acetate, propyl acetate, butyl acetate and the like; nitriles such as acetonitrile, propionitrile and the like; ketones such as acetone, 2-butanone and the like; water and the like can be mentioned. These solvents may be used in a mixture of two or more kinds thereof mixed at a suitable ratio.

When a carbodiimide condensation reagent is used as the condensation agent, the reaction efficiency can be enhanced by the use of a suitable condensation promoter (e.g., 1-hydroxy-7-azabenzotriazole, 1-hydroxybenzotriazole, N-hydroxysuccinimide, N-hydroxyphthalimide) as necessary.

When a phosphoric acid condensation reagent is used as the condensation agent, the reaction efficiency can be enhanced by generally adding an organic amine base such as triethylamine and the like and using water, an organic solvent or a mixed solvent thereof.

The carboxylic acid (B) or a salt thereof or a reactive derivative thereof can be used in a proportion of generally about 0.1-10 mol, preferably about 0.5-2 mol, per 1 mol of amine (A).

The condensation agent can be used in a proportion of about 0.1-10 mol, preferably about 1-2 mol, per 1 mol of amine (A).

The reaction temperature is generally about −20° C. to 50° C., preferably about 0° C. to 25° C. While the reaction time varies depending on the reagent, solvent and reaction temperature to be employed, it is generally about 5 min-100 hrs, preferably 1 hr-24 hrs.

As the reactive derivative, for example, acid anhydride, acid halide (e.g., acid chloride, acid bromide), imidazolide, mixed anhydride (e.g., anhydrides of methyl carbonate, ethyl carbonate, isobutyl carbonate), ester (4-nitrophenol ester) and the like can be used and these reactive derivatives can be produced from carboxylic acid (B).

When, for example, an acid halide is used as a reactive derivative, an acid halide as the reactive derivative can be produced by reacting carboxylic acid (B) with, for example, a halogenating agent such as thionyl chloride, thionyl bromide, phosphorus trichloride, phosphorus tribromide, phosphorus oxychloride, phosphorus pentachloride and the like. The amount of the halogenating agent to be used is generally, 0.1-10 molar equivalents, preferably 0.3-3 molar equivalents, relative to carboxylic acid (B) or a salt thereof. In a reaction using the halogenating agent, amides such as N,N-dimethylformamide, N,N-dimethylacetamide and the like can be used as a catalyst. The amount of the catalyst to be used is generally 0.0001-10 molar equivalents, preferably 0.001-3 molar equivalents, relative to carboxylic acid (B). Where necessary, these catalysts may be used as a solvent. As the solvent for acid halogenation, for example, amides such as N,N-dimethylformamide, N,N-dimethylacetamide, 1-methyl-2-pyrrolidone and the like; halogenated hydrocarbons such as chloroform, dichloromethane and the like; aromatic hydrocarbons such as benzene, toluene and the like; ethers such as tetrahydrofuran, dioxane, diethyl ether and the like; esters such as ethyl acetate, propyl acetate, butyl acetate and the like; nitriles such as acetonitrile, propionitrile and the like; ketones such as acetone, 2-butanone and the like and the like can be used. These solvents may be used in a mixture of two or more kinds thereof mixed at a suitable ratio. The reaction temperature is generally about −30° C. to 150° C. While the reaction time varies depending on the reagent, solvent and reaction temperature to be employed, it is generally 0.5-24 hr.

If compound (I) is obtained as a free compound, it can be converted into a desired salt by a method known per se or a modification thereof; conversely, if compound (I) is obtained as a salt, it can be converted into a free form or another desired salt by a method known per se or a modification thereof.

The compound (I) may be used as a prodrug. A prodrug of the compound (I) means a compound which is converted to the compound (I) of the present invention with a reaction due to an enzyme, an gastric acid, etc. under the physiological condition in the living body, that is, a compound which is converted to the compound (I) of the present invention with oxidation, reduction, hydrolysis, etc. according to an enzyme; a compound which is converted to the compound (I) of the present invention by hydrolysis etc. due to gastric acid, etc.

A prodrug for compound (I) may be a compound obtained by subjecting an amino group in compound (I) to an acylation, alkylation or phosphorylation (e.g., a compound obtained by subjecting an amino group in compound (I) to an eicosanoylation, alanylation, pentylaminocarbonylation, (5-methyl-2-oxo-1,3-dioxolen-4-yl)methoxycarbonylation, tetrahydrofuranylation, pyrrolidylmethylation, pivaloyloxymethylation and tert-butylation, etc.); a compound obtained by subjecting a hydroxy group in compound (I) to an acylation, alkylation, phosphorylation or boration (e.g., a compound obtained by subjecting an hydroxy group in compound (1) to an acetylation, palmitoylation, propanoylation, pivaloylation, succinylation, fumarylation, alanylation, dimethylaminomethylcarbonylation, etc.); a compound obtained by subjecting a carboxyl group in compound (I) to an esterification or amidation (e.g., a compound obtained by subjecting a carboxyl group in compound (I) to an ethyl esterification, phenyl esterification, carboxymethyl esterification, dimethylaminomethyl esterification, pivaloyloxymethyl esterification, ethoxycarbonyloxyethyl esterification, phthalidyl esterification, (5-methyl-2-oxo-1,3-dioxolen-4-yl)methyl esterification, cyclohexyloxycarbonylethyl esterification and methylamidation, etc.) and the like. Any of these compounds can be produced from compound (I) by a method known per se.

A prodrug for compound (I) may also be one which is converted into compound (I) under a physiological condition, such as those described in IYAKUHIN no KAIHATSU (Development of Pharmaceuticals), Vol. 7, Design of Molecules, p. 163-198, Published by HIROKAWA SHOTEN (1990).

The compound (I′) can be also used as a prodrug. As the prodrug of compound (I′), those similar to the prodrugs of compound (I) can be mentioned.

When compound (I) has isomers such as tautomer, optical isomer, stereoisomer, positional isomer, rotational isomer and the like, and any isomers and mixtures are encompassed in the compound of the present invention. For example, compound (I) and tautomer (Ic) thereof, which are represented by the formulas 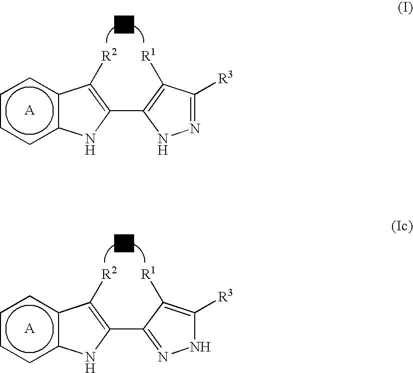
are encompassed in the compound of the present invention. Moreover, when compound (I) has an optical isomer, an optical isomer separated from a racemate is also encompassed in the compound (I). These isomers can be obtained as independent products by a synthesis means or a separation means (concentration, solvent extraction, column chromatography, recrystallization and the like) known per se.

The compound (I) may be a crystal, and both a single crystal and crystal mixtures are encompassed in compound (I). Crystals can be produced by crystallization according to crystallization methods known per se.

The compound (I) may be a solvate (e.g., hydrate etc.) or a non-solvate, both of which are encompassed in compound (I).

A compound labeled with an isotope (e.g., ³H, ¹⁴C, ³⁵S, ¹²⁵I and the like) and the like is also encompassed in compound (I).

The compound (I′) including compound (I) of the present invention and a prodrug thereof (hereinafter sometimes to be abbreviated as the compound of the present invention) have, for example, a kinase (phosphorylation enzyme) inhibitory action. As the kinase, for example, vascular endothelial growth factor receptor (VEGFR, Flt-1), fibroblast growth factor receptor (FGFR) and the like can be mentioned. As the vascular endothelial growth factor receptor (VEGFR), vascular endothelial growth factor receptor 1 (VEGFR1), vascular endothelial growth factor receptor 2 (VEGFR2), vascular endothelial growth factor receptor 3 (VEGFR3, Flt-4) and the like can be mentioned. Of these, vascular endothelial growth factor receptor 2 (VEGFR2) is preferable. As the fibroblast growth factor receptor (FGFR), fibroblast growth factor receptor 1 (FGFR1), fibroblast growth factor receptor 2 (FGFR2), fibroblast growth factor receptor 3 (FGFR3), fibroblast growth factor receptor 4 (FGFR4) and the like can be mentioned. Of these, fibroblast growth factor receptor (FGFR) 1 is preferable. Particularly, as the kinase, vascular endothelial growth factor receptor 2 (VEGFR2) is preferable. In addition, as the kinase, platelet derived growth factor receptor alpha (PDGFR alpha), platelet derived growth factor receptor beta (PDGFR beta), angiopoetin-1 receptor (TIE2), stem cell factor receptor (c-Kit), Aurora A, Aurora B, CDK, MEK1, MEK2, A-Raf, B-Raf, C-Raf, Akt, ERK, MAPK, Src, epidermal growth factor receptor (EGFR), epidermal growth factor receptor 2 (HER2), epidermal growth factor receptor 4 (HER4) and the like can be mentioned.

For example, the vascular endothelial growth factor receptor 2 inhibitory activity of the compound of the present invention can be determined according to Test Example 1, the vascular endothelial cell growth inhibitory activity can be determined according to Test Example 2, and the antitumor activity can be determined according to Test Example 3.

The compound of the present invention particularly shows high affinity for vascular endothelial growth factor receptor (VEGFR, Flt-1), and the selectivity for vascular endothelial growth factor receptor 2 (VEGFR2) is specifically high. In addition, since the compound of the present invention is also superior in the efficacy expression, pharmacokinetics (absorption, distribution, metabolism, excretion etc.), solubility (water-solubility etc.), interaction with other pharmaceutical products, safety (acute toxicity, chronic toxicity, genetic toxicity, reproductive toxicity, cardiotoxicity, carcinogenicity etc.) and stability (chemical stability, stability to enzyme etc.), it is useful as a pharmaceutical agent.

Therefore, the compound of the present invention is useful as an agent for inhibiting kinase, preferably an agent for inhibiting vascular endothelial growth factor receptor (VEGFR, Flt-1), an agent for inhibiting fibroblast growth factor receptor (FGFR), more preferably an agent for inhibiting vascular endothelial growth factor receptor 2 (VEGFR2), an agent for inhibiting fibroblast growth factor receptor (FGFR) 1, particularly preferably an agent for inhibiting vascular endothelial growth factor receptor 2 (VEGFR2), for mammals (e.g., mouse, rat, hamster, rabbit, cat, dog, bovine, sheep, monkey, human etc.), and is used as a pharmaceutical agent such as an agent for inhibiting angiogenesis, an agent for inhibiting vascular endothelial cell growth, or an agent for the prophylaxis or treatment of diseases possibly influenced by vascular endothelial growth factor, such as cancer (e.g., colorectal cancer, lung cancer, pancreatic cancer, gastric cancer, breast cancer, ovarian cancer, prostate cancer, liver cancer, thyroid cancer, kidney cancer, cerebral tumor, melanoma, urinary bladder cancer and the like), diabetic retinopathy, heumatoid arthritis, psoriasis, atherosclerosis, Kaposi sarcoma, COPD, pain, inflammation or hypertension, an agent for inhibiting growth of cancer, an agent for suppressing metastasis of cancer and the like. Particularly, the compound of the present invention is effective for patients having cancer with expression or high expression of vascular endothelial growth factor receptor (VEGFR, Flt-1) and/or fibroblast growth factor receptor (FGFR) 1 (e.g., colorectal cancer, lung cancer, pancreatic cancer, gastric cancer, breast cancer, ovarian cancer, prostate cancer, liver cancer, thyroid cancer, kidney cancer, cerebral tumor, melanoma, urinary bladder cancer and the like).

The compound of the present invention can be administered orally or parenterally as it is or after mixing with a pharmacologically acceptable carrier.

The dosage form of the compound of the present invention for oral administration, for example, tablet (including sugar-coated tablet, film-coated tablet), pill, granule, powder, capsule (including soft capsule, microcapsule), syrup, emulsion, suspension and the like, and the dosage form for parenteral administration is, for example, injection, injecting agent, instillation, suppository and the like. In addition, it is effective to make a sustained release preparation by combining with a suitable base (e.g., polymer of butyric acid, polymer of glycolic acid, copolymer of butyric acid-glycolic acid, a mixture of polymer of butyric acid and polymer of glycolic acid, polyglycerol fatty acid ester etc.).

As a method to produce the compound of the present invention in the above-mentioned dosage form, a known production method generally used in the pertinent field can be applied. When the above-mentioned dosage form is produced, suitable amounts of additives such as an excipients, a binder, a disintegrant, a lubricant, a sweetening agent, a surfactant, a suspending agent, an emulsifier and the like generally used in the pertinent field are appropriately added as necessary, and produced.

When the compound of the present invention is prepared into a tablet, for example, it can be produced by adding an excipient, a binder, a disintegrant, a lubricant and the like, and when a pill and a granule are to be prepared, they can be produced by adding an excipient, a binder, a disintegrant and the like. When a powder and a capsule are to be prepared, they can be produced by adding an excipient and the like, and when a syrup is to be prepared, it can be produced by adding a sweetener and the like, and when an emulsion or a suspension is to be prepared, it can be produced by adding a suspending agent, a surfactant, an emulsifier and the like.

Examples of the excipient include lactose, sucrose, glucose, starch, sucrose, microcrystalline cellulose, powdered glycyrrhiza, mannitol, sodium hydrogen carbonate, calcium phosphate, calcium sulfate and the like.

Examples of the binder include 5-10 wt % starch liquid paste, 10-20 wt % gum arabic solution or gelatin solution, 1-5 wt % tragacanth solution, carboxymethyl cellulose solution, sodium alginate solution, glycerin and the like.

Examples of the disintegrant include starch, calcium carbonate and the like.

Examples of the lubricant include magnesium stearate, stearic acid, calcium stearate, purified talc and the like.

Examples of the sweetener include glucose, fructose, invert sugar, sorbitol, xylitol, glycerin, simple syrup and the like.

Examples of the surfactant include sodium lauryl sulfate, polysorbate 80, sorbitan monofatty acid ester, polyoxyl 40 stearate and the like.

Examples of the suspending agent include gum arabic, sodium alginate, sodium carboxymethyl cellulose, methyl cellulose, bentonite and the like.

Examples of the emulsifier include gum arabic, tragacanth, gelatin, polysorbate 80 and the like.

Furthermore, when the compound (I) of the present invention is produced in the above-mentioned dosage form, a suitable amount of a coloring agent, a preservative, an aromatic, a corrigent, a stabilizer, viscous agents and the like typically used in the field of preparation can be added on demand.

As the injection, intravenous injection as well as subcutaneous injection, intracutaneous injection, intramuscular injection, instillation and the like are mentioned, and as a sustained release preparation, iontophoresis transdermal agent and the like are mentioned.

Such injections are prepared by methods known per se, or by dissolving, suspending or emulsifying the compound of the present invention in a sterilized solution or oily liquid. As an aqueous solution for injection, physiological saline, isotonic solutions containing glucose or other auxiliary drugs (e.g., D-sorbitol, D-mannitol, sodium chloride and the like) and the like can be mentioned, and they can be used in combination with suitable dissolution aids, such as alcohols (e.g., ethanol), polyalcohols (e.g., propylene glycol, polyethylene glycol), nonionic surfactants (e.g., polysorbate 80, HCO-50) and the like. As an oily liquid, sesame oil, soybean oil and the like can be mentioned, which may be used in combination with dissolution aids such as benzyl benzoate, benzyl alcohol and the like. In addition, buffers (e.g., phosphate buffer, sodium acetate buffer), soothing agents (e.g., benzalkonium chloride, procaine hydrochloride and the like), stabilizers (e.g., human serum albumin, polyethylene glycol and the like), preservatives (e.g., benzyl alcohol, phenol and the like) and the like. A prepared injection is generally filled in an ampoule.

While the content of the compound of the present invention in the pharmaceutical agent of the present invention varies depending on the form of the pharmaceutical preparation, it is generally about 0.01 to 100 wt %, preferably about 2 to 85 wt %, more preferably about 5 to 70 wt %, relative to the entire preparation.

While the content of the additive in the pharmaceutical agent of the present invention varies depending on the form of the pharmaceutical preparation, it is generally about 1 to 99.9 wt %, preferably about 10 to 90 wt %, relative to the entire preparation.

The compound of the present invention is stable and low toxic, and can be used safely. While the daily dose varies depending on the condition and body weight of patients, the kind of compound, administration route and the like, in the case of, for example, oral administration to patients for the treatment of cancer, the daily dose to an adult (body weight about 60 kg) is about 1 to 1000 mg, preferably about 3 to 300 mg, more preferably about 10 to 200 mg, as an active ingredient (the compound of the present invention), which can be given in a single administration or administered in 2 or 3 portions a day.

When the compound of the present invention is administered parenterally, it is generally administered in the form of a liquid (e.g., injection). While the dose varies depending on the subject of administration, target organ, symptom, administration method and the like, it is, for example, about 0.01 mg-about 100 mg, preferably about 0.01-about 50 mg, more preferably about 0.01-about 20 mg, in the form of an injection, relative to 1 kg of body weight, which is preferably given by intravenous injection.

The compound of the present invention can be used concurrently with other drugs. To be specific, the compound of the present invention can be used together with hormonal therapeutic agents, chemotherapeutic agents, immunotherapeutic agents, pharmaceutical agents inhibiting the action of cell growth factors or cell growth factor receptors and the like. In the following, the drugs that can be used in combination with the compound of the present invention are abbreviated as concomitant drugs.

As examples of the “hormonal therapeutic agents”, there can be used fosfestrol, diethylstylbestrol, chlorotrianisene, medroxyprogesterone acetate, megestrol acetate, chlormadinone acetate, cyproterone acetate, danazol, allylestrenol, gestrinone, mepartricin, raloxifene, ormeloxifene, levormeloxifene, anti-estrogens (e.g., tamoxifen citrate, toremifene citrate, and the like), pill preparations, mepitiostane, testrolactone, aminoglutethimide, LH-RH agonists (e.g., goserelin acetate, buserelin, leuprorelin, and the like), droloxifene, epitiostanol, ethinylestradiol sulfonate, aromatase inhibitors (e.g., fadrozole hydrochloride, anastrozole, retrozole, exemestane, vorozole, formestane, and the like), anti-androgens (e.g., flutamide, bicartamide, nilutamide, and the like), 5α-reductase inhibitors (e.g., finasteride, epristeride, and the like), adrenocorticohormone drugs (e.g., dexamethasone, prednisolone, betamethasone, triamcinolone, and the like), androgen synthesis inhibitors (e.g., abiraterone, and the like), retinoid and drugs that retard retinoid metabolism (e.g., liarozole, and the like), etc.

As examples of the “chemotherapeutic agents”, there can be used alkylating agents, antimetabolites, anticancer antibiotics, plant-derived anticancer agents, and the like.

As examples of the “alkylating agents”, there can be used nitrogen mustard, nitrogen mustard-N-oxide hydrochloride, chlorambutyl, cyclophosphamide, ifosfamide, thiotepa, carboquone, improsulfan tosylate, busulfan, nimustine hydrochloride, mitobronitol, melphalan, dacarbazine, ranimustine, estramustine phosphate sodium, triethylenemelamine, carmustine, lomustine, streptozocin, pipobroman, etoglucid, carboplatin, cisplatin, miboplatin, nedaplatin, oxaliplatin, altretamine, ambamustine, dibrospidium hydrochloride, fotemustine, prednimustine, pumitepa, ribomustin, temozolomide, treosulphan, trophosphamide, zinostatin stimalamer, adozelesin, cystemustine, bizelesin and the like.

As examples of the “antimetabolites”, there can be used mercaptopurine, 6-mercaptopurine riboside, thioinosine, methotrexate, enocitabine, cytarabine, cytarabine ocfosfate, ancitabine hydrochloride, 5-FU drugs (e.g., fluorouracil, tegafur, UFT, doxifluridine, carmofur, gallocitabine, emmitefur, and the like), aminopterine, leucovorin calcium, tabloid, butocine, folinate calcium, levofolinate calcium, cladribine, emitefur, fludarabine, gemcitabine, hydroxycarbamide, pentostatin, piritrexim, idoxuridine, mitoguazone, thiazophrine, ambamustine and the like.

As examples of the “anticancer antibiotics”, there can be used actinomycin-D, actinomycin-C, mitomycin-C, chromomycin-A3, bleomycin hydrochloride, bleomycin sulfate, peplomycin sulfate, daunorubicin hydrochloride, doxorubicin hydrochloride, aclarubicin hydrochloride, pirarubicin hydrochloride, epirubicin hydrochloride, neocarzinostatin, mithramycin, sarcomycin, carzinophilin, mitotane, zorubicin hydrochloride, mitoxantrone hydrochloride, idarubicin hydrochloride, and the like.

As examples of the “plant-derived anticancer agents”, there can be used etoposide, etoposide phosphate, vinblastine sulfate, vincristine sulfate, vindesine sulfate, teniposide, paclitaxel, docetaxel, vinorelbine, and the like.

As examples of the “immunotherapeutic agents (BRM)”, there can be used picibanil, krestin, sizofuran, lentinan, ubenimex, interferons, interleukins, macrophage colony-stimulating factor, granulocyte colony-stimulating factor, erythropoietin, lymphotoxin, BCG vaccine, Corynebacterium parvum, levamisole, polysaccharide K, procodazole, and the like.

As the “cell growth factor” of the “pharmaceutical agents inhibiting the action of cell growth factors or cell growth factor receptors”, there can be used any substances that promote cell proliferation, which are normally peptides having a molecular weight of not more than 20,000 that are capable of exhibiting their action at low concentrations by binding to a receptor, including (1) EGF (epidermal growth factor) or substances possessing substantially the same activity as it [e.g., EGF, heregulin (HER2 ligand), and the like], (2) insulin or substances possessing substantially the same activity as it [e.g., insulin, IGF (insulin-like growth factor)-1, IGF-2, and the like], (3) FGF (fibroblast growth factor) or substances possessing substantially the same activity as it [e.g., acidic FGF, basic FGF, KGF (keratinocyte growth factor), FGF-10, and the like], (4) other cell growth factors [e.g., CSF (colony stimulating factor), EPO (erythropoietin), IL-2 (interleukin-2), NGF (nerve growth factor), PDGF (platelet-derived growth factor), TGFβ (transforming growth factor β), HGF (hepatocyte growth factor), VEGF (vascular endothelial growth factor), and the like], and the like.

As examples of the “cell growth factor receptors”, there can be used any receptors capable of binding to the aforementioned cell growth factors, including EGF receptor, heregulin receptor (HER2), insulin receptor-1, insulin receptor-2, IGF receptor, FGF receptor-1 or FGF receptor-2, and the like.

As examples of the “pharmaceutical agents inhibiting the action of cell growth factor”, there can be used avastin (VEGF antibody), Herceptin (HER2 antibody), and the like.

In addition to the aforementioned drugs, L-asparaginase, aceglatone, procarbazine hydrochloride, protoporphyrin-cobalt complex salt, mercuric hematoporphyrin-sodium, topoisomerase I inhibitors (e.g., irinotecan, topotecan, and the like), topoisomerase II inhibitors (e.g., sobuzoxane, and the like), differentiation inducers (e.g., retinoid, vitamin D, and the like), other angiogenesis inhibitors (e.g., fumagillin, shark extract, COX-2 inhibitor and the like), α-blockers (e.g., tamsulosin hydrochloride), etc. can be used.

By combining the compound of the present invention and a concomitant drug, a superior effect such as

(1) the dose can be reduced as compared to single administration of the compound of the present invention or a concomitant drug,

(2) the drug to be combined with the compound of the present invention can be selected according to the condition of patients (mild case, severe case and the like),

(3) the period of treatment can be set longer by selecting a concomitant drug having different action and mechanism from the compound of the present invention,

(4) a sustained treatment effect can be designed by selecting a concomitant drug having different action and mechanism from the compound of the present invention,

(5) a synergistic effect can be afforded by a combined use of the compound of the present invention and a concomitant drug, and the like, can be achieved.

In the present specification, a pharmaceutical agent for use of the compound of the present invention and a concomitant drug in combination may be referred to as the “combination agent of the present invention”.

When using the combination agent of the present invention, the administration time of the compound of the present invention and the concomitant drug is not restricted, and the compound of the present invention or the concomitant drug can be administered to an administration subject simultaneously, or may be administered at different times. The dosage of the concomitant drug may be determined according to the administration amount clinically used, and can be appropriately selected depending on an administration subject, administration route, disease, combination and the like.

The administration mode of the compound of the present invention and the concomitant drug of the present invention include the following methods:

(1) The compound of the present invention and the concomitant drug are simultaneously produced to give a single preparation which is administered. (2) The compound of the present invention and the concomitant drug are separately produced to give two kinds of preparations which are administered simultaneously by the same administration route. (3) The compound of the present invention and the concomitant drug are separately produced to give two kinds of preparations which are administered by the same administration route only at the different times. (4) The compound of the present invention and the concomitant drug are separately produced to give two kinds of preparations which are administered simultaneously by the different administration routes. (5) The compound of the present invention and the concomitant drug are separately produced to give two kinds of preparations which are administered by the different administration routes only at different times (for example, the compound of the present invention and the concomitant drug are administered in this order, or in the reverse order). The dose of the concomitant drug can be appropriately determined based on the dose employed in clinical situations. The mixing ratio of the compound of the present invention and a concomitant drug can be appropriately determined depending on the administration subject, administration route, target disease, symptom, combination and the like. When the subject of administration is human, for example, a concomitant drug can be used in 0.01-100 parts by weight relative to 1 part by weight of the compound of the present invention.

A combination agent of the present invention has low toxicity, and for example, the compound of the present invention and/or the above-mentioned concomitant drug can be mixed, according to a method known per se, with a pharmacologically acceptable carrier to give pharmaceutical compositions, such as tablets (including sugar-coated tablet, film-coated tablet), powders, granules, capsules (including soft capsule), solutions, injections, suppositories, sustained release agents and the like, which can be safely administered orally or parenterally (e.g., local, rectum, vein, and the like). An injection can be administered by intravenous, intramuscular, subcutaneous or intra-tissue administration directly to the lesion.

As a pharmacologically acceptable carrier which may be used for preparing a preparation of a combination agent of the present invention, those similar to the aforementioned pharmacologically acceptable carriers that can be used for the production of the pharmaceutical agent of the present invention can be mentioned. Where necessary, the aforementioned additives that can be used for the production of the pharmaceutical agent of the present invention, such as preservatives, antioxidants, coloring agents, sweetening agents, adsorbents, wetting agents and the like can be also used in appropriate amounts.

The compounding ratio of the compound of the present invention to the concomitant drug in the combination agent of the present invention can be appropriately selected depending on an administration subject, administration route, diseases and the like.

For example, the content of the compound of the present invention in the combination agent of the present invention differs depending on the form of a preparation, and usually from about 0.01 to 100% by weight, preferably from about 0.1 to 50% by weight, further preferably from about 0.5 to 20% by weight, based on the preparation.

The content of the concomitant drug in the combination agent of the present invention differs depending on the form of a preparation, and usually from about 0.01 to 90% by weight, preferably from about 0.1 to 50% by weight, further preferably from about 0.5 to 20% by weight, based on the preparation.

The content of additives in the combination agent of the present invention differs depending on the form of a preparation, and usually from about 1 to 99.99% by weight, preferably from about 10 to 90% by weight, based on the preparation.

In the case when the compound of the present invention and the concomitant drug are separately prepared respectively, the same contents may be adopted.

These preparations can be produced by a method known per se usually used in a preparation process.

For example, the compound of the present invention and the concomitant drug can be made into an aqueous injection together with a dispersing agent (e.g., Tween 80 (manufactured by Atlas Powder, US), HCO 60 (manufactured by Nikko Chemicals), polyethylene glycol, carboxymethylcellulose, sodium alginate, hydroxypropylmethylcellulose, dextrin and the like), a stabilizer (e.g., ascorbic acid, sodium pyrosulfite, and the like), a surfactant (e.g., Polysorbate 80, macrogol and the like), a solubilizer (e.g., glycerin, ethanol and the like), a buffer (e.g., phosphoric acid and alkali metal salt thereof, citric acid and alkali metal salt thereof, and the like), an isotonizing agent (e.g., sodium chloride, potassium chloride, mannitol, sorbitol, glucose and the like), a pH regulator (e.g., hydrochloric acid, sodium hydroxide and the like), a preservative (e.g., ethyl p-oxybenzoate, benzoic acid, methylparaben, propylparaben, benzyl alcohol and the like), a dissolving agent (e.g., conc. glycerin, meglumine and the like), a dissolution aid (e.g., propylene glycol, sucrose and the like), a soothing agent (e.g., glucose, benzyl alcohol and the like), and the like, or can be dissolved, suspended or emulsified in a vegetable oil such as olive oil, sesame oil, cotton seed oil, corn oil and the like or a dissolution aid such as propylene glycol and prepared into an oily injection, whereby an injection is afforded.

In addition, an excipient (e.g., lactose, sucrose, starch and the like), a disintegrating agent (e.g., starch, calcium carbonate and the like), a binder (e.g., starch, gum Arabic, carboxymethylcellulose, polyvinylpyrrolidone, hydroxpropylcellulose and the like), a lubricant (e.g., talc, magnesium stearate, polyethylene glycol 6000 and the like) and the like, for example, can be added to the compound of the present invention or the concomitant drug, according to a method known per se, and the mixture can be compression-molded, then if desirable, the molder product can be coated by a method known per se for the purpose of masking of taste, enteric property or durability, to obtain a preparation for oral administration. As this coating agent, for example, hydroxypropylmethylcellulose, ethylcellulose, hydroxymethylcellulose, hydroxypropylcellulose, polyoxyethylene glycol, Tween 80, Pluronic F68, cellulose acetate phthalate, hydroxypropylmethylcellulose phthalate, hydroxymethylcellulose acetate succinate, Eudoragit (methacrylic acid•acrylic acid copolymer, manufactured by Rohm, DE), pigment (e.g., iron oxide red, titanium dioxide, etc.) and the like can be used. The preparation for oral administration may be any of a quick release preparation and a sustained release preparation.

Moreover, the compound of the present invention and the concomitant drug can be made into an oily or aqueous solid, semisolid or liquid suppository according to a method known per se, by mixing with an oily substrate, aqueous substrate or aqueous gel substrate. As the above-mentioned oily substrate, for example, glycerides of higher fatty acids [e.g., cacao butter, Witepsols (manufactured by Dynamit Nobel, Germany), etc.], glycerides of medium chain fatty acid [e.g., Miglyols (manufactured by Dynamit Nobel, Germany), etc.], or vegetable oils (e.g., sesame oil, soybean oil, cotton seed oil and the like), and the like are listed. Further, as the aqueous substrate, for example, polyethylene glycols, propylene glycol are listed, and as the aqueous gel substrate, for example, natural gums, cellulose derivatives, vinyl polymers, acrylic acid polymers and the like are listed.

As the above-mentioned sustained release preparation, sustained release microcapsules and the like are listed. The sustained release microcapsule can be produced by a method known per se, such as the method shown in the following [2].

The compound of the present invention is preferably molded into an oral administration preparation such as a solid preparation (e.g., powder, granule, tablet, capsule) and the like, or molded into a rectal administration preparation such as a suppository. Particularly, an oral administration preparation is preferable.

The concomitant drug can be made into the above-mentioned drug form depending on the kind of the drug.

[1] An injection of the compound of the present invention or the concomitant drug, and preparation thereof, [2] a sustained release preparation or quick release preparation of the compound of the present invention or the concomitant drug, and preparation thereof, [3] a sublingual, buccal or intraoral quick integrating agent of the compound of the present invention or the concomitant drug, and preparation thereof, will be described below specifically.

[1] Injection and Preparation Thereof

An injection prepared by dissolving the compound of the present invention or the concomitant drug into water is preferable. This injection may be allowed to contain a benzoate and/or salicylate.

The injection is obtained by dissolving the compound of the present invention or the concomitant drug, and if desirable, a benzoate and/or salicylate, into water.

As the above-mentioned salts of benzoic acid and salicylic acid, for example, salts of alkali metals such as sodium, potassium and the like, salts of alkaline earth metals such as calcium, magnesium and the like, ammonium salts, meglumine salts, salts with organic bases such as tromethamol and the like, etc. are listed.

The concentration of the compound of the present invention or the concomitant drug in an injection is from 0.5 to 50 w/v %, preferably from about 3 to 20 w/v %. The concentration of a benzoate or/and salicylate is from 0.5 to 50 w/v %, preferably from about 3 to 20 w/v %.

Into a injection of the present invention, additives usually used in an injection, for example, a stabilizer (e.g., ascorbic acid, sodium pyrosulfite and the like), a surfactant (e.g., Polysorbate 80, macrogol and the like), a solubilizer (e.g., glycerin, ethanol and the like), a buffer (e.g., phosphoric acid and alkali metal salt thereof, citric acid and alkali metal salt thereof, and the like), an isotonizing agent (e.g., sodium chloride, potassium chloride and the like), a dispersing agent (e.g., hydroxypropylmethylcellulose, dextrin), a pH regulator (e.g., hydrochloric acid, sodium hydroxide and the like), a preservative (e.g., ethyl p-oxybenzoate, benzoic acid and the like), a dissolving agent (e.g., conc. glycerin, meglumine and the like), a dissolution aid (e.g., propylene glycol, sucrose and the like), a soothing agent (e.g., glucose, benzyl alcohol and the like), and the like, can be appropriately blended. These additives are generally blended in a proportion usually used in an injection.

It is advantageous that pH of an injection is controlled from pH 2 to 12, preferably from pH 2.5 to 8.0 by addition of a pH regulator.

An injection is obtained by dissolving the compound of the present invention or the concomitant drug and if desirable, a benzoate and/or a salicylate, and if necessary, the above-mentioned additives into water. These may be dissolved in any order, and can be appropriately dissolved in the same manner as in a conventional method of producing an injection.

An aqueous solution for injection may be advantageously heated, alternatively, for example, filter sterilization, high pressure heat sterilization and the like can be conducted in the same manner as for a usual injection, to provide an injection.

It may be advantageous that an aqueous solution for injection is subjected to high pressure heat sterilization at 100 to 121° C. for 5 to 30 min.

Further, a preparation endowed with an antibacterial property of a solution may also be produced so that it can be used as a preparation which is divided and administered multiple-times.

[2] Sustained Release Preparation or Quick Release Preparation, and Preparation Thereof

A sustained release preparation is preferable which is obtained, if desirable, by coating a nucleus containing the compound of the present invention or the concomitant drug with a film agent such as a water-insoluble substance, swellable polymer and the like. For example, a sustained release preparation for oral administration of once administration per day type is preferable.

As the water-insoluble substance used in a film agent, there are listed, for example, cellulose ethers such as ethylcellulose, butylcellulose and the like, cellulose esters such as cellulose acetate, cellulose propionate and the like, polyvinyl esters such as polyvinyl acetate, polyvinyl butyrate and the like, acrylic acid/methacrylic acid copolymers, methyl methacrylate copolymers, ethoxyethyl methacrylate/cinnamoethyl methacrylate/aminoalkyl methacrylate copolymers, polyacrylic acid, polymethacrylic acid, methacrylic acid alkylamide copolymers, poly(methyl methacrylate), polymethacrylate, polymethacrylamide, aminoalkyl methacrylate copolymers, poly(methacrylic anhydride), glycidyl methacrylate copolymer, particularly, acrylic acid-based polymers such as Eudoragit (Rohm Pharma) such as Eudoragit RS-100, RL-100, RS-30D, RL-30D, RL-PO, RS-PO (ethyl acrylate/methyl methacrylate/trimethylammoniumethyl methacrylate chloride copolymer), Eudoragit NE-30D (methyl methacrylate/ethyl acrylate copolymer), and the like, hydrogenated oils such as hydrogenated castor oil (e.g., Lubri wax (Freund Corporation) and the like), waxes such as carnauba wax, glycerin fatty acid ester, paraffin and the like, polyglycerin fatty esters, and the like.

As the swellable polymer, polymers having an acidic dissociating group and showing pH dependent swell are preferable, and polymers having an acidic dissociating group, which manifest small swelling in acidic regions such as in stomach and large swelling in neutral regions such as in small intestine and large intestine, are preferable.

As such a polymer having an acidic dissociating group and showing pH dependent swell, cross-linkable polyacrylic acid copolymers such as, for example, Carbomer 934P, 940, 941, 974P, 980, 1342 and the like, polycarbophil, calcium polycarbophil (last two are manufactured by BF Goodrich), Hiviswako 103, 104, 105, 304 (all are manufactured by Wako Pure Chemical Industries, Ltd.), and the like, are listed.

The film agent used in a sustained release preparation may further contain a hydrophilic substance.

As the hydrophilic substance, for example, polysaccharides which may contain a sulfate group such as pullulan, dextrin, alkali metal alginate and the like, polysaccharides having a hydroxyalkyl group or carboxyalkyl group such as hydroxypropylcellulose, hydroxypropylmethylcellulose, carboxymethylcellulose sodium and the like, methylcellulose, polyvinylpyrrolidone, polyvinyl alcohol, polyethylene glycol and the like can be mentioned.

The content of a water-insoluble substance in the film agent of a sustained release preparation is from about 30 to about 90% (w/w), preferably from about 35 to about 80% (w/w), further preferably from about 40 to about 75% (w/w), the content of a swellable polymer is from about 3 to about 30% (w/w), preferably from about 3 to about 15% (w/w). The film agent may further contain a hydrophilic substance, and in which case, the content of a hydrophilic substance in the film agent is about 50% (w/w) or less, preferably about 5 to 40% (w/w), further preferably from about 5 to 35% (w/w). This % (w/w) indicates % by weight based on a film agent composition which is obtained by removing a solvent (e.g., water, lower alcohols such as methanol, ethanol and the like) from a film agent solution.

The sustained release preparation is produced by preparing a nucleus containing a drugs as exemplified below, then, coating the resulted nucleus with a film agent solution prepared by heat-solving a water-insoluble substance, swellable polymer and the like or by dissolving or dispersing it in a solvent.

I. Preparation of Nucleus Containing Drug

The form of nucleus containing a drug to be coated with a film agent (hereinafter, sometimes simply referred to as nucleus) is not particularly restricted, and preferably, the nucleus is formed into particles such as a granule or fine particle.

When the nucleus is composed of granules or fine particles, the average particle size thereof is preferably from about 150 to about 2000 μm, further preferably, from about 500 to about 1400 μm.

Preparation of the Nucleus can be Effected by a Usual production method. For example, a suitable excipient, binding agent, disintegrating agent, lubricant, stabilizer and the like are mixed with a drug, and the mixture is subjected to a wet extrusion granulating method, fluidized bed granulating method or the like, to prepare a nucleus.

The content of drugs in a nucleus is from about 0.5 to about 95% (w/w), preferably from about 5.0 to about 80% (w/w), further preferably from about 30 to about 70% (w/w).

As the excipient contained in the nucleus, for example, saccharides such as sucrose, lactose, mannitol, glucose and the like, starch, crystalline cellulose, calcium phosphate, corn starch and the like are used. Among them, crystalline cellulose, corn starch are preferable.

As the binding agent, for example, polyvinyl alcohol, hydroxypropylcellulose, polyethylene glycol, polyvinyl pyrrolidone, Pluronic F68, gum Arabic, gelatin, starch and the like are used. As the disintegrating agent, for example, carboxymethylcellulose calcium (ECG505), croscarmelose sodium (Ac-Di-Sol), crosslinked polyvinylpyrrolidone (Crospovidone), low substituted hydroxypropylcellulose (L-HPC) and the like are used. Among them, hydroxypropylcellulose, polyvinylpyrrolidone, lower substituted hydroxypropylcellulose are preferable. As the lubricant and coagulation inhibitor, for example, talc, magnesium stearate and inorganic salts thereof are used, and as the lubricant, polyethylene glycol and the like are used. As the stabilizer, acids such as tartaric acid, citric acid, succinic acid, fumaric acid, maleic acid and the like, are used.

A nucleus can also be prepared by, in addition to the above-mentioned, for example, a rolling granulation method in which a drug or a mixture of a drug with an excipient, lubricant and the like is added portionwise onto an inert carrier particle which is the core of the nucleus while spraying a binder dissolved in a suitable solvent such as water, lower alcohol (e.g., methanol, ethanol and the like) and the like, a pan coating method, a fluidized bed coating method or a melt granulating method. As the inert carrier particle, for example, those made of sucrose, lactose, starch, crystalline cellulose or waxes can be used, and the average particle size thereof is preferably from about 100 μm to about 1500 μm.

For separating a drug contained in a nucleus and a film agent, the surface of the nucleus may be coated with a protective agent. As the protective agent, for example, the above-mentioned hydrophilic substances, water-insoluble substances and the like are used. As the protective agent, preferably polyethylene glycol, and polysaccharides having a hydroxyalkyl group or carboxyalkyl group are used, more preferably, hydroxypropylmethylcellulose and hydroxypropylcellulose are used. The protective agent may contain, as stabilizer, acids such as tartaric acid, citric acid, succinic acid, fumaric acid, maleic acid and the like, and lubricants such as talc and the like. When the protective agent is used, the coating amount is from about 1 to about 15% (w/w), preferably from about 1 to about 10% (w/w), further preferably from about 2 to about 8% (w/w), based on the nucleus.

The protective agent can be coated by a usual coating method, and specifically, the protective agent can be coated by spray-coating the nucleus, for example, by a fluidized bed-coating method, pan coating method and the like.

II. Coating of Nucleus with Film Agent

A nucleus obtained in the above-mentioned step I is coated with a film agent solution obtained by heat-solving the above-mentioned water-insoluble substance and pH-dependent swellable polymer, and a hydrophilic substance, or by dissolving or dispersing them in a solvent, to give a sustained release preparation.

As the method for coating a nucleus with a film agent solution, for example, a spray coating method and the like are listed.

The composition ratio of a water-insoluble substance, swellable polymer or hydrophilic substance in a film agent solution is appropriately selected so that the contents of these components in a coated film are the above-mentioned contents, respectively.

The coating amount of a film agent is from about 1 to about 90% (w/w), preferably from about 5 to about 50% (w/w), further preferably from about 5 to about 35% (w/w), based on a nucleus (not including coating amount of protective agent).

As the solvent in a film agent solution, water or an organic solvent can be used alone or in admixture thereof. In the case of use in admixture, the mixing ratio of water to an organic solvent (water/organic solvent: by weight) can be varied in the range from 1 to 100%, and preferably from 1 to about 30%. The organic solvent is not particularly restricted providing it dissolves a water-insoluble substance, and for example, lower alcohols such as methyl alcohol, ethyl alcohol, isopropyl alcohol, n-butyl alcohol and the like, lower alkanone such as acetone and the like, acetonitrile, chloroform, methylene chloride and the like are used. Among them, lower alcohols are preferable, and ethyl alcohol and isopropyl alcohol are particularly preferable. Water, and a mixture of water with an organic solvent are preferably used as a solvent for a film agent. In this case, if necessary, an acid such as tartaric acid, citric acid, succinic acid, fumaric acid, maleic acid and the like may also be added into a film agent solution for stabilizing the film agent solution.

An operation of coating by spray coating can be effected by a usual coating method, and specifically, it can be effected by spray-coating a film agent solution onto a nucleus by a fluidized bed coating method, pan coating method and the like. In this case, if necessary, talc, titanium oxide, magnesium stearate, calcium stearate, light anhydrous silicic acid and the like may also be added as a lubricant, and glycerin fatty acid ester, hydrogenated castor oil, triethyl citrate, cetyl alcohol, stearyl alcohol and the like may also be added as a plasticizer.

After coating with a film agent, if necessary, an antistatic agent such as talc and the like may be mixed.

The quick release preparation may be liquid (solution, suspension, emulsion and the like) or solid (particle, pill, tablet and the like). As the quick release preparation, Oral agents and parenteral agents such as an injection and the like are used, and oral agents are preferable.

The quick release preparation, usually, may contain, in addition to an active component drug, also carriers, additives and excipients conventionally used in the production field (hereinafter, sometimes abbreviated as excipient) The excipient used is not particularly restricted providing it is an excipient ordinarily used as a preparation excipient. For example, as the excipient for an oral solid preparation, lactose, starch, corn starch, crystalline cellulose (Avicel PH101, manufactured by Asahi Kasei Corporation, and the like), powder sugar, granulated sugar, mannitol, light anhydrous silicic acid, magnesium carbonate, calcium carbonate, L-cysteine and the like are listed, and preferably, corn starch and mannitol and the like are listed. These excipients can be used alone or in combination of two or more. The content of the excipient is, for example, from about 4.5 to about 99.4 w/w %, preferably from about 20 to about 98.5 w/w %, further preferably from about 30 to about 97 w/w %, based on the total amount of the quick release preparation.

The content of a drug in the quick release preparation can be appropriately selected in the range from about 0.5 to about 95%, preferably from about 1 to about 60% based on the total amount of the quick release preparation.

When the quick release preparation is an oral solid preparation, it usually contains, in addition to the above-mentioned components, also an integrating agent. As this integrating agent, for example, carboxymethylcellulose calcium (ECG-505, manufactured by Gotoku Yakuhin), croscarmelose sodium (for example, Actisol, manufactured by Asahi Kasei Corporation), crospovidone (for example, Kollidon CL, manufactured by BASF), low substituted hydroxypropylcellulose (manufactured by Shin-Etsu Chemical Co., Ltd.), carboxymethylstarch (manufactured by Matsutani Kagaku K.K.), carboxymethylstarch sodium (Exprotab, manufactured by Kimura Sangyo), partially pregelatinized starch (PCS, manufactured by Asahi Kasei Corporation), and the like are used, and for example, those which disintegrate a granule by adsorbing water in contact with water, causing swelling, or making a channel between an effective ingredient constituting the nucleus and an excipient, can be used. These disintegrating agents can be used alone or in combination of two or more. The amount of the disintegrating agent used is appropriately selected depending on the kind and blending amount of a drug used, design of releasing property, and the like, and for example, from about 0.05 to about 30 w/w %, preferably from about 0.5 to about 15 w/w %, based on the total amount of the quick releasing agent.

When the quick release preparation is an oral solid preparation, it may further contain, in addition to the above-mentioned composition in the case of the oral solid preparation, if desired, additives conventional in solid preparations. As such an additive, there are used, for example, a binder (e.g., sucrose, gelatin, gum Arabic powder, methylcellulose, hydroxypropylcellulose, hydroxypropylmethylcellulose, carboxymethylcellulose, polyvinylpyrrolidone, pullulan, dextrin and the like), a lubricant (e.g., polyethylene glycol, magnesium stearate, talc, light anhydrous silicic acid (for example, Aerosil (Nippon Aerosil)), a surfactant (e.g., anionic surfactants such as sodium alkylsulfate and the like, nonionic surfactants such as polyoxyethylene fatty acid ester and polyoxyethylene sorbitan fatty acid ester, polyoxyethylene castor oil derivatives and the like), a coloring agent (e.g., tar coloring matter, caramel, iron oxide red, titanium oxide, riboflavins), if necessary, an appetizing agent (e.g., sweetening agent, flavoring agent and the like), an adsorbent, preservative, wetting agent, antistatic agent, and the like. Further, as the stabilizer, an organic acid such as tartaric acid, citric acid, succinic acid, fumaric acid and the like may also be added.

As the above-mentioned binder, hydroxypropylcellulose, polyethylene glycol and polyvinylpyrrolidone and the like are preferably used.

The quick release preparation can be prepared by, based on a usual technology of producing preparations, mixing the above-mentioned components, and if necessary, further kneading the mixture, and molding it. The above-mentioned mixing is conducted by generally used methods, for example, mixing, kneading and the like. Specifically, when a quick release preparation is formed, for example, into a particle, it can be prepared, according to the same means as in the above-mentioned method for preparing a nucleus of a sustained release preparation, by mixing the components using a vertical granulator, universal kneader (manufactured by Hata Tekkosho), fluidized bed granulator FD-5S (manufactured by Powrex Corporation), and the like, and then, granulating the mixture by a wet extrusion granulation method, fluidized bed granulation method and the like.

Thus obtained quick release preparation and sustained release preparation may be themselves made into products or made into products appropriately together with preparation excipients and the like, separately, by an ordinary method, then, may be administered simultaneously or may be administered in combination at any administration interval, or they may be themselves made into one oral preparation (e.g., granule, fine particle, tablet, capsule and the like) or made into one oral preparation appropriately together with preparation excipients and the like. It may also be permissible that they are made into granules or fine particles, and filled in the same capsule to be used as a preparation for oral administration.

[3] Sublingual, Buccal or Intraoral Quick Disintegrating Agent and Preparation Thereof

Sublingual, buccal or intraoral quick disintegrating agents may be a solid preparation such as tablet and the like, or may be an oral mucosa membrane patch (film).

As the sublingual, buccal or intraoral quick disintegrating agent, a preparation containing the compound of the present invention or the concomitant drug and an excipient is preferable. It may contain also auxiliary agents such as a lubricant, isotonizing agent, hydrophilic carrier, water-dispersible polymer, stabilizer and the like. Further, for easy absorption and increased in vivo use efficiency, β-cyclodextrin or β-cyclodextrin derivatives (e.g., hydroxypropyl-β-cyclodextrin and the like) and the like may also be contained.

As the above-mentioned excipient, lactose, sucrose, D-mannitol, starch, crystalline cellulose, light anhydrous silicic acid and the like are listed. As the lubricant, magnesium stearate, calcium stearate, talc, colloidal silica and the like are listed, and particularly, magnesium stearate and colloidal silica are preferable. As the isotonizing agent, sodium chloride, glucose, fructose, mannitol, sorbitol, lactose, saccharose, glycerin, urea and the like are listed, and particularly, mannitol is preferable. As the hydrophilic carrier, swellable hydrophilic carriers such as crystalline cellulose, ethylcellulose, crosslinkable polyvinylpyrrolidone, light anhydrous silicic acid, silicic acid, dicalcium phosphate, calcium carbonate and the like are listed, and particularly, crystalline cellulose (e.g., microcrystalline cellulose and the like) is preferable. As the water-dispersible polymer, gums (e.g., gum tragacanth, acacia gum, cyamoposis gum), alginates (e.g., sodium alginate), cellulose derivatives (e.g., methylcellulose, carboxymethylcellulose, hydroxymethylcellulose, hydroxypropylcellulose, hydroxypropylmethylcellulose), gelatin, water-soluble starch, polyacrylic acids (e.g., Carbomer), polymethacylic acid, polyvinyl alcohol, polyethylene glycol, polyvinylpyrrolidone, polycarbophil, ascorbate palmitates and the like are listed, and hydroxypropylmethylcellulose, polyacrylic acid, alginate, gelatin, carboxymethylcellulose, polyvinylpyrrolidone, polyethylene glycol and the like are preferable. Particularly, hydroxypropylmethylcellulose is preferable. As the stabilizer, cysteine, thiosorbitol, tartaric acid, citric acid, sodium carbonate, ascorbic acid, glycine, sodium sulfite and the like are listed, and particularly, citric acid and ascorbic acid are preferable.

The sublingual, buccal or intraoral quick disintegrating agent can be produced by mixing the compound of the present invention or the concomitant drug and an excipient by a method known per se. Further, if desired, auxiliary agents such as a lubricant, isotonizing agent, hydrophilic carrier, water-dispersible polymer, stabilizer, coloring agent, sweetening agent, preservative and the like may be mixed. The sublingual, buccal or intraoral quick disintegrating agent is obtained by mixing the above-mentioned components simultaneously or at a time interval, then subjecting the mixture to tablet-making molding under pressure. For obtaining suitable hardness, it may also be permissible that the materials are moistened by using a solvent such as water, alcohol and the like if desired before and after the tablet making process, and after the molding, the materials are dried, to obtain a product.

In the case of molding into a mucosa membrane patch (film), the compound of the present invention or the concomitant drug and the above-mentioned water-dispersible polymer (preferably, hydroxypropylcellulose, hydroxypropylmethylcellulose), excipient and the like are dissolved in a solvent such as water and the like, and the resulted solution is cast to give a film. Further, additives such as a plasticizer, stabilizer, antioxidant, preservative, coloring agent, buffer, sweetening agent and the like may also be added. For imparting suitable elasticity to the film, glycols such as polyethylene glycol, propylene glycol and the like may be contained, or for enhancing adhesion of the film to an intraoral mucosa membrane lining, a bio-adhesive polymer (e.g., polycarbophil, carbopol) may also be contained. In the casting, a solution is poured on the non-adhesive surface, spread to uniform thickness (preferably, about 10 to 1000 micron) by an application tool such as a doctor blade and the like, then, the solution is dried to form a film. It may be advantageous that thus formed film is dried at room temperature or under heat, and cut into given area.

As the preferable intraoral quick disintegrating agent, there are listed solid quick scattering dose agents composed of a network body comprising the compound of the present invention or the concomitant drug, and a water-soluble or water-diffusible carrier which is inert to the compound of the present invention or concomitant drug, are listed. This network body is obtained by sublimating a solvent from the solid composition constituted of a solution prepared by dissolving the compound of the present invention or the concomitant drug in a suitable solvent.

It is preferable that the composition of an intraoral quick disintegrating agent contains a matrix forming agent and a secondary component, in addition to the compound of the present invention or the concomitant drug.

Examples of the matrix forming agent include gelatins, dextrins, animal proteins or vegetable proteins such as soybean, wheat and psyllium seed protein and the like; rubber substances such as gum Arabic, guar gum, agar, xanthane gum and the like; polysaccharides; alginic acids; carboxymethylcelluloses; carageenans; dextrans; pectines; synthetic polymers such as polyvinylpyrrolidone and the like; substances derived from a gelatin-gum Arabic complex, and the like. Further, saccharides such as mannitol, dextrose, lactose, galactose, trehalose and the like; cyclic saccharides such as cyclodextrin and the like; inorganic salts such as sodium phosphate, sodium chloride and aluminum silicate and the like; amino acids having 2 to 12 carbon atoms such as glycine, L-alanine, L-aspartic acid, L-glutamic acid, L-hydroxyproline, L-isoleucine, L-leucine, L-phenylalanine and the like, are contained.

One or more of the matrix forming agents can be introduced in a solution or suspension before solidification. Such as matrix forming agent may be present in addition to a surfactant, or may be present while a surfactant being excluded. The matrix forming agents aid to maintain the compound of the present invention or the concomitant drug in the solution or suspension in diffused condition, in addition to formation of the matrix.

The composition may contain secondary components such as a preservative, antioxidant, surfactant, thickening agent, coloring agent, pH controlling agent, flavoring agent, sweetening agent, food taste masking agent and the like. As the suitable coloring agent, there are listed red, black and yellow iron oxides, and FD & C dyes such as FD & C Blue 2, FD & C Red 40 and the like manufactured by Ellis and Everard. Examples of the suitable flavoring agent include mint, raspberry, licorice, orange, lemon, grape fruit, caramel, vanilla, cherry, grape flavor and combinations thereof. Examples of the suitable pH controlling agent include citric acid, tartaric acid, phosphoric acid, hydrochloric acid and maleic acid. Examples of the suitable sweetening agent include aspartame, acesulfame K and thaumatin and the like. Examples of the suitable food taste masking agent include sodium bicarbonate, ion exchange resin, cyclodextrin-inclusion compounds, adsorbent substances and microcapsulated apomorphine.

The preparation contains the compound of the present invention or the concomitant drug in an amount usually from about 0.1 to about 50% by weight, preferably from about 0.1 to about 30% by weight, and preferable are preparations (such as the above-mentioned sublingual agent, buccal and the like) which can dissolve 90% or more of the compound of the present invention or the concomitant drug (into water) within the time range of about 1 to about 60 min, preferably of about 1 to about 15 min, more preferably of about 2 to about 5 min, and intraoral quick disintegrating preparations which are disintegrated within the range of 1 to 60 sec, preferably of 1 to 30 sec, further preferably of 1 to 10 sec after place in an oral cavity.

The content of the above-mentioned excipient in the whole preparation is from about 10 to about 99% by weight, preferably from about 30 to about 90% by weight. The content of β-cyclodextrin or β-cyclodextrin derivative in the whole preparation is from 0 to about 30% by weight. The content of the lubricant in the whole preparation is from about 0.01 to about 10% by weight, preferably from about 1 to about 5% by weight. The content of the isotonizing agent in the whole preparation is from about 0.1 to about 90% by weight, preferably, from about 10 to about 70% by weight. The content of the hydrophilic carrier in the whole preparation is from about 0.1 to about 50% by weight, preferably, from about 10 to about 30% by weight. The content of the water-dispersible polymer in the whole preparation is from about 0.1 to about 30% by weight, preferably, from about 10 to about 25% by weight. The content of the stabilizer in the whole preparation is from about 0.1 to about 10% by weight, preferably, from about 1 to 5% by weight. The above-mentioned preparation may further contain additives such as a coloring agent, sweetening agent, preservative and the like, if necessary.

The dosage of a combination agent of the present invention differs depending on the kind of a compound of the present invention, age, body weight, condition, drug form, administration method, administration period and the like, and for example, for one cancer patient (adult, body weight: about 60 kg), the combination agent is administered intravenously, at a dose of about 0.01 to about 1000 mg/kg/day, preferably about 0.01 to about 100 mg/kg/day, more preferably about 0.1 to about 100 mg/kg/day, particularly about 0.1 to about 50 mg/kg/day, especially about 1.5 to about 30 mg/kg/day, in terms of the compound of the present invention or the concomitant drug, respectively, once or several time in division a day. Of course, since the dose as described above varies depending on various conditions, amounts smaller than the above-mentioned dosage may sometimes be sufficient, further, amounts over that range sometimes have to be administered.

The amount of the concomitant drug can be set at any value unless side effects are problematical. The daily dosage in terms of the concomitant drug differs depending on the severity of the symptom, age, sex, body weight, sensitivity difference of the subject, administration period, interval, and nature, pharmacy, kind of the pharmaceutical preparation, kind of effective ingredient, and the like, and not particularly restricted, and the amount of a drug is, in the case of oral administration for example, usually from about 0.001 to 2000 mg, preferably from about 0.01 to 500 mg, further preferably from about 0.1 to 100 mg, per 1 kg of a mammal and this is usually administered once to 4-times in division a day.

In administration of a combination agent of the present invention, the compound of the present invention may be administered after administration of the concomitant drug or the concomitant drug may be administered after administration of the compound of the present invention, though they may be administered simultaneously. When administered at a time interval, the interval differs depending on the effective ingredient to be administered, drug form and administration method, and for example, when the concomitant drug is administered first, a method in which the compound of the present invention is administered within time range of from 1 min to 3 days, preferably from 10 min to 1 day, more preferably from 15 min to 1 hr after administration of the concomitant drug is exemplified. When the compound of the present invention is administered first, a method in which the concomitant drug is administered within time range of from 1 min to 1 day, preferably from 10 min to 6 hrs, more preferably from 15 min to 1 hr after administration of the compound of the present invention is exemplified.

In a preferable administration method, for example, the concomitant drug which has been formed into an oral administration preparation is administered orally at a daily dose of about 0.001 to 200 mg/kg, and about 15 min after, the compound of the present invention which has been formed into an oral administration preparation is administered orally at a daily dose of about 0.005 to 100 mg/kg.

Furthermore, the compound of the present invention or the combination agent of the present invention can be used concurrently with a non-drug therapy. To be precise, the compound of the present invention and the combination agent of the present invention can be combined with a non-drug therapy such as (1) surgery, (2) hypertensive chemotherapy using angiotensin II etc., (3) gene therapy, (4) thermotherapy, (5) cryotherapy, (6) laser cauterization, (7) radiotherapy, and the like.

For example, the compound of the present invention and the combination agent of the present invention inhibit an expression of resistance, extends disease-free survival, suppresses cancer metastasis or recurrence, prolongs survival and provide other benefits when used before or after the surgery, etc., or a combination treatment comprising 2 or 3 of these therapies.

Also, treatment with the compound of the present invention and the combination agent of the present invention can be combined with supportive therapies [e.g., (i) administration of antibiotics (e.g., β-lactams such as pansporin, macrolides such as clarytheromycin) to an combined expression of various infectious diseases, (ii) administration of total parenteral nutrition, amino acid preparations and general vitamin preparations for improvement of malnutrition, (iii) morphine administration for pain mitigation, (iv) administration of drugs which alleviate side effects such as nausea, vomiting, anorexia, diarrhea, leukopenia, thrombocytopenia, hemoglobin concentration reduction, hair loss, hepatopathy, renopathy, DIC and fever, (v) administration of drugs for inhibition of multiple drug resistance in cancer, and the like].

Preferably, the compound of the present invention or the combination agent of the present invention is administered orally (including sustained-release preparations), intravenously (including boluses, infusions and clathrates), subcutaneously and intramuscularly (including boluses, infusions and sustained-release preparations), transdermally, intratumorally or proximally before or after the above-described treatment is conducted.

As a period for administering the compound of the present invention or the combination agent of the present invention before the surgery, etc., for example, it can be administrated 1-time about 30 min to 24 hrs before the surgery, etc., or in 1 to 3 cycles about 3 months to 6 months before the surgery, etc. In this way, the surgery, etc. can be conducted easily because, for example, a cancer tissue would be reduced by administering the compound of the present invention or the combination agent of the present invention before the surgery, and the like.

As a period for administering the compound of the present invention or the combination agent of the present invention after the surgery, etc., for example, it can be administrated repeatedly per a few weeks to 3 months, about 30 min to 24 hrs after the surgery, and the like. In this way, it makes an effect of the surgery, etc. increasing by administering the compound of the present invention or the combination agent of the present invention after the surgery, and the like.

Since the compounds (I) and (I′) of the present invention, a salt thereof and a prodrug thereof show a superior inhibitory activity against kinase such as vascular endothelial growth factor receptor and the like, a clinically useful agent for the prophylaxis or treatment of diseases associated with an action of vascular endothelial growth factor in vivo (e.g., cancer and the like). In addition, the compounds (I) and (I′) of the present invention, a salt thereof and a prodrug thereof are also superior in the efficacy expression, pharmacokinetics, solubility, interaction with other pharmaceutical products, safety and stability, they are useful as pharmaceutical agents.

The present invention is explained in more detail in the following by referring to Reference Examples, Examples, Formulation Examples, Experimental Examples and Test Examples, which are not to be construed as limitative.

The “room temperature” in the following Reference Examples and Examples indicates normally about 10° C. to about 35° C. The “%” indicates percentage by weight unless otherwise indicated.

Abbreviations used elsewhere in the specification indicate the following meanings:

s: singlet, d: doublet, t: triplet, q: quartet, m: multiplet, br: broad, J: coupling constant

Genetic manipulation methods described in Experimental Example below are based on the methods described in Maniatis et al., Molecular Cloning, Cold Spring Harbor Laboratory, 1989, and the appended protocol.

### EXAMPLES

### Reference Example 1

### 2-(hydroxymethylene)cycloheptanone

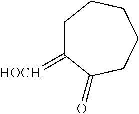

To a mixture of cycloheptanone (1.18 ml), diethyl ether (10 ml) and sodium methoxide (1.08 g) was added ethyl formate (1.089 ml), and the mixture was stirred at room temperature for 18 hrs. 1N Hydrochloric acid was added to the reaction mixture, and the mixture was extracted with ethyl acetate. The extract washed with saturated brine, dried over anhydrous sodium sulfate, filtered and concentrated to give the title compound (1.32 g) as a colorless liquid.

¹H-NMR (CDCl₃) δ: 1.50-1.85 (6H, m), 2.20-2.30 (2H, m), 2.50-2.60 (2H, m), 7.62 (1H, d, J=8.7 Hz).

### Reference Example 2

### cycloheptane-1,2-dione phenylhydrazone

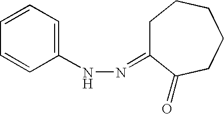

A mixture of aniline (0.913 ml), concentrated hydrochloric acid (2.06 g), water (6 ml) and sodium nitrite (690 mg) was stirred at 0° C. for 20 min. This mixture was added to a mixed solution of 2-(hydroxymethylene)cycloheptanone (1.40 g) in ethanol (16 ml) and a solution of potassium hydroxide (561 mg) in water (0.6 ml) at 0° C., and the mixture was stirred at 0° C. for 10 min and at room temperature for 1 hr and added to water. The precipitate was collected by filtration and washed with water to give the title compound (2.03 g) as a yellow solid.

¹H-NMR (CDCl₃) δ: 1.65-1.90 (6H, m), 2.50-2.75 (4H, m), 6.90-7.05 (1H, m), 7.15-7.40 (4H, m).

### Reference Example 3

### 7,8,9,10-tetrahydrocyclohepta[b]indol-6(5H)-one

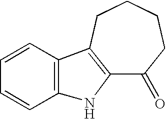

A mixture of cycloheptane-1,2-dione phenylhydrazone (1.90 g) and formic acid (20 ml) was stirred at 100° C. for 30 min, and the mixture was allowed to cool. Water was added to the reaction mixture, and the mixture was extracted with ethyl acetate. The extract washed with water, saturated aqueous sodium hydrogen carbonate solution and saturated brine, dried over anhydrous sodium sulfate, filtered and concentrated. The residue washed with a mixed solution of diethyl ether and hexane to give the title compound (0.98 g) as a colorless solid.

¹H-NMR (CDCl₃) δ: 1.95-2.20 (4H, m), 2.84 (2H, t, J=5.7 Hz), 3.16 (2H, t, J=6.3 Hz), 7.05-7.70 (4H, m), 8.87 (1H, brs).

### Reference Example 4

### 7-[(dimethylamino)methylene]-7,8,9,10-tetrahydrocyclohepta[b]indol-6(5H)-one

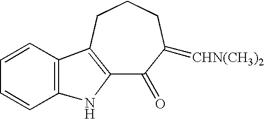

A mixture of 7,8,9,10-tetrahydrocyclohepta[b]indol-6(5H)-one (200 mg), tris(dimethylamino)methane (436 mg) and dimethylformamide (2 ml) was stirred at 70° C. for 3 hrs. After allowing to cool, water was added to the reaction mixture, and the mixture was extracted with ethyl acetate. The extract was washed with water, dried over anhydrous sodium sulfate, filtered and concentrated to give the title compound (195 mg) as a yellow solid.

¹H-NMR (CDCl₃) δ: 2.00-2.15 (2H, m), 2.70-2.85 (2H, m), 3.00-3.20 (2H, m), 3.13 (6H, s), 7.00-7.65 (4H, m), 7.66 (1H, s), 9.17 (1H, brs).

### Reference Example 5

### ethyl 4-[2-(2-oxocycloheptylidene)hydrazino]benzoate

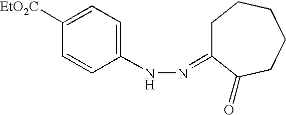

The title compound was obtained by a method similar to that in Reference Example 2 and using ethyl 4-aminobenzoate instead of aniline.

¹H-NMR (CDCl₃) δ: 1.38 (3H, t, J=7.2 Hz), 1.70-1.90 (6H, m), 2.50-2.80 (4H, m), 4.34 (2H, q, J=7.2 Hz), 7.15-7.30 (2H, m), 7.90 (1H, brs), 7.90-8.00 (2H, m).

### Reference Example 6

### ethyl 6-oxo-5,6,7,8,9,10-hexahydrocyclohepta[b]indole-2-carboxylate

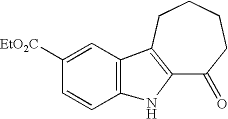

The title compound was obtained by a method similar to that in Reference Example 3 and using ethyl 4-[2-(2-oxocycloheptylidene)hydrazino]benzoate instead of cycloheptane-1,2-dione phenylhydrazone.

¹H-NMR (CDCl₃) δ: 1.43 (3H, t, J=7.0 Hz), 1.90-2.20 (4H, m), 2.86 (2H, t, J=5.2 Hz), 3.20 (2H, t, J=6.6 Hz), 4.41 (2H, q, J=7.0 Hz), 7.30-7.45 (1H, m), 8.00-8.10 (1H, m), 8.46 (1H, m), 9.13 (1H, brs).

### Reference Example 7

### ethyl 7-[(dimethylamino)methylene]-6-oxo-5,6,7,8,9,10-hexahydrocyclohepta[b]indole-2-carboxylate

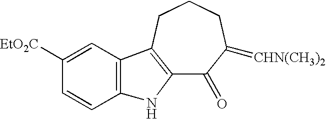

The title compound was obtained by a method similar to that in Reference Example 4 and using ethyl 6-oxo-5,6,7,8,9,10-hexahydrocyclohepta[b]indole-2-carboxylate instead of 7,8,9,10-tetrahydrocyclohepta[b]indol-6(5H)-one.

¹H-NMR (CDCl₃) δ: 1.42 (3H, t, J=7.2 Hz), 2.00-2.20 (2H, m), 2.70-2.85 (2H, m), 3.10-3.20 (2H, m), 3.16 (6H, s), 4.40 (2H, q, J=7.2 Hz), 7.37 (1H, d, J=8.1 Hz), 7.70 (1H, s), 7.97 (1H, dd, J=8.1, 1.5 Hz), 8.40 (1H, d, J=1.5 Hz), 9.35 (1H, brs).

### Reference Example 8

### methyl 4-methyl-3-[2-(2-oxocycloheptylidene)hydrazino]benzoate

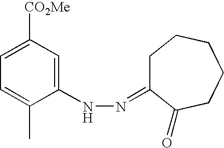

The title compound was obtained by a method similar to that in Reference Example 2 and using methyl 3-amino-4-methylbenzoate instead of aniline.

¹H-NMR (CDCl₃) δ: 1.70-1.85 (6H, m), 2.33 (3H, s), 2.60-2.90 (4H, m), 3.92 (3H, s), 7.18 (1H, d, J=7.8 Hz), 7.26 (1H, s), 7.58 (1H, dd, J=7.8, 1.7 Hz), 8.30 (1H, d, J=1.7 Hz), 13.73 (1H, s).

### Reference Example 9

### methyl 4-methyl-6-oxo-5,6,7,8,9,10-hexahydrocyclohepta[b]indole-1-carboxylate

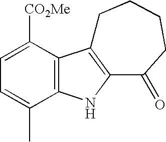

The title compound was obtained by a method similar to that in Reference Example 3 and using methyl 4-methyl-3-[2-(2-oxocycloheptylidene)hydrazino]benzoate instead of cycloheptane-1,2-dione phenylhydrazone.

¹H-NMR (CDCl₃) δ: 1.90-2.10 (4H, m), 2.52 (3H, s), 2.80-2.90 (2H, m), 3.15-3.25 (2H, m), 3.96 (3H, s), 7.13 (1H, d, J=7.5 Hz), 7.49 (1H, d, J=7.5 Hz), 9.05 (1H, brs).

### Reference Example 10

### methyl 7-[(dimethylamino)methylene]-4-methyl-6-oxo-5,6,7,8,9,10-hexahydrocyclohepta[b]indole-1-carboxylate

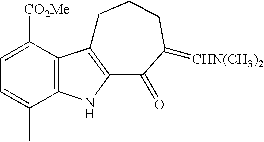

The title compound was obtained by a method similar to that in Reference Example 4 and using methyl 4-methyl-6-oxo-5,6,7,8,9,10-hexahydrocyclohepta[b]indole-1-carboxylate instead of 7,8,9,10-tetrahydrocyclohepta[b]indol-6(5H)-one.

¹H-NMR (CDCl₃) δ: 1.95-2.10 (2H, m), 2.52 (3H, s), 2.65-2.75 (2H, m), 3.05-3.15 (2H, m), 3.16 (6H, s), 3.94 (3H, s), 7.04 (1H, d, J=7.2 Hz), 7.47 (1H, d, J=7.2 Hz), 7.69 (1H, s), 9.29 (1H, brs).

### Reference Example 11

### 2-(hydroxymethylene)cyclooctanone

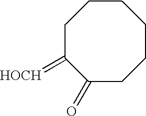

The title compound was obtained by a method similar to that in Reference Example 1 and using cyclooctanone instead of cycloheptanone.

¹H-NMR (CDCl₃) δ: 1.45-1.70 (6H, m), 1.70-1.85 (2H, m), 2.33 (2H, t, J=6.6 Hz), 2.48 (2H, t, J=6.6 Hz), 8.15-8.25 (1H, m).

### Reference Example 12

### ethyl 4-[2-(2-oxocyclooctylidene)hydrazino]benzoate

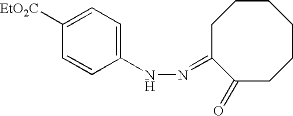

The title compound was obtained by a method similar to that in Reference Example 2 and using ethyl 4-aminobenzoate instead of aniline and 2-(hydroxymethylene)cyclooctanone instead of 2-(hydroxymethylene)cycloheptanone.

¹H-NMR (CDCl₃) δ: 1.39 (3H, t, J=7.2 Hz), 1.50-1.90 (8H, m), 2.70 (2H, t, J=6.6 Hz), 2.76 (2H, t, J=6.0 Hz), 4.35 (2H, q, J=7.2 Hz), 7.20-7.30 (2H, m), 7.90-8.10 (2H, m).

### Reference Example 13

### ethyl 6-oxo-6,7,8,9,10,11-hexahydro-5H-cycloocta[b]indole-2-carboxylate

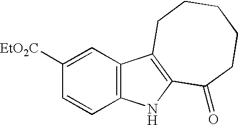

The title compound was obtained by a method similar to that in Reference Example 3 and using ethyl 4-[2-(2-oxocyclooctylidene)hydrazino]benzoate instead of cycloheptane-1,2-dione phenylhydrazone.

¹H-NMR (CDCl₃) δ: 1.43 (3H, t, J=7.2 Hz), 1.35-1.55 (2H, m), 1.75-1.95 (4H, m), 3.03 (2H, t, J=7.2 Hz), 3.35 (2H, t, J=7.2 Hz), 4.42 (2H, q, J=7.2 Hz), 7.40 (1H, d, J=7.8 Hz), 8.04 (1H, dd, J=1.5, 7.8 Hz), 8.50 (1H, d, J=1.5 Hz), 9.21 (1H, brs).

### Reference Example 14

### ethyl 7-[(dimethylamino)methylene]-6-oxo-6,7,8,9,10,11-hexahydro-5H-cycloocta[b]indole-2-carboxylate

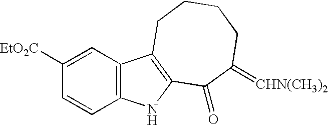

The title compound was obtained by a method similar to that in Reference Example 4 and using ethyl 6-oxo-6,7,8,9,10,11-hexahydro-5H-cycloocta[b]indole-2-carboxylate instead of 7,8,9,10-tetrahydrocyclohepta[b]indol-6(5H)-one.

¹H-NMR (CDCl₃) δ: 1.42 (3H, t, J=7.2 Hz), 1.70-1.90 (2H, m), 2.00-2.15 (2H, m), 2.75-2.85 (2H, m), 3.05-3.15 (2H, m), 3.20 (6H, s), 4.00 (2H, q, J=7.2 Hz), 7.32 (1H, d, J=7.8 Hz), 7.76 (1H, s), 7.95 (1H, d, J=7.8 Hz), 8.37 (1H, s), 9.05 (1H, s).

### Reference Example 15

### methyl 2-acetyl-3-methyl-1H-indole-5-carboxylate

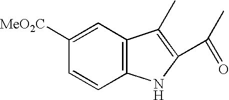

To a solution of ethyl 2-ethylacetoacetate (28.8 g) in ethanol (52.6 ml) were added a solution of sodium hydroxide (8.75 g) in water (26.3 ml) and water (400 ml), and the mixture was stirred at room temperature for 6 hrs and washed with diethyl ether. To 4-aminobenzoic acid (25 g) was added a solution of 2N hydrochloric acid (250 ml), 36% hydrochloric acid (50 ml) and sodium nitrite (13.2 g) in water (25 ml) at 0° C., and the mixture was stirred at 0° C. for 30 min and added to the above-mentioned reaction mixture. Sodium acetate (75 g) was further added and the mixture was stirred at room temperature for 12 hrs. The precipitate was collected by filtration and washed with water to give 4-[2-(1-ethyl-2-oxopropylidene)hydrazino]benzoic acid (45 g).

Hydrochloric acid (135 ml) was added to 4-[2-(1-ethyl-2-oxopropylidene)hydrazino]benzoic acid (45 g), and the mixture was heated under reflux for 2 hrs. After allowing to cool, water (810 ml) was added, and the precipitate was collected by filtration. Methanol (180 ml) and sulfuric acid (9.0 ml) were added to the precipitate, and the mixture was heated under reflux for 16 hrs. After allowing to cool, the reaction mixture was concentrated, and the residue was extracted with a mixed solution of ethyl acetate and tetrahydrofuran, washed with saturated aqueous sodium hydrogen carbonate solution and saturated brine, dried over magnesium sulfate, filtered and concentrated. The residue washed with a mixed solution of ethyl acetate and methanol to give the title compound (4.2 g).

¹H-NMR (DMSO-d₆) δ: 2.60 (3H, s), 2.61 (3H, s), 3.86 (3H, s), 7.81 (1H, d, J=8.7 Hz), 7.87 (1H, d, J=8.7 Hz), 8.39 (1H, s), 11.83 (1H, s).

### Reference Example 16

### methyl 2-acetyl-3-propyl-1H-indole-5-carboxylate

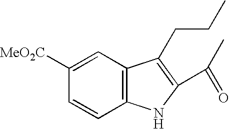

The title compound was obtained by a method similar to that in Reference Example 15 and using ethyl 2-butylacetoacetate instead of ethyl 2-ethylacetoacetate.

¹H-NMR (CDCl₃) δ: 1.07 (3H, t, J=7.5 Hz), 1.70-1.84 (2H, m), 2.67 (3H, s), 3.08-3.14 (2H, m), 3.95 (3H, s), 7.38 (1H, d, J=9.0 Hz), 8.01 (1H, dd, J=8.7, 1.8 Hz), 8.48 (1H, s), 9.18 (1H, brs).

### Reference Example 17

### 6-chloro-5-nitro-2,3,4,9-tetrahydro-1H-carbazol-1-one

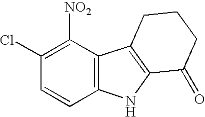

6-Chloro-2,3,4,9-tetrahydro-1H-carbazol-1-one (100 mg) was added to nitric acid (density 1.42, 4 ml) at −20° C., and the mixture was heated to 0° C. The reaction mixture was poured into saturated aqueous sodium hydrogen carbonate solution and extracted with ethyl acetate. The extract was dried over anhydrous magnesium sulfate, filtered and concentrated. The residue washed with a mixed solution of ethyl acetate and hexane to give the title compound (103 mg).

¹H-NMR (DMSO-d₆) δ: 2.11-2.17 (2H, m), 2.51-2.72 (4H, m), 7.53 (1H, d, J=9.2 Hz), 7.67 (1H, d, J=9.2 Hz).

### Reference Example 18

### 5-amino-6-chloro-2,3,4,9-tetrahydro-1H-carbazol-1-one

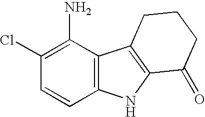

To a suspension of zinc (21 g) in 78% water-containing ethanol (80 ml) was added 67% aqueous calcium chloride solution (1.2 ml), and the mixture was heated under reflux for 5 min. A solution of 6-chloro-5-nitro-2,3,4,9-tetrahydro-1H-carbazol-1-one (940 mg) in ethanol (10 ml) was added and the mixture was further heated under reflux for 1 hr. After allowing to cool, the reaction mixture was filtered and concentrated. The residue was extracted with ethyl acetate, and the extract washed with saturated aqueous sodium hydrogen carbonate solution, dried over anhydrous sodium sulfate, filtered and concentrated. The residue washed with a mixed solution of diethyl ether and hexane to give the title compound (730 mg).

¹H-NMR (DMSO-d₆) δ: 2.05-2.18 (2H, m), 2.41-2.55 (2H, m), 3.16-3.23 (2H, m), 6.62 (1H, d, J=8.8 Hz), 7.08 (1H, d, J=8.8 Hz), 11.50 (1H, brs).

### Reference Example 19

### tert-butyl 4-(4-fluorophenyl)-4-hydroxypiperidine-1-carboxylate

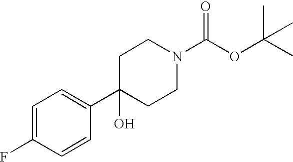

A mixture of magnesium (486.8 mg), iodine (9.6 mg), 1-bromo-4-fluorobenzene (3.50 g) and tetrahydrofuran (20 ml) was stirred at 70° C. for 1 hr. A solution of tert-butyl 4-oxopiperidine-1-carboxylate (2.00 g) in tetrahydrofuran (20 ml) was added to the reaction mixture at 0° C., and the mixture was stirred at 0° C. for 30 min and at room temperature for 1.5 hrs. A saturated aqueous ammonium chloride solution was added to the reaction mixture at 0° C., and the mixture was extracted with ethyl acetate. The extract washed with saturated brine, dried over anhydrous sodium sulfate, filtered and concentrated. Separately from this reaction, a mixture of magnesium (2.43 g), iodine (22.8 mg), 1-bromo-4-fluorobenzene (17.50 g) and tetrahydrofuran (100 ml) was stirred at 70° C. for 1.5 hrs. A solution of tert-butyl 4-oxopiperidine-1-carboxylate (10.03 g) in tetrahydrofuran (100 ml) was added to the reaction mixture at 0° C., and the mixture was stirred at 0° C. for 1 hr and at room temperature for 1 hr. A saturated aqueous ammonium chloride solution was added to the reaction mixture at 0° C., and the mixture was extracted with ethyl acetate. The extract washed with saturated brine, dried over anhydrous magnesium sulfate, filtered and concentrated. The residue was purified by silica gel column chromatography (eluted with a mixed solution of hexane and ethyl acetate at a mixing ratio of 19:1-2:1) together with the above-mentioned residue to give the title compound (16.40 g).

¹H-NMR (CDCl₃) δ: 1.48 (9H, s), 1.57 (1H, s), 1.67-1.77 (2H, m), 1.88-2.06 (2H, m), 3.15-3.31 (2H, m), 3.92-4.12 (2H, m), 7.00-7.08 (2H, m), 7.40-7.48 (2H, m).

### Reference Example 20

### 4-(4-fluorophenyl)piperidin-4-ol

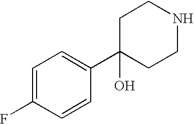

To a solution of tert-butyl 4-(4-fluorophenyl)-4-hydroxypiperidine-1-carboxylate (16.40 g) in methanol (60 ml) was added a solution (70 ml) of 4N hydrogen chloride in ethyl acetate, and the mixture was stirred at room temperature for 2 hrs. The reaction mixture was cooled to 0° C., 4N aqueous sodium hydroxide solution (100 ml) was added, and the mixture was extracted with ethyl acetate. The extract washed with saturated brine, dried over anhydrous sodium sulfate, filtered and concentrated. The residue was crystallized (a mixed solvent of ethyl acetate and diisopropyl ether) to give the title compound (8.21 g).

¹H-NMR (CDCl₃) δ: 1.68-1.83 (2H, m), 2.00-2.20 (2H, m), 2.61-2.85 (2H, m), 2.97-3.27 (3H, m), 6.97-7.10 (2H, m), 7.41-7.54 (2H, m).

### Reference Example 21

### tert-butyl 4-hydroxy-4-[2-(trifluoromethyl)phenyl]piperidine-1-carboxylate

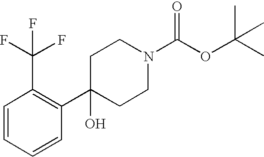

A mixture of magnesium (1.62 g), iodine (2.16 mg), 1-bromo-2-(trifluoromethyl)benzene (15.0 g) and tetrahydrofuran (230 ml) was stirred at 70° C. for 1 hr. tert-Butyl 4-oxopiperidine-1-carboxylate (6.70 g) was added to the reaction mixture at 0° C., and the mixture was stirred at 0° C. for 1 hr. A saturated aqueous ammonium chloride solution was added to the reaction mixture at 0° C., and the mixture was extracted with ethyl acetate. The extract washed with saturated brine, dried over anhydrous sodium sulfate, filtered and concentrated. The residue was purified by silica gel column chromatography (eluted with a mixed solution of hexane and ethyl acetate at a mixing ratio of 5:1) to give the title compound (3.74 g).

¹H-NMR (CDCl₃) δ: 1.49 (9H, s), 1.83-1.88 (2H, m), 1.95 (1H, s), 2.04-2.21 (2H, m), 3.16-3.24 (2H, m), 4.01-4.16 (2H, m), 7.35-7.41 (1H, m), 7.51-7.53 (2H, m), 7.78-7.80 (1H, m).

### Reference Example 22

### tert-butyl 4-hydroxy-4-(3-methoxyphenyl)piperidine-1-carboxylate

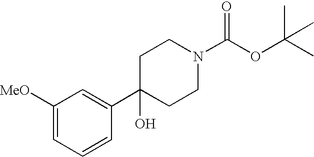

The title compound was synthesized by a method similar to that in Reference Example 21 and using 1-bromo-3-methoxybenzene instead of 1-bromo-2-(trifluoromethyl)benzene.

¹H-NMR (CDCl₃) δ: 1.48 (9H, s), 1.67-1.73 (3H, m), 1.80-2.14 (2H, m), 3.12-3.32 (2H, m), 3.76 (3H, s), 3.92-4.16 (2H, m), 7.09-7.52 (4H, m).

### Reference Example 23

### tert-butyl 4-(3,4-difluorophenyl)-4-hydroxypiperidine-1-carboxylate

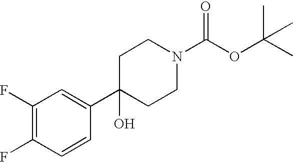

The title compound was synthesized by a method similar to that in Reference Example 21 and using 1-bromo-3,4-difluorobenzene instead of 1-bromo-2-(trifluoromethyl)benzene.

¹H-NMR (CDCl₃) δ: 1.47 (9H, s), 1.68-1.74 (2H, m), 1.87-1.97 (2H, m), 2.04 (1H, s), 3.16-3.24 (2H, m), 3.73-4.08 (2H, m), 7.08-7.19 (2H, m), 7.27-7.36 (1H, m).

### Reference Example 24

### tert-butyl 4-(2-chlorophenyl)-4-hydroxypiperidine-1-carboxylate

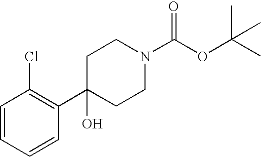

To a solution of 1-bromo-2-chlorobenzene (5.00 g) in tetrahydrofuran (25 ml) was added a solution of isopropylmagnesium bromide in tetrahydrofuran (1M, 26 ml) at 0° C., and the mixture was stirred for 1 hr. tert-Butyl 4-oxopiperidine-1-carboxylate (3.12 g) was added to the reaction mixture at 0° C., and the mixture was stirred at 0° C. for 1 hr. A saturated aqueous ammonium chloride solution was added to the reaction mixture at 0° C., and the mixture was extracted with ethyl acetate. The extract was dried over anhydrous magnesium sulfate, filtered and concentrated. The residue was purified by silica gel column chromatography (eluted with a mixed solution of hexane and ethyl acetate at a mixing ratio of 5:1) to give the title compound (270 mg).

¹H-NMR (CDCl₃) δ: 1.47 (9H, s), 1.61-1.91 (3H, m), 1.80-2.16 (2H, m), 3.08-3.36 (2H, m), 3.91-4.22 (2H, m), 7.02-7.52 (3H, m), 7.61-7.85 (1H, m).

### Reference Example 25

### tert-butyl 4-(2-fluorophenyl)-4-hydroxypiperidine-1-carboxylate

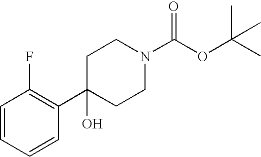

The title compound was synthesized by a method similar to that in Reference Example 24 and using 1-bromo-2-fluorobenzene instead of 1-bromo-2-chlorobenzene.

¹H-NMR (CDCl₃) δ: 1.48 (9H, s), 1.78-1.83 (2H, m), 2.11-2.24 (3H, m), 3.18-3.32 (2H, m), 4.01-4.15 (2H, m), 7.01-7.29 (3H, m), 7.43-7.49 (1H, m).

### Reference Example 26

### tert-butyl 4-(3-chlorophenyl)-4-hydroxypiperidine-1-carboxylate

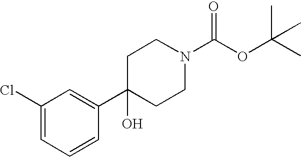

The title compound was synthesized by a method similar to that in Reference Example 24 and using 1-bromo-3-chlorobenzene instead of 1-bromo-2-chlorobenzene.

¹H-NMR (CDCl₃) δ: 1.48 (9H, s), 1.67-1.73 (3H, m), 1.80-2.14 (2H, m), 3.12-3.32 (2H, m), 3.92-4.16 (2H, m), 7.24-7.35 (3H, m), 7.48 (1H, s).

### Reference Example 27

### tert-butyl 4-(3-cyanophenyl)-4-hydroxypiperidine-1-carboxylate

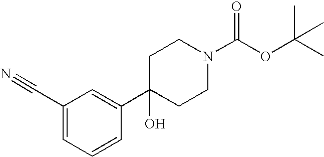

The title compound was synthesized by a method similar to that in Reference Example 24 and using 3-bromobenzonitrile instead of 1-bromo-2-chlorobenzene.

¹H-NMR (CDCl₃) δ: 1.49 (9H, s), 1.64-1.75 (2H, m), 1.88 (1H, s), 1.83-2.14 (2H, m), 3.16-3.29 (2H, m), 4.03-4.18 (2H, m), 7.21-7.48 (4H, m).

### Reference Example 28

### tert-butyl 4-(4-cyanophenyl)-4-hydroxypiperidine-1-carboxylate

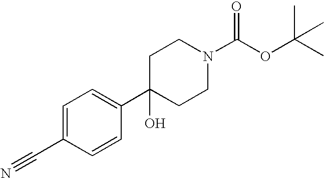

To a mixed solution of 4-bromobenzonitrile (10.0 g) in tetrahydrofuran (260 ml) and n-hexane (70 ml) was added dropwise a solution (1.6M, 34 ml) of n-butyllithium in hexane at −100° C., and the mixture was stirred at −100° C. for 1 hr. tert-Butyl 4-oxopiperidine-1-carboxylate (7.65 g) was added to the reaction mixture at −100° C., and the mixture was stirred at −100° C. for 1 hr and heated from −100° C. to 0° C. over 5 hrs. A saturated aqueous ammonium chloride solution was added to the reaction mixture at 0° C., and the mixture was extracted with ethyl acetate. The extract was dried over anhydrous magnesium sulfate, filtered and concentrated. The residue was purified by silica gel column chromatography (eluted with a mixed solution of hexane and ethyl acetate at a mixing ratio of 5:1) to give the title compound (4.20 g).

¹H-NMR (CDCl₃) δ: 1.48 (9H, s), 1.67-1.72 (2H, m), 1.86 (1H, s), 1.93-2.04 (2H, m), 3.17-3.25 (2H, m), 4.06-4.15 (2H, m), 7.57-7.66 (4H, m).

### Reference Example 29

### 1-benzyl-4-(3-fluorophenyl)piperidin-4-ol

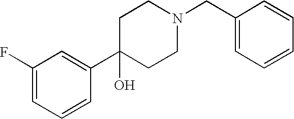

The title compound was synthesized by a method similar to that in Reference Example 21 and using 1-bromo-3-fluorobenzene instead of 1-bromo-2-(trifluoromethyl)benzene and 1-benzylpiperidin-4-one instead of tert-butyl 4-oxopiperidine-1-carboxylate.

¹H-NMR (CDCl₃) δ: 1.60-1.80 (2H, m), 2.05-2.25 (2H, m), 2.40-2.55 (2H, m), 2.75-2.90 (2H, m), 3.58 (2H, s), 6.90-7.40 (9H,m).

### Reference Example 30

### 4-(3-fluorophenyl)piperidin-4-ol

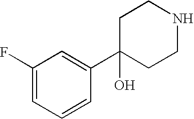

A mixture of 1-benzyl-4-(3-fluorophenyl)piperidin-4-ol (3.00 g), palladium hydroxide on carbon (1 g) and methanol (30 ml) was stirred under a hydrogen atmosphere at room temperature for 18 hrs. The reaction mixture was filtered and concentrated to give the title compound (1.27 g).

¹H-NMR (CDCl₃) δ: 1.60-1.80 (2H, m), 1.85-2.10 (2H, m), 2.85-3.20 (4H, m), 6.85-7.40 (4H, m).

### Reference Example 31

### 1-benzyl-4-[4-(trifluoromethoxy)phenyl]piperidin-4-ol

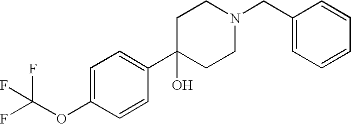

The title compound was synthesized by a method similar to that in Reference Example 21 and using 1-bromo-4-(trifluoromethoxy)benzene instead of 1-bromo-2-(trifluoromethyl)benzene and 1-benzylpiperidin-4-one instead of tert-butyl 4-oxopiperidine-1-carboxylate.

¹H-NMR (CDCl₃) δ: 1.65-1.80 (2H, m), 2.05-2.25 (2H, m), 2.40-2.60 (2H, m), 2.75-2.90 (2H, m), 3.58 (2H, s), 7.15-7.60 (9H, m).

### Reference Example 32

### 4-[4-(trifluoromethoxy)phenyl]piperidin-4-ol

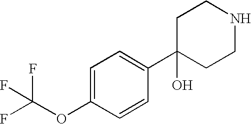

The title compound was synthesized by a method similar to that in Reference Example 30 and using 1-benzyl-4-[4-(trifluoromethoxy)phenyl]piperidin-4-ol instead of 1-benzyl-4-(3-fluorophenyl)piperidin-4-ol.

¹H-NMR (CDCl₃) δ: 1.60-1.80 (2H, m), 1.85-2.10 (2H, m), 2.85-3.20 (4H, m), 7.10-7.60 (4H, m).

### Reference Example 33

### ethyl 4-hydroxy-4-[4-(trifluoromethyl)phenyl]piperidine-1-carboxylate

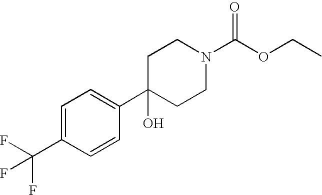

The title compound was synthesized by a method similar to that in Reference Example 21 and using 1-bromo-4-(trifluoromethyl)benzene instead of 1-bromo-2-(trifluoromethyl)benzene and ethyl 4-oxopiperidine-1-carboxylate instead of tert-butyl 4-oxopiperidine-1-carboxylate.

¹H-NMR (CDCl₃) δ: 1.27 (3H, t, J=6.9 Hz), 1.65-1.80 (2H, m), 1.90-2.10 (2H, m), 3.20-3.40 (2H, m), 4.00-4.20 (2H, m), 4.13 (2H, q, J=6.9 Hz), 7.50-7.65 (4H, m).

### Reference Example 34

### 4-[4-(trifluoromethyl)phenyl]piperidin-4-ol

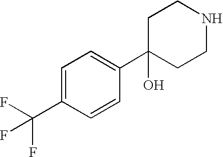

To a solution of ethyl 4-hydroxy-4-[4-(trifluoromethyl)phenyl]piperidine-1-carboxylate (9.51 g) in ethanol (150 ml) was added a solution of potassium hydroxide (11.78 g) in water (45 ml) at room temperature, and the mixture was heated under reflux for 90 hrs. After allowing to cool, the reaction mixture was extracted with ethyl acetate.

The extract was extracted with 1N hydrochloric acid. The extract washed with diethyl ether and treated with a 1N aqueous sodium hydroxide solution. The mixture was extracted with ethyl acetate, and the extract was dried over anhydrous sodium sulfate, filtered and concentrated to give the title compound (3.50 g).

¹H-NMR (CDCl₃) δ: 1.65-1.80 (2H, m), 1.90-2.10 (2H, m), 2.90-3.20 (4H, m), 7.50-7.70 (4H, m).

### Reference Example 35

### ethyl 4-(4-fluorobenzyl)-4-hydroxypiperidine-1-carboxylate

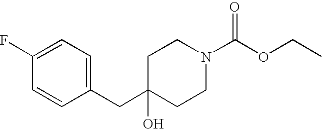

The title compound was synthesized by a method similar to that in Reference Example 21 and using 1-(chloromethyl)-4-fluorobenzene instead of 1-bromo-2-(trifluoromethyl)benzene and ethyl 4-oxopiperidine-1-carboxylate instead of tert-butyl 4-oxopiperidine-1-carboxylate.

¹H-NMR (CDCl₃) δ: 1.25 (3H, t, J=6.9 Hz), 1.40-1.70 (4H, m), 2.73 (2H, s), 3.05-3.20 (2H, m), 3.80-4.00 (2H, m), 4.12 (2H, q, J=6.9 Hz), 6.90-7.20 (4H, m).

### Reference Example 36

### 4-(4-fluorobenzyl)piperidin-4-ol

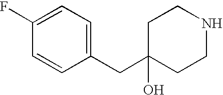

The title compound was synthesized by a method similar to that in Reference Example 34 and using ethyl 4-(4-fluorobenzyl)-4-hydroxypiperidine-1-carboxylate instead of ethyl 4-hydroxy-4-[4-(trifluoromethyl)phenyl]piperidine-1-carboxylate.

¹H-NMR (CDCl₃) δ: 1.45-1.70 (4H, m), 2.73 (2H, s), 2.80-3.00 (4H, m), 6.90-7.10 (2H, m), 7.10-7.20 (2H, m).

### Reference Example 37

### ethyl 4-(3-fluorobenzyl)-4-hydroxypiperidine-1-carboxylate

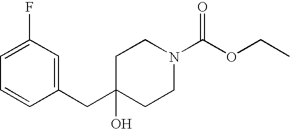

The title compound was synthesized by a method similar to that in Reference Example 21 and using 1-(chloromethyl)-3-fluorobenzene instead of 1-bromo-2-(trifluoromethyl)benzene and ethyl 4-oxopiperidine-1-carboxylate instead of tert-butyl 4-oxopiperidine-1-carboxylate.

¹H-NMR (CDCl₃) δ: 1.25 (3H, t, J=7.2 Hz), 1.44-1.68 (4H, m), 2.75 (2H, s), 3.06-3.21 (2H, m), 3.85-3.96 (2H, m), 4.12 (2H, q, J=7.2 Hz), 6.89-7.01 (3H, m), 7.21-7.34 (1H, m).

### Reference Example 38

### 4-(3-fluorobenzyl)piperidin-4-ol

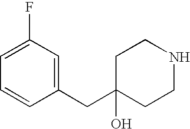

The title compound was synthesized by a method similar to that in Reference Example 34 and using ethyl 4-(3-fluorobenzyl)-4-hydroxypiperidine-1-carboxylate instead of ethyl 4-hydroxy-4-[4-(trifluoromethyl)phenyl]piperidine-1-carboxylate.

¹H-NMR (CDCl₃) δ: 1.43-1.71 (4H, m), 2.76 (2H, s), 2.84-3.02 (4H, m), 6.90-7.00 (3H, m), 7.21-7.32 (1H, m).

### Reference Example 39

### ethyl 4-(2-fluorobenzyl)-4-hydroxypiperidine-1-carboxylate

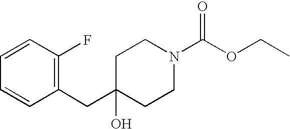

The title compound was synthesized by a method similar to that in Reference Example 21 and using 1-(chloromethyl)-2-fluorobenzene instead of 1-bromo-2-(trifluoromethyl)benzene and ethyl 4-oxopiperidine-1-carboxylate instead of tert-butyl 4-oxopiperidine-1-carboxylate.

¹H-NMR (CDCl₃) δ: 1.26 (3H, t, J=7.0 Hz), 1.42-1.72 (4H, m), 2.82 (2H, s), 3.07-3.22 (2H, m), 3.77-3.95 (2H, m), 4.11 (2H, q, J=7.0 Hz), 7.00-7.13 (2H, m), 7.18-7.29 (2H, m).

### Reference Example 40

### 4-(2-fluorobenzyl)piperidin-4-ol

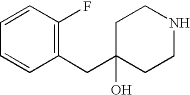

The title compound was synthesized by a method similar to that in Reference Example 34 and using ethyl 4-(2-fluorobenzyl)-4-hydroxypiperidine-1-carboxylate instead of ethyl 4-hydroxy-4-[4-(trifluoromethyl)phenyl]piperidine-1-carboxylate.

¹H-NMR (CDCl₃) δ: 1.47-1.74 (4H, m), 2.82 (2H, s), 2.82-3.03 (4H, m), 7.00-7.13 (2H, m), 7.18-7.28 (2H, m).

### Reference Example 41

### ethyl 4-(4-chlorobenzyl)-4-hydroxypiperidine-1-carboxylate

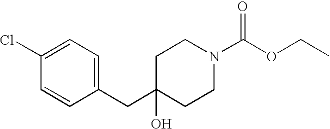

The title compound was synthesized by a method similar to that in Reference Example 21 and using 1-chloro-4-(chloromethyl)benzene instead of 1-bromo-2-(trifluoromethyl)benzene and ethyl 4-oxopiperidine-1-carboxylate instead of tert-butyl 4-oxopiperidine-1-carboxylate.

¹H-NMR (CDCl₃) δ: 1.25 (3H, t, J=7.2 Hz), 1.40-1.70 (4H, m), 2.72 (2H, s), 3.00-3.20 (2H, m), 3.80-4.00 (2H, m), 4.12 (2H, q, J=7.2 Hz), 7.05-7.35 (4H, m).

### Reference Example 42

### 4-(4-chlorobenzyl)piperidin-4-ol

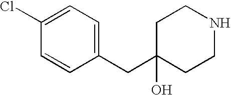

The title compound was synthesized by a method similar to that in Reference Example 34 and using ethyl 4-(4-chlorobenzyl)-4-hydroxypiperidine-1-carboxylate instead of ethyl 4-hydroxy-4-[4-(trifluoromethyl)phenyl]piperidine-1-carboxylate.

¹H-NMR (CDCl₃) δ: 1.40-1.70 (4H, m), 2.72 (2H, s), 2.75-3.00 (4H, m), 7.10-7.35 (4H, m).

### Reference Example 43

### ethyl 4-(3,4-difluorobenzyl)-4-hydroxypiperidine-1-carboxylate

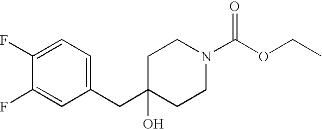

The title compound was synthesized by a method similar to that in Reference Example 21 and using 4-(chloromethyl)-1,2-difluorobenzene instead of 1-bromo-2-(trifluoromethyl)benzene and ethyl 4-oxopiperidine-1-carboxylate instead of tert-butyl 4-oxopiperidine-1-carboxylate.

¹H-NMR (CDCl₃) δ: 1.26 (3H, t, J=7.2 Hz), 1.45-1.62 (4H, m), 2.71 (2H, s), 3.04-3.21 (2H, m), 3.82-3.98 (2H, m), 4.11 (2H, q, J=7.2 Hz), 6.86-6.92 (1H, m), 6.99-7.14 (2H, m).

### Reference Example 44

### 4-(3,4-difluorobenzyl)piperidin-4-ol

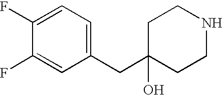

The title compound was synthesized by a method similar to that in Reference Example 34 and using ethyl 4-(3,4-difluorobenzyl)-4-hydroxypiperidine-1-carboxylate instead of ethyl 4-hydroxy-4-[4-(trifluoromethyl)phenyl]piperidine-1-carboxylate.

¹H-NMR (CDCl₃) δ: 1.45-1.65 (4H, m), 2.71 (2H, s), 2.83-2.97 (4H, m), 6.88-6.93 (1H, m), 7.00-7.13 (2H, m).

### Reference Example 45

### ethyl 4-hydroxy-4-[3-(trifluoromethyl)benzyl]piperidine-1-carboxylate

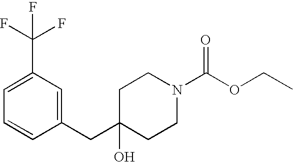

The title compound was synthesized by a method similar to that in Reference Example 21 and using 1-(chloromethyl)-3-(trifluoromethyl)benzene instead of 1-bromo-2-(trifluoromethyl)benzene and ethyl 4-oxopiperidine-1-carboxylate instead of tert-butyl 4-oxopiperidine-1-carboxylate.

¹H-NMR (CDCl₃) δ: 1.25 (3H, t, J=7.2 Hz), 1.46-1.62 (4H, m), 2.82 (2H, s), 3.08-3.18 (2H, m), 3.85-4.02 (2H, m), 4.12 (2H, q, J=7.2 Hz), 7.37-7.54 (4H, m).

### Reference Example 46

### 4-[3-(trifluoromethyl)benzyl]piperidin-4-ol

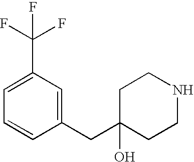

The title compound was synthesized by a method similar to that in Reference Example 34 and using ethyl 4-hydroxy-4-[3-(trifluoromethyl)benzyl]piperidine-1-carboxylate instead of ethyl 4-hydroxy-4-[4-(trifluoromethyl)phenyl]piperidine-1-carboxylate.

¹H-NMR (CDCl₃) δ: 1.36-1.58 (4H, m), 2.79 (2H, s), 2.83-2.87 (4H, m), 4.59 (1H, brs), 7.47-7.58 (4H, m).

### Reference Example 47

### tert-butyl 4-hydroxy-4-(phenylethynyl)piperidine-1-carboxylate

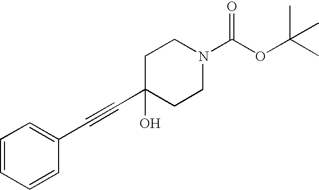

To a solution of ethynylbenzene (6.6 ml) in tetrahydrofuran (110 ml) was added dropwise a solution (1.6M, 38 ml) of n-butyllithium in hexane at −20° C., and the mixture was stirred at −20° C. for 1 hr. The reaction mixture was cooled to −60° C., a solution of tert-butyl 4-oxopiperidine-1-carboxylate (10.0 g) in tetrahydrofuran (70 ml) was added dropwise at −60° C., and the mixture was stirred at −60° C. for 30 min, and at room temperature for 2 hrs. A 10% aqueous ammonium chloride solution was added to the reaction mixture at room temperature, and the mixture was extracted with ethyl acetate. The extract washed with saturated brine, dried over anhydrous magnesium sulfate, filtered and concentrated. The residue was purified by silica gel column chromatography (eluted with a mixed solution of hexane and ethyl acetate at a mixing ratio of 9:1-1:1) to give the title compound (14.27 g)).

¹H-NMR (CDCl₃) δ: 1.46 (9H, s), 1.68-1.83 (2H, m), 1.95-2.02 (2H, m), 2.44 (1H, s), 3.27-3.36 (2H, m), 3.69-3.85 (2H, m), 7.27-7.35 (3H, m), 7.38-7.45 (2H, m).

### Reference Example 48

### 4-(phenylethynyl)piperidin-4-ol

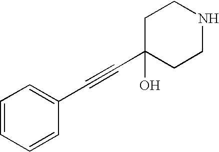

The title compound was synthesized by a method similar to that in Reference Example 20 and using tert-butyl 4-hydroxy-4-(phenylethynyl)piperidine-1-carboxylate instead of tert-butyl 4-(4-fluorophenyl)-4-hydroxypiperidine-1-carboxylate.

¹H-NMR (CDCl₃) δ: 1.64-1.73 (2H, m), 1.86-1.93 (2H, m), 2.75-2.83 (2H, m), 2.92 (2H, m), 4.99 (2H, m), 7.35-7.44 (5H, m).

### Reference Example 49

### 4-[(E)-2-phenylvinyl]piperidin-4-ol

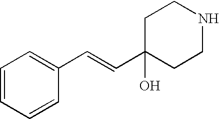

A solution of 4-(phenylethynyl)piperidin-4-ol (1.00 g) in tetrahydrofuran (25 ml) was cooled to 0° C., lithium aluminum hydride (290 mg) was added, and the mixture was stirred at room temperature for 2 hrs, and at 60° C. for 1.5 hrs. Lithium aluminum hydride (293 mg) was further added, and the mixture was stirred at 60° C. for 30 min. The reaction mixture was cooled to 0° C., sodium sulfate 10 hydrate (4.78 g) was added, and the mixture was stirred at room temperature for 30 min. Magnesium sulfate was added to the mixture, and the mixture was filtered and concentrated. The residue was purified by basic silica gel column chromatography (eluted with a mixed solution of ethyl acetate and methanol at a mixing ratio of 1:0-7:3) to give the title compound (460 mg).

¹H-NMR (CDCl₃) δ: 1.40-1.50 (2H, m), 1.51-1.61 (2H, m), 2.62-2.67 (2H, m), 2.78-2.86 (2H, m), 4.52 (1H, s), 6.35 (1H, d, J=15.9 Hz), 6.52 (1H, d, J=15.9 Hz), 7.15-7.23 (1H, m), 7.26-7.34 (2H, m), 7.36-7.44 (2H, m).

### Reference Example 50

### tert-butyl 4-[(4-fluorophenyl)ethynyl]-4-hydroxypiperidine-1-carboxylate

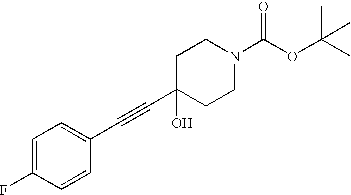

The title compound was synthesized by a method similar to that in Reference Example 47 and using 1-ethynyl-4-fluorobenzene instead of ethynylbenzene.

¹H-NMR (CDCl₃) δ: 1.47 (9H, s), 1.70-1.85 (2H, m), 1.90-2.05 (2H, m), 2.10 (1H, s), 3.32 (2H, ddd, J=3.6, 9.6, 13.4 Hz), 3.75-3.90 (2H, m), 6.95-7.10 (2H, m), 7.35-7.45 (2H, m).

### Reference Example 51

### 4-[(4-fluorophenyl)ethynyl]piperidin-4-ol

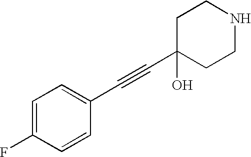

The title compound was synthesized by a method similar to that in Reference Example 20 and using tert-butyl 4-[(4-fluorophenyl)ethynyl]-4-hydroxypiperidine-1-carboxylate instead of tert-butyl 4-(4-fluorophenyl)-4-hydroxypiperidine-1-carboxylate.

¹H-NMR (CDCl₃) δ: 1.75 (2H, ddd, J=3.7, 9.6, 12.9 Hz), 1.95-2.10 (2H, m), 2.91 (2H, ddd, J=3.0, 9.9, 12.9 Hz), 3.09 (2H, td, J=4.6, 13.1 Hz), 6.95-7.05 (2H, m), 7.35-7.45 (2H, m).

### Reference Example 52

### 4-[(E)-2-(4-fluorophenyl)vinyl]piperidin-4-ol

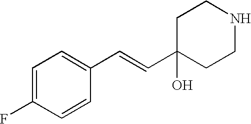

The title compound was synthesized by a method similar to that in Reference Example 49 and using 4-[(4-fluorophenyl)ethynyl]piperidin-4-ol instead of 4-(phenylethynyl)piperidin-4-ol.

¹H-NMR (CDCl₃) δ: 1.55-1.70 (2H, m), 1.79 (2H, ddd, J=4.1, 10.4, 14.0 Hz), 2.90 (2H, td, J=4.3, 12.5 Hz), 3.04 (2H, ddd, J=2.8, 10.1, 12.8 Hz), 6.23 (1H, d, J=16.2 Hz), 6.61 (1H, d, J=16.2 Hz), 6.95-7.05 (2H, m), 7.30-7.40 (2H, m).

### Reference Example 53

### 4-[2-(4-fluorophenyl)ethyl]piperidin-4-ol

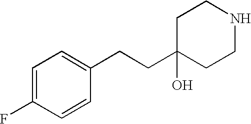

A mixture of 4-[(4-fluorophenyl)ethynyl]piperidin-4-ol (1096 mg), methanol (50 ml) and 10% palladium carbon (438 mg) was stirred under a hydrogen atmosphere at room temperature for 18 hrs. The reaction mixture was filtered and concentrated. The residue was purified by basic silica gel column chromatography (eluted with a mixed solution of ethyl acetate and methanol at a mixing ratio of 1:0-7:3) to give the title compound (754 mg).

¹H-NMR (CDCl₃) δ: 1.50-1.70 (4H, m), 1.70-1.80 (2H, m), 2.65-2.75 (2H, m), 2.80-3.05 (4H, m), 6.90-7.05 (2H, m), 7.10-7.20 (2H, m).

### Reference Example 54

### tert-butyl 1-oxa-6-azaspiro[2,5]octane-6-carboxylate

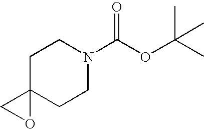

To a suspension of sodium hydride (60% suspension in mineral oil, 6.02 g) in dimethyl sulfoxide (150 ml) was added trimethylsulfoxonium iodide (33.13 g) at 5° C.-10° C., and the mixture was stirred at room temperature for 1 hr. A solution of tert-butyl 4-oxopiperidine-1-carboxylate (25.00 g) in dimethyl sulfoxide (50 ml) was added to the reaction mixture at 5° C.-10° C., and the mixture was stirred at 50° C. for 1.5 hrs. Water was added to the reaction mixture, and the mixture was extracted with dichloromethane. The extract was dried over anhydrous sodium sulfate, filtered and concentrated. The residue was purified by basic silica gel column chromatography (eluted with a mixed solution of hexane and ethyl acetate at a mixing ratio of 4:1-2:3) to give the title compound (15.99 g).

¹H-NMR (CDCl₃) δ: 1.35-1.55 (2H, m), 1.48 (9H, s), 1.80 (2H, ddd, J=4.4, 9.3, 13.7 Hz), 2.69 (2H, s), 3.43 (2H, ddd, J=3.7, 9.5, 13.3 Hz), 3.65-3.80 (2H, m).

### Reference Example 55

### tert-butyl 4-[(4-fluorophenoxy)methyl]-4-hydroxypiperidine-1-carboxylate

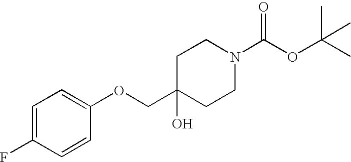

A mixture of tert-butyl 1-oxa-6-azaspiro[2,5]octane-6-carboxylate (2.13 g), 4-fluorophenol (1.23 g), potassium carbonate (1.52 g) and dimethylformamide (20 ml) was stirred at 80° C. for 15 hrs and concentrated. Water was added to the residue, and the mixture was extracted with ethyl acetate. The extract washed with saturated brine, dried over anhydrous sodium sulfate, filtered and concentrated. The residue was purified by silica gel column chromatography (eluted with a mixed solution of hexane and ethyl acetate at a mixing ratio of 9:1-1:1) to give the title compound (2.94 g).

¹H-NMR (CDCl₃) δ: 1.47 (9H, s), 1.50-1.80 (4H, m), 2.16 (1H, s), 3.15-3.30 (2H, m), 3.77 (2H, s), 3.80-4.00 (2H, m), 6.80-6.90 (2H, m), 6.90-7.05 (2H, m).

### Reference Example 56

### 4-[(4-fluorophenoxy)methyl]piperidin-4-ol

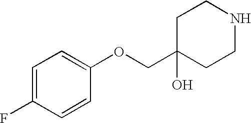

The title compound was synthesized by a method similar to that in Reference Example 20 and using tert-butyl 4-[(4-fluorophenoxy)methyl]-4-hydroxypiperidine-1-carboxylate instead of tert-butyl 4-(4-fluorophenyl)-4-hydroxypiperidine-1-carboxylate.

¹H-NMR (CDCl₃) δ: 1.85-2.10 (4H, m), 3.25-3.40 (4H, m), 3.84 (2H, s), 6.90-7.05 (4H, m).

### Reference Example 57

### tert-butyl 4-(1-benzothien-2-yl)-4-hydroxypiperidine-1-carboxylate

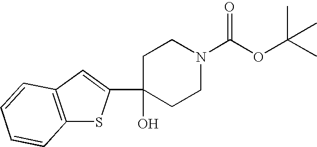

A solution (1.6M, 15.6 ml) of n-butyllithium in hexane was added dropwise to a solution of 1-benzothiophene (3.36 g) in tetrahydrofuran (50 ml) at −78° C., and the mixture was stirred at −78° C. for 10 min and at 0° C. for 40 min, and then cooled again to −78° C. To the reaction mixture was added dropwise a solution of tert-butyl 4-oxopiperidine-1-carboxylate (3.99 g) in tetrahydrofuran (30 ml) at −78° C., and the mixture was stirred at −78° C. for 2 hrs. To the reaction mixture was added 10% aqueous sodium hydrogen carbonate solution, and the mixture was extracted with ethyl acetate. The extract washed with saturated brine, dried over anhydrous magnesium sulfate, filtered and concentrated. The residue was purified by silica gel column chromatography (eluted with a mixed solution of hexane and ethyl acetate at a mixing ratio of 9:1-3:1) to give the title compound (6.41 g).

¹H-NMR (CDCl₃) δ: 1.46 (9H, s), 1.90-2.20 (4H, m), 3.20-3.40 (2H, m), 3.80-4.10 (2H, m), 7.18 (1H, s), 7.20-7.40 (2H, m), 7.65-7.85 (2H, m).

### Reference Example 58

### 4-(1-benzothien-2-yl)piperidin-4-ol

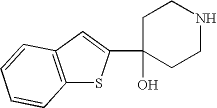

The title compound was synthesized by a method similar to that in Reference Example 20 and using tert-butyl 4-(1-benzothien-2-yl)-4-hydroxypiperidine-1-carboxylate instead of tert-butyl 4-(4-fluorophenyl)-4-hydroxypiperidine-1-carboxylate.

¹H-NMR (CDCl₃) δ: 1.90-2.05 (2H, m), 2.05-2.20 (2H, m), 2.85-3.20 (4H, m), 7.19 (1H, s), 7.25-7.40 (2H, m), 7.65-7.85 (2H, m).

### Reference Example 59

### tert-butyl 4-[(3-fluorophenyl)ethynyl]-4-hydroxypiperidine-1-carboxylate

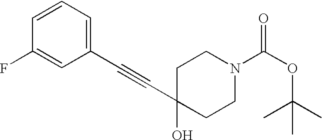

The title compound was synthesized by a method similar to that in Reference Example 47 and using 1-ethynyl-3-fluorobenzene instead of ethynylbenzene.

¹H-NMR (CDCl₃) δ: 1.47 (9H, s), 1.71-1.88 (2H, m), 1.92-2.04 (2H, m), 2.20 (1H, s), 3.22-3.42 (2H, m), 3.75-3.91 (2H, m), 6.99-7.16 (2H, m), 7.16-7.34 (2H, m).

### Reference Example 60

### 4-[(3-fluorophenyl)ethynyl]piperidin-4-ol

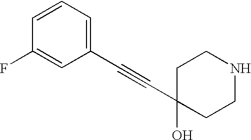

The title compound was synthesized by a method similar to that in Reference Example 20 and using tert-butyl 4-[(3-fluorophenyl)ethynyl]-4-hydroxypiperidine-1-carboxylate instead of tert-butyl 4-(4-fluorophenyl)-4-hydroxypiperidine-1-carboxylate.

¹H-NMR (DMSO-d₆) δ: 1.56-1.75 (2H, m), 1.79-1.95 (2H, m), 2.67-2.86 (2H, m), 2.86-3.02 (2H, m), 3.57-4.62 (1H, m), 5.29-6.19 (1H, m), 7.16-7.33 (3H, m), 7.35-7.55 (1H, m).

### Example 1

### 4,5,6,11-tetrahydro-1H-pyrazolo[4′,3′:6,7]cyclohepta[1,2-b]indole

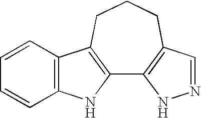

A mixture of 7-[(dimethylamino)methylene]-7,8,9,10-tetrahydrocyclohepta[b]indol-6(5H)-one (195 mg), hydrazine monohydrate (0.15 ml) and ethanol (5 ml) was heated under reflux for 1 hr. After allowing to cool, water was added to the reaction mixture, and the mixture was extracted with ethyl acetate. The extract washed with saturated brine, dried over anhydrous sodium sulfate, filtered and concentrated. The residue washed with hexane to give the title compound (152 mg) as a colorless solid.

¹H-NMR (CDCl₃) δ: 2.00-2.20 (2H, m), 2.92 (2H, t, J=5.6 Hz), 3.15 (2H, t, J=5.8 Hz), 7.00-7.60 (5H, m), 8.84 (1H, brs).

### Example 2

### ethyl 4,5,6,11-tetrahydro-1H-pyrazolo[4′,3′:6,7]cyclohepta[1,2-b]indole-8-carboxylate

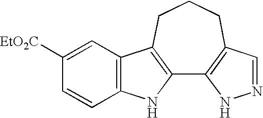

In the same manner as in Example 1 except that ethyl 7-[(dimethylamino)methylene]-6-oxo-5,6,7,8,9,10-hexahydrocyclohepta[b]indole-2-carboxylate was used instead of 7-[(dimethylamino)methylene]-7,8,9,10-tetrahydrocyclohepta[b]indol-6(5H)-one, the title compound was obtained.

¹H-NMR (CDCl₃) δ: 1.42 (3H, t, J=6.9 Hz), 2.05-2.20 (2H, m), 2.93 (2H, t, J=5.7 Hz), 3.18 (2H, t, J=5.4 Hz), 4.40 (2H, q, J=6.9 Hz), 7.29 (1H, d, J=7.8 Hz), 7.35 (1H, s), 7.88 (1H, dd, J=7.8, 1.2 Hz), 8.29 (1H, d, J=1.2 Hz), 9.12 (1H, s).

### Example 3

### 4,5,6,11-tetrahydro-1H-pyrazolo[4′,3′:6,7]cyclohepta[1,2-b]indole-8-carboxylic acid

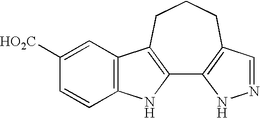

To a mixed solution of ethyl 4,5,6,11-tetrahydro-1H-pyrazolo[4′,3′:6,7]cyclohepta[1,2-b]indole-8-carboxylate (1.47 g) in methanol (20 ml) and tetrahydrofuran (10 ml) was added a 3N aqueous sodium hydroxide solution (15 ml), and the mixture was stirred at room temperature for 90 hrs. To the reaction mixture was added a 1N aqueous hydrochloric acid solution (100 ml), and the mixture was extracted with ethyl acetate. The extract washed with saturated brine, dried over anhydrous sodium sulfate, filtered and concentrated. The residue was washed with a mixed solution of diethyl ether and hexane to give the title compound (1.26 g) as a colorless solid.

¹H-NMR (5% CD₃OD-containing CDCl₃) δ: 2.00-2.20 (2H, m), 2.93 (2H, t, J=6.0 Hz), 3.18 (2H, t, J=5.7 Hz), 7.34 (1H, s), 7.30-7.45 (2H, m), 7.85-7.95 (1H, m), 8.33 (1H, s).

### Example 4

### methyl 10-methyl-4,5,6,11-tetrahydro-1H-pyrazolo[4′,3′:6,7]cyclohepta[1,2-b]indole-7-carboxylate

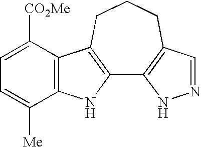

In the same manner as in Example 1 except that methyl 7-[(dimethylamino)methylene]-4-methyl-6-oxo-5,6,7,8,9,10-hexahydrocyclohepta[b]indole-1-carboxylate was used instead of 7-[(dimethylamino)methylene]-7,8,9,10-tetrahydrocyclohepta[b]indol-6(5H)-one, the title compound was obtained.

¹H-NMR (CDCl₃) δ: 2.00-2.15 (2H, m), 2.53 (3H, s), 2.85-2.95 (2H, m), 3.15-3.25 (2H, m), 3.95 (3H, s), 6.97 (1H, d, J=7.5 Hz), 7.36 (1H, s), 7.44 (1H, d, J=7.5 Hz), 8.91 (1H, s).

### Example 5

### 10-methyl-4,5,6,11-tetrahydro-1H-pyrazolo[4′,3′:6,7]cyclohepta[1,2-b]indole-7-carboxylic acid

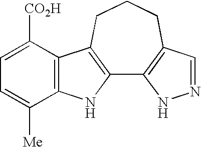

In the same manner as in Example 3 except that methyl 10-methyl-4,5,6,11-tetrahydro-1H-pyrazolo[4′,3′:6,7]cyclohepta[1,2-b]indole-7-carboxylate was used instead of ethyl 4,5,6,11-tetrahydro-1H-pyrazolo[4′,3′:6,7]cyclohepta[1,2-b]indole-8-carboxylate, the title compound was obtained.

¹H-NMR (DMSO-d₆) δ: 1.85-2.00 (2H, m), 2.54 (3H, s), 2.75-2.90 (2H, m), 3.05-3.20 (2H, m), 6.88 (1H, d, J=7.5 Hz), 7.21 (1H, d, J=7.5 Hz), 7.51 (1H, s), 10.83 (1H, s), 12.58 (1H, brs).

### Example 6

### 1-(4,5,6,11-tetrahydro-1H-pyrazolo[4′,3′:6,7]cyclohepta[1,2-b]indol-8-ylcarbonyl)piperidin-4-ol

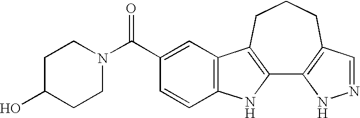

A mixture of 4,5,6,11-tetrahydro-1H-pyrazolo[4′,3′:6,7]cyclohepta[1,2-b]indole-8-carboxylic acid (400 mg), piperidin-4-ol (180 mg), triethylamine (1.04 ml), diethyl cyanophosphate (0.34 ml) and dimethylformamide (4 ml) was stirred at room temperature for 18 hrs. The reaction mixture was concentrated, and the residue was purified by silica gel column chromatography (eluted with a mixed solution of ethyl acetate and methanol at a mixing ratio of 90:10-80:20) and recrystallized (a mixed solution of methanol and ethyl acetate) to give the title compound (393 mg) as a colorless solid.

¹H-NMR (DMSO-d₆) δ: 1.25-1.45 (2H, m), 1.65-1.85 (2H, m), 1.90-2.10 (2H, m), 2.80-3.30 (8H, m), 3.65-3.85 (1H, m), 7.07 (1H, d, J=8.1 Hz), 7.33 (1H, d, J=8.1 Hz), 7.46 (1H, s), 7.56 (1H, s), 11.20 (1H, s), 12.70 (1H, s).

### Example 7

### 8-(piperidin-1-ylcarbonyl)-4,5,6,11-tetrahydro-1H-pyrazolo[4′,3′:6,7]cyclohepta[1,2-b]indole

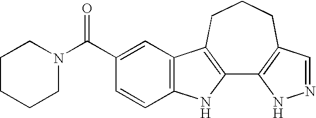

In the same manner as in Example 6 except that piperidine was used instead of piperidin-4-ol, the title compound was obtained.

¹H-NMR (CDCl₃) δ: 1.50-1.75 (6H, m), 2.00-2.25 (2H, m), 2.91 (2H, t, J=5.7 Hz), 3.11 (2H, t, J=5.7 Hz), 3.20-3.80 (4H, m), 7.15-7.40 (3H, m), 7.62 (1H, s).

### Example 8

### N-ethyl-N-(2-thienylmethyl)-4,5,6,11-tetrahydro-1H-pyrazolo[4′,3′:6,7]cyclohepta[1,2-b]indole-8-carboxamide

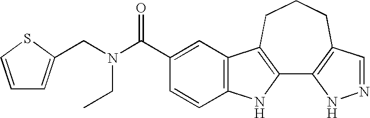

In the same manner as in Example 6 except that N-(2-thienylmethyl)ethaneamine was used instead of piperidin-4-ol, the title compound was obtained.

¹H-NMR (CDCl₃) δ: 1.20 (3H, t, J=6.9 Hz), 2.00-2.15 (2H, m), 2.85-2.95 (2H, m), 3.05-3.15 (2H, m), 3.35-3.55 (2H, m), 4.84 (2H, brs), 6.90-7.30 (6H, m), 7.67 (1H, s), 9.15 (1H, brs).

### Example 9

### 8-[(4-pyrrolidin-1-ylpiperidin-1-yl)carbonyl]-4,5,6,11-tetrahydro-1H-pyrazolo[4′,3′:6,7]cyclohepta[1,2-b]indole hydrochloride

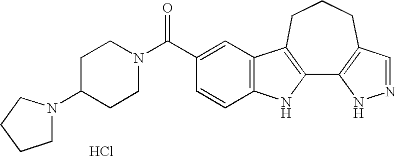

The title compound was obtained by treating, with hydrochloric acid, a compound obtained in the same manner as in Example 6 and using 4-pyrrolidin-1-ylpiperidine instead of piperidin-4-ol.

¹H-NMR (DMSO-d₆) δ: 1.60-2.20 (10H, m), 2.80-3.60 (13H, m), 7.10 (1H, dd, J=1.2, 8.4 Hz), 7.37 (1H, d, J=8.4 Hz), 7.50 (1H, d, J=1.2 Hz), 7.56 (1H, s), 10.95 (1H, brs), 11.29 (1H, s).

### Example 10

### 8-(1,4′-bipiperidin-1′-ylcarbonyl)-4,5,6,11-tetrahydro-1H-pyrazolo[4′,3′:6,7]cyclohepta[1,2-b]indole hydrochloride

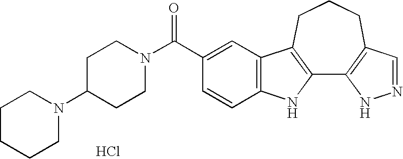

The title compound was obtained by treating, with hydrochloric acid, a compound obtained in the same manner as in Example 6 and using 1,4′-bipiperidine instead of piperidin-4-ol.

¹H-NMR (DMSO-d₆) δ: 1.30-2.20 (12H, m), 2.80-3.50 (13H, m), 7.11 (1H, dd, J=1.8, 8.7 Hz), 7.36 (1H, d, J=8.7 Hz), 7.50 (1H, d, J=1.8 Hz), 7.55 (1H, s), 10.43 (1H, brs), 11.29 (1H, s).

### Example 11

### 8-[(4-azepan-1-ylpiperidin-1-yl)carbonyl]-4,5,6,11-tetrahydro-1H-pyrazolo[4′,3′:6,7]cyclohepta[1,2-b]indole hydrochloride

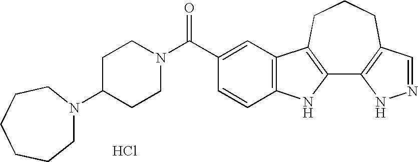

The title compound was obtained by treating, with hydrochloric acid, a compound obtained in the same manner as in Example 6 and using 1-piperidin-4-ylazepane dihydrochloride instead of piperidin-4-ol.

¹H-NMR (DMSO-d₆) δ: 1.50-2.40 (12H, m), 2.80-3.60 (13H, m), 7.10-7.60 (4H, m), 10.45 (1H, brs), 11.29 (1H, s).

### Example 12

### 8-[(4-azocan-1-ylpiperidin-1-yl)carbonyl]-4,5,6,11-tetrahydro-1H-pyrazolo[4′,3′:6,7]cyclohepta[1,2-b]indole hydrochloride

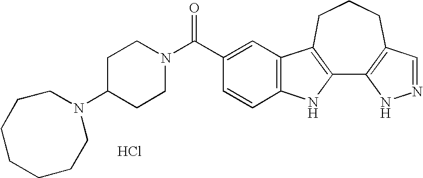

The title compound was obtained by treating, with hydrochloric acid, a compound obtained in the same manner as in Example 6 and using 1-piperidin-4-ylazocane dihydrochloride instead of piperidin-4-ol.

¹H-NMR (DMSO-d₆) δ: 1.40-4.50 (29H, m), 7.10 (1H, d, J=8.4 Hz), 7.37 (1H, d, J=8.4 Hz), 7.52 (1H, s), 7.56 (1H, s).

### Example 13

### 8-[(4-azonan-1-ylpiperidin-1-yl)carbonyl]-4,5,6,11-tetrahydro-1H-pyrazolo[4′,3′:6,7]cyclohepta[1,2-b]indole hydrochloride

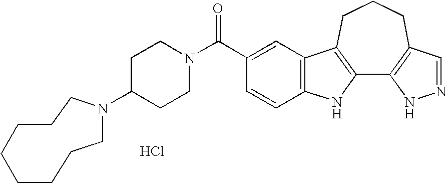

The title compound was obtained by treating, with hydrochloric acid, a compound obtained in the same manner as in Example 6 and using 1-piperidin-4-ylazonane dihydrochloride instead of piperidin-4-ol.

¹H-NMR (DMSO-d₆) δ: 1.40-2.40 (18H, m), 2.80-3.60 (13H, m), 7.13 (1H, d, J=8.1 Hz), 7.37 (1H, d, J=8.1 Hz), 7.53 (1H, s), 7.56 (1H, s).

### Example 14

### 8-[(4-morpholin-4-ylpiperidin-1-yl)carbonyl]-4,5,6,11-tetrahydro-1H-pyrazolo[4′,3′:6,7]cyclohepta[1,2-b]indole hydrochloride

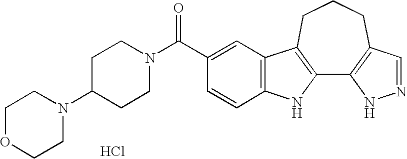

The title compound was obtained by treating, with hydrochloric acid, a compound obtained in the same manner as in Example 6 and using 4-piperidin-4-ylmorpholine dihydrochloride instead of piperidin-4-ol.

¹H-NMR (DMSO-d₆) δ: 1.60-2.25 (6H, m), 2.80-4.50 (17H, m), 7.11 (1H, d, J=8.1 Hz), 7.37 (1H, d, J=8.1 Hz), 7.51 (1H, s), 7.57 (1H, s).

### Example 15

### 8-[(4-thiomorpholin-4-ylpiperidin-1-yl)carbonyl]-4,5,6,11-tetrahydro-1H-pyrazolo[4′,3′:6,7]cyclohepta[1,2-b]indole hydrochloride

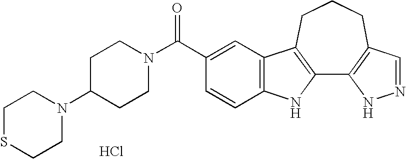

The title compound was obtained by treating, with hydrochloric acid, a compound obtained in the same manner as in Example 6 and using 4-piperidin-4-ylthiomorpholine dihydrochloride instead of piperidin-4-ol.

¹H-NMR (DMSO-d₆) δ: 1.60-2.25 (6H, m), 2.75-4.50 (17H, m), 7.11 (1H, d, J=8.1 Hz), 7.35 (1H, d, J=8.1 Hz), 7.51 (1H, s), 7.55 (1H, s).

### Example 16

### N,N-diethyl-1-(4,5,6,11-tetrahydro-1H-pyrazolo[41, 31:6,7]cyclohepta[1,2-b]indol-8-ylcarbonyl)piperidin-4-amine fumarate

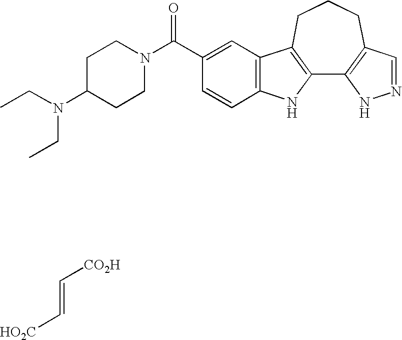

The title compound was obtained by treating, with fumaric acid, a compound obtained in the same manner as in Example 6 and using N,N-diethylpiperidin-4-amine dihydrochloride instead of piperidin-4-ol.

¹H-NMR (DMSO-d₆) δ: 1.09 (6H, t, J=6.9 Hz), 1.40-2.10 (6H, m), 2.65-4.20 (11H, m), 6.54 (2H, s), 7.10 (1H, d, J=8.4 Hz), 7.35 (1H, d, J=8.4 Hz), 7.49 (1H, s), 7.54 (1H, s), 11.28 (1H, s).

### Example 17

### N-benzyl-1-(4,5,6,11-tetrahydro-1H-pyrazolo[4′,3′:6,7]cyclohepta[1,2-b]indol-8-ylcarbonyl)piperidin-4-amine hydrochloride

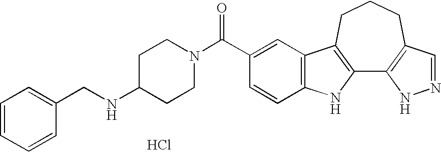

The title compound was obtained by treating, with hydrochloric acid, a compound obtained in the same manner as in Example 6 and using N-benzylpiperidin-4-amine dihydrochloride instead of piperidin-4-ol.

¹H-NMR (DMSO-d₆) δ: 1.50-4.30 (17H, m), 7.08 (1H, d, J=8.1 Hz), 7.36 (1H, d, J=8.1 Hz), 7.40-7.65 (7H, m), 11.29 (1H, s).

### Example 18

### N-benzyl-N-methyl-1-(4,5,6,11-tetrahydro-1H-pyrazolo[4′,3′:6,7]cyclohepta[1,2-b]indol-8-ylcarbonyl)piperidin-4-amine hydrochloride

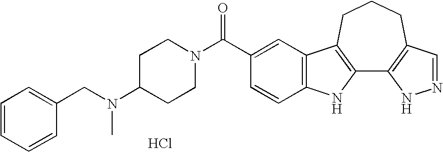

The title compound was obtained by treating, with hydrochloric acid, a compound obtained in the same manner as in Example 6 and using N-benzyl-N-methylpiperidin-4-amine dihydrochloride instead of piperidin-4-ol.

¹H-NMR (DMSO-d₆) δ: 1.70-4.55 (20H, m), 7.05-7.70 (9H, m), 10.50 (1H, s), 11.27 (1H, s).

### Example 19

### N-benzyl-N-ethyl-1-(4,5,6,11-tetrahydro-1H-pyrazolo[4′,3′:6,7]cyclohepta[1,2-b]indol-8-ylcarbonyl)piperidin-4-amine hydrochloride

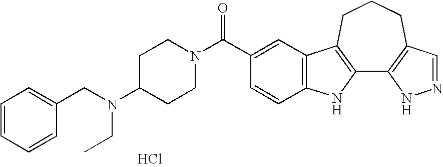

The title compound was obtained by treating, with hydrochloric acid, a compound obtained in the same manner as in Example 6 and using N-benzyl-N-ethylpiperidin-4-amine dihydrochloride instead of piperidin-4-ol.

¹H-NMR (DMSO-d₆) δ: 1.22 (3H, t, J=6.9 Hz), 1.70-4.60 (19H, m), 7.14 (1H, d, J=8.4 Hz), 7.37 (1H, d, J=8.4 Hz), 7.45-7.50 (3H, m), 7.52 (1H, s), 7.56 (1H, s), 7.60-7.75 (1H, m), 10.39 (1H, brs), 11.29 (1H, s).

### Example 20

### 2-[N-benzyl-N-[1-(4,5,6,11-tetrahydro-1H-pyrazolo[4′,3′:6,7]cyclohepta[1,2-b]indol-8-ylcarbonyl)piperidin-4-yl]amino]ethanol hydrochloride

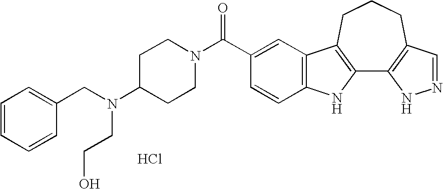

The title compound was obtained by treating, with hydrochloric acid, a compound obtained in the same manner as in Example 6 and using 2-[N-benzyl-N-(piperidin-4-yl)amino]ethanol dihydrochloride instead of piperidin-4-ol.

¹H-NMR (DMSO-d₆) δ: 1.70-4.60 (21H, m), 7.13 (1H, d, J=8.4 Hz), 7.36 (1H, d, J=8.4 Hz), 7.40-7.50 (3H, m), 7.52 (1H, s), 7.55 (1H, s), 7.60-7.80 (2H, m), 10.19 (1H, brs), 11.28 (1H, s).

### Example 21

### N-ethyl-1-(4,5,6,11-tetrahydro-1H-pyrazolo[4′,3′:6,7]cyclohepta[1,2-b]indol-8-ylcarbonyl)-N-(2-thienylmethyl)piperidin-4-amine fumarate

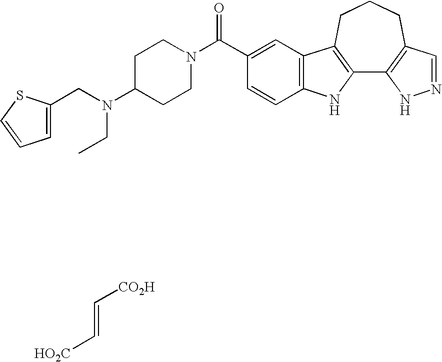

The title compound was obtained by treating, with fumaric acid, a compound obtained in the same manner as in Example 6 and using N-ethyl-N-(2-thienylmethyl)piperidin-4-amine dihydrochloride instead of piperidin-4-ol.

¹H-NMR (DMSO-d₆) δ: 0.99 (3H, t, J=6.9 Hz), 1.35-2.10 (6H, m), 2.40-3.60 (11H, m), 3.83 (2H, s), 6.61 (2H, s), 6.90-7.00 (2H, m), 7.08 (1H, d, J=8.4 Hz), 7.30-7.40 (2H, m), 7.46 (1H, s), 7.53 (1H, s), 11.18 (1H, s).

### Example 22

### N-[2-(dimethylamino)ethyl]-4,5,6,11-tetrahydro-1H-pyrazolo[4′,3′:6,7]cyclohepta[1,2-b]indole-8-carboxamide hydrochloride

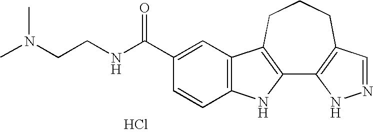

The title compound was obtained by treating, with hydrochloric acid, a compound obtained in the same manner as in Example 6 and using N,N-dimethylethane-1,2-diamine instead of piperidin-4-ol.

¹H-NMR (DMSO-d₆) δ: 1.90-2.10 (2H, m), 2.83 (3H, s), 2.84 (3H, s), 2.80-2.95 (2H, m), 3.05-3.15 (2H, m), 3.20-3.35 (2H, m), 3.60-3.75 (2H, m), 7.35 (1H, d, J=8.7 Hz), 7.57 (1H, s), 7.67 (1H, dd, J=0.9, 8.7 Hz), 8.15 (1H, d, J=0.9 Hz), 8.65-8.80 (1H, m), 10.35 (1H, brs).

### Example 23

### N-[3-(dimethylamino)propyl]-4,5,6,11-tetrahydro-1H-pyrazolo[4′,3′:6,7]cyclohepta[1,2-b]indole-8-carboxamide hydrochloride

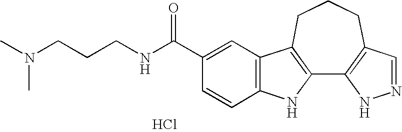

The title compound was obtained by treating, with hydrochloric acid, a compound obtained in the same manner as in Example 6 and using N,N-dimethylpropane-1,3-diamine instead of piperidin-4-ol.

¹H-NMR (DMSO-d₆) δ: 1.85-2.10 (4H, m), 2.74 (3H, s), 2.76 (3H, s), 2.85-3.45 (8H, m), 7.35 (1H, d, J=8.4 Hz), 7.58 (1H, s), 7.64 (1H, dd, J=0.9, 8.4 Hz), 8.50-8.65 (1H, m), 10.40 (1H, brs).

### Example 24

### N-[4-(dimethylamino)butyl]-4,5,6,11-tetrahydro-1H-pyrazolo[4′,3′:6,7]cyclohepta[1,2-b]indole-8-carboxamide hydrochloride

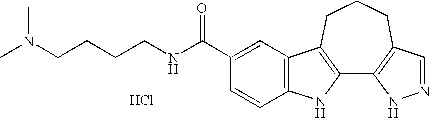

The title compound was obtained by treating, with hydrochloric acid, a compound obtained in the same manner as in Example 6 and using N,N-dimethylbutane-1,4-diamine instead of piperidin-4-ol.

¹H-NMR (DMSO-d₆) δ: 1.50-1.80 (4H, m), 1.90-2.10 (2H, m), 2.71 (3H, s), 2.73 (3H, s), 2.80-3.40 (8H, m), 7.34 (1H, d, J=8.4 Hz), 7.60 (1H, s), 7.63 (1H, d, J=8.4 Hz), 8.07 (1H, s), 8.20-8.25 (1H, m), 10.45 (1H, brs), 11.37 (1H, s).

### Example 25

### N-(2-piperidin-1-ylethyl)-4,5,6,11-tetrahydro-1H-pyrazolo[4′,3′:6,7]cyclohepta[1,2-b]indole-8-carboxamide hydrochloride

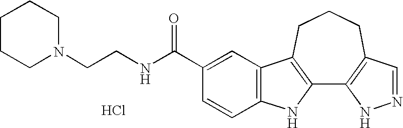

The title compound was obtained by treating, with hydrochloric acid, a compound obtained in the same manner as in Example 6 and using (2-piperidin-1-ylethyl)amine instead of piperidin-4-ol.

¹H-NMR (DMSO-d₆) δ: 1.30-2.10 (8H, m), 2.80-3.80 (12H, m), 7.35 (1H, d, J=8.4 Hz), 7.57 (1H, s), 7.67 (1H, d, J=8.4 Hz), 8.16 (1H, s), 8.70-8.80 (1H, m), 10.30 (1H, brs).

### Example 26

### N-(2-morpholin-4-ylethyl)-4,5,6,11-tetrahydro-1H-pyrazolo[4′,3′:6,7]cyclohepta[1,2-b]indole-8-carboxamide hydrochloride

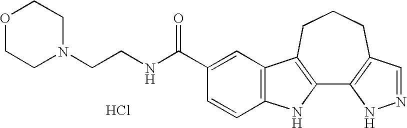

The title compound was obtained by treating, with hydrochloric acid, a compound obtained in the same manner as in Example 6 and using (2-morpholin-4-ylethyl)amine instead of piperidin-4-ol.

¹H-NMR (DMSO-d₆) δ: 1.95-2.10 (2H, m), 2.80-4.05 (16H, m), 7.35 (1H, d, J=8.4 Hz), 7.57 (1H, s), 7.68 (1H, dd, J=1.2, 8.4 Hz), 8.16 (1H, d, J=1.2 Hz), 8.70-8.80 (1H, m), 11.00 (1H, brs).

### Example 27

### N-(3-morpholin-4-ylpropyl)-4,5,6,11-tetrahydro-1H-pyrazolo[4′,3′:6,7]cyclohepta[1,2-b]indole-8-carboxamide hydrochloride

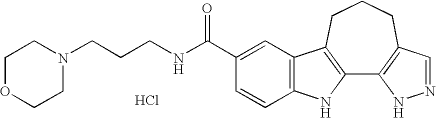

The title compound was obtained by treating, with hydrochloric acid, a compound obtained in the same manner as in Example 6 and using (3-morpholin-4-ylpropyl)amine instead of piperidin-4-ol.

¹H-NMR (DMSO-d₆) δ: 1.90-2.10 (4H, m), 2.80-3.50 (12H, m), 3.75-4.00 (4H, m), 7.35 (1H, d, J=8.7 Hz), 7.61 (1H, s), 7.65 (1H, d, J=8.7 Hz), 8.09 (1H, s), 8.50-8.70 (1H, m), 11.12 (1H, brs), 11.39 (1H, s).

### Example 28

### N-(pyridin-2-ylmethyl)-4,5,6,11-tetrahydro-1H-pyrazolo[4′,3′:6,7]cyclohepta[1,2-b]indole-8-carboxamide hydrochloride

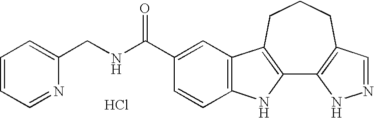

The title compound was obtained by treating, with hydrochloric acid, a compound obtained in the same manner as in Example 6 and using (pyridin-2-ylmethyl)amine instead of piperidin-4-ol.

¹H-NMR (DMSO-d₆) δ: 1.95-2.10 (2H, m), 2.85-2.95 (2H, m), 3.05-3.20 (2H, m), 4.83 (2H, d, J=5.7 Hz), 7.38 (1H, d, J=8.4 Hz), 7.56 (1H, s), 7.65-8.00 (3H, m), 8.15-8.20 (1H, m), 8.40-9.40 (3H, m).

### Example 29

### N-(pyridin-3-ylmethyl)-4,5,6,11-tetrahydro-1H-pyrazolo[4′,3′:6,7]cyclohepta[1,2-b]indole-8-carboxamide hydrochloride

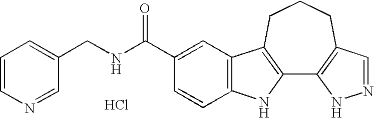

The title compound was obtained by treating, with hydrochloric acid, a compound obtained in the same manner as in Example 6 and using (pyridin-3-ylmethyl)amine instead of piperidin-4-ol.

¹H-NMR (DMSO-d₆) δ: 1.95-2.10 (2H, m), 2.85-2.95 (2H, m), 3.05-3.20 (2H, m), 4.65 (2H, d, J=5.7 Hz), 7.10-7.20 (1H, m), 7.35 (1H, d, J=8.4 Hz), 7.54 (1H, s), 7.85-7.95 (1H, m), 8.12 (1H, s), 8.30-8.45 (1H, m), 8.70-9.20 (3H, m).

### Example 30

### N-(pyridin-4-ylmethyl)-4,5,6,11-tetrahydro-1H-pyrazolo[4′,3′:6,7]cyclohepta[1,2-b]indole-8-carboxamide hydrochloride

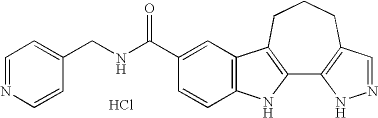

The title compound was obtained by treating, with hydrochloric acid, a compound obtained in the same manner as in Example 6 and using (pyridin-4-ylmethyl)amine instead of piperidin-4-ol.

¹H-NMR (DMSO-d₆) δ: 1.95-2.10 (2H, m), 2.80-2.95 (2H, m), 3.05-3.20 (2H, m), 4.76 (2H, d, J=5.4 Hz), 7.15-7.25 (1H, m), 7.39 (1H, d, J=8.4 Hz), 7.58 (1H, s), 8.00 (2H, d, J=6.6 Hz), 8.18 (1H, s), 8.87 (2H, d, J=6.6 Hz), 9.35-9.45 (1H, m).

### Example 31

### 10-methyl-N-(3-morpholin-4-ylpropyl)-4,5,6,11-tetrahydro-1H-pyrazolo[4′,3′:6,7]cyclohepta[1,2-b]indole-7-carboxamide fumarate

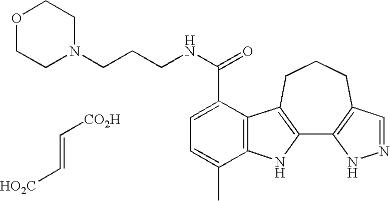

The title compound was obtained by treating, with fumaric acid, a compound obtained in the same manner as in Example 6 and using 10-methyl-4,5,6,11-tetrahydro-1H-pyrazolo[4′,3′:6,7]cyclohepta[1,2-b]indole-7-carboxylic acid instead of 4,5,6,11-tetrahydro-1H-pyrazolo[4′,3′:6,7]cyclohepta[1,2-b]indole-8-carboxylic acid and (3-morpholin-4-ylpropyl)amine instead of piperidin-4-ol.

¹H-NMR (DMSO-d₆) δ: 1.60-3.70 (20H, m), 2.41 (3H, s), 6.60 (2H, s), 6.80-6.90 (2H, m), 7.48 (1H, s), 8.20-8.30 (1H, m), 10.71 (1H, s).

### Example 32

### ethyl 1,4,5,6,7,12-hexahydropyrazolo[4′,3′:7,8]cycloocta[1,2-b]indole-9-carboxylate

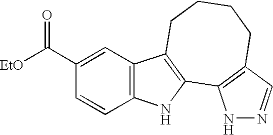

In the same manner as in Example 1 except that ethyl 7-[(dimethylamino)methylene]-6-oxo-6,7,8,9,10,11-hexahydro-5H-cycloocta[b]indole-2-carboxylate was used instead of 7-[(dimethylamino)methylene]-7,8,9,10-tetrahydrocyclohepta[b]indol-6(5H)-one, the title compound was obtained.

¹H-NMR (CDCl₃) δ: 1.42 (3H, t, J=7.2 Hz), 1.75-2.00 (4H, m), 2.78 (2H, t, J=5.7 Hz), 3.07 (2H, t, J=5.4 Hz), 4.40 (2H, q, J=7.2 Hz), 7.27 (1H, d, J=7.8 Hz), 7.38 (1H, s), 7.88 (1H, d, J=7.8 Hz), 8.33 (1H, s), 8.94 (1H, brs).

### Example 33

### 1,4,5,6,7,12-hexahydropyrazolo[4′,3′:7,8]cycloocta[1,2-b]indole-9-carboxylic acid

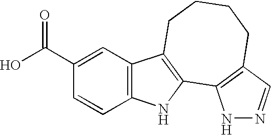

In the same manner as in Example 3 except that ethyl 1,4,5,6,7,12-hexahydropyrazolo[4′,3′:7,8]cycloocta[1,2-b]indole-9-carboxylate was used instead of ethyl 4,5,6,11-tetrahydro-1H-pyrazolo[4′,3′:6,7]cyclohepta[1,2-b]indole-8-carboxylate, the title compound was obtained.

¹H-NMR (5% CD₃OD-containing CDCl₃) δ: 1.90-2.00 (4H, m), 2.75-2.90 (2H, m), 3.05-3.15 (2H, m), 7.45 (1H, d, J=8.4 Hz), 7.58 (1H, s), 7.96 (1H, d, J=8.4 Hz), 8.38 (1H, s).

### Example 34

### 9-[(4-pyrrolidin-1-ylpiperidin-1-yl)carbonyl]-1,4,5,6,7,12-hexahydropyrazolo[4′,3′:7,8]cycloocta[1,2-b]indole hydrochloride

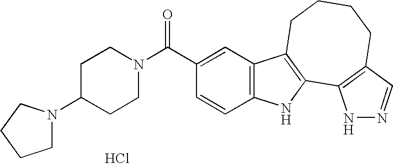

The title compound was obtained by treating, with hydrochloric acid, a compound obtained in the same manner as in Example 6 and using 1,4,5,6,7,12-hexahydropyrazolo[4′,3′:7,8]cycloocta[1,2-b]indole-9-carboxylic acid instead of 4,5,6,11-tetrahydro-1H-pyrazolo[4′,3′:6,7]cyclohepta[1,2-b]indole-8-carboxylic acid and 4-pyrrolidin-1-ylpiperidine instead of piperidin-4-ol.

¹H-NMR (DMSO-d₆) δ: 1.60-3.60 (25H, m), 7.12 (1H, d, J=6.9 Hz), 7.37 (1H, d, J=6.9 Hz), 7.54 (1H, s), 7.59 (1H, s), 11.00 (1H, s), 11.19 (1H, s).

### Example 35

### 2-[1-[[3-methyl-2-(1H-pyrazol-5-yl)-1H-indol-5-yl]carbonyl]piperidin-4-yl]-1,2,3,4-tetrahydroisoquinoline

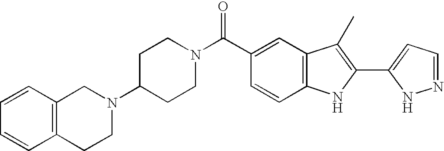

N,N-Dimethylformamide dimethyl acetal (15 ml) was added to methyl 2-acetyl-3-methyl-1H-indole-5-carboxylate (3.0 g), and the mixture was stirred at 80° C. for 16 hrs. After allowing to cool, the precipitate was collected by filtration and washed with a mixed solution of hexane and ethyl acetate. To the obtained precipitate were added methanol (54 ml), acetic acid (0.81 ml) and hydrazine monohydrate (0.69 ml), and the mixture was stirred at room temperature for 24 hrs. The reaction mixture was extracted with a mixed solution of ethyl acetate and tetrahydrofuran. The extract washed with a saturated aqueous sodium hydrogen carbonate solution and saturated brine, dried over magnesium sulfate, filtered and concentrated. The residue washed with a mixed solution of hexane and ethyl acetate to give methyl 3-methyl-2-(1H-pyrazol-5-yl)-1H-indole-5-carboxylate (2.2 g).

To a mixed solution of methyl 3-methyl-2-(1H-pyrazol-5-yl)-1H-indole-5-carboxylate (1.05 g) in tetrahydrofuran (10.5 ml) and methanol (10.5 ml) was added 3N aqueous sodium hydroxide solution (10.5 ml), and the mixture was stirred at 50° C. for 6 hrs. After allowing to cool, pH was adjusted to about 3 with 2N hydrochloric acid, and the mixture was extracted with a mixed solution of ethyl acetate and tetrahydrofuran. The extract washed with saturated brine, dried over magnesium sulfate, filtered and concentrated. The solvent was removed by under reduced pressure, and the residue washed with a mixed solution of hexane and ethyl acetate to give 3-methyl-2-(1H-pyrazol-5-yl)-1H-indole-5-carboxylic acid (0.80 g).

In the same manner as in Example 6 except that 2-piperidin-4-yl-1,2,3,4-tetrahydroisoquinoline dihydrochloride was used instead of piperidin-4-ol and 3-methyl-2-(1H-pyrazol-5-yl)-1H-indole-5-carboxylic acid was used instead of 4,5,6,11-tetrahydro-1H-pyrazolo[4′,3′:6,7]cyclohepta[1,2-b]indole-8-carboxylic acid, the title compound was obtained.

¹H-NMR (DMSO-d₆) δ: 1.42-1.63 (2H, m), 1.76-1.91 (2H, m), 2.38-2.47 (2H, m), 2.50 (3H, s), 2.65-2.80 (5H, m), 2.83-3.06 (2H, m), 3.71 (2H, s), 6.70 (1H, s), 6.99-7.14 (5H, m), 7.35 (1H, d, J=8.4 Hz), 7.56 (1H, s), 7.86 (1H, s), 11.26 (1H, s), 13.05 (1H, brs).

### Example 36

### 2-[1-[[3-propyl-2-(1H-pyrazol-5-yl)-1H-indol-5-yl]carbonyl]piperidin-4-yl]-1,2,3,4-tetrahydroisoquinoline

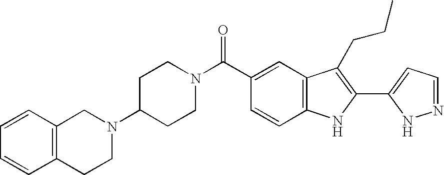

The title compound was obtained by a method similar to that in Example 35 and using methyl 2-acetyl-3-propyl-1H-indole-5-carboxylate instead of methyl 2-acetyl-3-methyl-1H-indole-5-carboxylate.

¹H-NMR (DMSO-d₆) δ: 0.95 (3H, t, J=7.5 Hz), 1.45-1.70 (4H, m), 1.77-1.92 (2H, m), 2.48-2.51 (2H, m), 2.65-2.80 (5H, m), 2.98 (2H, t, J=7.2 Hz), 3.71 (2H, s), 6.68 (1H, s), 7.01-7.13 (5H, m), 7.36 (1H, d, J=8.1 Hz), 7.57 (1H, s), 7.85 (1H, s), 11.26 (1H, s), 13.05 (1H, brs).

### Example 37

### tert-butyl 4-(4,5,6,11-tetrahydro-1H-pyrazolo[4′,3′:6,7]cyclohepta[1,2-b]indol-8-ylcarbonyl)piperazine-1-carboxylate

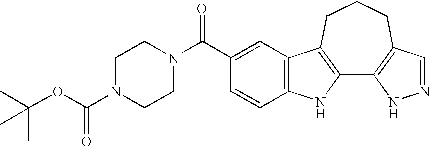

The title compound was obtained by a method similar to that in Example 6 and using tert-butyl piperazine-1-carboxylate instead of piperidin-4-ol.

¹H-NMR (CDCl₃) δ: 1.48 (9H, m), 2.00-2.20 (2H, m), 2.85-3.00 (2H, m), 3.05-3.20 (2H, m), 3.48 (4H, brs), 3.65 (4H, brs), 7.15-7.35 (2H, m), 7.35 (1H, s), 7.65 (1H, s), 9.02 (1H, brs), 10.04 (1H, brs).

### Example 38

### 8-(piperazin-1-ylcarbonyl)-4,5,6,11-tetrahydro-1H-pyrazolo[4′,3′:6,7]cyclohepta[1,2-b]indole hydrochloride

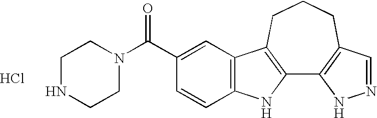

A mixture of tert-butyl 4-(4,5,6,11-tetrahydro-1H-pyrazolo[4′,3′:6,7]cyclohepta[1,2-b]indol-8-ylcarbonyl)piperazine-1-carboxylate (246 mg), methanol (10 ml) and a solution (10 ml) of 4N hydrogen chloride in ethyl acetate was stirred at room temperature for 4 hrs. The reaction mixture was concentrated, and the residue was collected by filtration and washed with ethyl acetate to give the title compound.

¹H-NMR (CD₃OD) δ: 2.05-2.20 (2H, m), 2.95-3.05 (2H, m), 3.15-3.40 (6H, m), 3.85-4.00 (4H, m), 7.37 (1H, dd, J=1.5, 8.4 Hz), 7.54 (1H, d, J=8.4 Hz), 7.75-7.80 (1H, m), 7.92 (1H, s).

### Example 39

### N-(7-chloro-1,4,5,10-tetrahydropyrazolo[3,4-a]carbazol-6-yl)nicotinamide

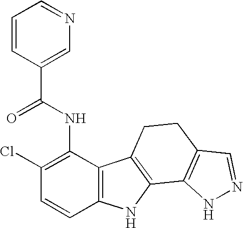

To a solution of 5-amino-6-chloro-2,3,4,9-tetrahydro-1H-carbazol-1-one (117 mg) in pyridine (4.5 ml) was added nicotinoyl chloride hydrochloride (105 mg) at 0° C., and the mixture was stirred at room temperature for 16 hrs. The reaction mixture was concentrated and extracted with ethyl acetate. The extract washed with saturated aqueous sodium hydrogen carbonate solution, dried over anhydrous sodium sulfate, filtered and concentrated. To a solution of the residue in dimethylformamide (2 ml) was added tris(dimethylamino)methane (209 mg), and the mixture was stirred at 70° C. for 15 hrs and concentrated. To the residue were added ethanol (3 ml) and hydrazine monohydrate (72 mg), and the mixture was heated under reflux for 2 hrs. The reaction mixture was concentrated, and the residue was recrystallized (ethanol) to give the title compound (77 mg).

¹H-NMR (DMSO-d₆) δ: 2.55-3.20 (4H, m), 7.16 (1H, d, J=8.4 Hz), 7.34 (1H, d, J=8.4 Hz), 7.51 (1H, s), 7.61 (1H, dd, J=8.2, 4.8 Hz), 8.39 (1H, d, J=8.2 Hz), 8.80 (1H, d, J=4.8 Hz), 9.20 (1H, s), 10.48 (1H, brs).

### Example 40

### 4-phenyl-1-(4,5,6,11-tetrahydro-1H-pyrazolo[4′,3′:6,7]cyclohepta[1,2-b]indol-8-ylcarbonyl)piperidin-4-ol

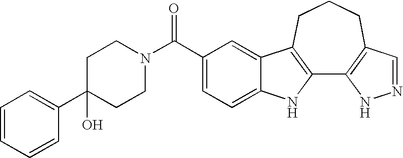

A mixture of 4,5,6,11-tetrahydro-1H-pyrazolo[4′,3′:6,7]cyclohepta[1,2-b]indole-8-carboxylic acid (801 mg), 4-phenylpiperidin-4-ol (532 mg), triethylamine (2.1 ml), 1H-1,2,3-benzotriazol-1-ol (486 mg), N-[3-(dimethylamino)propyl]-N′-ethylcarbodiimide hydrochloride (690 mg) and dimethylformamide (9 ml) was stirred at room temperature for 18 hrs. To the reaction mixture was added a saturated aqueous sodium hydrogen carbonate solution, and the mixture was extracted with ethyl acetate. The extract was washed with saturated brine, dried over anhydrous sodium sulfate, filtered and concentrated. The residue was purified by silica gel column chromatography (eluted with ethyl acetate) and recrystallized (a mixed solution of ethyl acetate and hexane) to give the title compound (751 mg).

¹H-NMR (CDCl₃) δ: 1.20-2.20 (8H, m), 2.85-2.95 (2H, m), 3.05-3.15 (2H, m), 3.30-3.65 (2H, m), 7.10-7.55 (8H, m), 7.66 (1H, s), 9.34 (1H, brs).

### Example 41

### 4-(4-fluorophenyl)-1-(4,5,6,11-tetrahydro-1H-pyrazolo[4′,3′:6,7]cyclohepta[1,2-b]indol-8-ylcarbonyl)piperidin-4-ol

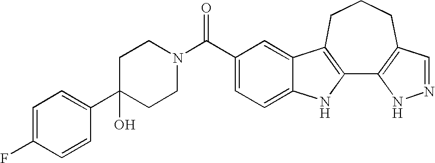

The title compound was synthesized by a method similar to that in Example 40 and using 4-(4-fluorophenyl)piperidin-4-ol instead of 4-phenylpiperidin-4-ol.

¹H-NMR (DMSO-d₆) δ: 1.50-2.10 (6H, m), 2.80-3.50 (8H, m), 7.05-7.65 (8H, m), 11.17 (1H, s), 12.68 (1H, s).

### Example 42

### 4-(3-fluorophenyl)-1-(4,5,6,11-tetrahydro-1H-pyrazolo[4′,3′:6,7]cyclohepta[1,2-b]indol-8-ylcarbonyl)piperidin-4-ol

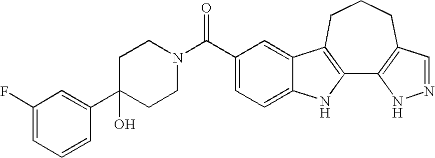

The title compound was synthesized by a method similar to that in Example 40 and using 4-(3-fluorophenyl)piperidin-4-ol instead of 4-phenylpiperidin-4-ol.

¹H-NMR (DMSO-d₆) δ: 1.50-2.10 (6H, m), 2.80-3.50 (8H, m), 5.30 (1H, s), 7.00-7.60 (8H, m), 11.17 (1H, s), 12.68 (1H, s).

### Example 43

### 4-(2-fluorophenyl)-1-(4,5,6,11-tetrahydro-1H-pyrazolo[4′,3′:6,7]cyclohepta[1,2-b]indol-8-ylcarbonyl)piperidin-4-ol

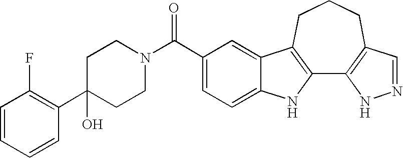

The title compound was synthesized by a method similar to that in Example 40 and using 4-(2-fluorophenyl)piperidin-4-ol instead of 4-phenylpiperidin-4-ol. 4-(2-Fluorophenyl)piperidin-4-ol was synthesized from tert-butyl 4-(2-fluorophenyl)-4-hydroxypiperidine-1-carboxylate by a method similar to that in Reference Example 20.

¹H-NMR (DMSO-d₆) δ: 1.50-1.71 (2H, m), 1.81-2.07 (4H, m), 2.80-2.89 (2H, m), 3.03-3.10 (2H, m), 3.12-3.41 (2H, m), 3.54-4.51 (2H, m), 5.15 (1H, s), 6.78-6.82 (1H, m), 7.08-7.37 (5H, m), 7.48-7.57 (2H, m), 11.19 (1H, s), 12.70 (1H, s).

### Example 44

### 4-(4-chlorophenyl)-1-(4,5,6,11-tetrahydro-1H-pyrazolo[4′,3′:6,7]cyclohepta[1,2-b]indol-8-ylcarbonyl)piperidin-4-ol

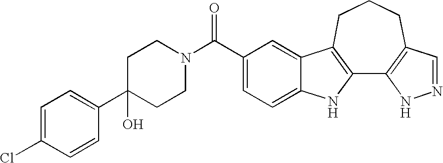

The title compound was synthesized by a method similar to that in Example 40 and using 4-(4-chlorophenyl)piperidin-4-ol instead of 4-phenylpiperidin-4-ol.

¹H-NMR (DMSO-d₆) δ: 1.50-2.10 (6H, m), 2.80-3.50 (8H, m), 5.26 (1H, s), 7.10-7.65 (8H, m), 11.17 (1H, s), 12.68 (1H, s).

### Example 45

### 4-(3-chlorophenyl)-1-(4,5,6,11-tetrahydro-1H-pyrazolo[4′,3′:6,7]cyclohepta[1,2-b]indol-8-ylcarbonyl)piperidin-4-ol

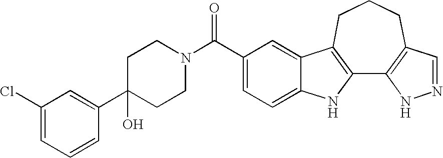

The title compound was synthesized by a method similar to that in Example 40 and using 4-(3-chlorophenyl)piperidin-4-ol instead of 4-phenylpiperidin-4-ol. 4-(3-Chlorophenyl)piperidin-4-ol was synthesized from tert-butyl 4-(3-chlorophenyl)-4-hydroxypiperidine-1-carboxylate by a method similar to that in Reference Example 20.

¹H-NMR (DMSO-d₆) δ: 1.52-1.71 (2H, m), 1.86-2.04 (4H, m), 2.81-2.89 (2H, m), 3.03-3.10 (2H, m), 3.12-3.41 (2H, m), 3.54-4.51 (2H, m), 5.32 (1H, s), 7.10-7.19 (1H, m), 7.21-7.40 (3H, m), 7.48-7.62 (4H, m), 11.19 (1H, s), 12.70 (1H, s).

### Example 46

### 4-(2-chlorophenyl)-1-(4,5,6,11-tetrahydro-1H-pyrazolo[4′,3′:6,7]cyclohepta[1,2-b]indol-8-ylcarbonyl)piperidin-4-ol

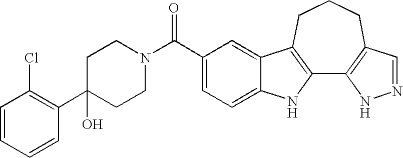

The title compound was synthesized by a method similar to that in Example 40 and using 4-(2-chlorophenyl)piperidin-4-ol instead of 4-phenylpiperidin-4-ol. 4-(2-Chlorophenyl)piperidin-4-ol was synthesized from tert-butyl 4-(2-chlorophenyl)-4-hydroxypiperidine-1-carboxylate by a method similar to that in Reference Example 20.

¹H-NMR (DMSO-d₆) δ: 1.48-1.71 (2H, m), 1.60-2.07 (4H, m), 2.80-2.89 (2H, m), 3.03-3.10 (2H, m), 3.12-3.41 (2H, m), 3.54-4.51 (2H, m), 5.08 (1H, s), 6.78-6.82 (1H, m), 7.05-7.58 (7H, m), 11.18 (1H, s), 12.70 (1H, s).

### Example 47

### 1-(4,5,6,11-tetrahydro-1H-pyrazolo[4′,3′:6,7]cyclohepta[1,2-b]indol-8-ylcarbonyl)-4-[4-(trifluoromethyl)phenyl]piperidin-4-ol

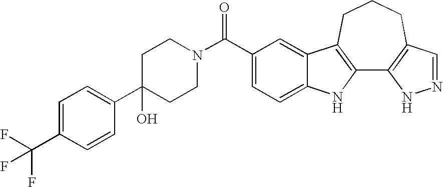

The title compound was synthesized by a method similar to that in Example 40 and using 4-[4-(trifluoromethyl)phenyl]piperidin-4-ol instead of 4-phenylpiperidin-4-ol.

¹H-NMR (DMSO-d₆) δ: 1.60-2.10 (6H, m), 2.70-3.50 (8H, m), 7.10-7.40 (2H, m), 7.50-7.85 (6H, m).

### Example 48

### 1-(4,5,6,11-tetrahydro-1H-pyrazolo[4′,3′:6,7]cyclohepta[1,2-b]indol-8-ylcarbonyl)-4-[3-(trifluoromethyl)phenyl]piperidin-4-ol

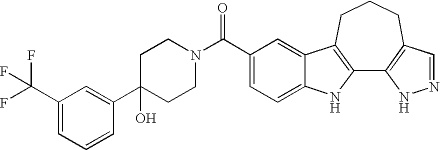

The title compound was synthesized by a method similar to that in Example 40 and using 4-[3-(trifluoromethyl)phenyl]piperidin-4-ol instead of 4-phenylpiperidin-4-ol.

¹H-NMR (DMSO-d₆) δ: 1.50-1.80 (2H, m), 1.85-2.20 (4H, m), 2.70-4.60 (8H, m), 5.42 (1H, s), 7.10-8.00 (8H, m), 11.19 (1H, s), 2.69 (1H, s).

### Example 49

### 1-(4,5,6,11-tetrahydro-1H-pyrazolo[4′,3′:6,7]cyclohepta[1,2-b]indol-8-ylcarbonyl)-4-[2-(trifluoromethyl)phenyl]piperidin-4-ol

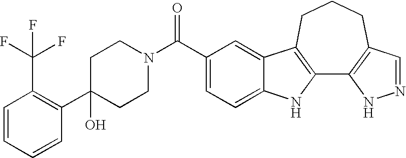

The title compound was synthesized by a method similar to that in Example 40 and using 4-[2-(trifluoromethyl)phenyl]piperidin-4-ol instead of 4-phenylpiperidin-4-ol. 4-[(2-Trifluoromethyl)phenyl]piperidin-4-ol was synthesized from tert-butyl 4-hydroxy-4-[2-(trifluoromethyl)phenylpiperidine-1-carboxylate by a method similar to that in Reference Example 20.

¹H-NMR (DMSO-d₆) δ: 1.67-1.90 (2H, m), 1.90-2.18 (4H, m), 2.81-2.89 (2H, m), 3.03-3.08 (2H, m), 3.12-3.41 (2H, m), 3.51-4.51 (2H, m), 5.22 (1H, s), 7.11-7.18 (1H, m), 7.34-7.79 (5H, m), 7.56-7.79 (2H, m), 11.19 (1H, s), 12.69 (1H, s).

### Example 50

### 1-(4,5,6,11-tetrahydro-1H-pyrazolo[4′,3′:6,7]cyclohepta[1,2-b]indol-8-ylcarbonyl)-4-[4-(trifluoromethoxy)phenyl]piperidin-4-ol

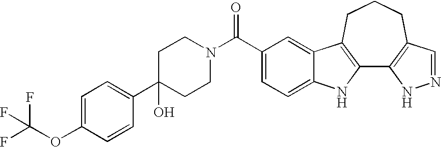

The title compound was synthesized by a method similar to that in Example 40 and using 4-[4-(trifluoromethoxy)phenyl]piperidin-4-ol instead of 4-phenylpiperidin-4-ol.

¹H-NMR (DMSO-d₆) δ: 1.50-1.70 (2H, m), 1.85-2.10 (4H, m), 2.80-3.60 (8H, m), 5.30 (1H, s), 7.10-7.70 (8H, m), 11.17 (1H, s), 12.68 (1H, s).

### Example 51

### 4-[4-hydroxy-1-(4,5,6,11-tetrahydro-1H-pyrazolo[4′,3′:6,7]cyclohepta[1,2-b]indol-8-ylcarbonyl)piperidin-4-yl]benzonitrile

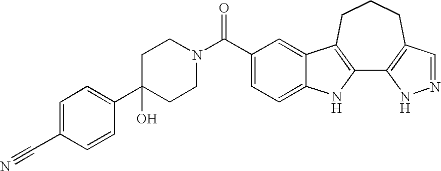

The title compound was synthesized by a method similar to that in Example 40 and using 4-(4-hydroxypiperidin-4-yl)benzonitrile instead of 4-phenylpiperidin-4-ol. 4-(4-Hydroxypiperidin-4-yl)benzonitrile was synthesized from tert-butyl 4-(4-cyanophenyl)-4-hydroxypiperidine-1-carboxylate by a method similar to that in Reference Example 20.

¹H-NMR (DMSO-d₆) δ: 1.53-1.73 (2H, m), 1.88-2.08 (4H, m), 2.81-2.89 (2H, m), 3.03-3.12 (2H, s), 3.13-3.40 (2H, m), 3.53-4.54 (2H, m), 5.45 (1H, s), 7.15-7.18 (1H, m), 7.34-7.37 (1H, m), 7.53-7.58 (2H, m), 7.74-7.83 (4H, m), 11.19 (1H, s), 12.70 (1H, s).

### Example 52

### 3-[4-hydroxy-1-(4,5,6,11-tetrahydro-1H-pyrazolo[4′,3′:6,7]cyclohepta[1,2-b]indol-8-ylcarbonyl)piperidin-4-yl]benzonitrile

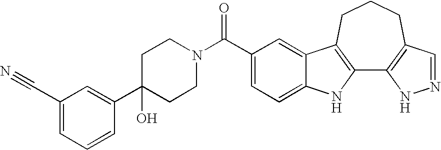

The title compound was synthesized by a method similar to that in Example 40 and using 3-(4-hydroxypiperidin-4-yl)benzonitrile instead of 4-phenylpiperidin-4-ol. 3-(4-Hydroxypiperidin-4-yl)benzonitrile was synthesized from tert-butyl 4-(3-cyanophenyl)-4-hydroxypiperidine-1-carboxylate by a method similar to that in Reference Example 20.

¹H-NMR (DMSO-d₆) δ: 1.42-1.56 (2H, m), 1.85-2.11 (4H, m), 2.81-2.89 (2H, m), 3.02-3.08 (2H, s), 3.44-3.59 (2H, m), 3.53-4.54 (2H, m), 5.42 (1H, s), 7.02-7.11 (1H, m), 7.34-7.37 (1H, m), 7.52-7.63 (2H, m), 7.70-7.97 (4H, m), 11.19 (1H, s), 12.70 (1H, s).

### Example 53

### 4-(3-methoxyphenyl)-1-(4,5,6,11-tetrahydro-1H-pyrazolo[4′,3′:6,7]cyclohepta[1,2-b]indol-8-ylcarbonyl)piperidin-4-ol

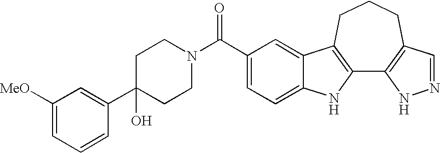

The title compound was synthesized by a method similar to that in Example 40 and using 4-(3-methoxyphenyl)piperidin-4-ol instead of 4-phenylpiperidin-4-ol. 4-(3-Methoxyphenyl)piperidin-4-ol was synthesized from tert-butyl 4-hydroxy-4-(3-methoxyphenyl)piperidine-1-carboxylate by a method similar to that in Reference Example 20.

¹H-NMR (DMSO-d₆) δ: 1.52-1.71 (2H, m), 1.86-2.03 (4H, m), 2.82-2.89 (2H, m), 3.03-3.08 (2H, m), 3.12-3.41 (2H, m), 3.76 (3H, s), 3.97-4.51 (2H, m), 5.15 (1H, s), 6.78-6.81 (1H, m), 7.07-7.37 (5H, m), 7.51-7.58 (2H, m), 11.19 (1H, s), 12.70 (1H, s).

### Example 54

### 4-(3,4-difluorophenyl)-1-(4,5,6,11-tetrahydro-1H-pyrazolo[4′,3′:6,7]cyclohepta[1,2-b]indol-8-ylcarbonyl)piperidin-4-ol

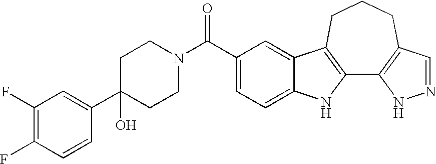

The title compound was synthesized by a method similar to that in Example 40 and using 4-(3,4-difluorophenyl)piperidin-4-ol instead of 4-phenylpiperidin-4-ol. 4-(3,4-Difluorophenyl)piperidin-4-ol was synthesized from tert-butyl 4-(3,4-difluorophenyl)-4-hydroxypiperidine-1-carboxylate by a method similar to that in Reference Example 20.

¹H-NMR (DMSO-d₆) δ: 1.52-1.69 (2H, m), 1.90-2.08 (4H, m), 2.80-2.89 (2H, m), 3.01-3.10 (2H, m), 3.12-3.41 (2H, m), 3.67-4.51 (2H, m), 5.35 (1H, s), 7.11-7.18 (1H, m), 7.33-7.39 (4H, m), 7.53-7.62 (2H, m), 11.19 (1H, s), 12.70 (1H, s).

### Example 55

### 4-benzyl-1-(4,5,6,11-tetrahydro-1H-pyrazolo[4′,3′:6,7]cyclohepta[1,2-b]indol-8-ylcarbonyl)piperidin-4-ol

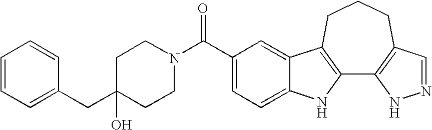

The title compound was synthesized by a method similar to that in Example 40 and using 4-benzylpiperidin-4-ol instead of 4-phenylpiperidin-4-ol.

¹H-NMR (DMSO-d₆) δ: 1.20-1.60 (4H, m), 1.90-2.10 (2H, m), 2.72 (2H, s), 2.80-3.40 (8H, m), 4.46 (1H, s), 7.00-7.40 (7H, m), 7.44 (1H, s), 7.55 (1H, s), 11.18 (1H, s).

### Example 56

### 4-(4-fluorobenzyl)-1-(4,5,6,11-tetrahydro-1H-pyrazolo[4′,3′:6,7]cyclohepta[1,2-b]indol-8-ylcarbonyl)piperidin-4-ol

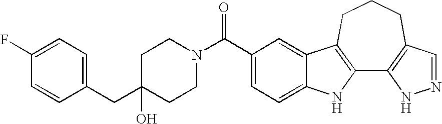

The title compound was synthesized by a method similar to that in Example 40 and using 4-(4-fluorobenzyl)piperidin-4-ol instead of 4-phenylpiperidin-4-ol.

¹H-NMR (DMSO-d₆) δ: 1.30-2.20 (6H, m), 2.60-3.40 (10H, m), 7.00-7.40 (6H, m), 7.43 (1H, s), 7.54 (1H, s), 11.18 (1H, s), 12.68 (1H, s).

### Example 57

### 4-(3-fluorobenzyl)-1-(4,5,6,11-tetrahydro-1H-pyrazolo[4′,3′:6,7]cyclohepta[1,2-b]indol-8-ylcarbonyl)piperidin-4-ol

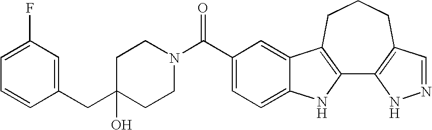

The title compound was synthesized by a method similar to that in Example 40 and using 4-(3-fluorobenzyl)piperidin-4-ol instead of 4-phenylpiperidin-4-ol.

¹H-NMR (DMSO-d₆) δ: 1.38-1.63 (4H, m), 1.93-2.04 (2H, m), 2.76 (2H, s), 2.82-2.87 (2H, m), 3.00-3.05 (2H, m), 3.12-3.28 (2H, m), 3.50-4.30 (2H, m), 4.56 (1H, s), 7.02-7.14 (3H, m), 7.20-7.35 (3H, m), 7.42 (1H, s), 7.54 (1H, s), 11.17 (1H, s), 12.68 (1H, s).

### Example 58

### 4-(2-fluorobenzyl)-1-(4,5,6,11-tetrahydro-1H-pyrazolo[4′,3′:6,7]cyclohepta[1,2-b]indol-8-ylcarbonyl)piperidin-4-ol

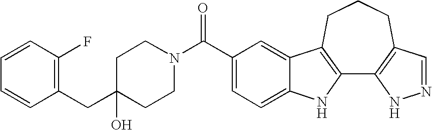

The title compound was synthesized by a method similar to that in Example 40 and using 4-(2-fluorobenzyl)piperidin-4-ol instead of 4-phenylpiperidin-4-ol.

¹H-NMR (DMSO-d₆) δ: 1.38-1.63 (4H, m), 1.93-2.04 (2H, m), 2.76 (2H, s), 2.82-2.87 (2H, m), 3.00-3.05 (2H, m), 3.12-3.28 (2H, m), 3.50-4.30 (2H, m), 4.56 (1H, s), 7.02-7.14 (3H, m), 7.20-7.35 (3H, m), 7.42 (1H, s), 7.54 (1H, s), 11.17 (1H, s), 12.68 (1H, s).

### Example 59

### 4-(4-chlorobenzyl)-1-(4,5,6,11-tetrahydro-1H-pyrazolo[4′,3′:6,7]cyclohepta[1,2-b]indol-8-ylcarbonyl)piperidin-4-ol

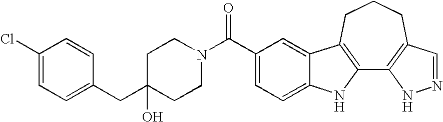

The title compound was synthesized by a method similar to that in Example 40 and using 4-(4-chlorobenzyl)piperidin-4-ol instead of 4-phenylpiperidin-4-ol.

¹H-NMR (DMSO-d₆) δ: 1.30-2.10 (6H, m), 2.65-3.40 (10H, m), 7.00-7.40 (6H, m), 7.43 (1H, s), 7.54 (1H, s), 11.17 (1H, s), 12.68 (1H, s).

### Example 60

### 4-(3,4-difluorobenzyl)-1-(4,5,6,11-tetrahydro-1H-pyrazolo[4′,3′:6,7]cyclohepta[1,2-b]indol-8-ylcarbonyl)piperidin-4-ol

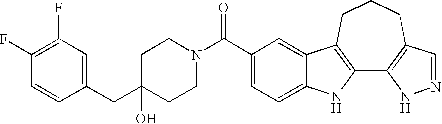

The title compound was synthesized by a method similar to that in Example 40 and using 4-(3,4-difluorobenzyl)piperidin-4-ol instead of 4-phenylpiperidin-4-ol.

¹H-NMR (DMSO-d₆) δ: 1.35-1.61 (4H, m), 1.96-2.03 (2H, m), 2.71 (2H, s), 2.83-2.88 (2H, m), 3.02-3.07 (2H, m), 3.16-3.31 (2H, m), 3.60-4.20 (2H, m), 4.53 (1H, s), 7.05-7.09 (2H, m), 7.23-7.36 (3H, m), 7.45 (1H, s), 7.55 (1H, s), 11.19 (1H, s), 12.70 (1H, s).

### Example 61

### 1-(4,5,6,11-tetrahydro-1H-pyrazolo[4′,3′:6,7]cyclohepta[1,2-b]indol-8-ylcarbonyl)-4-[3-(trifluoromethyl)benzyl]piperidin-4-ol

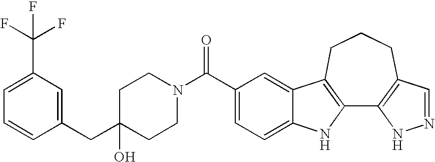

The title compound was synthesized by a method similar to that in Example 40 and using 4-[3-(trifluoromethyl)benzyl]piperidin-4-ol instead of 4-phenylpiperidin-4-ol.

¹H-NMR (DMSO-d₆) δ: 1.28-1.44 (2H, m), 1.48-1.59 (2H, m), 1.96-2.03 (2H, m), 2.82 (2H, s), 2.82-2.88 (2H, m), 3.01-3.04 (2H, m), 3.11-3.31 (2H, m), 3.60-4.20 (2H, m), 4.59 (1H, s), 7.06 (1H, d, J=8.1 Hz), 7.33 (1H, d, J=8.1 Hz), 7.44-7.60 (6H, m), 11.18 (1H, s), 12.69 (1H, s).

### Example 62

### 4-(phenylethynyl)-1-(4,5,6,11-tetrahydro-1H-pyrazolo[4′,3′:6,7]cyclohepta[1,2-b]indol-8-ylcarbonyl)piperidin-4-ol

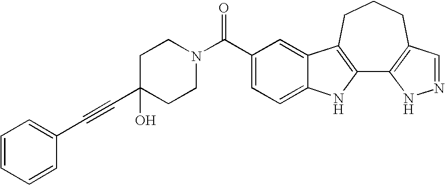

The title compound was synthesized by a method similar to that in Example 40 and using 4-(phenylethynyl)piperidin-4-ol instead of 4-phenylpiperidin-4-ol.

¹H-NMR (DMSO-d₆) δ: 1.62-1.82 (2H, m), 1.83-2.04 (4H, m), 2.76-2.93 (2H, m), 3.00-3.13 (2H, m), 3.40-3.60 (2H, m), 3.65-3.95 (2H, m), 5.81 (1H, s), 7.11 (1H, d, J=8.2 Hz), 7.25-7.64 (8H, m), 11.20 (1H, s), 12.68 (1H, s).

### Example 63

### 4-[(E)-2-phenylvinyl]-1-(4,5,6,11-tetrahydro-1H-pyrazolo[4′,3′:6,7]cyclohepta[1,2-b]indol-8-ylcarbonyl)piperidin-4-ol

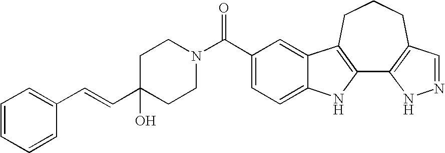

The title compound was synthesized by a method similar to that in Example 40 and using 4-[(E)-2-phenylvinyl]piperidin-4-ol instead of 4-phenylpiperidin-4-ol.

¹H-NMR (DMSO-d₆) δ: 1.44-1.83 (4H, m), 1.90-2.04 (2H, m), 2.78-2.93 (2H, m), 2.99-3.13 (2H, m), 3.21-3.46 (2H, m), 3.52-4.56 (2H, m), 4.89 (1H, s), 6.44 (1H, d, J=16.2 Hz), 6.60 (1H, d, J=16.2 Hz), 7.02-7.65 (9H, m), 11.19 (1H, s), 12.68 (1H, s).

### Example 64

### 4-[(4-fluorophenyl)ethynyl]-1-(4,5,6,11-tetrahydro-1H-pyrazolo[4′,3′:6,7]cyclohepta[1,2-b]indol-8-ylcarbonyl)piperidin-4-ol

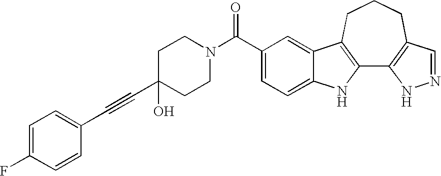

The title compound was synthesized by a method similar to that in Example 40 and using 4-[(4-fluorophenyl)ethynyl]piperidin-4-ol instead of 4-phenylpiperidin-4-ol.

¹H-NMR (DMSO-d₆) δ: 1.65-2.05 (6H, m), 2.80-2.90 (2H, m), 3.00-3.10 (2H, m), 3.40-3.55 (2H, m), 3.65-3.95 (2H, m), 5.81 (1H, s), 7.11 (1H, d, J=8.1 Hz), 7.15-7.30 (2H, m), 7.35 (1H, d, J=8.1 Hz), 7.45-7.60 (4H, m), 11.21 (1H, s), 12.70 (1H, s).

### Example 65

### 4-[(E)-2-(4-fluorophenyl)vinyl]-1-(4,5,6,11-tetrahydro-1H-pyrazolo[4′,3′:6,7]cyclohepta[1,2-b]indol-8-ylcarbonyl)piperidin-4-ol

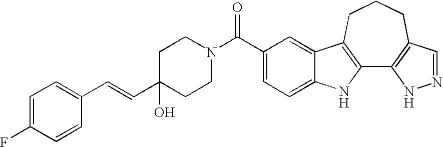

The title compound was synthesized by a method similar to that in Example 40 and using 4-[(E)-2-(4-fluorophenyl)vinyl]piperidin-4-ol instead of 4-phenylpiperidin-4-ol.

¹H-NMR (DMSO-d₆) δ: 1.45-1.85 (4H, m), 1.90-2.05 (2H, m), 2.80-2.90 (2H, m), 3.00-3.10 (2H, m), 3.20-3.50 (2H, m), 3.50-4.50 (2H, m), 4.89 (1H, s), 6.40 (1H, d, J=16.2 Hz), 6.61 (1H, d, J=16.2 Hz), 7.05-7.20 (3H, m), 7.35 (1H, d, J=8.4 Hz), 7.40-7.60 (4H, m), 11.20 (1H, s), 12.70 (1H, s).

### Example 66

### 4-[2-(4-fluorophenyl)ethyl]-1-(4,5,6,11-tetrahydro-1H-pyrazolo[4′,3′:6,7]cyclohepta[1,2-b]indol-8-ylcarbonyl)piperidin-4-ol

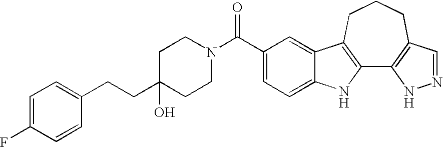

The title compound was synthesized by a method similar to that in Example 40 and using 4-[2-(4-fluorophenyl)ethyl]piperidin-4-ol instead of 4-phenylpiperidin-4-ol.

¹H-NMR (DMSO-d₆) δ: 1.45-1.60 (4H, m), 1.60-1.75 (2H, m), 1.90-2.05 (2H, m), 2.60-2.70 (2H, m), 2.80-2.90 (2H, m), 3.00-3.10 (2H, m), 3.20-3.40 (2H, m), 3.40-4.50 (2H, m), 4.43 (1H, s), 7.00-7.15 (3H, m), 7.15-7.30 (2H, m), 7.34 (1H, d, J=8.1 Hz), 7.47 (1H, s), 7.55 (1H, s), 11.19 (1H, s), 12.70 (1H, s).

### Example 67

### 4-[(4-fluorophenoxy)methyl]-1-(4,5,6,11-tetrahydro-1H-pyrazolo[4′,3′:6,7]cyclohepta[1,2-b]indol-8-ylcarbonyl)piperidin-4-ol

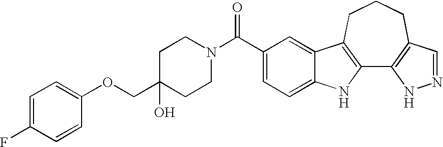

The title compound was synthesized by a method similar to that in Example 40 and using 4-[(4-fluorophenoxy)methyl]piperidin-4-ol instead of 4-phenylpiperidin-4-ol.

¹H-NMR (DMSO-d₆) δ: 1.50-1.80 (4H, m), 1.90-2.05 (2H, m), 2.80-2.90 (2H, m), 3.00-3.10 (2H, m), 3.10-3.40 (2H, m), 3.40-4.50 (2H, m), 3.79 (2H, s), 4.83 (1H, s), 6.90-7.05 (2H, m), 7.05-7.20 (3H, m), 7.35 (1H, d, J=8.7 Hz), 7.48 (1H, s), 7.55 (1H, s), 11.19 (1H, s), 12.70 (1H, s).

### Example 68

### 4-(1-benzothien-2-yl)-1-(4,5,6,11-tetrahydro-1H-pyrazolo[4′,3′:6,7]cyclohepta[1,2-b]indol-8-ylcarbonyl)piperidin-4-ol

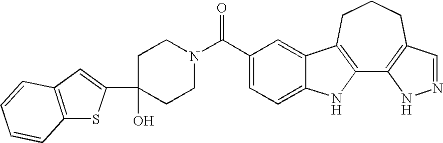

The title compound was synthesized by a method similar to that in Example 40 and using 4-(1-benzothien-2-yl)piperidin-4-ol instead of 4-phenylpiperidin-4-ol.

¹H-NMR (DMSO-d₆) δ: 1.80-2.15 (6H, m), 2.80-3.50 (8H, m), 5.86 (1H, s), 7.10-7.95 (9H, m), 11.19 (1H, s), 12.68 (1H, s).

### Example 69

### 4-[(3-fluorophenyl)ethynyl]-1-(4,5,6,11-tetrahydro-1H-pyrazolo[4′,3′:6,7]cyclohepta[1,2-b]indol-8-ylcarbonyl)piperidin-4-ol

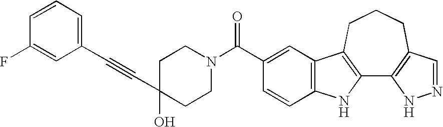

The title compound was synthesized by a method similar to that in Example 40 and using 4-[(3-fluorophenyl)ethynyl]-piperidin-4-ol instead of 4-phenylpiperidin-4-ol.

¹H-NMR (DMSO-d₆) δ: 1.63-1.83 (2H, m), 1.83-2.07 (4H, m), 2.79-2.93 (2H, m), 3.00-3.14 (2H, m), 3.40-3.55 (2H, m), 3.67-3.95 (2H, m), 5.85 (1H, s), 7.12 (1H, d, J=8.3 Hz), 7.18-7.63 (7H, m), 11.21 (1H, s), 12.70 (1H, s).

### Example 70

### 4-(4-fluorophenyl)-1-(4,5,6,11-tetrahydro-1H-pyrazolo[4′,3′:6,7]cyclohepta[1,2-b]indol-8-ylcarbonyl)piperidin-4-ol methanesulfonate

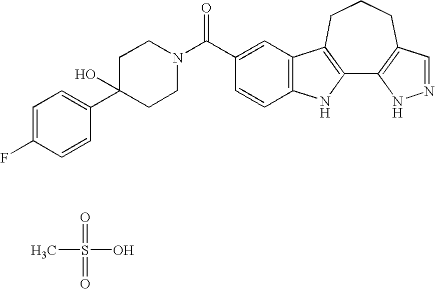

To a solution of 4-(4-fluorophenyl)-1-(4,5,6,11-tetrahydro-1H-pyrazolo[4′,3′:6,7]cyclohepta[1,2-b]indol-8-ylcarbonyl)piperidin-4-ol (222 mg) in methanol (10 ml) was added a solution of methanesulfonic acid (0.032 ml) in methanol (0.5 ml), and the mixture was concentrated. The residue was dissolved in methanol (2 ml), and diethyl ether (4 ml) was added. The precipitate was collected by filtration to give the title compound (210 mg).

¹H-NMR (DMSO-d₆) δ: 1.50-1.75 (2H, m), 1.85-2.10 (4H, m), 2.41 (3H, s), 2.80-4.60 (2H, m), 7.05-7.65 (8H, m), 11.18 (1H, s).

### Example 71

### 1-(4,5,6,11-tetrahydro-1H-pyrazolo[4′,3′:6,7]cyclohepta[1,2-b]indol-8-ylcarbonyl)-4-[3-(trifluoromethyl)phenyl]piperidin-4-ol methanesulfonate

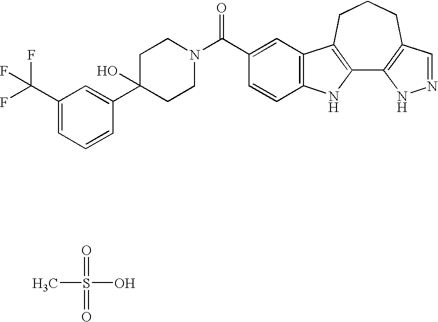

To a solution of 1-(4,5,6,11-tetrahydro-1H-pyrazolo[4′,3′:6,7]cyclohepta[1,2-b]indol-8-ylcarbonyl)-4-[3-(trifluoromethyl)phenyl]piperidin-4-ol (4.95 g) in methanol (100 ml) was added a solution of methanesulfonic acid (961 mg) in methanol (10 ml), and the mixture was concentrated. The residue was dissolved in methanol (20 ml), and the precipitate was collected by filtration (5.12 g). The obtained precipitate (4.00 g) was suspended in ethyl acetate (60 ml), and the suspension was heated under reflux for 24 hrs. The precipitate was collected by filtration to give the title compound (3.65 g).

¹H-NMR (DMSO-d₆) δ: 1.50-1.80 (2H, m), 1.90-2.25 (4H, m), 2.37 (3H, s), 2.80-4.40 (2H, m), 7.10-8.00 (8H, m), 11.17 (1H, s).

### Example 72

### 1-(4,5,6,11-tetrahydro-1H-pyrazolo[4′,3′:6,7]cyclohepta[1,2-b]indol-8-ylcarbonyl)-4-[3-(trifluoromethyl)phenyl]piperidin-4-ol ½ sulfate

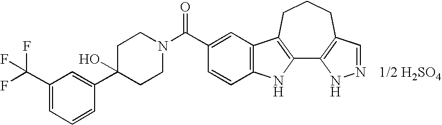

1-(4,5,6,11-tetrahydro-1H-pyrazolo[4′,3′:6,7]cyclohepta[1,2-b]indol-8-ylcarbonyl)-4-[3-(trifluoromethyl)phenyl]piperidin-4-ol (6.0 g) was dissolved in tetrahydrofuran (40 ml), sulfuric acid (0.74 ml) was added, and the mixture was concentrated. To the residue was added a mixed solution of hexane and ethyl acetate, and the precipitate was collected by filtration. The obtained precipitate was crystallized from ethanol to give the title compound (2.9 g).

¹H-NMR (DMSO-d₆) δ: 1.55-1.76 (2H, m), 1.92-2.11 (4H, m), 2.78-2.89 (2H, m), 3.02-3.10 (2H, m), 3.33 (2H, m), 3.61-4.58 (2H, m), 7.19 (1H, dd, J=8.4, 1.2 Hz), 7.37 (1H, d, J=8.4 Hz), 7.54-7.61 (4H, m), 7.84-7.91 (2H, m), 11.18 (1H, s).

### Formulation Example 1

An agent for inhibiting kinase, which comprises the compound of the present invention as an active ingredient can be produced by, for example, the following formulations.

| 1. Capsule | |
|---|---|
| (1) Compound obtained in Example 1 | 40 mg |
| (2) Lactose | 70 mg |
| (3) Microcrystalline cellulose | 9 mg |
| (4) Magnesium stearate | 1 mg |
| 1 capsule | 120 mg |

(1), (2), (3) and ½ of (4) are admixed and the mixture is granulated. The rest of (4) is added and the whole is sealed in a gelatin capsule.

| 2. Tablet | |
|---|---|
| (1) Compound obtained in Example 1 | 40 mg |
| (2) Lactose | 58 mg |
| (3) Cornstarch | 18 mg |
| (4) Microcrystalline cellulose | 3.5 mg |
| (5) Magnesium stearate | 0.5 mg |
| 1 tablet | 120 mg |

(1), (2), (3), ⅔ of (4) and ½ of (5) are admixed and the mixture is granulated. The rest of (4) and (5) is added and the mixture is compression molded to give a tablet.

### Formulation Example 2

The compound obtained in Example 1 (50 mg) is dissolved in distilled water for injection (50 ml) in the Japanese Pharmacopoeia, and distilled water for injection in the Japanese Pharmacopoeia is added to the amount of 100 ml. This solution is filtered under sterile conditions, and 1 ml of this solution is taken, filled in a vial for injection under sterile conditions, lyophilized and sealed.

### Experimental Example 1

### Cloning of Human VEGF Receptor 2 (VEGFR2) Gene and Preparation of Recombinant Baculovirus

Human VEGF receptor 2 (hereinafter to be abbreviated as VEGFR2) gene was cloned by PCR using cDNA Libraries Human Placenta (Clontech) as a template. The primer used for PCR was prepared from nucleotide sequence (Genbank Accession AF035121) information of VEGFR2 gene by adding a nucleotide sequence encoding flag peptide and a restriction enzyme recognition sequence to a nucleotide sequence (2671-4374 of Genbank Accession AF035121) encoding the VEGFR2 intracellular domain region, so that the protein contains an N-terminal flag tag. The primer nucleotide sequence is shown below.

| | |
|---|---|
| VEGFR2-U: | |
| 5′- | (SEQ ID NO: 1) |
| AATTAAGTCGACATGGACTACAAGGATGACGATGACAAGAAGCGGGCCAATGGAGGGGAACT | |
| GAAGACA-3′ | |
| and | |
| VEGFR2-L: | |
| 5′-AATTAAGCATGCTTAAACAGGAGGAGAGCTCAGTGTGGTCCC-3′ | (SEQ ID NO: 2) |

The PCR reaction was conducted using a KOD-plus kit (TOYOBO). The obtained PCR product was electrophoresed on agarose gel (1%), the DNA fragment amplified by PCR was recovered from the gel, and then digested with restriction enzymes Sal I and Sph I. The DNA treated with the restriction enzymes was electrophoresed on agarose gel (1%), and the obtained DNA fragment was recovered and ligated with plasmid pFASTBAC1 (Invitrogen) digested with restriction enzymes Sal I and Sph I to give expression plasmid pFB-VEGFR2. The nucleotide sequence of the insertion fragment was confirmed and found to be identical with the nucleotide sequence (2671-4374 of Genbank Accession AF035121) of VEGFR2 intracellular domain. Furthermore, using BAC-TO-BAC Baculovirus Expression System (Invitrogen), virus stock BAC-VEGFR2 of recombinant baculovirus was prepared.

### Experimental Example 2

### Preparation of VEGF Receptor 2 (VEGFR2) Intracellular Domain Protein

SF-21 cells were sown at 1×10⁶ cells/ml to Sf-900II SFM medium (1 L, Invitrogen) containing 10% fetal bovine serum (trace), 50 mg/L gentamicin (Invitrogen) and 0.1% Pluronic F-68 (Invitrogen), and shaking culture was performed using a 2 L volume Erlenmeyer flask at 27° C., 100 rpm. After culturing for 24 hrs, recombinant baculovirus BAC-VEGFR2 (13.4 mL) was added, and the mixture was further cultured for 3 days. The culture medium was centrifuged at 2,000 rpm for 5 min to give virus-infected cells. The infected cells were washed with a phosphate buffered saline (Invitrogen), centrifuged under the same conditions, and the cells were preserved at −80° C. The cryopreserved cells were thawed in ice, suspended in buffer A (50 mM Tris buffer (30 mL, pH 7.4) containing 20% glycerol, 0.15 M NaCl) supplemented with Complete Protease Inhibitor (Boehringer), and ruptured 3 times with a Polytron homogenizer (Kinematica) at 20,000 rpm for 30 sec. The rupture medium was clarified by centrifugation at 40,000 rpm for 30 min and filtered with a 0.45 μm filter. The filtrate was passed through a column packed with Anti-FLAG M2 Affinity Gel (4 mL, Sigma) at a flow rate of about 0.5 mL/min. The column was washed with buffer A, and eluted with buffer A containing 100 μg/mL of FLAG peptide. The eluate was concentrated with Vivaspin 20 (Vivascience) having a molecular weight cut off of 30K. The buffer of this concentrate was exchanged using NAP™ 25 column (Amersham Bioscience) equilibrated with buffer A. The fractions containing VEGFR2 intracellular domain protein were collected, glycerol was added to the final concentration of 50% and cryopreserved at −80° C.

### Test Example 1

### Determination of VEGF Receptor 2 Kinase (VEGFR2) Inhibitory Activity

A test compound dissolved in dimethyl sulfoxide (DMSO) was diluted with a buffer (50 mM Tris-HCl (pH 7.5), 5 mM MgCl₂, 5 mM MnCl₂, 2 mM dithiothreitol, 0.01% Tween-20). To this compound solution (5 μl) was added a buffer (1011) containing 50 ng/ml of VEGFR2 intracellular domain protein and 250 ng/ml of biotin labeled polypeptide biotinyl-poly-Glu:Tyr (4:1) (CIS bio International). To the obtained mixture was added a buffer (10 μl) containing ATP (25 μM), the mixture was allowed to react at 25° C. for 5 min and the reaction was quenched with 25 μl of a stop solution (100 mM EDTA disodium salt, 62.5 mM HEPES buffer (pH 7.4), 250 mM NaCl, 0.1% bovine serum albumin, 10 μg/ml AlphaScreen assay streptavidin donor beads (Streptavidin Donor beads: PerkinElmer), 10 μg/ml AlphaScreen assay anti-phosphotyrosine recognition antibody PY-100 binding acceptor beads (Anti-phosphotyrosine (P-Tyr-100) Acceptor beads: PerkinElmer)). The reaction solution was allowed to stand at 25° C. for 16 hrs, and the cells were counted using a plate reader Fusion™ (manufactured by PerkinElmer). The kinase inhibitory rate (%) of the test compound was calculated by the following formula:
Inhibitory rate (%)=(1−(count of test compound−blank)÷(control−blank))×100

The count of the solution reacted without addition of the compound was used as a “control”, and the count of the solution without the compound and ATP was used as a “blank”.

The inhibitory rate of the compounds of Examples 11, 15 and 17 at 1 μM was not less than 90%, and the inhibitory rate of the compound Example 41, 42, 43, 44, 45, 46, 47, 48 and 49 at 1 μM was not less than 80%.

### Test Example 2

### Inhibitory Test of Vascular Endothelial Cell Growth

The human umbilical vein-derived vascular endothelial cells (HUVEC purchased from KURABO) are cultured in a vascular endothelial cell medium (Invitrogen) containing 5% fetal bovine serum and 2.5 ng/mL basic fibroblast growth factor in an incubator (37° C., 5% CO₂). To be specific, HUVECs are suspended in the aforementioned vascular endothelial cell medium containing 5% fetal bovine serum and sown to each well (50 μL, 3000 cells) of a 96-well flat-bottomed plate. After culturing overnight, test substances at various concentrations and vascular endothelial growth factor (VEGF, final concentration 120 ng/mL) are dissolved in the vascular endothelial cell medium containing 5% fetal bovine serum and added to each well (50 μL). After culturing for 5 days, an XTT reagent (Wako Pure Chemical Industries, Ltd.) is added to each well (10 μL) and allowed to react in an incubator (37° C., 5% CO₂) for 2-3 hrs. The absorbance at 450 nm is measured on a microtiter plate reader to determine the cell growth inhibitory activity.

### Test Example 3

### Antitumor Test

The cancer cells are cultured in a culture medium containing 10% fetal bovine serum in an incubator (37° C., 5% CO₂). The cells are isolated by treating with trypsin, washed with HBSS (HANK's Balanced Saline Solution), and adjusted to the cell density of 1×10⁸ cells/mL with HBSS. The cell suspension (0.1 mL, 1×10⁷ cells) is implanted by subcutaneous injection to the abdomen of 6-week-old female nude mice (BALB/c nu/nu, CLEA Japan, Inc.). When the tumor volume reaches 100-200 mm³, the mice are grouped, and various doses of test substance are orally administered for 14 consecutive days from the next day. The tumor volume is calculated by tumor volume=major axis×minor axis×minor axis×0.5, based on the time-course measurement of the major axis and minor axis of the tumor.

### INDUSTRIAL APPLICABILITY

Since compound (I′), a salt thereof and a prodrug thereof of the present invention show a superior inhibitory action on kinase such as vascular endothelial growth factor receptor and the like, a clinically useful agent for the prophylaxis or treatment of diseases (e.g., cancer and the like) associated with in vivo actions of vascular endothelial growth factor can be provided. In addition, since compound (I′), a salt thereof and a prodrug thereof of the present invention are also superior in the efficacy expression, pharmacokinetics, solubility, interaction with other pharmaceutical products, safety and stability, they are useful as pharmaceutical agents.

This application is based on a patent application Nos. 165012/2004 and 355947/2004 filed in Japan, the contents of which are hereby incorporated by reference.

1. A compound represented by the formula 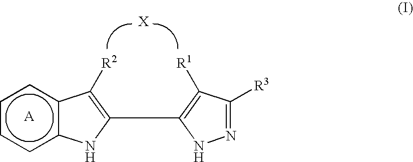
wherein A is a benzene ring optionally having substituents, R¹, R² and R³ are each a hydrogen atom, a hydrocarbon group optionally having substituents or a heterocyclic group optionally having substituents, provided that R² is not a 4-hydroxyphenyl group optionally having substituents selected from the group consisting of a halogen atom, a C₁₋₆ alkyl group and a C₁₋₆ alkoxy group; a 4-methoxyphenyl group optionally having substituents selected from the group consisting of a halogen atom, a C₁₋₆ alkyl group and a C₁₋₆ alkoxy group; and a 6-hydroxypyridin-3-yl group optionally having substituents selected from the group consisting of a halogen atom, a C₁₋₆ alkyl group and a C₁₋₆ alkoxy group, R¹ and R² optionally form a ring via X and when R¹ and R² form a ring via X, then R¹ and R² are each a bond or a divalent C₁₋₆ acyclic hydrocarbon group optionally having substituents, and X is a bond, an oxygen atom, an optionally oxidized sulfur atom or an imino group optionally having a substituent, provided that R¹, R² and X are not bonds at the same time, or a salt thereof (excluding 7-methyl-3-phenyl-1,4,5,10-tetrahydropyrazolo[3,4-a]carbazole hydrochloride, 7-methoxy-1,10-dihydropyrazolo[3,4-a]carbazole, 9-methoxy-1,10-dihydropyrazolo[3,4-a]carbazole, 8-methoxy-4,5,6,11-tetrahydro-1H-pyrazolo[4′,3′:6,7]cyclohepta[1,2-b]indole, 1,4,5,10-tetrahydropyrazolo[3,4-a]carbazole, 7-chloro-1,4,5,10-tetrahydropyrazolo[3,4-a]carbazole, 7-bromo-1,4,5,10-tetrahydropyrazolo[3,4-a]carbazole, 7-methyl-1,4,5,10-tetrahydropyrazolo[3,4-a]carbazole, 7-cyclohexyl-1,4,5,10-tetrahydropyrazolo[3,4-a]carbazole, 1,10-dihydropyrazolo[3,4-a]carbazole, 9-methyl-1,10-dihydropyrazolo[3,4-a]carbazole, 8-methyl-1,10-dihydropyrazolo[3,4-a]carbazole, 7-methyl-1,10-dihydropyrazolo[3,4-a]carbazole, 7-chloro-1,10-dihydropyrazolo[3,4-a]carbazole and 7-bromo-1,10-dihydropyrazolo[3,4-a]carbazole).

2. The compound of claim 1, which is represented by the formula 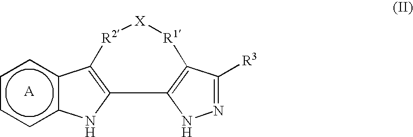
wherein R¹′ and R²′ are each a bond or a divalent C₁₋₅ acyclic hydrocarbon group optionally having substituents, and A, R³ and X are as defined in claim 1, provided that R¹′, R²′ and X are not bonds at the same time.

3. The compound of claim 1, which is represented by the formula 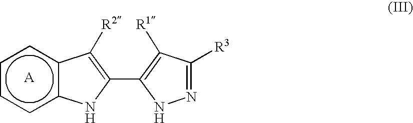
wherein R¹″ and R²″ are each a hydrogen atom, a hydrocarbon group optionally having substituents or a heterocyclic group optionally having substituents, and A and R³ are as defined in claim 1, provided that R²″ is not a 4-hydroxyphenyl group optionally having substituents selected from the group consisting of a halogen atom, a C₁₋₆ alkyl group and a C₁₋₆ alkoxy group; a 4-methoxyphenyl group optionally having substituents selected from the group consisting of a halogen atom, a C₁₋₆ alkyl group and a C₁₋₆ alkoxy group; and a 6-hydroxypyridin-3-yl group optionally having substituents selected from the group consisting of a halogen atom, a C₁₋₆ alkyl group and a C₁₋₆ alkoxy group.

4. The compound of claim 3, which is represented by the formula 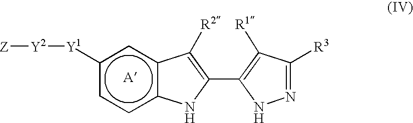
wherein A′ is a benzene ring optionally having substituents, Y¹ and Y² are each a bond, an oxygen atom, an optionally oxidized sulfur atom, an imino group optionally having a substituent or a carbonyl group, Z is a hydrogen atom, a hydrocarbon group optionally having substituents or a heterocyclic group optionally having substituents, and R¹″, R²″ and R³ are as defined in claim 3.

5. The compound of claim 1, wherein the substituent of the benzene ring for A is a carbamoyl group optionally having substituents or an optionally substituted heterocycle-carbonyl group.

6. The compound of claim 2, wherein R¹′ is a —CH₂CH₂CH₂— group optionally having substituents, and R²′ and X are bonds.

7. The compound of claim 1, wherein, when X is a bond and R¹ and R² form a 7 or more-membered ring via X, then the substituent of the benzene ring for A is a heterocycle-carbonyl group having substituent(s).

8. The compound of claim 1, wherein, when X is a bond and R¹ and R² form a 7 or more-membered ring via X, then the substituent of the benzene ring for A is a 1-piperidinylcarbonyl group having substituent(s).

9. A compound represented by the formula 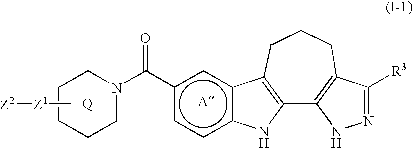
wherein Z¹ is a bond, methylene (CH₂), ethylene (CH₂CH₂), vinylene (CHCH) or ethynylene (CC), Z² is a phenyl group optionally having substituents or a heterocyclic group optionally having substituents, A″ is a benzene ring optionally further having substituents, R³ is a hydrogen atom, a hydrocarbon group optionally having substituents or a heterocyclic group optionally having substituents, and Q is a piperidine ring optionally further having substituents other than -Z¹-Z², or a salt thereof.

10. The compound of claim 9, which is represented by the formula 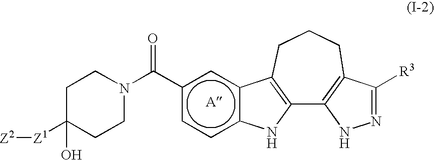
wherein each symbol is as defined in claim 9.

11. The compound of claim 4, wherein Y¹ is a carbonyl group, Y² is a bond, and Z is 1-piperidinyl optionally having substituents.

12. The compound of claim 1, wherein R² is a hydrocarbon group optionally having substituents or a heterocyclic group optionally having substituents.

13. The compound of claim 1, which is represented by the formula 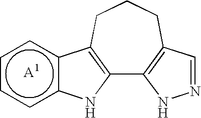
wherein A¹ is a benzene ring optionally having substituents selected from the following (i) to (vii):
(i) a C₁₋₆ alkyl group;
(ii) a carboxyl group;
(iii) a C₁₋₆ alkoxy-carbonyl group;
(iv) 5 to 7-membered heterocycle-carbonyl containing, besides carbon atom, 1 to 4 hetero atoms of 1 or 2 kinds selected from a nitrogen atom, a sulfur atom and an oxygen atom, which is optionally substituted by substituents selected from the following (a) to (g);
(a) hydroxy,
(b) C₁₋₆ alkoxy-carbonyl,
(c) a group represented by the formula -Z¹-Z²
wherein Z¹ is a bond, methylene (CH₂), ethylene (CH₂CH₂), vinylene (CHCH), ethynylene (CC) or methyleneoxy (CH₂O),
Z² is (i) a phenyl group optionally having substituents selected from the group consisting of (a) a halogen atom, (b) cyano, (c) an optionally halogenated C₁₋₆ alkyl group, (d) an optionally halogenated C₁₋₆ alkoxy group, (e) carbamoyl and (f) sulfamoyl, or
(ii) a 5 to 10-membered monocyclic or bicyclic heterocyclic group containing, besides carbon atom, 1 to 4 hetero atoms of 1 or 2 kinds selected from a nitrogen atom, a sulfur atom and an oxygen atom, which optionally has substituents selected from the group consisting of (a) a halogen atom, (b) cyano, (c) an optionally halogenated C₁₋₆ alkyl group, (d) an optionally halogenated C₁₋₆ alkoxy group, (e) carbamoyl and (f) sulfamoyl,
(d) mono- or di-C₁₋₆ alkylamino,
(e) mono- or di-C₇₋₁₆ aralkylamino,
(f) 5 to 7-membered heterocycle-C₁₋₆ alkyl(C₁₋₆ alkyl)amino wherein the heterocycle contains, besides carbon atom, 1 to 4 hetero atoms of 1 or 2 kinds selected from a nitrogen atom, a sulfur atom and an oxygen atom, and
(g) C₁₋₆ alkyl(C₇₋₁₆ aralkyl)amino wherein the C₁₋₆ alkyl is optionally substituted by hydroxy;
(v) a carbamoyl group optionally having 1 or 2 substituents selected from the following (a) to (c);
(a) C₁₋₆ alkyl,
(b) C₁₋₆ alkyl substituted by mono- or di-C₁₋₆ alkylamino, and
(c) C₁₋₆ alkyl substituted by a 5 to 7-membered heterocyclic group containing, besides carbon atom, 1 to 4 hetero atoms of 1 or 2 kinds selected from a nitrogen atom, a sulfur atom and an oxygen atom;
(vi) a halogen atom; and
(vii) 5 to 7-membered heterocycle-carbonyl-amino containing, besides carbon atom, 1 to 4 hetero atoms of 1 or 2 kinds selected from a nitrogen atom, a sulfur atom and an oxygen atom.

14. The compound of claim 1, which is represented by the formula 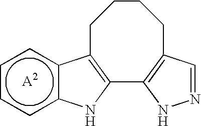
wherein A² is a benzene ring optionally having substituents selected from (i) a carboxyl group, (ii) a C₁₋₆ alkoxy-carbonyl group and (iii) 5 to 7-membered heterocycle-carbonyl containing, besides carbon atom, 1 to 4 hetero atoms of 1 or 2 kinds selected from a nitrogen atom, a sulfur atom and an oxygen atom, which is optionally substituted by a 5 to 7-membered heterocyclic group containing, besides carbon atom, 1 to 4 hetero atoms of 1 or 2 kinds selected from a nitrogen atom, a sulfur atom and an oxygen atom.

15. The compound of claim 1, which is represented by the formula 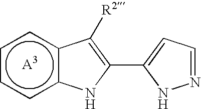
wherein R²″′ is a C₁₋₆ alkyl group, and A³ is a benzene ring optionally having 5 to 7-membered heterocycle-carbonyl containing, besides carbon atom, 1 to 4 hetero atoms of 1 or 2 kinds selected from a nitrogen atom, a sulfur atom and an oxygen atom, which is optionally substituted by a 5 to 10-membered heterocyclic group containing, besides carbon atom, 1 to 4 hetero atoms of 1 or 2 kinds selected from a nitrogen atom, a sulfur atom and an oxygen atom.

16. The compound of claim 1, which is represented by the formula 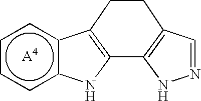
wherein A⁴ is a benzene ring optionally having substituents selected from a halogen atom and 5 to 7-membered heterocycle-carbonyl-amino containing, besides carbon atom, 1 to 4 hetero atoms of 1 or 2 kinds selected from a nitrogen atom, a sulfur atom and an oxygen atom.

17. The compound of claim 1, which is
(i) 4-(4-fluorophenyl)-1-(4,5,6,11-tetrahydro-1H-pyrazolo[4′,3′:6,7]cyclohepta[1,2-b]indol-8-ylcarbonyl)piperidin-4-ol,
(ii) 4-(3-fluorophenyl)-1-(4,5,6,11-tetrahydro-1H-pyrazolo[4′,3′:6,7]cyclohepta[1,2-b]indol-8-ylcarbonyl)piperidin-4-ol,
(iii) 4-(2-fluorophenyl)-1-(4,5,6,11-tetrahydro-1H-pyrazolo[4′,3′:6,7]cyclohepta[1,2-b]indol-8-ylcarbonyl)piperidin-4-ol,
(iv) 4-(4-chlorophenyl)-1-(4,5,6,11-tetrahydro-1H-pyrazolo[4′,3′:6,7]cyclohepta[1,2-b]indol-8-ylcarbonyl)piperidin-4-ol,
(v) 4-(3-chlorophenyl)-1-(4,5,6,11-tetrahydro-1H-pyrazolo[4′,3′:6,7]cyclohepta[1,2-b]indol-8-ylcarbonyl)piperidin-4-ol,
(vi) 4-(2-chlorophenyl)-1-(4,5,6,11-tetrahydro-1H-pyrazolo[4′,3′:6,7]cyclohepta[1,2-b]indol-8-ylcarbonyl)piperidin-4-ol,
(vii) 1-(4,5,6,11-tetrahydro-1H-pyrazolo[4′,3′:6,7]cyclohepta[1,2-b]indol-8-ylcarbonyl)-4-[4-(trifluoromethyl)phenyl]piperidin-4-ol,
(viii) 1-(4,5,6,11-tetrahydro-1H-pyrazolo[4′,3′:6,7]cyclohepta[1,2-b]indol-8-ylcarbonyl)-4-[3-(trifluoromethyl)phenyl]piperidin-4-ol,
(ix) 1-(4,5,6,11-tetrahydro-1H-pyrazolo[4′,3′:6,7]cyclohepta[1,2-b]indol-8-ylcarbonyl)-4-[2-(trifluoromethyl)phenyl]piperidin-4-ol, or a salt thereof.

18. A compound represented by the formula 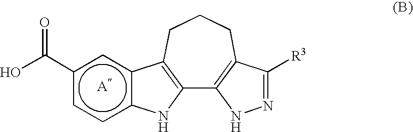
wherein A″ is a benzene ring optionally further having substituents, and R³ is a hydrogen atom, a hydrocarbon group optionally having substituents or a heterocyclic group optionally having substituents, or a salt thereof or a reactive derivative thereof.

19. The compound of claim 18, which is
(i) ethyl 4,5,6,11-tetrahydro-1H-pyrazolo[4′,3′:6,7]cyclohepta[1,2-b]indole-8-carboxylate,
(ii) 4,5,6,11-tetrahydro-1H-pyrazolo[4′,3′:6,7]cyclohepta[1,2-b]indole-8-carboxylic acid, or a salt thereof.

20. A prodrug of the compound of claim 1.

21. A pharmaceutical composition comprising an effective amount of the compound of claim 1 or a prodrug thereof, and a pharmaceutically acceptable carrier.

22. A method of producing a compound represented by the formula 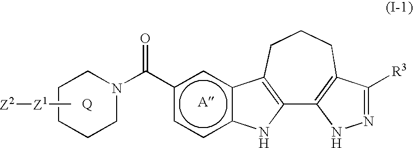
wherein Z¹ is a bond, methylene (CH₂), ethylene (CH₂CH₂), vinylene (CHCH) or ethynylene (CC), Z² is a phenyl group optionally having substituents or a heterocyclic group optionally having substituents, Q is a piperidine ring optionally further having substituents other than -Z¹-Z², A″ is a benzene ring optionally further having substituents, and R³ is a hydrogen atom, a hydrocarbon group optionally having substituents or a heterocyclic group optionally having substituents, or a salt thereof, which comprises reacting a compound represented by the formula 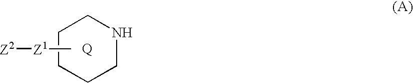
wherein Z¹, Z² and Q are as defined above, or a salt thereof, with a compound represented by the formula 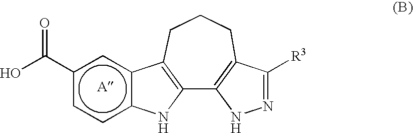
wherein A″ and R³ are as defined above, or a salt thereof or a reactive derivative thereof.

23. A method for inhibiting kinase (phosphorylation enzyme) in a mammal, which comprises administering, to said mammal, an effective amount of a compound represented by the formula 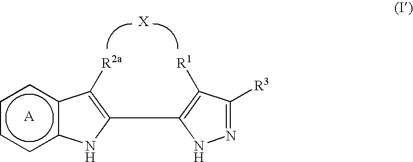
wherein A is a benzene ring optionally having substituents, R¹, R^{2a} and R³ are each a hydrogen atom, a hydrocarbon group optionally having substituents or a heterocyclic group optionally having substituents, R¹ and R^{2a} optionally form a ring via X, and when R¹ and R^{2a} form a ring via X, R¹ and R^{2a} are each a bond or a divalent C₁₋₅ acyclic hydrocarbon group optionally having substituents, and X is a bond, an oxygen atom, an optionally oxidized sulfur atom or an imino group optionally having a substituent, provided that R¹, R^{2a} and X are not bonds at the same time, or a salt thereof, or a prodrug thereof.

24. The method of claim 23, wherein the kinase is a vascular endothelial growth factor receptor (VEGFR).

25. The method of claim 23, wherein the kinase is a vascular endothelial growth factor receptor (VEGFR) 2.

26. The method of claim 23, wherein the kinase is a fibroblast growth factor receptor (FGFR) 1.

27. A method for inhibiting angiogenesis in a mammal, which comprises administering to said mammal, an effective amount of a compound represented by the formula 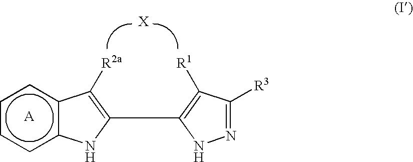
wherein A is a benzene ring optionally having substituents, R¹, R^{2a} and R³ are each a hydrogen atom, a hydrocarbon group optionally having substituents or a heterocyclic group optionally having substituents, R¹ and R^{2a} optionally form a ring via X, and when R¹ and R², form a ring via X, R¹ and R^{2a} are each a bond or a divalent C₁₋₅ acyclic hydrocarbon group optionally having substituents, and X is a bond, an oxygen atom, an optionally oxidized sulfur atom or an imino group optionally having a substituent, provided that R¹, R^{2a} and X are not bonds at the same time, or a salt thereof, or a prodrug thereof.

28. (canceled)

29. A method for inhibiting growth of cancer in a mammal, which comprises administering, to said mammal, an effective amount of a compound represented by the formula 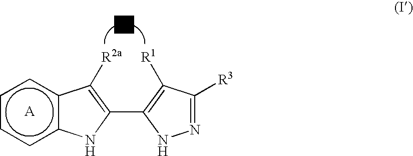
wherein A is a benzene ring optionally having substituents, R¹, R^{2a} and R³ are each a hydrogen atom, a hydrocarbon group optionally having substituents or a heterocyclic group optionally having substituents, R¹ and R^{2a} optionally form a ring via X, and when R¹ and R^{2a} form a ring via X, R¹ and R^{2a} are each a bond or a divalent C₁₋₅ acyclic hydrocarbon group optionally having substituents, and X is a bond, an oxygen atom, an optionally oxidized sulfur atom or an imino group optionally having a substituent, provided that R¹, R^{2a} and X are not bonds at the same time, or a salt thereof, or a prodrug thereof.

30. A method for suppressing metastasis of cancer in a mammal, which comprises administering, to said mammal, an effective amount of a compound represented by the formula 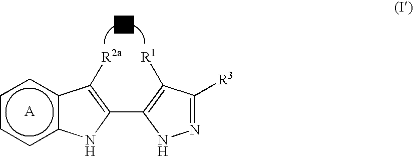
wherein A is a benzene ring optionally having substituents, R¹, R^{2a} and R³ are each a hydrogen atom, a hydrocarbon group optionally having substituents or a heterocyclic group optionally having substituents, R¹ and R^{2a} optionally form a ring via X, and when R¹ and R^{2a} form a ring via X, R¹ and R^{2a} are each a bond or a divalent C₁₋₅ acyclic hydrocarbon group optionally having substituents, and X is a bond, an oxygen atom, an optionally oxidized sulfur atom or an imino group optionally having a substituent, provided that R¹, R^{2a} and X are not bonds at the same time, or a salt thereof, or a prodrug thereof.

31. A method for the prophylaxis or treatment of cancer in a mammal, which comprises administering, to said mammal, an effective amount of a compound represented by the formula 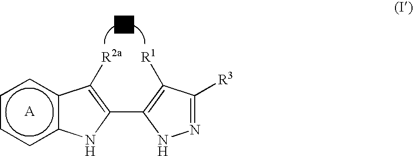
wherein A is a benzene ring optionally having substituents, R¹, R^{2a} and R³ are each a hydrogen atom, a hydrocarbon group optionally having substituents or a heterocyclic group optionally having substituents, R¹ and R^{2a} optionally form a ring via X, and when R¹ and R^{2a} form a ring via X, R¹ and R^{2a} are each a bond or a divalent C₁₋₅ acyclic hydrocarbon group optionally having substituents, and X is a bond, an oxygen atom, an optionally oxidized sulfur atom or an imino group optionally having a substituent, provided that R¹, R^{2a} and X are not bonds at the same time, or a salt thereof, or a prodrug thereof.

32. (canceled)

33. A prodrug of the compound of claim 9.

34. A pharmaceutical composition comprising an effective amount of the compound of claim 9 or a prodrug thereof, and a pharmaceutically acceptable carrier.


SEQUENCE LISTING (2 sequences)

SEQ ID NO: 1 - DNA, 69 bp, Artificial Sequence
  primer for cloning human VEGFR2 gene
aattaagtcgacatggactacaaggatgacgatgacaagaagcgggccaatggaggggaactgaagaca

SEQ ID NO: 2 - DNA, 42 bp, Artificial Sequence
  primer for cloning human VEGFR2 gene
aattaagcatgcttaaacaggaggagagctcagtgtggtccc